SEQUENCE LISTING (252 sequences)

SEQ ID NO: 1 - protein, 120 aa, synthetic construct
EVQLVESGGGLVQPGGSLRLSCAASGFTFSSYAMSWVRQAPGKEPEWVSSISGGGTSTYYADSLEGRFTISRDNSKNTLYLQMNSLRAEDTAVYYCSKDPRFGEPPFDYWGQGTTVTVSS

SEQ ID NO: 2 - protein, 114 aa, synthetic construct
QVQLVESGGGLVQPGGSLRLSCAASGFTFSNYLMHWVRQAPGKGLEWLSNINSDGSSTYYADSVKGRFTISRDNSKNTLYLQMNSLRAEDTAVYYCTVGAFDYWGQVTTGTVSS

SEQ ID NO: 3 - protein, 114 aa, synthetic construct
EVQLVESGGGVVQPGRSLRLSCAASGFTFSNYLMQWVRQAPGKGLVWLSNINSDGSSTDYADSVKGRFTISRDNAKNTLYLQMNSLRAEDTAVYYCRVGAFDYWGQGTLVTVSS

SEQ ID NO: 4 - protein, 120 aa, synthetic construct
QVQLVESGGGLVQPGGSLRLSCAASGFTFSSYAMSWVRQAPGKESEWVSSISGSGSSTYYADSLKGRFTISRDNSKNTLYLQMNSLRAEDTAVYYCSKDPRFGEPPFDYWGQGTTVTVSS

SEQ ID NO: 5 - protein, 120 aa, synthetic construct
EVQLVESGGGLVQPGGSLRLSCAASGFTFSSYAMSWVRQAPGKEPEWVSSISGGGSSAYYADSLKGRFTISRDNSKNTLYLQMNSLRAEDTAVYYCSRDPRFGEPPFDYWGQGTTVTVSS

SEQ ID NO: 6 - protein, 120 aa, synthetic construct
QVQLVESGGGLVQPGGSLRLSCAASGFIFSSYAMSWVRQAPGKEPEWVSSISGGGSSTYYADSLEGRFTISRDNSKNTLYLQMNSLRAEDTAVYYCSKDPRFGEPPFDYWGQGTTVTVSS

SEQ ID NO: 7 - protein, 120 aa, synthetic construct
EVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTTVTVSS

SEQ ID NO: 8 - protein, 120 aa, synthetic construct
EVQLVESGGGVVQPGRSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTTVTVSS

SEQ ID NO: 9 - protein, 120 aa, synthetic construct
QVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRFGEPPLDYWGQGTTVTVSS

SEQ ID NO: 10 - protein, 120 aa, synthetic construct
QVQLVESGGGLVQPGGSLRLSCAASGFTFSSSAMSWVRQAPGKGLEWVSAISGSGGSTNYVDSVKGRFTVSRDNSKNTLYLQMNRLRAEDTAVYYCAKESMVRGGPFDYWGQGTLVTVSS

SEQ ID NO: 11 - protein, 120 aa, synthetic construct
EVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRFREPPFDYWGQGTLVTVSS

SEQ ID NO: 12 - protein, 120 aa, synthetic construct
EVQLVESGGGVVQPGRSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTLVTVSS

SEQ ID NO: 13 - protein, 120 aa, synthetic construct
EVQLVESGGGLVQPGGSLRLSCAASGFTFSSYAMSWVRQAPGKEPEWVSSISGGGGSTYYADSLKGRFTISRDNSKNTLYLQMNRLRAEDTAVYYCARDPMFGERPFDYWGQGTLVTVSS

SEQ ID NO: 14 - protein, 116 aa, synthetic construct
QVQLVESGGGLVQPGGSLRLSCAASGFNFSNYWMHWVRQAPGKELVWVSRINSDGSSTSYADPVKGRFTISRDNANNMLYLQMNSLRAEDTAMYYCVRVALGFDFWGQGTLVTVSS

SEQ ID NO: 15 - protein, 118 aa, synthetic construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKEALTGVFDYWGQGTTVTVSS

SEQ ID NO: 16 - protein, 120 aa, synthetic construct
EVQLVESGGGLVQPGGSLRLSCAASGFTFSSSAMSWVRQAPGKGLEWVSAISGSGGSTNYVDSVKGRFTVSRDNSKNTLYLQMNRLRAEDTAVYYCAKESMVRGGPFDYWGQGTTVTVSS

SEQ ID NO: 17 - protein, 118 aa, synthetic construct
EVQLVESGGGLVQPGGSLRLSCAASGFTFSSYAMSWVRQAPGKGLEWVSAIYSGGSTYYADSVKGRFTISRDNSKNTLYLQMNSLRAEDTAVYYCARGDSSSSRFDYWGQGTLVTVSS

SEQ ID NO: 18 - protein, 120 aa, synthetic construct
QVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRLGEPPFDYWGQGTTVTVSS

SEQ ID NO: 19 - protein, 120 aa, synthetic construct
EEQLVESGGGLVQPGGSLRLSCAASGFTFSSYAMSWVRQAPGKEPEWVSSISGGGGSTYYADSLKGRFTISRDNSKNTLYLQMNSLRAEDTAVYYCARDPRYGEPPFDYRCQGTTVTVSS

SEQ ID NO: 20 - protein, 5 aa, synthetic construct
SYAMS

SEQ ID NO: 21 - protein, 17 aa, synthetic construct
SISGGGTSTYYADSLEG

SEQ ID NO: 22 - protein, 11 aa, synthetic construct
DPRFGEPPFDY

SEQ ID NO: 23 - protein, 5 aa, synthetic construct
NYLMH

SEQ ID NO: 24 - protein, 17 aa, synthetic construct
NINSDGSSTYYADSVKG

SEQ ID NO: 25 - protein, 5 aa, synthetic construct
GAFDY

SEQ ID NO: 26 - protein, 5 aa, synthetic construct
NYLMQ

SEQ ID NO: 27 - protein, 17 aa, synthetic construct
NINSDGSSTDYADSVKG

SEQ ID NO: 28-29 - skipped (2 entries)

SEQ ID NO: 30 - protein, 17 aa, synthetic construct
SISGSGSSTYYADSLKG

SEQ ID NO: 31-32 - skipped (2 entries)

SEQ ID NO: 33 - protein, 17 aa, synthetic construct
SISGGGSSAYYADSLKG

SEQ ID NO: 34-35 - skipped (2 entries)

SEQ ID NO: 36 - protein, 17 aa, synthetic construct
SISGGGSSTYYADSLEG

SEQ ID NO: 37-38 - skipped (2 entries)

SEQ ID NO: 39 - protein, 17 aa, synthetic construct
SISGGGSSTYYADSLKG

SEQ ID NO: 40-45 - skipped (6 entries)

SEQ ID NO: 46 - protein, 11 aa, synthetic construct
DPRFGEPPLDY

SEQ ID NO: 47 - protein, 5 aa, synthetic construct
SSAMS

SEQ ID NO: 48 - protein, 17 aa, synthetic construct
AISGSGGSTNYVDSVKG

SEQ ID NO: 49 - protein, 11 aa, synthetic construct
ESMVRGGPFDY

SEQ ID NO: 50-51 - skipped (2 entries)

SEQ ID NO: 52 - protein, 11 aa, synthetic construct
DPRFREPPFDY

SEQ ID NO: 53-56 - skipped (4 entries)

SEQ ID NO: 57 - protein, 17 aa, synthetic construct
SISGGGGSTYYADSLKG

SEQ ID NO: 58 - protein, 11 aa, synthetic construct
DPMFGERPFDY

SEQ ID NO: 59 - protein, 5 aa, synthetic construct
NYWMH

SEQ ID NO: 60 - protein, 19 aa, synthetic construct
VSRINSDGSSTSYADPVKG

SEQ ID NO: 61 - protein, 7 aa, synthetic construct
VALGFDF

SEQ ID NO: 62 - skipped

SEQ ID NO: 63 - protein, 17 aa, synthetic construct
AISGSGGSTYYADSVKG

SEQ ID NO: 64 - protein, 9 aa, synthetic construct
EALTGVFDY

SEQ ID NO: 65-68 - skipped (4 entries)

SEQ ID NO: 69 - protein, 16 aa, synthetic construct
AIYSGGSTYYADSVKG

SEQ ID NO: 70 - protein, 10 aa, synthetic construct
GDSSSSRFDY

SEQ ID NO: 71-72 - skipped (2 entries)

SEQ ID NO: 73 - protein, 11 aa, synthetic construct
DPRLGEPPFDY

SEQ ID NO: 74-75 - skipped (2 entries)

SEQ ID NO: 76 - protein, 11 aa, synthetic construct
DPRYGEPPFDY

SEQ ID NO: 77-79 - skipped (3 entries)

SEQ ID NO: 80 - protein, 120 aa, synthetic construct
EVQLVESGGGLVQPGGSLRLSCAASGFTFSSYAMSWVRQAPGKEPEWISSISGSGSSTYYADSLKGRFTISRDNSKNTLYLQMNSLRAEDTAVYYCSKDPRFGEPPFDYWGQGTTVTVSS

SEQ ID NO: 81 - protein, 120 aa, synthetic construct
QVQLVESGGGLVQPGGSLRLSCAASGFTFSSYAMSWVRQAPGKEPEWISSISGSGSSTYYADSLKGRFTISRDNSKNTLYLQMNSLRAEDTAVYYCSKDPRFGEPPFDYWGQGTLVTVSS

SEQ ID NO: 82 - protein, 120 aa, synthetic construct
EVQLVESGGGLVQPGGSLRLSCAASGFTFSSYAMSWVRQAPGKEPEWVSSISGGGSSTYYADSLKGRFTISRDNSKNTLYLQMDSLRAEDTAVYYCSRDPRFGEPPFDYWGQGTLVTVSS

SEQ ID NO: 83 - protein, 120 aa, synthetic construct
EVQLVESGGGLVQPGGSLRLSCAASGFTFSSYAMSWVRQAPGKESEWVSSISGGGSSTYYADSLKGRFTISRDNSKNTLYLQMNSLRAEDTAVYYCSKDPRFGEPPFDYWGQGTLVTVSS

SEQ ID NO: 84 - protein, 120 aa, synthetic construct
EVQLVESGGGLVQPGGSLRLSCAASGFTFSSYAMSWVRQAPGKEPEWVSSISGGGTSTYYADSLEGRFTISRDNSKNTLYLQMNSLRAEDTAVYYCSKDPRFGEPPFDYWGQGTLVTVSS

SEQ ID NO: 85 - protein, 120 aa, synthetic construct
EVQLVESGGGLVQPGGSLRLSCAASGFTFSSYAMSWVRQAPGKESEWVSSISGSGSSTYYADSLKGRFTISRDNSKNTLYLQMNSLRAEDTAVYYCSKDPRFGEPPFDYWGQGTLVTVSS

SEQ ID NO: 86 - protein, 120 aa, synthetic construct
EVQLVESGGGLVQPGGSLRLSCAASGFTFSSYAMSWVRQAPGKEPEWISSISGSGSSTYYADSLKGRFTISRDNSKNTLYLQMNSLRAEDTAVYYCSKDPRFGEPPFDYWGQGTLVTVSS

SEQ ID NO: 87 - protein, 120 aa, synthetic construct
EVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTLVTVSS

SEQ ID NO: 88 - protein, 120 aa, synthetic construct; identical to SEQ ID NO: 8

SEQ ID NO: 89 - protein, 120 aa, synthetic construct
QVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTLVTVSS

SEQ ID NO: 90 - protein, 120 aa, synthetic construct
QVQLVESGGGLVQPGGSLRLSCAASGFIFSSYAMSWVRQAPGKEPEWVSSISGGGSSTYYADSLEGRFTISRDNSKNTLYLQMNSLRAEDTAVYYCSKDPRFFEPPFDYWGQGTLVTVSS

SEQ ID NO: 91 - protein, 123 aa, synthetic construct
QVQLVESGGGVVQPGRSLRLSCAASGFTFSSYGMHWVRQAPGKGLEWVAVIWYDGNHKYYADSVKGRFTISRDNSKNTLYLQMNSLRAEDTAVYYCARDKGGITGTTRNFQHWGQGTTVTVSS

SEQ ID NO: 92 - protein, 118 aa, synthetic construct
QVQLVESGGGLVQPGGSLRLSCAASGFTFSSFAMSWVRQAPGKGLEWVSAISGSGGRTHYADSVKGRFTISRDNSKNTLYLQMNSLRAEDTAVYYCAKEALTGVFDYWGQGTLVTVSS

SEQ ID NO: 93 - protein, 120 aa, synthetic construct
QVQLVESGGGLVQPGGSLRLSCAASGFTFSSSAMSWVRQAPGKGLEWVSAISGSGGSTNYVDSVKGRFTVSRDNSKNTLYLQMNRLRAEDTAVYYCAKESMVRGGPFDYWGQGTTVTVSS

SEQ ID NO: 94 - protein, 120 aa, synthetic construct
QVQLVESGGGLVRPGGSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTLVTVSS

SEQ ID NO: 95 - protein, 120 aa, synthetic construct
QVQLVESGGGVVQPGRSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTLVTVSS

SEQ ID NO: 96 - protein, 120 aa, synthetic construct
QVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTTVTVSS

SEQ ID NO: 97 - protein, 11 aa, synthetic construct
DPRFFEPPFDY

SEQ ID NO: 98 - protein, 5 aa, synthetic construct
SYGMH

SEQ ID NO: 99 - protein, 17 aa, synthetic construct
VIWYDGNHKYYADSVKG

SEQ ID NO: 100 - protein, 14 aa, synthetic construct
DKGGITGTTRNFQH

SEQ ID NO: 101 - protein, 5 aa, synthetic construct
SFAMS

SEQ ID NO: 102 - protein, 17 aa, synthetic construct
AISGSGGRTHYADSVKG

SEQ ID NO: 103-152 - skipped (50 entries)

SEQ ID NO: 153 - protein, 195 aa, synthetic construct
MALPVTALLLPLALLLHAARPDDYKDDDDKQVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTLVTVSSTTTPAPRPPTPAPTIASQPLSLRPEACRPAAGGAVHTRGLDFACD

SEQ ID NO: 154 - protein, 535 aa, Homo sapiens
QPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVH

SEQ ID NO: 155 - protein, 533 aa, Mus musculus
QPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQMMNPGLPESVLDINECLRGARRDLKVFGSFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNGGKLTTTIGKLCAHSQQRQYRSAYYPEDLFIDKKILKVAHVEHEETLSSRRRELIQKLKSFINFYSALPGYICSHSPVAENDTLCWNGQELVERYSQKAARNGMKNQFNLHELKMKGPEPVVSQIIDKLKHINQLLRTMSVPKGKVLDKSLDEEGLESGDCGDDEDECIGSSGDGMVKVKNQLRFLAELAYDLDVDDAPGNKQHGNQKDNEITTSHSVGNM

SEQ ID NO: 156-157 - skipped (2 entries)

SEQ ID NO: 158 - protein, 417 aa, synthetic construct
MALPVTALLLPLALLLHAARPDDYKDDDDKQVQLVESGGGLVQPGGSLRLSCAASGFIFSSYAMSWVRQAPGKEPEWVSSISGGGSSTYYADSLEGRFTISRDNSKNTLYLQMNSLRAEDTAVYYCSKDPRFGEPPFDYWGQGTTVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 413 aa, synthetic construct
MALPVTALLLPLALLLHAARPDDYKDDDDKQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 417 aa, synthetic construct
MALPVTALLLPLALLLHAARPDDYKDDDDKQVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - DNA, 585 bp, synthetic construct
  source 1..585: mol_type = other DNA
atggccctgcctgtgaccgccctgctgctgcctctggccctgctcctgcacgccgccagacctgacgactacaaggacgacgacgacaagcaggtgcagctggtggagagcggcggcggcctggtgcagcctggcggcagcctgagactgagctgcgccgccagcggcttcaccttcagcagctacgccatgagctgggtgagacaggcccctggcaaggagcctgagtgggtgagcagcatctctggtggaggcagcagcacctactacgccgacagcctgaagggcagattcaccatcagcagagacaacagcaagaacaccctgtacctgcagatgaacagcctgagagccgaggacaccgccgtgtactactgcagcagagatcctagattcggcgagcctcctttcgactactggggccagggcaccctggtgaccgtgagcagcaccaccacccctgcccctagacctcctaccccagcccctaccatcgccagccagcctctgagcctgcgccctgaagcctgtaggccagccgccggaggagccgtgcacaccagaggcctggacttcgcctgcgac

SEQ ID NO: 162-165 - skipped (4 entries)

SEQ ID NO: 166 - DNA, 1251 bp, synthetic construct
  source 1..1251: mol_type = other DNA
atggccctgcctgtgaccgccctgctgctgcctctggcattgctattgcacgccgccagacctgacgactacaaggacgacgacgacaagcaggtgcagctggtggagagcggcggcggcctggtgcagcctggcggcagcctgagactgagctgcgccgccagcggcttcatcttcagcagctacgccatgagctgggtgagacaggcccctggcaaggagcctgagtgggtgagcagcatctcaggtggcggcagcagcacctactacgccgacagcctggagggcagattcaccatcagcagagacaacagcaagaacaccctgtacctgcagatgaacagcctgagagccgaggacaccgccgtgtactactgcagcaaggaccctagattcggcgagcctcctttcgactactggggccagggcaccaccgtgaccgtgagcagcaccaccacccctgcccctagacctcctacgcctgcccctaccatcgccagccagcctctgagcttgaggcctgaagcctgtcggccagctgccggtggcgctgtacacaccagaggcctggacttcgcctgcgacttctgggtgctggtggtggtgggcggcgtgctggcctgctacagcctgctggtgaccgtggccttcatcatcttctgggtgagaagcaagagaagcagactgctgcacagcgactacatgaacatgacccctagaagacctggccctaccagaaagcactaccagccttacgcccctcctagagacttcgccgcctacagatccaagaggggcagaaagaagctgctgtacatcttcaagcagcctttcatgcggcctgtgcagaccacccaggaggaggacggctgcagctgcagattccctgaggaggaggagggcggctgcgagctgagagtgaagttcagcagaagcgccgacgcccctgcctacaagcagggccagaaccagctgtacaacgagctgaacctgggcagaagagaggagtacgacgtgctggacaagagaagaggccgtgaccctgagatgggcggcaagcctagaagaaagaaccctcaggagggcttgtacaacgaacttcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgcctcctaga

SEQ ID NO: 167 - DNA, 1239 bp, synthetic construct
  source 1..1239: mol_type = other DNA
atggccctgcctgtgaccgccctgctgctgcctctggctttgctcctccacgccgccagacctgacgactacaaggacgacgacgacaagcaggtgcagctggtgcagagcggcggcggcctggtgcagcctggcggcagcctgagactgagctgcgccgccagctacttcgacttcgacagctacgagatgagctgggtgagacaggcccctggcaagggcctggagtggatcggcagcatctaccacagcggcagcacctactacaaccctagcctgaagtccagagtgaccatcagcagagacaacagcaagaacaccctgtacctgcagatgaataccctgagagccgaggacaccgcaacgtattactgcgccagagtgaacatggacagattcgactactggggccagggcaccctggtgaccgtgagcagcaccaccacccctgcccctagacctcctaccccagccccgaccatcgccagccagcctctgtctctgcggcctgaagcctgcagaccggctgcaggcggagcagtgcacaccagaggcctggacttcgcctgcgacttctgggtgctggtggtggtgggcggcgtgctggcctgctacagcctgttagtaactgtggccttcatcattttctgggtcagaagcaagagaagcagactgctgcacagcgactacatgaacatgacccctagaagacctggccctaccagaaagcactaccagccttacgcccctcctagagacttcgccgcctacaggagtaagagaggcagaaagaagctgctgtacatcttcaagcagcctttcatgaggcctgtgcagaccacccaggaggaggacggctgcagctgcagattccctgaggaagaggaaggcggctgcgagctgagagtgaagttcagcagaagcgccgacgcccctgcctacaagcagggccagaaccagctgtacaacgagctgaacctgggcagaagagaggagtacgacgtgctggacaagagaagaggcagggatcctgagatgggcggcaagcctagaagaaagaaccctcaggagggcctatacaacgaactgcagaaggacaagatggccgaggcctacagcgagatcggcatgaagggcgagagacggaggggaaagggacacgacggcctgtaccagggcctgtccaccgccaccaaggacacctacgacgccctgcacatgcaggcccttcctcctaga

SEQ ID NO: 168 - DNA, 1251 bp, synthetic construct
  source 1..1251: mol_type = other DNA
atggccctgcctgtgaccgccctgctgctgcctctggccctgttactacacgccgccagacctgacgactacaaggacgacgacgacaagcaggtgcagctggtggagagcggcggcggcctggtgcagcctggcggcagcctgagactgagctgcgccgccagcggcttcaccttcagcagctacgccatgagctgggtgagacaggcccctggcaaggagcctgagtgggtgagcagcatctctggaggaggcagcagcacctactacgccgacagcctgaagggcagattcaccatcagcagagacaacagcaagaacaccctgtacctgcagatgaacagcctgagagccgaggacaccgccgtgtactactgcagcagggatcctagattcggcgagcctcctttcgactactggggccagggcaccctggtgaccgtgagcagcaccaccacccctgcccctagacctcctacaccagcacctaccatcgccagccagcctctgagcctcaggcctgaggcctgccgcccagctgctggcggagccgtgcacaccagaggcctggacttcgcctgcgacttctgggtgctggtggtggtgggcggcgtgctggcctgctacagcctgctggttactgttgccttcatcatcttctgggtgagaagcaagagaagcagactgctgcacagcgactacatgaacatgacccctagaagacctggccctaccagaaagcactaccagccttacgcccctcctagagacttcgccgcctacagaagcaagcgaggcagaaagaagctgctgtacatcttcaagcagcctttcatgcggcctgtgcagaccacccaggaggaggacggctgcagctgcagattccctgaggaggaggagggcggctgcgagctgagagtgaagttcagcagaagcgccgacgcccctgcctacaagcagggccagaaccagctgtacaacgagctgaacctgggcagaagagaggagtacgacgtgctggacaagagaagaggcagagatcctgagatgggcggcaagcctagaagaaagaaccctcaggagggcttgtataacgagttgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgcctcctaga

SEQ ID NO: 169 - protein, 608 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 170 - protein, 609 aa, Homo sapiens [sequence omitted]

SEQ ID NO: 171 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 172 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 173 - protein, 229 aa, synthetic construct
ESKYGPPCPSCPAPEFEGGPSVFLFPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 174 - protein, 201 aa, synthetic construct
PKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 175 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 176 - protein, 42 aa, synthetic construct
KRGRKKLLYIFKQPFMRPVQTTQEEDGCSCRFPEEEEGGCEL

SEQ ID NO: 177 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - protein, 22 aa, unidentified
MLLLVTSLLLCELPHPAFLLIP

SEQ ID NO: 179 - protein, 334 aa, synthetic construct
CGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 180 - protein, 13 aa, synthetic construct
LEGGGEGRGSLLT

SEQ ID NO: 181 - protein, 21 aa, synthetic construct
LCYLLDGILFIYGVILTALFL

SEQ ID NO: 182 - protein, 27 aa, synthetic construct
FWVLVVVGGVLACYSLLVTVAFIIFWV

SEQ ID NO: 183 - protein, 28 aa, synthetic construct
MFWVLVVVGGVLACYSLLVTVAFIIFWV

SEQ ID NO: 184 - protein, 22 aa, synthetic construct
MALIVLGGVAGLLLFIGLGIFF

SEQ ID NO: 185 - protein, 21 aa, synthetic construct
IYIWAPLAGTCGVLLLSLVIT

SEQ ID NO: 186 - protein, 23 aa, synthetic construct
IYIWAPLAGTCGVLLLSLVITLY

SEQ ID NO: 187 - protein, 24 aa, synthetic construct
IYIWAPLAGTCGVLLLSLVITLYC

SEQ ID NO: 188 - protein, 93 aa, synthetic construct
SALSNSIMYFSHFVPVFLPAKPTTTPAPRPPTPAPTIASQPLSLRPEACRPAAGGAVHTRGLDFACDIYIWAPLAGTCGVLLLSLVITLYCNH

SEQ ID NO: 189 - protein, 86 aa, synthetic construct
MYFSHFVPVFLPAKPTTTPAPRPPTPAPTIASQPLSLRPEACRPAAGGAVHTRGLDFACDIYIWAPLAGTCGVLLLSLVITLYCNH

SEQ ID NO: 190 - protein, 24 aa, synthetic construct
IISFFLALTSTALLFLLFFLTLRF

SEQ ID NO: 191 - protein, 187 aa, synthetic construct
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 192 - protein, 257 aa, synthetic construct
EVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 193 - protein, 15 aa, synthetic construct
GGGGSGGGGSGGGGS

SEQ ID NO: 194 - protein, 10 aa, synthetic construct
GGGGSGGGGS

SEQ ID NO: 195 - protein, 5 aa, synthetic construct
GGGGS

SEQ ID NO: 196 - protein, 10 aa, synthetic construct
GGGSSGGGSG

SEQ ID NO: 197 - protein, 12 aa, synthetic construct
ESKYGPPCPSCP

SEQ ID NO: 198 - protein, 12 aa, synthetic construct
ESKYGPPCPPCP

SEQ ID NO: 199 - protein, 22 aa, synthetic construct
ESKYGPPCPPCPGGGSSGGGSG

SEQ ID NO: 200 - protein, 39 aa, synthetic construct
IEVMYPPPYLDNEKSNGTIIHVKGKHLCPSPLFPGPSKP

SEQ ID NO: 201 - protein, 48 aa, synthetic construct
AKPTTTPAPRPPTPAPTIASQPLSLRPEACRPAAGGAVHTRGLDFACD

SEQ ID NO: 202 - protein, 45 aa, synthetic construct
TTTPAPRPPTPAPTIASQPLSLRPEACRPAAGGAVHTRGLDFACD

SEQ ID NO: 203 - protein, 129 aa, synthetic construct; identical to SEQ ID NO: 172

SEQ ID NO: 204 - protein, 107 aa, synthetic construct; identical to SEQ ID NO: 175

SEQ ID NO: 205 - protein, 41 aa, synthetic construct
RSKRSRLLHSDYMNMTPRRPGPTRKHQYPYAPPRDFAAYRS

SEQ ID NO: 206 - protein, 42 aa, synthetic construct
ALYLLRRDQRLPPDAHKPPGGGSFRTPIQEEQADAHSTLAKI

SEQ ID NO: 207 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 208 - protein, 7 aa, synthetic construct
GFTFSSY

SEQ ID NO: 209 - protein, 10 aa, synthetic construct
GFTFSSYAMS

SEQ ID NO: 210 - protein, 6 aa, synthetic construct
SSYAMS

SEQ ID NO: 211 - protein, 8 aa, synthetic construct
GFTFSSYA

SEQ ID NO: 212 - protein, 6 aa, synthetic construct
SGGGTS

SEQ ID NO: 213 - protein, 10 aa, synthetic construct
SISGGGTSTY

SEQ ID NO: 214 - protein, 13 aa, synthetic construct
WVSSISGGGTSTY

SEQ ID NO: 215 - protein, 8 aa, synthetic construct
ISGGGTST

SEQ ID NO: 216 - protein, 12 aa, synthetic construct
SKDPRFGEPPFD

SEQ ID NO: 217 - protein, 13 aa, synthetic construct
SKDPRFGEPPFDY

SEQ ID NO: 218 - protein, 7 aa, synthetic construct
GFTFSNY

SEQ ID NO: 219 - protein, 10 aa, synthetic construct
GFTFSNYLMH

SEQ ID NO: 220 - protein, 6 aa, synthetic construct
SNYLMH

SEQ ID NO: 221 - protein, 8 aa, synthetic construct
GFTFSNYL

SEQ ID NO: 222 - protein, 6 aa, synthetic construct
NSDGSS

SEQ ID NO: 223 - protein, 10 aa, synthetic construct
NINSDGSSTY

SEQ ID NO: 224 - protein, 13 aa, synthetic construct
WLSNINSDGSSTY

SEQ ID NO: 225 - protein, 8 aa, synthetic construct
INSDGSST

SEQ ID NO: 226 - protein, 6 aa, synthetic construct
TVGAFD

SEQ ID NO: 227 - protein, 7 aa, synthetic construct
TVGAFDY

SEQ ID NO: 228 - protein, 10 aa, synthetic construct
GFTFSNYLMQ

SEQ ID NO: 229 - protein, 6 aa, synthetic construct
SNYLMQ

SEQ ID NO: 230 - protein, 10 aa, synthetic construct
NINSDGSSTD

SEQ ID NO: 231 - protein, 13 aa, synthetic construct
WLSNINSDGSSTD

SEQ ID NO: 232 - protein, 6 aa, synthetic construct
RVGAFD

SEQ ID NO: 233 - protein, 7 aa, synthetic construct
RVGAFDY

SEQ ID NO: 234 - protein, 7 aa, synthetic construct
GFIFSSY

SEQ ID NO: 235 - protein, 10 aa, synthetic construct
GFIFSSYAMS

SEQ ID NO: 236 - protein, 8 aa, synthetic construct
GFIFSSYA

SEQ ID NO: 237 - protein, 6 aa, synthetic construct
SGGGSS

SEQ ID NO: 238 - protein, 10 aa, synthetic construct
SISGGGSSTY

SEQ ID NO: 239 - protein, 13 aa, synthetic construct
WVSSISGGGSSTY

SEQ ID NO: 240 - protein, 8 aa, synthetic construct
ISGGGSST

SEQ ID NO: 241 - protein, 12 aa, synthetic construct
SRDPRFGEPPFD

SEQ ID NO: 242 - protein, 13 aa, synthetic construct
SRDPRFGEPPFDY

SEQ ID NO: 243 - protein, 22 aa, unidentified
MALPVTALLLPLALLLHAARPD

SEQ ID NO: 244 - protein, 8 aa, synthetic construct
DYKDDDDK

SEQ ID NO: 245 - DNA, 92 bp, synthetic construct
  source 1..92: mol_type = other DNA
atggccctgcctgtgaccgccctgctgctgcctctggccctgctcctgcacgccgccagacctgacgactacaaggacgacgacgacaagca

SEQ ID NO: 246 - protein, 20 aa, synthetic construct
DPGYCSSTSCYNYYYYGMDV

SEQ ID NO: 247 - protein, 387 aa, synthetic construct
QVQLVESGGGLVQPGGSLRLSCAASGFIFSSYAMSWVRQAPGKEPEWVSSISGGGSSTYYADSLEGRFTISRDNSKNTLYLQMNSLRAEDTAVYYCSKDPRFGEPPFDYWGQGTTVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 248 - protein, 383 aa, synthetic construct
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 249 - protein, 387 aa, synthetic construct
QVQLVESGGGLVQPGGSLRLSCAASGFTFSSYAMSWVRQAPGKEPEWVSSISGGGSSTYYADSLKGRFTISRDNSKNTLYLQMNSLRAEDTAVYYCSRDPRFGEPPFDYWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 250 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 251 - RNA, 250 nt, synthetic construct
  source 1..250: mol_type = other RNA
  misc_difference 1..250: note = This sequence may encompass 100-250 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 252 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0191905 A1
(43) Pub. Date: Jul. 9, 2026

(54) GPC3-SPECIFIC ANTIBODIES, BINDING DOMAINS, AND RELATED PROTEINS AND USES THEREOF

(71) Applicant: ModernaTX, Inc., Cambridge, MA (US)

(72) Inventors: Graham Farrington, Acton, MA (US); Eric Yi-Chun Huang, Boston, MA (US); Zhen Zhang, Brighton, MA (US)

(21) Appl. No.: 19/133,791

(22) PCT Filed: Nov. 30, 2023
(86) PCT No.: PCT/US2023/081732

§ 371 Date: May 29, 2025

Related U.S. Application Data
(60) Provisional application No. 63/429,401, filed on Dec. 1, 2022.

(51) Int. Cl. A61K 35/17; A61K 31/7105; A61K 40/11; A61K 40/15; A61K 40/31; A61K 40/42; A61P 35/00; B82Y 5/00; C07K 16/30; C12N 15/88

(52) U.S. Cl. CPC A61K 35/17; A61K 31/7105; A61K 40/11; A61K 40/15; A61K 40/31; A61K 40/4202; A61P 35/00; C07K 16/303; C12N 15/88; A61K 2239/13; A61K 2239/21; A61K 2239/22; A61K 2239/29; A61K 2239/53; B82Y 5/00; C07K 2317/14; C07K 2317/31; C07K 2317/565; C07K 2317/569; C12N 2830/50

(57) ABSTRACT

Described herein are anti-Glypican 3 (GPC3) antibodies and binding proteins including such binding domains, as well as nucleic acids encoding the same. The disclosure also features methods of using such antibodies, antigen-binding fragments, binding proteins, and nucleic acid to treat various diseases, such as cancer (e.g., HCC).

### CROSS-REFERENCE TO RELATED APPLICATION

This application claims the benefit of priority of U.S. Provisional Appl. No. 63/429,401 filed on Dec. 1, 2022, the contents of which are incorporated by reference in their entirety herein.

### SEQUENCE LISTING

The instant application contains a Sequence Listing which has been submitted electronically in XML file format and is hereby incorporated by reference in its entirety. Said XML copy, created on Nov. 22, 2023, is named 45817-0155W01_SL.xml and is 177,434 bytes in size.

### FIELD

The present disclosure relates generally to antibodies, binding domains, related proteins (e.g., chimeric antigen receptors, bispecific antibodies, etc.) and binding fragments thereof that specifically bind Glypican 3 (GPC3) and nucleic acids encoding the same. The present disclosure further relates to methods of producing the disclosed antibodies, binding domains, proteins (e.g., purified anti-GPC3 binding proteins or chimeric molecules comprising such binding proteins) and nucleic acid molecules encoding such binding proteins, as well as medical applications and treatments utilizing the disclosed antibodies, binding domains, proteins, and nucleic acid molecules.

### BACKGROUND

The following description of the background of the present technology is provided simply as an aid in understanding the present technology and is not admitted to describe or constitute prior art to the present technology.

Glypican 3 (GPC3) is a cell-surface glycoprotein comprising heparan sulfate glycosaminoglycan chains and an inner protein core. It has important functions in cellular signaling, modulating regulation of cellular functions such as cell growth, embryogenesis, and differentiation. GPC3 has been associated with a variety of diseases, disorders, and/or conditions, including, for example, cancer. GPC3 is thus an important therapeutic target. Accordingly, there remains a need for antibodies, binding domains, and related proteins (e.g., chimeric antigen receptors or “CARs”, bispecific antibodies, etc.) that bind GPC3 and nucleic acids encoding the same.

### SUMMARY OF THE INVENTION

The present disclosure provides, among other things, antibodies, binding domains, and related proteins that bind Glypican 3 (GPC3) and nucleic acids encoding the same. The binding molecules of this disclosure encompass a single domain antibody that binds human and mouse GPC3 and comprise the three CDRs of any variable domain of a heavy chain antibody set forth in Table 8. Such antibodies, binding domains, and related proteins and the nucleic acids encoding these proteins are useful in the treatment of a subject in need thereof with a disease, disorder, and/or condition, including, for example, cancer, inflammatory diseases, and autoimmune diseases.

In one aspect, the present disclosure provides a single-domain antibody that specifically binds Glypican 3 (GPC3) and comprises the following complementarity-determining regions (CDRs): (a) a CDR-1 comprising the amino acid sequence SYAMS (SEQ ID NO: 20), a CDR-2 comprising the amino acid sequence SISGGGTSTYYADSLEG (SEQ ID NO: 21), and a CDR-3 comprising the amino acid sequence DPRFGEPPFDY (SEQ ID NO: 22); (b) a CDR-1 comprising the amino acid sequence NYLMH (SEQ ID NO: 23), a CDR-2 comprising the amino acid sequence NINSDGSSTYYADSVKG (SEQ ID NO: 24), and a CDR-3 comprising the amino acid sequence GAFDY (SEQ ID NO: 25); (c) a CDR-1 comprising the amino acid sequence NYLMQ (SEQ ID NO: 26), a CDR-2 comprising the amino acid sequence NINSDGSSTDYADSVKG (SEQ ID NO: 27), and a CDR-3 comprising the amino acid sequence GAFDY (SEQ ID NO: 25); (d) a CDR-1 comprising the amino acid sequence SYAMS (SEQ ID NO: 20), a CDR-2 comprising the amino acid sequence SISGSGSSTYYADSLKG (SEQ ID NO: 30), and a CDR-3 comprising the amino acid sequence DPRFGEPPFDY (SEQ ID NO: 22); (e) a CDR-1 comprising the amino acid sequence SYAMS (SEQ ID NO: 20), a CDR-2 comprising the amino acid sequence SISGGGSSAYYADSLKG (SEQ ID NO: 33), and a CDR-3 comprising the amino acid sequence DPRFGEPPFDY (SEQ ID NO: 22); (f) a CDR-1 comprising the amino acid sequence SYAMS (SEQ ID NO: 20), a CDR-2 comprising the amino acid sequence SISGGGSSTYYADSLEG (SEQ ID NO: 36), and a CDR-3 comprising the amino acid sequence DPRFGEPPFDY (SEQ ID NO: 22): (g) a CDR-1 comprising the amino acid sequence SYAMS (SEQ ID NO: 20), a CDR-2 comprising the amino acid sequence SISGGGSSTYYADSLKG (SEQ ID NO: 39), and a CDR-3 comprising the amino acid sequence DPRFGEPPFDY (SEQ ID NO: 22); (h) a CDR-1 comprising the amino acid sequence SYAMS (SEQ ID NO: 20), a CDR-2 comprising the amino acid sequence SISGGGSSTYYADSLKG (SEQ ID NO: 39), and a CDR-3 comprising the amino acid sequence DPRFGEPPLDY (SEQ ID NO: 46); (i) a CDR-1 comprising the amino acid sequence SSAMS (SEQ ID NO: 47), a CDR-2 comprising the amino acid sequence AISGSGGSTNYVDSVKG (SEQ ID NO: 48), and a CDR-3 comprising the amino acid sequence ESMVRGGPFDY (SEQ ID NO: 49); (j) a CDR-1 comprising the amino acid sequence SYAMS (SEQ ID NO: 20), a CDR-2 comprising the amino acid sequence SISGGGSSTYYADSLKG (SEQ ID NO: 39), and a CDR-3 comprising the amino acid sequence DPRFREPPFDY (SEQ ID NO: 52); (k) a CDR-1 comprising the amino acid sequence SYAMS (SEQ ID NO: 20), a CDR-2 comprising the amino acid sequence SISGGGGSTYYADSLKG (SEQ ID NO: 57), and a CDR-3 comprising the amino acid sequence DPMFGERPFDY (SEQ ID NO: 58); (1) a CDR-1 comprising the amino acid sequence NYWMH (SEQ ID NO: 59), a CDR-2 comprising the amino acid sequence VSRINSDGSSTSYADPVKG (SEQ ID NO: 60), and a CDR-3 comprising the amino acid sequence VALGFDF (SEQ ID NO: 61): (m) a CDR-1 comprising the amino acid sequence SYAMS (SEQ ID NO: 20), a CDR-2 comprising the amino acid sequence AISGSGGSTYYADSVKG (SEQ ID NO: 63), and a CDR-3 comprising the amino acid sequence EALTGVFDY (SEQ ID NO: 64), (n) a CDR-1 comprising the amino acid sequence SYAMS (SEQ ID NO: 20), a CDR-2 comprising the amino acid sequence AIYSGGSTYYADSVKG (SEQ ID NO: 69), and a CDR-3 comprising the amino acid sequence GDSSSSRFDY (SEQ ID NO: 70); (o) a CDR-1 comprising the amino acid sequence SYAMS (SEQ ID NO: 20), a CDR-2 comprising the amino acid sequence SISGGGSSTYYADSLKG (SEQ ID NO: 39), and a CDR-3 comprising the amino acid sequence DPRLGEPPFDY (SEQ ID NO: 73); (p) a CDR-1 comprising the amino acid sequence SYAMS (SEQ ID NO: 20), a CDR-2 comprising the amino acid sequence SISGGGGSTYYADSLKG (SEQ ID NO: 57), and a CDR-3 comprising the amino acid sequence DPRYGEPPFDY (SEQ ID NO: 76); (q) a CDR-1 comprising the amino acid sequence SYAMS (SEQ ID NO: 20), a CDR-2 comprising the amino acid sequence SISGGGSSTYYADSLEG (SEQ ID NO: 36), and a CDR-3 comprising the amino acid sequence DPRFFEPPFDY (SEQ ID NO: 97); (r) a CDR-1 comprising the amino acid sequence SYGMH (SEQ ID NO: 98), a CDR-2 comprising the amino acid sequence VIWYDGNHKYYADSVKG (SEQ ID NO: 99), and a CDR-3 comprising the amino acid sequence DKGGITGTTRNFQH (SEQ ID NO: 100); or (s) a CDR-1 comprising the amino acid sequence SFAMS (SEQ ID NO: 101), a CDR-2 comprising the amino acid sequence AISGSGGRTHYADSVKG (SEQ ID NO: 102), and a CDR-3 comprising the amino acid sequence EALTGVFDY (SEQ ID NO: 64).

In some embodiments, the single-domain antibody comprises a heavy chain comprising an amino acid sequence that is at least 85% identical to the amino acid sequence of any one of SEQ ID NOs: 1-19 or 80-96.

In some embodiments, the single-domain antibody comprises a heavy chain comprising an amino acid sequence that is at least 95% identical to the amino acid sequence of any one of SEQ ID NOs: 1-19 or 80-96.

In some embodiments, the single-domain antibody comprises a heavy chain comprising an amino acid sequence of any one of SEQ ID NOs: 1-19 or 80-96.

In some embodiments, the single-domain antibody binds GPC3 with a K_{D} of 20 nM or less. In some embodiments, the single-domain antibody binds GPC3 with a K_{D} of 10 nM or less, optionally wherein the single-domain antibody binds GPC3 with a K_{D} of 5 nM or less, optionally wherein the single-domain antibody binds GPC3 with a K_{D} of 1 nM or less, optionally wherein the single-domain antibody binds GPC3 with a K_{D} of 0.5 nM or less, optionally wherein the single-domain antibody binds GPC3 with a K_{D} of 0.1 nM or less, optionally wherein the single-domain antibody binds GPC3 with a K_{D} of 0.05 nM or less, optionally wherein the single-domain antibody binds GPC3 with a K_{D} of 0.01 nM or less.

In some embodiments, the single-domain antibody binds GPC3 with a kₒₙ of from 10³ M⁻¹s⁻¹ to 10⁵ M⁻¹s⁻¹.

In some embodiments, the single-domain antibody binds GPC3 with a k_{off} of from 10⁻³ s⁻¹ to about 10⁻⁵ s⁻¹.

In some embodiments, the single-domain antibody is a multispecific antibody, comprising a GPC3-binding domain comprising a single-domain antibody described herein and a second binding domain that specifically binds a second antigen. In some embodiments, the second antigen is selected from CD1a, CD1b, CD1c. CD1d, CD2, CD37, CD3E, CD4, CD5, CD6, CD7, CD8, CD9, CD10, CD11a, CD11b, CD13, CD14, CD15, CD15s, CD15u, CD16, CDw17, CD19, CD20, CD21, CD22, CD23, CD24, CD25, CD26, CD27, CD28, CD30. CD31, CD32, CD33, CD35, CD36, CD37, CD38, CD39, CD40. CD45RO, CD47, CD49a, CD49b, CD49c, CD49d, CD49e, CD49f, CD50, CD52, CD53, CD56, CD62, CD64, CD68, CD70, CD72, CD73, CD75, CD83, CD85, CD96, CD119, CD124, CD138, CD139, CD152, CD153, CD155, CD180, CD205, CD209, CD244, CD245, and CD247.

In some embodiments, the multispecific antibody is a bispecific antibody.

In some embodiments, the multispecific antibody is a bispecific antibody that specifically binds to human and murine GPC3 and to an antigen on a T cell or a NK cell.

In some embodiments, the present disclosure provides a chimeric antigen receptor (CAR), comprising a GPC3-binding domain comprising a single-domain antibody described herein.

In some embodiments, a CAR of the present disclosure further comprises a transmembrane domain, at least one costimulatory domain, and an intracellular signaling domain.

In some embodiments, the transmembrane domain is derived from an alpha chain of a T cell receptor, a beta chain of a T cell receptor, a zeta chain of a T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L). Toll-like receptor 1 (TLR1). TLR2, TLR3, TLR4, TLR5, TLR6, TLR7, TLR8, or TLR9.

In some embodiments, the at least one costimulatory domain comprises a costimulatory region of CD3, CD4, CD8, T cell receptor (TCR), CD27. CD28, 4-1BB (CD137). OX40, CD30, CD40. PD-1. ICOS, lymphocyte function-associated antigen-1 (LFA-1), CD2, CD7, LIGHT, NKG2C, B7-H3, or a fragment thereof.

In some embodiments, the intracellular signaling domain comprises a fragment or domain from one or more molecules selected from a T cell receptor (TCR), CD3 zeta. CD3 gamma. CD3 delta, CD3 epsilon, CD86, common FcR gamma, FcR beta (Fc Epsilon Rib), CD79a, CD79b, Fcgamma R11a, DAP 10, DAP 12, T cell receptor (TCR), CD8, CD27, CD28, 4-1BB (CD137), OX9, 0X40, CD30, CD40, PD-l, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD 160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f. ITGAD, CD1 Id, ITGAE, CD 103, ITGAL, CD 11 a, LFA-1, ITGAM, CD lib, ITGAX, CD 1 lc, ITGB1, CD29, ITGB2, CD 18, LFA-1, ITGB7, TNFR2, TRANCE/RANKL, DNAM1 (CD226), SLAMF4 (CD244, 2B4), CD84, CD 96 (Tactile), CEACAM1, CRT AM, Ly9 (CD229), CD160 (BY55), PSGL1, CD 100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, TLR10, TLR11, TLR12, and TLR13.

In some embodiments, the CAR comprises an amino acid sequence selected from any one of SEQ ID NOs: 158-160, but lacking both the signal sequence and the Flag tag.

In some embodiments, the present disclosure provides a T cell expressing a CAR described herein.

In one aspect, the present disclosure provides a binding protein that specifically binds to Glypican 3 (GPC3) and comprises the following complementarity-determining regions (CDRs): (a) a CDR-1 comprising the amino acid sequence SYAMS (SEQ ID NO: 20), a CDR-2 comprising the amino acid sequence SISGGGTSTYYADSLEG (SEQ ID NO: 21), and a CDR-3 comprising the amino acid sequence DPRFGEPPFDY (SEQ ID NO: 22); (b) a CDR-1 comprising the amino acid sequence NYLMH (SEQ ID NO: 23), a CDR-2 comprising the amino acid sequence NINSDGSSTYYADSVKG (SEQ ID NO: 24), and a CDR-3 comprising the amino acid sequence GAFDY (SEQ ID NO: 25); (c) a CDR-1 comprising the amino acid sequence NYLMQ (SEQ ID NO: 26), a CDR-2 comprising the amino acid sequence NINSDGSSTDYADSVKG (SEQ ID NO: 27), and a CDR-3 comprising the amino acid sequence GAFDY (SEQ ID NO: 25); (d) a CDR-1 comprising the amino acid sequence SYAMS (SEQ ID NO: 20), a CDR-2 comprising the amino acid sequence SISGSGSSTYYADSLKG (SEQ ID NO: 30), and a CDR-3 comprising the amino acid sequence DPRFGEPPFDY (SEQ ID NO: 22); (e) a CDR-1 comprising the amino acid sequence SYAMS (SEQ ID NO: 20), a CDR-2 comprising the amino acid sequence SISGGGSSAYYADSLKG (SEQ ID NO: 33), and a CDR-3 comprising the amino acid sequence DPRFGEPPFDY (SEQ ID NO: 22): (f) a CDR-1 comprising the amino acid sequence SYAMS (SEQ ID NO: 20), a CDR-2 comprising the amino acid sequence SISGGGSSTYYADSLEG (SEQ ID NO: 36), and a CDR-3 comprising the amino acid sequence DPRFGEPPFDY (SEQ ID NO: 22); (g) a CDR-1 comprising the amino acid sequence SYAMS (SEQ ID NO: 20), a CDR-2 comprising the amino acid sequence SISGGGSSTYYADSLKG (SEQ ID NO: 39), and a CDR-3 comprising the amino acid sequence DPRFGEPPFDY (SEQ ID NO: 22); (h) a CDR-1 comprising the amino acid sequence SYAMS (SEQ ID NO: 20), a CDR-2 comprising the amino acid sequence SISGGGSSTYYADSLKG (SEQ ID NO: 39), and a CDR-3 comprising the amino acid sequence DPRFGEPPLDY (SEQ ID NO: 46); (i) a CDR-1 comprising the amino acid sequence SSAMS (SEQ ID NO: 47), a CDR-2 comprising the amino acid sequence AISGSGGSTNYVDSVKG (SEQ ID NO: 48), and a CDR-3 comprising the amino acid sequence ESMVRGGPFDY (SEQ ID NO: 49); (j) a CDR-1 comprising the amino acid sequence SYAMS (SEQ ID NO: 20), a CDR-2 comprising the amino acid sequence SISGGGSSTYYADSLKG (SEQ ID NO: 39), and a CDR-3 comprising the amino acid sequence DPRFREPPFDY (SEQ ID NO: 52); (k) a CDR-1 comprising the amino acid sequence SYAMS (SEQ ID NO: 20), a CDR-2 comprising the amino acid sequence SISGGGGSTYYADSLKG (SEQ ID NO: 57), and a CDR-3 comprising the amino acid sequence DPMFGERPFDY (SEQ ID NO: 58); (1) a CDR-1 comprising the amino acid sequence NYWMH (SEQ ID NO: 59), a CDR-2 comprising the amino acid sequence VSRINSDGSSTSYADPVKG (SEQ ID NO: 60), and a CDR-3 comprising the amino acid sequence VALGFDF (SEQ ID NO: 61); (m) a CDR-1 comprising the amino acid sequence SYAMS (SEQ ID NO: 20), a CDR-2 comprising the amino acid sequence AISGSGGSTYYADSVKG (SEQ ID NO: 63), and a CDR-3 comprising the amino acid sequence EALTGVFDY (SEQ ID NO: 64); (n) a CDR-1 comprising the amino acid sequence SYAMS (SEQ ID NO: 20), a CDR-2 comprising the amino acid sequence AIYSGGSTYYADSVKG (SEQ ID NO: 69), and a CDR-3 comprising the amino acid sequence GDSSSSRFDY (SEQ ID NO: 70); (o) a CDR-1 comprising the amino acid sequence SYAMS (SEQ ID NO: 20), a CDR-2 comprising the amino acid sequence SISGGGSSTYYADSLKG (SEQ ID NO: 39), and a CDR-3 comprising the amino acid sequence DPRLGEPPFDY (SEQ ID NO: 73): (p) a CDR-1 comprising the amino acid sequence SYAMS (SEQ ID NO: 20), a CDR-2 comprising the amino acid sequence SISGGGGSTYYADSLKG (SEQ ID NO: 57), and a CDR-3 comprising the amino acid sequence DPRYGEPPFDY (SEQ ID NO: 76): (q) a CDR-1 comprising the amino acid sequence SYAMS (SEQ ID NO: 20), a CDR-2 comprising the amino acid sequence SISGGGSSTYYADSLEG (SEQ ID NO: 36), and a CDR-3 comprising the amino acid sequence DPRFFEPPFDY (SEQ ID NO: 97); (r) a CDR-1 comprising the amino acid sequence SYGMH (SEQ ID NO: 98), a CDR-2 comprising the amino acid sequence VIWYDGNHKYYADSVKG (SEQ ID NO: 99), and a CDR-3 comprising the amino acid sequence DKGGITGTTRNFQH (SEQ ID NO: 100); or (s) a CDR-1 comprising the amino acid sequence SFAMS (SEQ ID NO: 101), a CDR-2 comprising the amino acid sequence AISGSGGRTHYADSVKG (SEQ ID NO: 102), and a CDR-3 comprising the amino acid sequence EALTGVFDY (SEQ ID NO: 64).

In some embodiments, the binding protein comprises a heavy chain comprising an amino acid sequence that is at least 85% identical to the amino acid sequence of any one of SEQ ID NOs: 1-19 or 80-96.

In some embodiments, the binding protein comprises a heavy chain comprising an amino acid sequence that is at least 95% identical to the amino acid sequence of any one of SEQ ID NOs: 1-19 or 80-96.

In some embodiments, the binding protein comprises a heavy chain comprising an amino acid sequence of any one of SEQ ID NOs: 1-19 or 80-96.

In some embodiments, the binding protein binds GPC3 with a K_{D} of 20 nM or less.

In some embodiments, the binding protein binds GPC3 with a K_{D} of 10 nM or less, optionally wherein the binding protein binds GPC3 with a K_{D} of 5 nM or less, optionally wherein the binding protein binds GPC3 with a K_{D} of 1 nM or less, optionally wherein the binding protein binds GPC3 with a K_{D} of 0.5 nM or less, optionally wherein the binding protein binds GPC3 with a K_{D} of 0.1 nM or less, optionally wherein the binding protein binds GPC3 with a K_{D} of 0.05 nM or less, optionally wherein the binding protein binds GPC3 with a K_{D} of 0.01 nM or less.

In some embodiments, the binding protein binds GPC3 with a kₒₙ of from 10³ M⁻¹s⁻¹ to 10⁵ M⁻¹s⁻¹.

In some embodiments, the binding protein binds GPC3 with a k_{off} of from 10⁻³ s⁻¹ to about 10⁻⁵ s⁻¹.

In some embodiments, the binding protein is a monoclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, an antibody-like protein scaffold, a single-domain antibody, a Fv fragment, a Fab fragment, a F(ab′)₂ molecule, a tandem scFv (taFv), a fusion protein, or a chimeric antigen receptor.

In some embodiments, the present disclosure provides a nucleic acid encoding a single-domain antibody described herein; a multispecific antibody described herein; a CAR described herein, or a binding protein described herein.

In some embodiments, the nucleic acid is an mRNA.

In some embodiments, the nucleic acid comprises, in the 5′-to-3′ direction: (a) a 5′ cap structure; (b) a 5′ untranslated region (UTR); (c) an open reading frame encoding a protein sequence comprising the binding domain of the single-domain antibody, multispecific antibody, CAR, or binding protein, wherein the open reading frame consists of nucleosides is selected from the group consisting of (i) uridine or a modified uridine, (ii) cytidine or a modified cytidine, (iii) adenosine or a modified adenosine, and (iv) guanosine or a modified guanosine; (d) a 3′ UTR; and (e) a 3′ tailing sequence of linked nucleosides.

In some embodiments, the open reading frame of nucleosides is selected from the group consisting of (i) a modified uridine, (ii) cytidine, (iii) adenosine, and (iv) guanosine.

In some embodiments, the modified uridine is 1-methylpseudouridine, pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine, 3-methyl-uridine. 5-methoxy-uridine, uridine 5-oxyacetic acid, uridine 5-oxyacetic acid methyl ester. 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine. 5-methoxy carbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine. 5-methyl-2-thio-uridine. 1-methyl-4-thio-pseudouridine, 4-thio-1-methyl-pseudouridine, 3-methylpseudouridine, 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine. 2-thio-dihydrouridine, 2-thio-dihy dropseudouridine, 2-methoxy-uridine. 2-methoxy-4-thio-uridine. 4-methoxy-pseudouridine. 4-methoxy-2-thio-pseudouridine, N1-methyl-pseudouridine, 3-(3-amino-3-carboxypropyl)uridine, 1-methyl-3-(3-amino-3-carboxypropyl)pseudouridine, 5-(isopentenylaminomethyl)uridine, 5-(isopentenylaminomethyl)-2-thio-uridine, α-thio-uridine. 2′-O-methyl-uridine. 5,2′-O-dimethyl-uridine. 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, or 5-[3-(1-E-propenylamino)uridine.

In some embodiments, the modified uridine is 1-methylpseudouridine.

In some embodiments, the modified cytidine is 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine. 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine. 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, or 2′-OH-ara-cytidine.

In some embodiments, the modified adenosine is 2-amino-purine, 2, 6-diaminopurine, 2-amino-6-halo-purine, 6-halo-purine, 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl)adenosine, 2-methylthio-N6-(cis-hydroxyisopentenyl)adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6,N6-dimethyl-adenosine, N6-hydroxynorvalylcarbamoyl-adenosine, 2-methylthio-N6-hydroxynorvalylcarbamoyl-adenosine, N6-acetyl-adenosine. 7-methyl-adenine, 2-methylthio-adenine, 2-methoxy-adenine, α-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine. 2-amino-N6-methyl-purine, 1-thio-adenosine, 8-azido-adenosine, 2′-F-ara-adenosine, 2′-F-adenosine, 2′-OH-ara-adenosine, or N6-(19-amino-pentaoxanonadecyl)-adenosine.

In some embodiments, the modified guanosine is inosine, 1-methyl-inosine, wyosine, methylwyosine, 4-demethyl-wyosine, isowyosine, wybutosine, peroxywybutosine, hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2,N2,7-dimethyl-guanosine, 8-oxo-guanosine. 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2,7-dimethyl-2′-O-methyl-guanosine, 2′-O-methyl-inosine, 1,2′-O-dimethyl-inosine, 2′-O-ribosylguanosine, 1-thio-guanosine, O6-methyl-guanosine, 2′-F-ara-guanosine, or 2′-F-guanosine.

In some embodiments, the 3′ tailing sequence of linked nucleosides is a poly-adenylate (polyA) tail or a polyA-G quartet.

In some embodiments, the 3′ tailing sequence of linked nucleosides is a polyA tail.

In some embodiments, the 5′ cap structure is Cap0, Cap1, ARCA, inosine, 1-methyl-guanosine, 2′fluoroguanosine, 7-deaza-guanosine, 8-oxo-guanosine, 2-amino-guanosine, LNA-guanosine, or 2-azidoguanosine.

In some embodiments, the present disclosure provides a pharmaceutical composition comprising a single-domain antibody described herein; a multispecific antibody described herein; a CAR described herein, a binding protein described herein; or a nucleic acid described herein.

In some embodiments, the pharmaceutical composition further comprises one or more pharmaceutically acceptable carriers, diluents, excipients, or any combination thereof.

In some embodiments, the pharmaceutical composition comprises a plurality of lipid nanoparticles encapsulating the nucleic acid.

In some embodiments, the plurality of lipid nanoparticles has a mean particle size of from 80 nm to 160 nm.

In some embodiments, the plurality of lipid nanoparticles has a polydispersity index (PDI) of from 0.02 to 0.2 and/or a lipid:nucleic acid ratio of from 10 to 20.

In some embodiments, the lipid nanoparticles comprise a neutral lipid, a cationic lipid, a polyethyleneglycol (PEG) lipid, and/or a sterol.

In some embodiments, the neutral lipid is 1,2-distearoyl-sn-glycero-3-phosphocholine.

In some embodiments, the cationic lipid comprises Formula I.

In some embodiments, the PEG lipid is PEG 2000 dimyristoyl glycerol.

In some embodiments, the sterol is cholesterol, adosterol, agosterol A, atheronals, avenasterol, azacosterol, blazein, cerevisterol, colestolone, cycloartenol, daucosterol, 7-dehydrocholesterol. 5-dehydroepisterol, 7-dehydrositosterol. 20α, 22R-dihydroxycholesterol, dinosterol, epibrassicasterol, episterol, ergosterol, ergosterol, fecosterol, fucosterol, fungisterol, ganoderenic acid, ganoderic acid, ganoderiol, ganodermadiol, 7α-hydroxycholesterol, 22R-hydroxy cholesterol, 27-hydroxycholesterol, inotodiol, lanosterol, lathosterol, lichesterol, lucidadiol, lumisterol, oxycholesterol, oxysterol, parkeol, saringosterol, spinasterol, sterol ester, trametenolic acid, zhankuic acid, or zymosterol.

In some embodiments, the sterol is cholesterol.

In some embodiments, the present disclosure provides a host cell comprising a single-domain antibody described herein; a multispecific antibody described herein; or a CAR described herein, a binding protein described herein; or a nucleic acid described herein.

In some embodiments, the host cell is a eukaryotic cell. In some embodiments, the eukaryotic cell is a mammalian cell. In some embodiments, the mammalian cell is a CHO cell or HEK cell.

In some embodiments, the present disclosure provides a method of treating cancer, comprising administering to a subject in need thereof comprising a single-domain antibody described herein; a multispecific antibody described herein; a CAR described herein, a CAR-T described herein; a binding protein described herein; a nucleic described herein; or a pharmaceutical composition described herein.

In some embodiments, the subject is human.

In some embodiments, the cancer is selected from adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia, acute lymphocytic leukemia (ALL), acute myeloid leukemia (AML), chronic lymphocytic leukemia (CLL), chronic myelogenous leukemia (CML), hairy cell leukemia, myelogenous leukemia, and myeloid leukemia, lymphoma, cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma, myelodysplastic syndrome, papillomatosis, paraganglioma, pheochromocytoma, pleuropulmonary blastoma, retinoblastoma, sarcoma, Ewing sarcoma, Kaposi sarcoma, osteosarcoma, rhabdomyosarcoma, uterine sarcoma, vascular sarcoma, Wilms' tumor, and cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney, larynx, liver, lung, mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

In one aspect, the disclosure provides a binding molecule comprising a polypeptide that specifically binds GPC3. The polypeptide comprises a VHH-CDR1, a VHH-CDR2, and a VHH-CDR3 of any one of the VHHs set forth in SEQ ID NOs.: 1-19 or 80-96. In some cases, the polypeptide specifically binds human and mouse GPC3. In some cases, the VHH-CDR1, the VHH-CDR2, and the VHH-CDR3 are based on the Kabat, Chothia, enhanced Chothia, Contact, Aho, or IMGT CDR definition. In some cases, the polypeptide comprises three VHH CDRs according to any one definition set forth in Tables A through E.

In some instances, the polypeptide comprises a VHH that has an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of the VHHs set forth in SEQ ID NOs.: 1-19 or 80-96. In some cases, polypeptide comprises a VHH that has an amino acid sequence of any one of the VHHs set forth in SEQ ID NOs.: 1-19 or 80-96, except having 1, 2, 3, 4, 5, 6, 7, 8, 9, or 10 amino acid substitutions. In some cases, the substitutions are conservative substitutions. In certain cases, one, two, three, or four (in any combination) of Kabat positions 37, 44, 45, and 47 of the VHH are not substituted. In certain cases, one or two of Kabat positions 37 and 47 of the VHH are not substituted. In some cases, one, two, three, four, five, six, seven, or eight (in any combination) of Kabat positions 62, 65, 67, 72, 76, 89, 95, and 117 of the VHH are not substituted.

In some instances, the polypeptide is a VHH and is linked or conjugated to a scFv, Fab, whole antibody, or second VHH, that binds an antigen other than GPC3. The linking may be via a peptide linker or a chemical linker. The peptide linker can be a glycine linker, a seine linker, or a glycine-serine linker. In some cases, the linker is (G₄S)n wherein n is 1, 2, 3, 4, 5, 6, 7, 8, 9, or 10 (SEQ ID NO: 207).

In certain instances, the polypeptide is linked to a half-life extension moiety. In some cases, the half-life extension moiety is a serum albumin (e.g., HSA), polyethylene glycol, XTEN, or a second VHH that specifically binds human serum albumin.

In some instances, the binding molecule is a multispecific binding molecule (e.g., a bispecific binding molecule.)

In another aspect, the disclosure features a bispecific antibody comprising a means for binding human and murine GPC3 and a binding moiety that binds to an antigen other than GPC3. In some cases, the binding moiety is a scFv, Fab, F(ab)₂, or a second VHH. The means for binding human and murine GPC3 can be a single-chain antibody comprising the three VHH CDRs of any one antibody of Table 9. In some cases, the means for binding human and murine GPC3 can be a single-chain antibody comprising the amino acid sequence of any one antibody of Table 8. In certain cases, the antigen other than GPC3 is an antigen on a T cell or a NK cell.

In another aspect, the disclosure features a CAR comprising a binding molecule or bispecific antibody described herein, a transmembrane domain, a costimulatory domain, and an intracellular signaling domain. In some cases, the CAR comprises a hinge region or domain. In certain cases, the hinge domain links the binding molecule or bispecific antibody to the transmembrane domain. In some cases, the transmembrane domain is from an alpha chain of a T cell receptor, a beta chain of a T cell receptor, a zeta chain of a T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5. TLR6, TLR7, TLR8, or TLR9. In certain cases, the costimulatory domain comprises a costimulatory region of CD3, CD4, CD8, T cell receptor (TCR), CD27, CD28, 4-1BB (CD137), 0X40, CD30, CD40, PD-1, ICOS, lymphocyte function-associated antigen-1 (LFA-1), CD2, CD7, LIGHT, NKG2C, B7-H3, or a functional fragment thereof. In some cases, the intracellular signaling domain comprises a fragment or domain from one or more molecules selected from the group consisting of a T cell receptor (TCR), CD3 zeta, CD3 gamma, CD3 delta, CD3 epsilon, CD86, common FcR gamma, FcR beta (Fc Epsilon Rib), CD79a, CD79b, Fcgamma Rlla, DAP 10, DAP 12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR. BAFFR, HVEM (LIGHTR). SLAMF7, NKp80 (KLRF1), CD127, CD 160, CD19. CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1 Id, ITGAE, CD 103, ITGAL, CD 11 a, LFA-1, ITGAM, CD lib, ITGAX, CD 1 lc, ITGB1, CD29, ITGB2, CD 18, LFA-1, ITGB7, TNFR2, TRANCE/RANKL, DNAM1 (CD226). SLAMF4 (CD244, 2B4), CD84, CD 96 (Tactile), CEACAM1, CRT AM, Ly9 (CD229), CD160 (BY55), PSGL1, CD 100 (SEMA4D), CD69, SLAMF6 (NTB-A, Lyl08), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, TLR10, TLR11, TLR12, and TLR13. In certain cases, the CAR comprises an amino acid sequence set forth in any one of SEQ ID NOs.: 158-160 but lacking both the signal sequence and Flag tag.

In another aspect, the disclosure features a CAR comprising a means for binding human and murine GPC3, a transmembrane domain, a costimulatory domain, and an intracellular signaling domain. In some cases, the CAR further comprises a hinge domain, wherein the hinge domain links the means for binding human and murine GPC3 to the transmembrane domain. The means for binding human and murine GPC3 can be a single-chain antibody comprising the three VHH CDRs of any one antibody of Table 9. In some cases, the means for binding human and murine GPC3 can be a single-chain antibody comprising the amino acid sequence of any one antibody of Table 8.

In yet another aspect, the disclosure provides a T cell comprising a CAR disclosed herein.

In another aspect, the disclosure features a NK cell comprising a CAR disclosed herein.

In a further aspect, the disclosure provides a nucleic acid or nucleic acids encoding a binding molecule, a bispecific antibody, or a CAR described herein.

In one aspect, the disclosure relates to an expression vector or vectors comprising the nucleic acid or nucleic acids described herein.

In another aspect, the disclosure provides a host cell comprising the nucleic acid or nucleic acids, or the expression vector or vectors, described herein. In some cases, the host cell is a CHO, COS, HeLa, NIH 3T3, or 293 cell.

In yet another aspect, the disclosure features a method of making a binding molecule or bispecific antibody described herein, The method comprises culturing a host cell comprising a nucleic acid or nucleic acids encoding the binding molecule or the bispecific antibody described herein under conditions that facilitate expression of the binding molecule or bispecific antibody and isolating the binding molecule or bispecific antibody. In some cases, the method further comprises formulating the binding molecule as a sterile pharmaceutical composition.

In one aspect, the disclosure relates to a pharmaceutical composition comprising a binding molecule or bispecific antibody described herein, and a pharmaceutically acceptable carrier.

In another aspect, the disclosure provides a pharmaceutical composition comprising a means for binding human and murine GPC3, and a pharmaceutically acceptable carrier. The means for binding human and murine GPC3 can be a single-chain antibody comprising the three VHH CDRs of any one antibody of Table 9. In some cases, the means for binding human and murine GPC3 can be a single-chain antibody comprising the amino acid sequence of any one antibody of Table 8.

In one aspect, the disclosure features a method of treating a cancer in a human subject in need thereof. The method comprises administering to the human subject a therapeutically effective amount of a binding molecule, a bispecific antibody, a CAR-T cell, or a CAR-NK cell described herein. In some cases, the cancer is a Hepatocellular Cell Carcinoma (HCC), a squamous cell lung cancer, a head and neck squamous cell cancer, or a breast cancer. In one case, the cancer is HCC.

In a different aspect, the disclosure provides a polynucleotide comprising an mRNA comprising: (i) a 5′ UTR; (ii) an open reading frame (ORF) encoding a binding molecule or the bispecific antibody, or chimeric antigen receptor described herein; (iii) a stop codon; and (iv) a 3′ UTR.

In some instances, the mRNA comprises a microRNA (miR) binding site. In some cases, the microRNA is expressed in an immune cell of hematopoietic lineage or a cell that expresses TLR7 and/or TLR8 and secretes pro-inflammatory cytokines and/or chemokines. In certain cases, the microRNA binding site is for a microRNA selected from miR-126, miR-142, miR-144, miR-146, miR-150, miR-155, miR-16, miR-21, miR-223, miR-24, miR-27, miR-26a, or any combination thereof. In other cases, the microRNA binding site is for a microRNA selected from miR126-3p, miR-142-3p, miR-142-5p, miR-155, or any combination thereof. In some cases, the microRNA binding site is located in the 3′ UTR of the mRNA.

In certain instances, the mRNA comprises a 5′ terminal cap. In some cases, the 5′ terminal cap comprises a Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In some instances, the mRNA comprises a poly-A region. In certain cases, the poly-A region is at least about 10, at least about 20, at least about 30, at least about 40, at least about 50, at least about 60, at least about 70, at least about 80, at least about 90 nucleotides in length, or at least about 100 nucleotides in length. In other cases, the poly-A region is about 10 to about 200, about 20 to about 180, about 50 to about 160, about 70 to about 140, or about 80 to about 120 nucleotides in length.

In certain instances, the mRNA comprises at least one chemically modified nucleobase, sugar, backbone, or any combination thereof. In some cases, the at least one chemically modified nucleobase is selected from the group consisting of pseudouracil (ψ). N1-methylpseudouracil (m¹ψ), 1-ethylpseudouracil, 2-thiouracil (s2U), 4′-thiouracil, 5-methylcytosine. 5-methyluracil, 5-methoxyuracil, and any combination thereof. In some cases, at least about 25%, at least about 30%, at least about 40%, at least about 50%, at least about 60%, at least about 70%, at least about 80%, at least about 90%, at least about 95%, at least about 99%, or 100% of the uracils are N1-methylpseudouracils.

In certain instances, the open reading frame consists of nucleosides selected from the group consisting of (i) uridine or a modified uridine, (ii) cytidine or a modified cytidine, (iii) adenosine or a modified adenosine, and (iv) guanosine or a modified guanosine. In some cases, the modified uridine is 1-methylpseudouridine.

In some instances, the mRNA comprises a 5′terminal cap comprising Cap1 and a poly-A region 100 nucleotides in length (SEQ ID NO: 250). In some cases, all uracils of the polynucleotide are N1-methylpseudouracils.

In another aspect, the disclosure features a pharmaceutical composition comprising the polynucleotide described herein, and a delivery agent. In some cases, the delivery agent comprises a lipid nanoparticle. In certain cases, the lipid nanoparticle has a mean particle size of from 80 nm to 160 nm. In some cases, the lipid nanoparticle has a polydispersity index (PDI) of from 0.02 to 0.2 and/or a lipid:nucleic acid ratio of from 10 to 20. In some cases, the lipid nanoparticle comprises a neutral lipid, an ionizable amino lipid, a polyethyleneglycol (PEG) lipid, and/or a sterol. In some cases, the lipid nanoparticle comprises a neutral lipid that is 1,2-distearoyl-sn-glycero-3-phosphocholine. In certain cases, the lipid nanoparticle comprises an ionizable amino lipid. In some instances, the lipid nanoparticle comprises a PEG lipid that is PEG 2000 dimyristoyl glycerol or OL56. In some cases, the lipid nanoparticle comprises a sterol that is cholesterol, adosterol, agosterol A, atheronals, avenasterol, azacosterol, blazein, cerevisterol, colestolone, cycloartenol, daucosterol, 7-dehydrocholesterol. 5-dehydroepisterol, 7-dehydrositosterol. 20α,22R-dihydroxycholesterol, dinosterol, epibrassicasterol, episterol, ergosterol, ergosterol, fecosterol, fucosterol, fungisterol, ganoderenic acid, ganoderic acid, ganoderiol, ganodermadiol, 7α-hydroxycholesterol, 22R-hydroxy cholesterol, 27-hydroxycholesterol, inotodiol, lanosterol, lathosterol, lichesterol, lucidadiol, lumisterol, oxycholesterol, oxysterol, parkeol, saringosterol, spinasterol, sterol ester, trametenolic acid, zhankuic acid, or zymosterol. In one instance, the sterol is cholesterol.

In another aspect, the disclosure provides a method of treating a cancer in a human subject in need thereof. The method comprises administering to the human subject a therapeutically effective amount of a polynucleotide or pharmaceutical composition described herein. In some cases, the cancer is a Hepatocellular Cell Carcinoma (HCC), a squamous cell lung cancer, a head and neck squamous cell cancer, or a breast cancer. In one case, the cancer is HCC.

In yet another aspect, the disclosure features a kit comprising (i) a binding molecule; a bispecific antibody; a CAR; a pharmaceutical composition; or a polynucleotide described herein, and (ii) a package insert instructing a user of the kit to administer the binding molecule, bispecific antibody, CAR, pharmaceutical composition, or polynucleotide to a human subject in need thereof. In some instances, the human subject has a cancer. In some cases, the cancer is a Hepatocellular Cell Carcinoma (HCC), a squamous cell lung cancer, a head and neck squamous cell cancer, or a breast cancer. In one case, the cancer is HCC.

The following drawings and detailed description are examples and explanatory, but it is not intended to be limiting.

### BRIEF DESCRIPTION OF THE DRAWINGS

FIG. 1 shows results of an antibody titer assay using an ELISA-based method. HCAb mice that were preconditioned with Flt3 generated moderate Ab titers.

FIG. 2 shows phage display library generation and quality control using a two-step PCR protocol.

FIG. 3 shows an example of a phage display panning strategy.

FIG. 4 shows enrichment after a first round of phage display panning. The first round of panning showed about a 10-fold enrichment over PBS.

FIG. 5 shows enrichment after a second round of phage display panning using human GPC3.

FIG. 6 shows enrichment after a second round of phage display panning using mouse GPC3.

FIG. 7 shows an example of master plate generation.

FIG. 8A-8B shows binding of periplasmic extracts comprising soluble VHH domains assessed using ELISA. Results demonstrated that periplasmic extracts comprising soluble VHH domains had a high positive rate for binding to both human GPC3 (FIG. 8A) and mouse GPC3 (FIG. 8B).

FIG. 9 shows binding of phage with surface displayed VHH domains was also assessed using ELISA. Results demonstrated that Phage with surface displayed VHH domains had a high positive rate for binding to both human GPC3 and mouse GPC3.

FIG. 10 shows a sequencing summary. 156 ELISA-positive clones for GPC3 binding were sequenced. 146 valid VHH sequences were recovered and in total, 36 unique VHH domain sequences were identified belonging to 10 different CDR3 families.

FIG. 11 shows K_{off} of 32 unique VHH domains identified were then evaluated using periplasmic extracts.

FIG. 12 shows binding assessment of phage with surface displayed VHH domains to HepG2 cells using FACS. From the 32 selected clones, 30 showed binding to HepG2 cells (with percentage of binding between 13.71%-88.98%). None of the clones showed binding to A-431 cells.

FIG. 13 shows binding assessment of periplasmic extracts comprising soluble VHH domains to HepG2 cells using FACS. None of the clones demonstrated binding to HepG2 cells as periplasmic extract.

FIG. 14A shows an example of a phage display procedure that was repeated to find additional binders in the library. FIG. 14B shows generation of master plate 5 and an example of selection conditions.

FIG. 15 shows examples of anti-GPC3 VHH domains selected for recloning into a mammalian expression vector comprising a His tag. Figure discloses SEQ ID NOS 98, 59, 20, 20, 20, 20, 20, 20, 20, 20, 20, 20, 47, 47, 47, 26, 26, 20, 20, 20, 99, 60, 33, 39, 21, 39, 39, 36, 30, 39, 39, 63, 48, 48, 48, 27, 27, 69, 39, 63, 100, 61, 22, 22, 22, 22, 22, 22, 22, 22, 46, 64, 49, 49, 49, 25, 25, 70, 73, and 246, respectively, in order of columns.

FIG. 16 demonstrates 19 of the selected anti-GPC3 VHH domains were produced as a His fusion domain as evidenced by a band of approximately 15 kDa, corresponding to VHH-His tagged domains, observed under reducing conditions.

FIG. 17 shows a summary of the production of the anti-GPC3 VHH-His tagged domains.

FIG. 18 summarizes results of affinities of the 19 anti-GPC3 VHH-His tagged domains determined by BiaCore assay.

FIG. 19 summarizes results for the epitope-binding sites of each of the 19 anti-GPC3 VHH-His tag domains. All VHHs were binned into two groups based on the epitopes. VHH2 and VHH3 bind the same epitope and all other VHHs bind the second epitope.

FIG. 20A-20K shows SEC profiles of anti-GPC3 VHH domains tested indicated all, except VHH31 and VHH15, were relatively homogenous.

FIG. 21A shows differential scanning calorimetry (DSC) for a subset of anti-GPC3 VHH domains. FIG. 21B shows melting temperatures of the tested VHH domains were determined to be between about 47° C. and 66° C.

FIG. 22 demonstrates results of BiaCore binding assays. All anti-GPC3 VHH domains were cross human and mouse GPC3 binders with K_{D} at single digit nM range.

FIG. 23A-23L demonstrates FACS results utilized to determine K_{D} values in binding assays to the Jurkat cell line E6.1 (TB152 ATCC) expressing human GPC3 and compared with 293 cells as a negative control. The data were plotted as a function of mean channel fluorescence versus the concentration of the receptor. K_{D} and IC₅₀ values were determined from the half-maximal values of 4-parameter fits of the data using Delta Graph (Red Rock Software, Salt Lake City, UT) or GraphPad Prism (San Diego, CA).

FIG. 24A-24B demonstrates mRNAs encoding the anti-GPC3 VHH CARs transfects activated T cells.

FIG. 25 shows anti-GPC3 VHH CAR T cells inhibited Hep3B cell growth compared to controls (control CAR T, no CAR, no T cells).

FIG. 26 shows that anti-GPC3 VHH CAR T cells induced HepG2 apoptosis.

FIG. 27 demonstrates that anti-GPC3 VHH29 VHH CAR T cell induction of HepG2 apoptosis was dose-dependent for all 4 PBMCC donors tested.

FIG. 28 demonstrates that the killing efficiency of anti-GPC3 VHH CAR T cells peaked about 6 hours post-transfection and decreased over time.

FIG. 29 shows that FLAG-tagged anti-GPC3 VHH containing CAR reached peak expression in blood 24 hours post injection.

FIG. 30 demonstrates that shows that FLAG-tagged anti-GPC3 VHH CAR reached peak expression spleen 6 hours post injection.

### DETAILED DESCRIPTION

The compositions and methods of the disclosure feature Glypican 3 (GPC3) heavy chain variable domains (VHH) (anti-GPC3 VHH domains) and complementarity determining regions (CDRs) thereof, as well as antibodies (e.g., VHH antibodies or “nanobodies”), and other related binding proteins, such as chimeric antigen receptors (CARs) comprising the disclosed VHH domains or CDRs. The disclosure also provides nucleic acids encoding the disclosed proteins, and methods of using the disclosed antibodies, CARs, and nucleic acids. In some embodiments, the antibodies are single-domain antibodies (e.g., a VHH). In some embodiments, the antibodies are single-domain antibodies or single chain Fv (scFv) molecules, among other antigen-binding fragments described herein. In some embodiments, the antibodies are bispecific antibodies (i.e., engagers) that bind to GPC3 and another antigenic target (e.g., an antigen on a T cell or NK cell). In some embodiments, the binding protein is a CAR.

The compositions and methods of the disclosure exhibit a series of beneficial biochemical properties. For example, VHH domains and CDRs described herein and antibodies, antigen-binding fragments, and binding proteins (e.g., CARs) comprising such VHH domains or CDRs are capable of binding GPC3 with high affinity and/or specificity. Furthermore, antibodies and binding proteins (e.g., CARs) disclosed herein may induce cytokine production, such as the production of interferon gamma (IFNγ)).

### Definitions

As used herein, the term “about” refers to a stated numerical term and a value that is no more than 10% above or below the value being described. For example, the term “about 5 nM” indicates disclosure of both the stated value of 5 nM and a range of from 4.5 nM to 5.5 nM.

As used herein, the term “GPC3 antibody” or “anti-GPC3 antibody” refers to an antibody or fragment thereof that specifically binds to or is immunologically reactive with Glypican 3 (GPC3). Similarly, a “GPC3 binding protein” or “anti-GPC3 binding protein” refers to any protein comprising at least one domain (such as a VH domain disclosed herein) that specifically binds to or is immunologically reactive with GPC3. Accordingly, a “GPC3 binding protein” or “anti-GPC3 binding protein” includes, for example, anti-GPC3 antibodies (monospecific, bispecific, or multispecific), chimeric antigen receptors (CARs) that bind to GPCs, and other constructs that bind to GPC3.

As used herein, the term “antibody” (Ab) refers to an immunoglobulin molecule, or a molecule having an immunoglobulin-like scaffold, that specifically binds to, or is immunologically reactive with, a particular antigen. The term “antibody” includes polyclonal, monoclonal, genetically engineered, and otherwise modified forms of antibodies, including, but not limited to, chimeric antibodies, humanized antibodies, and heteroconjugate antibodies (e.g., bi-, tri-, quad-, and multispecific antibodies, diabodies, triabodies, and tetrabodies).

The term “antigen-binding fragment.” as used herein, refers to one or more fragments of an antibody that retain the ability to specifically bind to a target antigen. The antigen-binding function of an antibody can be performed by fragments of a full-length antibody. The antibody fragments can be, e.g., a single-domain antibody (sdAb), Fab, F(ab′)₂, Fab Fv, V_{H}H, scFv, SMIP, diabody, a triabody, an affibody, an aptamer, or recombinant fragments thereof. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, e.g., Bird et al., Science 242:423-426 (1988), and Huston et al., Proc. Natl. Acad. Sci. USA 85:5879-5883 (1988)). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “bispecific antibodies” refers to monoclonal, often human or humanized antibodies that have binding specificities for at least two different antigens. Bispecific GPC3 antibodies of the invention may have binding specificities that are directed towards GPC3 and any other antigen, e.g., for a cell-surface protein, receptor, receptor subunit, or tissue-specific antigen. A bispecific antibody may also be an antibody or antigen-binding fragment thereof that includes two separate antigen-binding domains (e.g., two scFvs joined by a linker). The scFvs may bind the same antigen or different antigens. For the purposes of the present disclosure, the term “engager” may be used interchangeably with “bispecific antibody.”

As used herein, the term “chimeric” antibody refers to an antibody having portions of its sequence derived from at least two different sources, such as variable domain sequences (e.g., CDR sequences) derived from an immunoglobulin of one source organism, such as rat or mouse, and constant regions derived from an immunoglobulin of a different organism (e.g., a human, another primate, pig, goat, rabbit, hamster, cat, dog, guinea pig, member of the bovidae family (such as cattle, bison, buffalo, elk, and yaks, among others), cow, sheep, horse, or bison, among others). Methods for producing chimeric antibodies are known in the art. See, e.g., Morrison, Science 229(4719): 1202-7 (1985); Oi et al., BioTechniques 4:214-221 (1986): Gillies et al., J. Immunol. Methods 125:191-202 (1985): U.S. Pat. Nos. 5,807,715; 4,816,567; and 4,816,397; incorporated herein by reference.

As used herein, the term “chimeric antigen receptor” or “CAR,” refers to an artificial receptor that is engineered to be expressed on a T cell and specifically bind a target protein or antigen (e.g., GPC3). CARs may be used as a therapy with adoptive cell transfer, in which T cells are removed from a patient and modified so that they express a CAR and are then re-introduced into the patient. In some embodiments, the CARs have specificity to a selected target. e.g., cells expressing a prostate-specific membrane antigen. CARs may also comprise an intracellular activation domain, a transmembrane domain and an extracellular domain comprising a tumor associated antigen binding region.

As used herein, the phrase “co-stimulatory ligand,” includes a molecule on an antigen presenting cell (e.g., an artificial APC (aAPC), dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, 0X40, CD30, CD40, PD-1, ICOS, lymphocyte function-associated antigen-1 (LFA-1), CD2, CD7. LIGHT, NKG2C, B7-H3, and a ligand that specifically binds with CD83.

As used herein, a “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

As used herein, a “co-stimulatory signal”, refers to a signal, which in combination with a primary signal, such as TCR/CD3 ligation, leads to T cell proliferation and/or upregulation or downregulation of key molecules.

As used herein, the term “complementarity determining region” or “CDR” refers to a hypervariable region found in the light chain and/or the heavy chain variable domains of an antibody. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The invention includes antibodies comprising modifications in these hybrid hypervariable positions. The variable domains of native heavy and light chains each comprise four framework regions that primarily adopt a β-sheet configuration, connected by three CDRs, which form loops that connect, and in some cases form part of, the β-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see, Kabat et al., Sequences of Proteins of Immunological Interest (National Institute of Health, Bethesda, Md. (1987); incorporated herein by reference). As used herein, numbering of immunoglobulin amino acid residues is performed according to the immunoglobulin amino acid residue numbering system of Kabat et al., unless otherwise indicated.

As used herein, the terms “conservative mutation,” “conservative substitution,” “conservative amino acid substitution,” and the like refer to a substitution of one or more amino acids for one or more different amino acids that exhibit similar physicochemical properties, such as polarity, electrostatic charge, and/or steric volume. These properties are summarized for each of the twenty naturally-occurring amino acids in Table 1 below.

TABLE 1
Representative physicochemical properties of naturally-occurring
amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume† |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | equilibrium | and cationic | |
| | | | at pH 7.4 | forms in | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

†based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “conjugate” refers to a compound formed by the chemical bonding of a reactive functional group of one molecule with an appropriately reactive functional group of another molecule. Conjugates may additionally be produced, e.g., as two polypeptide domains covalently bound to one another as part of a single polypeptide chain that is synthesized by the translation of a single RNA transcript encoding both polypeptides in frame with one another.

As used herein in the context of a GPC3-binding protein, the term “construct” refers to a fusion protein containing a first polypeptide domain bound to a second polypeptide domain. The polypeptide domains may each independently be anti-GPC3 single chain polypeptides, for instance, as described herein. The first polypeptide domain may be covalently bound to the second polypeptide domain, for instance, by way of a linker, such as a peptide linker or a disulfide bridge, among others. Examples of linkers that may be used to join the polypeptide domains of a GPC3 construct include, without limitation, those that are described in Leriche et al., Bioorg. Med. Chem., 20:571-582 (2012), the disclosure of which is incorporated herein by reference in its entirety.

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, and/or linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies comprising two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structure, peptides configured in this way typically trimerize so as to position the V_{H} and V_{L} domains of neighboring peptide chains spatially proximal to one another to permit proper folding (see, Holliger et al., Proc. Natl. Acad. Sci. USA 90:6444-48 (1993); incorporated herein by reference).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “fusion protein” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582 (2012)).

As used herein, the term “heterospecific antibodies” refers to monoclonal (e.g., human or humanized) antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537 (1983)). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986); incorporated herein by reference. Heterospecific antibodies can include Fc mutations that enforce correct chain association in multi-specific antibodies, as described by Klein et al., mAbs 4(6):653-663 (2012); incorporated herein by reference.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Human antibodies can be made by a variety of methods known in the art including phage display methods using antibody libraries derived from human immunoglobulin sequences. See, U.S. Pat. Nos. 4,444,887 and 4,716,111; and PCT publications WO 1998/46645: WO 1998/50433; WO 1998/24893; WO 1998/16654: WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, or immunoglobulin chains or fragments thereof (such as Fv, Fab, Fab′, F(ab′)₂ or other target-binding subdomains of antibodies), which contain minimal sequences derived from non-human immunoglobulin. In general, a humanized antibody will contain substantially all of at least one, and typically two, variable domains, in which all or substantially all of the CDRs correspond to those of a non-human immunoglobulin. All or substantially all of the FRs may also be those of a human immunoglobulin sequence. The humanized antibody may also contain at least a portion of an immunoglobulin constant region (Fc), typically that of a human immunoglobulin consensus sequence. Methods of antibody humanization are known in the art. See, e.g., Riechmann et al., Nature 332:323-7 (1988); U.S. Pat. Nos. 5,530,101; 5,585,089; 5,693,761; 5,693,762; and 6,180,370 to Queen et al; EP239400; PCT publication WO 91/09967: U.S. Pat. No. 5,225,539; EP592106; and EP519596; the disclosure of each of which is incorporated herein by reference.

As used herein, the term “lipid nanoparticle” refers to a transfer vehicle including one or more lipids (e.g., cationic lipids, non-cationic lipids, and PEG-modified lipids). Examples of lipid nanoparticles are formulated to deliver one or more mRNA to one or more target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Lipid nanoparticles may contain a cationic lipid, or a lipid species with a net positive charge at a selected pH (e.g., physiological pH), to encapsulate and/or enhance the delivery of mRNA into the target cells.

As used herein, the terms “messenger RNA” or “mRNA” refer to any polynucleotide which encodes a polypeptide of interest and which is capable of being translated to produce the encoded polypeptide of interest in vitro, in vivo, in situ, or ex vivo. Traditionally, the basic components of an mRNA molecule include a coding region, a 5′UTR, a 3′UTR, a 5′ cap, and a poly-A tail.

As used herein, the terms “modified messenger RNA” or “modified mRNA” refer to mRNA polynucleotides that include naturally occurring and/or non-naturally occurring modifications, for example, of a sugar, a nucleobase, or an internucleoside linkage (e.g., to a linking phosphate, to a phosphodiester linkage, or to the phosphodiester backbone). Non-natural modified nucleotides may be introduced during synthesis of post-synthesis of the polynucleotides to achieve desired functions or properties. The modifications may be present on an internucleoside linkage, purine or pyrimidine base, or sugar. The modification may be introduced with chemical synthesis or with a polymerase enzyme at the terminal of a chain or anywhere else in the chain. Any of the regions of a polynucleotide may be chemically modified.

As used herein, the term “monoclonal antibody” refers to an antibody that is derived from a single clone, including any eukaryotic, prokaryotic, or phage clone, and not the method by which it is produced.

As used herein, the term “multi-specific antibodies” refers to antibodies that exhibit affinity for more than one target antigen, for example, bispecific antibodies. Multispecific anti-GPC3 antibodies of the disclosure may have binding specificities that are directed towards GPC3 and any other antigen(s). The disclosed “multispecific antibodies” may be monoclonal and are often human or humanized. Multi-specific antibodies can have structures similar to full immunoglobulin molecules and include Fc regions, for example IgG Fc regions. A multispecific antibody may also be an antibody or antigen-binding fragment thereof that includes multiple separate antigen-binding domains (e.g., two scFvs joined by a linker). Such structures can include, but not limited to, IgG-Fv, IgG-(scFv)₂, DVD-Ig, (scFv)₂-(scFv)₂-Fc and (scFv)₂-Fc-(scFv)₂. In case of IgG-(scFv)₂, the scFv can be attached to either the N-terminal or the C-terminal end of either the heavy chain or the light chain. Examples of multi-specific molecules that include Fc regions and into which GPC3 antibodies or antigen-binding fragments thereof can be incorporated have been reviewed by Kontermann, 2012, mAbs 4(2):182-197, Yazaki et al., Protein Engineering, Design & Selection 26(3):187-193 (2013), and Grote et al., in Proetzel & Ebersbach (eds.), Antibody Methods and Protocols, Methods in Molecular Biology vol. 901, chapter 16:247-263 (2012); incorporated herein by reference. In some embodiments, antibody fragments can be components of multi-specific molecules without Fc regions, based on fragments of IgG or DVD or scFv. Examples of multi-specific molecules that lack Fc regions and into which antibodies or antibody fragments can be incorporated include scFv dimers (diabodies), trimers (triabodies) and tetramers (tetrabodies). Fab dimers (conjugates by adhesive polypeptide or protein domains) and Fab trimers (chemically conjugated), are described by Hudson and Souriau, 2003, Nature Medicine 9:129-134; incorporated herein by reference.

As used herein, the term “nucleic acid” includes any compound containing a continuous segment of nucleosides joined by way of one or more internucleoside linkages (e.g., polymers of nucleosides linked by way of phosphodiester bonds). Examples of nucleic acids include ribonucleic acids (RNA), deoxyribonucleic acids (DNA), threose nucleic acids (TNA), glycol nucleic acids (GNA), peptide nucleic acids (PNA), locked nucleic acids (LNA), or hybrids thereof. Nucleic acids also include RNAi inducers, RNAi agents, siRNAs, shRNAs, miRNAs, antisense RNAs, ribozymes, catalytic DNAs, tRNAs, RNAs that induce triple spiral formation, aptamers, vectors, and the like. In a preferred embodiment, the nucleic acid is one or more modified messenger RNAs (modified mRNAs).

As used herein, the terms “percent (%) sequence identity,” “percent (%) identity,” and the like, with respect to a reference polynucleotide or polypeptide sequence, is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as: 100 multiplied by (the fraction X/Y) where X is the number of nucleotides or amino acids scored as identical matches by a sequence alignment program (e.g., BLAST) in that program's alignment of A and B. and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “primatized antibody” refers to an antibody comprising framework regions from primate-derived antibodies and other regions, such as CDRs and/or constant regions, from antibodies of a non-primate source. Methods for producing primatized antibodies are known in the art. See, e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “operatively linked” in the context of a polynucleotide fragment is intended to mean that the two polynucleotide fragments are joined such that the amino acid sequences encoded by the two polynucleotide fragments remain in-frame.

As used herein, the term “pharmacokinetic profile” refers to the absorption, distribution, metabolism, and clearance of a therapeutic agent (e.g., a polypeptide, such as an anti-GPC3 antibody, antigen-binding fragment thereof, single-chain polypeptide, or construct of the disclosure) over time following administration of the drug to a patient.

As used herein, the term “regulatory sequence” includes promoters, enhancers, and other expression control elements (e.g., polyadenylation signals) that control the transcription or translation, e.g., of antibody chain genes. Such regulatory sequences are described, for example, in Goeddel, Gene Expression Technology: Methods in Enzymology 185 (Academic Press, San Diego, CA, 1990); incorporated herein by reference.

As used herein, the term “scFv” refers to a single-chain Fv antibody in which the variable domains of the heavy chain and the light chain from an antibody have been joined to form one chain. ScFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). ScFv molecules are known in the art and are described, e.g., in U.S. Pat. No. 5,892,019, Flo et al., Gene 77:51 (1989); Bird et al., Science 242:423 (1988); Pantoliano et al., Biochemistry 30:10117 (1991); Milenic et al., Cancer Research 51:6363 (1991); and Takkinen et al., Protein Engineering 4:837 (1991). The V_{L} and V_{H} domains of a scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. ScFv fragments are described, for example, in WO 2011/084714; incorporated herein by reference.

As used herein, the terms “single-domain antibody,” “sdAb,” “nanobody,” and “VHH antibody” are used interchangeably to refer to a single-chain antibody fragment that contains only a single heavy-chain variable domain. Unlike a traditional, full-length antibody, which includes heavy chains and light chains, each containing a corresponding variable domain (i.e., a heavy chain variable domain, V_{H}, and a light chain variable domain, V_{L}) having three CDRs, a single-domain antibody only includes one heavy-chain variable domain having a total of three CDRs (referred to herein as CDR-H1, CDR-H2, and CDR-H3).

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen will bind to the antigen with a K_{D} of less than 100 nM. For example, an antibody or antigen-binding fragment thereof that specifically binds to an antigen via the antigen binding domain will bind to the antigen with a K_{D} of up to 100 nM (e.g., between 1 pM and 100 nM). An antibody or antigen-binding fragment thereof that does not exhibit specific binding to a particular antigen or epitope thereof will exhibit a K_{D} of greater than 100 nM (e.g., greater than 500 nm, 1 μM, 100 μM, 500 μM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the terms “subject” and “patient” refer to an organism that receives treatment (e.g., by administration of an GPC3 polypeptide, such as an antibody, antigen-binding fragment thereof, single-chain polypeptide, or construct described herein) for a particular disease or condition, such as a cancer or an immunological disorder (e.g., an autoimmune disease). Examples of subjects and patients include mammals, such as humans, primates, pigs, goats, rabbits, hamsters, cats, dogs, guinea pigs, members of the bovidae family (such as cattle, bison, buffalo, and yaks, among others), sheep, and horses, among others, receiving treatment for a cancer, immunological diseases or conditions, such as autoimmune disorders (e.g., allograft rejection) and graft-versus-host disease, among others. A patient that may be treated using the compositions and methods described herein may have an established disease, in which case the patient has been diagnosed as having the disease and has shown symptoms of the disease for a prolonged period of time (e.g., over the course of days, weeks, months, or years). Alternatively, a patient may be symptomatic for a particular disease, but has yet to be diagnosed with the disease by a physician. Other patients that may be treated using the compositions and methods described herein include those that have been diagnosed as having a disease or disorder, and may or may not be showing symptoms of the disease as of yet.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation. DEAE-dextran transfection and the like.

As used herein, the terms “treat” or “treatment” refer to therapeutic treatment, in which the object is to inhibit or slow down (lessen) an undesired physiological change or disorder, such as a cancer or an immunological disorder (e.g., autoimmune disorders (e.g., allograft rejection) and graft-versus-host disease, among others). Beneficial or desired clinical results of treatment include, without limitation, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already having the condition or disorder, as well as those prone to have the condition or disorder or those in which the condition or disorder is to be inhibited.

As used herein, the term “Glypican 3” or “GPC3” refers to a cell surface heparan sulfate proteoglycan comprising a membrane-associated protein core substituted with a variable number of heparan sulfate chains. The encoded protein is important in cellular signaling and modulates a plurality of cellular functions, including, for example, cell growth, embryogenesis, and differentiation. Non-limiting examples of this polypeptide or underlying gene may be found under the Gene Card IDs: GC0XM133535, GC0XM127315, GC0XM129515, GC0XM130614. GC0XM131375, GC0XM132395, GC0XM132497, GC0XM132669, GC0XM122070 (retrieved from genecards.org/cgi-bin/carddisp.pl?gene=GPC3), HGNC: 4451 (genenames.org/data/gene-symbol-report/#!/hgnc_id/4451). NCBI Entrez Gene: 2719 (ncbi.nlm.nih.gov/gene/2719). Ensembl: ENSG00000147257 (useast.ensembl.org/Homo_sapiens/Gene/Summary?g=ENSG00000147257;r=X:1335 35745-133987100), OMIM@: 300037 (omim.org/entry/300037), or UniProtKB/Swiss-Prot: P51654 (uniprot.org/uniprotkb/P51654), which are incorporated by reference herein.

As used herein the term “variable region CDR” includes amino acids in a CDR or complementarity determining region as identified using sequence or structure-based methods. As used herein, the term “CDR” or “complementarity determining region” refers to the noncontiguous antigen-binding sites found within the variable regions of both heavy and light chain polypeptides. These particular regions have been described by Kabat et al., J. Biol. Chem. 252:6609-6616 (1977) and Kabat, et al., Sequences of Proteins of Immunological Interest, Fifth Edition. U.S. Department of Health and Human Services, NIH Publication No. 91-3242 (1991); by Chothia et al., J. Mol. Biol. 196:901-917 (1987), and by MacCallum et al., J. Mol. Biol. 262:732-745 (1996) where the definitions include overlapping or subsets of amino acid residues when compared against each other. In certain embodiments, the term “CDR” is a CDR as defined by Kabat based on sequence comparisons.

As used herein, the terin “vector” includes a nucleic acid vector, e.g., a DNA vector, such as a plasmid, an RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of antibodies, antibody fragments, and/or CARs described herein include plasmids that contain regulatory sequences, such as promoter and enhancer regions, which direct gene transcription. Other useful vectors for expression of antibodies, antibody fragments, and/or CARs contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “V_{H}” refers to the variable region of an immunoglobulin heavy chain of an antibody, including the heavy chain of an Fv, scFv, or Fab. References to “V_{L}” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150.000 Daltons, composed of two identical light (L) chains and two identical heavy (H) chains. Each heavy chain of a native antibody has at the amino terminus a variable domain (V_{II}) followed by a number of constant domains. Each light chain of a native antibody has a variable domain at the amino terminus (V_{L}) and a constant domain at the carboxy terminus.

As used herein, the term “alkyl,” “alkyl group,” or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl,” “alkenyl group,” or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. For example, Cis alkenyl may include one or more double bonds. A Cis alkenyl group including two double bonds may be a linoleyl group. Unless otherwise specified, an alkenyl group described herein refers to both unsubstituted and substituted alkenyl groups.

As used herein, the term “alkynyl,” “alkynyl group,” or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, Cis alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups. i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl,” “heteroalkenyl,” or “heteroalkynyl” refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—. —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the Formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)2R″″, in which each OR are alkoxy groups that can be the same or different and R″″ is an alkyl or alkenyl group), a phosphate (e.g., P(O)₄³⁻), a thiol (e.g., SH), a sulfoxide (e.g., S(O)R), a sulfinic acid (e.g., S(O)OH), a sulfonic acid (e.g., S(O)₂OH), a thial (e.g., C(S)H), a sulfate (e.g., S(O)₄²⁻), a sulfonyl (e.g., S(O)₂), an amide (e.g., C(O)NR₂, or N(R)C(O)R), an azido (e.g., N₃), a nitro (e.g., NO₂), a cyano (e.g., CN), an isocyano (e.g., NC), an acyloxy (e.g., OC(O)R), an amino (e.g., NR₂, NRH, or NH₂), a carbamoyl (e.g., OC(O)NR₂, OC(O)NRH, or OC(O)NH₂), a sulfonamide (e.g., S(O)₂NR₂, S(O)₂NRH, S(O)₂NH₂, N(R)S(O)₂R, N(H)S(O)₂R, N(R)S(O)₂H, or N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C₁₋₆ alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N→O or N+—O—). Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

### Structural Characteristics of Examples of Anti-GPC3 Antibodies and Binding Proteins

Among the molecular features of anti-GPC3 antibodies and binding proteins (e.g., CARs) comprising anti-GPC3 VHH domains described herein, it will be appreciated by one of skill in the art that the CDRs are those regions that predominantly dictate the GPC3-binding properties of the molecule. This disclosure provides amino acid sequence information for the CDRs of anti-GPC3 VHH domains and antibodies and binding proteins comprising such anti-GPC3 VHH domains.

In one instance, the anti-GPC3 antibody binds human and murine GPC3 and comprises a VHH-CDR1, VHH-CDR2, and a VHH-CDR3 of a VHH disclosed herein.

In some instances, the disclosure features an antibody or binding protein (e,g., bispecific molecule, CAR) that binds human and mouse GPC3 and comprises the three VHH CDRs of any one antibody of Table 8. In some cases, the VHH CDRs are based on Kabat, Chothia, enhanced Chothia. Contact, Aho, or IMGT definitions.

For example, the Tables below provides exemplary CDRs for five of the antibodies of the disclosure.

TABLE A
VHH1 Exemplary CDRs

| | | | Enhanced | | |
|---|---|---|---|---|---|
| Antibody | Kabat | Chothia | Chothia | Contact | IMGT |
| VHH- | [symbol] | [symbol] | [symbol] | [symbol] | [symbol] |
| CDR1 | (SEQ ID NO: | (SEQ ID NO: | (SEQ ID NO: | (SEQ ID NO: | (SEQ ID NO: |
| | 20) | 208) | 209) | 210) | 211) |
| VHH- | [symbol] | [symbol] | [symbol] | [symbol] | [symbol] |
| CDR2 | (SEQ ID NO: | (SEQ ID NO: | (SEQ ID NO: | (SEQ ID NO: | (SEQ ID NO: |
| | 21) | [symbol] ) | 213) | 214) | 215) |
| VHH- | [symbol] | [symbol] | [symbol] | [symbol] | [symbol] |
| CDR3 | (SEQ ID NO: | (SEQ ID NO: | (SEQ ID NO: | (SEQ ID NO: | (SEQ ID NO: |
| | [symbol] ) | 22) | [symbol] ) | [symbol] ) | [symbol] ) |

[symbol]

indicates data missing or illegible when filed

TABLE B
VHH2 Exemplary CDRs

| | | | Enhanced | | |
|---|---|---|---|---|---|
| Antibody | Kabat | Chothia | Chothia | Contact | IMGT |
| VHH- | NYLMH | GFTFSNY | GFTFSNYLMH | SNYLMH | GFTFSNYL |
| CDR1 | (SEQ ID NO: | (SEQ ID NO: | (SEQ ID NO: | (SEQ ID NO: | (SEQ ID NO: |
| | 23) | NO: 218) | 219) | 220) | 221) |
| VHH- | NINSDGSSTYYADSVKG | NSDGSS | NINSDGSSTY | WLSNINSDGSSTY | INSDGSST |
| CDR2 | (SEQ ID NO: | (SEQ ID NO: | (SEQ ID NO: | (SEQ ID NO: | (SEQ ID NO: |
| | 24) | 222) | 223) | 224) | 225) |
| VIIII- | GAFDY | GAFDY | GAFDY | TVGAFD | TVGAFDY |
| CDR3 | (SEQ ID NO: | (SEQ ID NO: | (SEQ ID NO: | (SEQ ID NO: | (SEQ ID NO: |
| | 25) | 25) | 25) | 226) | 227) |

TABLE C
VHH3 Exemplary CDRs

| | | | Enhanced | | |
|---|---|---|---|---|---|
| Antibody | Kabat | Chothia | Chothia | Contact | IMGT |
| VHH- | NYLMQ | GFTFSNY | GFTESNYLMQ | SNYLMQ | GFTFSNYL |
| CDR1 | (SEQ ID NO: | (SEQ ID NO: | (SEQ ID NO: | (SEQ ID NO: | (SEQ ID NO: |
| | 26) | 218) | 228) | 229) | 221) |
| VHH- | NINSDGSSTDYADSVKG | NSDGSS | NINSDGSSTD | WLSNINSDGSSTD | INSDGSST |
| CDR2 | (SEQ ID NO: | (SEQ ID NO: | (SEQ ID NO: | (SEQ ID NO: | (SEQ ID NO: |
| | 27) | 222) | 230) | 231) | 225) |
| VHH- | GAFDY | GAFDY | GAFDY | RVGAFD | RVGAFDY |
| CDR3 | (SEQ ID NO: | (SEQ ID NO: | (SEQ ID NO: | (SEQ ID NO: | (SEQ ID NO: |
| | 25) | 25) | 25) | 232) | 233) |

TABLE D
VHH6 Exemplary CDRs

| | | | Enhanced | | |
|---|---|---|---|---|---|
| Antibody | Kabat | Chothia | Chothia | Contact | IMGT |
| VHH- | SYAMS | GFIFSSY | GFIESSYAMS | SSYAMS | GFIFSSYA |
| CDR1 | (SEQ ID NO: | (SEQ ID NO: | (SEQ ID NO: | (SEQ ID NO: | (SEQ ID NO: |
| | 20) | 234) | 235) | 210) | 236) |
| VHH- | SISGGGSSTYYADSLEG | SGGGSS | SISGGGSSTY | WVSSISGGGSSTY | ISGGGSST |
| CDR2 | (SEQ ID NO: | (SEQ ID NO: | (SEQ ID NO: | (SEQ ID NO: | (SEQ ID NO: |
| | 36) | 237) | 238) | 239) | 240) |
| VHH- | DPREGEPPEDY | DPREGEPPEDY | DPREGEPPFDY | SKDPRFGEPPED | SKDPREGEPPEDY |
| CDR3 | (SEQ ID NO: | (SEQ ID NO: | (SEQ ID NO: | (SEQ ID NO: | (SEQ ID NO: |
| | 22) | 22) | 22) | 216) | 217) |

TABLE E
VHH29 Exemplary CDRs

| | | | Enhanced | | |
|---|---|---|---|---|---|
| Antibody | Kabat | Chothia | Chothia | Contact | IMGT |
| VHH- | SYAMS | GFTESSY | GFTESSYAMS | SSYAMS | GETESSYA |
| CDR1 | (SEQ ID NO: | (SEQ ID NO: | (SEQ ID NO: | (SEQ ID NO: | (SEQ ID NO: |
| | 20) | 208) | 209) | 210) | 211) |
| VHH- | SISGGGSSTYYADSLKG | SGGGSS | SISGGGSSTY | WVSSISGGGSSTY | ISGGGSST |
| CDR2 | (SEQ ID NO: | (SEQ ID NO: | (SEQ TD NO: | (SEQ ID NO: | (SEQ ID NO: |
| | 39) | 237) | 238) | 239) | 240) |
| VHH- | DPREGEPPEDY | DPREGEPPEDY | DPREGEPPEDY | SRDPREGEPPED | SRDPREGEPPEDY |
| CDR3 | (SEQ ID NO: | (SEQ ID NO: | (SBQ ID NO: | (SEQ ID NO: | (SEQ ID NO: |
| | 22) | 22) | 22) | 241) | 242) |

In some instances, the disclosure features an antibody or binding protein (e.g., bispecific molecule, CAR) that binds human and mouse GPC3 and comprises the three VHH CDRs based on any single CDR definition set forth in Table A. In some instances, the disclosure features an antibody or binding protein (e,g., bispecific molecule, CAR) that binds human and mouse GPC3 and comprises the three VHH CDRs based on any single CDR definition set forth in Table B. In some instances, the disclosure features an antibody or binding protein (e,g., bispecific molecule, CAR) that binds human and mouse GPC3 and comprises the three VHH CDRs based on any single CDR definition set forth in Table C. In some instances, the disclosure features an antibody or binding protein (e,g., bispecific molecule, CAR) that binds human and mouse GPC3 and comprises the three VHH CDRs based on any single CDR definition set forth in Table D. In some instances, the disclosure features an antibody or binding protein (e,g., bispecific molecule, CAR) that binds human and mouse GPC3 and comprises the three VHH CDRs based on any single CDR definition set forth in Table E.

In some embodiments, the disclosure provides an antibody or binding protein (e.g., bispecific molecule, CAR) that binds human and mouse GPC3 and comprises one, two, or three of the CDRs described in Table 9, below.

For example, in some embodiments, an anti-GPC3 antibody or binding protein of the disclosure can comprise one or more of the following CDRs:
(a) a CDRT having the amino acid sequence selected from the group consisting of SYAMS (SEQ ID NO: 20), NYLMH (SEQ ID NO: 23), NYLMQ (SEQ ID NO: 26), SSAMS (SEQ ID NO: 47), NYWMH (SEQ ID NO: 59), SYGMH (SEQ ID NO: 98), and SFAMS (SEQ ID NO: 101) or an amino acid sequence having up to two amino acid substitutions (e.g., conservative amino acid substitutions) relative to SEQ ID NO: 20, SEQ ID NO; 23, SEQ ID NO: 47, SEQ ID NO: 59, SEQ ID NO: 98, or SEQ ID NO: 101; and
(b) a CDR2 having the amino acid sequence selected from the group consisting of SISGGGTSTYYADSLEG (SEQ ID NO: 21), NINSDGSSTYYADSVKG (SEQ ID NO: 24), NINSDGSSTDYADSVKG (SEQ ID NO: 27), SISGSGSSTYYADSLKG (SEQ ID NO: 30), SISGGGSSAYYADSLKG (SEQ ID NO: 33), SISGGGSSTYYADSLEG (SEQ ID NO: 36), SISGGGSSTYYADSLKG (SEQ ID NO: 39), AISGSGGSTNYVDSVKG (SEQ ID NO: 48), SISGGGGSTYYADSLKG (SEQ ID NO: 57). VSRINSDGSSTSYADPVKG (SEQ ID NO: 60), AISGSGGSTYYADSVKG (SEQ ID NO: 63), AIYSGGSTYYADSVKG (SEQ ID NO: 69), VIWYDGNHKYYADSVKG (SEQ ID NO: 99), and AISGSGGRTHYADSVKG (SEQ ID NO: 102), or an amino acid sequence having up to two amino acid substitutions (e.g., conservative amino acid substitutions) relative to SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 48, SEQ ID NO: 57, SEQ ID NO: 60, SEQ ID NO: 63, SEQ ID NO: 69, SEQ ID NO: 99, or SEQ ID NO: 102; and
(c) a CDR3 having the amino acid sequence selected from the group consisting of: DPRFGEPPFDY (SEQ ID NO: 22), GAFDY (SEQ ID NO: 25), DPRFGEPPLDY (SEQ ID NO: 46), ESMVRGGPFDY (SEQ ID NO: 49), DPRFREPPFDY (SEQ ID NO: 52), DPMFGERPFDY (SEQ ID NO: 58), VALGFDF (SEQ ID NO: 61), EALTGVFDY (SEQ ID NO: 64), GDSSSSRFDY (SEQ ID NO: 70), DPRLGEPPFDY (SEQ ID NO: 73), DPRYGEPPFDY (SEQ ID NO: 76), DPRFFEPPFDY (SEQ ID NO: 97), and DKGGITGTTRNFQH (SEQ ID NO: 100), or an amino acid sequence having up to two amino acid substitutions (e.g., conservative amino acid substitutions) relative to SEQ ID NO: 22, SEQ ID NO; 25, SEQ ID NO: 46, SEQ ID NO: 49, SEQ ID NO: 52, SEQ ID NO: 58, SEQ ID NO: 61, SEQ ID NO: 64, SEQ ID NO: 70, SEQ ID NO: 73, SEQ ID NO: 76, SEQ ID NO: 97, or SEQ ID NO: 100.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH1, shown below (CDR sequences shown in bold):

(VHH1, SEQ ID NO: 1)
EVQLVESGGGLVQPGGSLRLSCAASGFTFSSYAMSWVRQAPGKEPEWVSSI
SGGGTSTYYADSLEGRFTISRDNSKNTLYLQMNSLRAEDTAVYYCSKDPRF
GEPPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 1. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 1. In some embodiments, the antibody or binding protein contains a VHH domain having the amino acid sequence of SEQ ID NO: 1. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH2, shown below (CDR sequences shown in bold):

(VHH2, SEQ ID NO: 2)
QVQLVESGGGLVQPGGSLRLSCAASGFTFSNYLMHWVRQAPGKGLEWLSNI
NSDGSSTYYADSVKGRFTISRDNSKNTLYLQMNSLRAEDTAVYYCTVGAFD
YWGQVTTGTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 2. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 2. In some embodiments, the antibody or binding protein contains a VHH domain having the amino acid sequence of SEQ ID NO: 2. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH3, shown below (CDR sequences shown in bold):

(VHH3, SEQ ID NO: 3)
EVQLVESGGGVVQPGRSLRLSCAASGFTFSNYLMQWVRQAPGKGL
VWLSNINSDGSSTDYADSVKGRFTISRDNAKNTLYLQMNSLRAED
TAVYYCRVGAFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 3. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 3. In some embodiments, the antibody or binding protein contains a VHH domain having the amino acid sequence of SEQ ID NO: 3. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH4, shown below (CDR sequences shown in bold):

(VHH4, SEQ ID NO: 4)
QVQLVESGGGLVQPGGSLRLSCAASGFTFSSYAMSWVRQAPGKES
EWVSSISGSGSSTYYADSLKGRFTISRDNSKNTLYLQMNSLRAED
TAVYYCSKDPRFGEPPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 4. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 4. In some embodiments, the antibody or binding protein contains a VHH domain having the amino acid sequence of SEQ ID NO: 4. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH5, shown below (CDR sequences shown in bold):

(VHH5, SEQ ID NO: 5)
EVQLVESGGGLVQPGGSLRLSCAASGFTFSSYAMSWVRQAPGKEP
EWVSSISGGGSSAYYADSLKGRFTISRDNSKNTLYLQMNSLRAED
TAVYYCSRDPRFGEPPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 5. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 5. In some embodiments, the antibody or binding protein contains a VHH domain having the amino acid sequence of SEQ ID NO: 5. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH6, shown below (CDR sequences shown in bold):

(VHH6, SEQ ID NO: 6)
QVQLVESGGGLVQPGGSLRLSCAASGFIFSSYAMSWVRQAPGKEP
EWVSSISGGGSSTYYADSLEGRFTISRDNSKNTLYLQMNSLRAED
TAVYYCSKDPRFGEPPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 6. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 6. In some embodiments, the antibody or binding protein contains a VHH domain having the amino acid sequence of SEQ ID NO: 6. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH7, shown below (CDR sequences shown in bold):

(VHH7, SEQ ID NO: 7)
EVQLVESGGGLVQPGGSLRLSCAASGFTFSSYAMSWVRQAPGKEP
EWVSSISGGGSSTYYADSLKGRFTISRDNSKNTLYLQMNSLRAED
TAVYYCSRDPRFGEPPFDYWGQGTTVTVSS.

In some embodiments, the antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 7. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 7. In some embodiments, the antibody or binding protein contains a VHH domain having the amino acid sequence of SEQ ID NO: 7. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH8, shown below (CDR sequences shown in bold):

(VHH8, SEQ ID NO: 8)
EVQLVESGGGVVQPGRSLRLSCAASGFTFSSYAMSWVRQAPGKEP
EWVSSISGGGSSTYYADSLKGRFTISRDNSKNTLYLQMNSLRAED
TAVYYCSRDPRFGEPPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 8. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 8. In some embodiments, the antibody or binding protein contains a VHH domain having the amino acid sequence of SEQ ID NO: 8. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH9, shown below (CDR sequences shown in bold):

(VHH9, SEQ ID NO: 9)
QVQLVESGGGLVQPGGSLRLSCAASGFTFSSYAMSWVRQAPGKEP
EWVSSISGGGSSTYYADSLKGRFTISRDNSKNTLYLQMNSLRAED
TAVYYCSRDPRFGEPPLDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 9. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 9. In some embodiments, the antibody or binding protein contains a VHH domain having the amino acid sequence of SEQ ID NO: 9. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH10, shown below (CDR sequences shown in bold):

(VHH10, SEQ ID NO: 10)
QVQLVESGGGLVQPGGSLRLSCAASGFTFSSSAMSWVRQAPGKGL
EWVSAISGSGGSTNYVDSVKGRFTVSRDNSKNTLYLQMNRLRAED
TAVYYCAKESMVRGGPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 10. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 10. In some embodiments, the antibody or binding protein contains a VHH domain having the amino acid sequence of SEQ ID NO: 10. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH11, shown below (CDR sequences shown in bold):

(VHH11, SEQ ID NO: 11)
EVQLVESGGGLVQPGGSLRLSCAASGFTFSSYAMSWVRQAPGKEP
EWVSSISGGGSSTYYADSLKGRFTISRDNSKNTLYLQMNSLRAED
TAVYYCSRDPRFREPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 11. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 11. In some embodiments, the antibody or binding protein contains a VHH domain having the amino acid sequence of SEQ ID NO: 11. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH12, shown below (CDR sequences shown in bold):

(VHH12, SEQ ID NO: 12)
EVQLVESGGGVVQPGRSLRLSCAASGFTFSSYAMSWVRQAPGKEP
EWVSSISGGGSSTYYADSLKGRFTISRDNSKNTLYLQMNSLRAED
TAVYYCSRDPRFGEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 12. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 12. In some embodiments, the antibody or binding protein contains a VHH domain having the amino acid sequence of SEQ ID NO: 12. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH13, shown below (CDR sequences shown in bold):

(VHH13, SEQ ID NO: 13)
EVQLVESGGGLVQPGGSLRLSCAASGFTFSSYAMSWVRQAPGKEP
EWVSSISGGGGSTYYADSLKGRFTISRDNSKNTLYLQMNRLRAED
TAVYYCARDPMFGERPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 13. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 13. In some embodiments, the antibody or binding protein contains a VHH domain having the amino acid sequence of SEQ ID NO: 13. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH14, shown below (CDR sequences shown in bold):

(VHH14, SEQ ID NO: 14)
QVQLVESGGGLVQPGGSLRLSCAASGFNFSNYWMHWVRQAPGKEL
VWVSRINSDGSSTSYADPVKGRFTISRDNANNMLYLQMNSLRAED
TAMYYCVRVALGFDFWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 14. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 14. In some embodiments, the antibody or binding protein contains a VHH domain having the amino acid sequence of SEQ ID NO: 14. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH15, shown below (CDR sequences shown in bold):

(VHH15, SEQ ID NO: 15)
QVQLVESGGGLVQPGGSLRLSCAASGFTFSSYAMSWVRQAPGKGL
EWVSAISGSGGSTYYADSVKGRFTISRDNSKNTLYLQMNSLRAED
TAVYYCAKEALTGVFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 15. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 15. In some embodiments, the antibody or binding protein contains a VHH domain having the amino acid sequence of SEQ ID NO: 15. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH16, shown below (CDR sequences shown in bold):

(VHH16, SEQ ID NO: 16)
EVQLVESGGGLVQPGGSLRLSCAASGFTFSSSAMSWVRQAPGKGL
EWVSAISGSGGSTNYVDSVKGRFTVSRDNSKNTLYLQMNRLRAED
TAVYYCAKESMVRGGPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 16. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 16. In some embodiments, the antibody or binding protein contains a VHH domain having the amino acid sequence of SEQ ID NO: 16. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH17, shown below (CDR sequences shown in bold):

(VHH17, SEQ ID NO: 17)
EVQLVESGGGLVQPGGSLRLSCAASGFTFSSYAMSWVRQAPGKGL
EWVSAIYSGGSTYYADSVKGRFTISRDNSKNTLYLQMNSLRAEDT
AVYYCARGDSSSSRFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 17. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 17. In some embodiments, the antibody or binding protein contains a VHH domain having the amino acid sequence of SEQ ID NO: 17. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH18, shown below (CDR sequences shown in bold):

(VHH18, SEQ ID NO: 18)
QVQLVESGGGLVQPGGSLRLSCAASGFTFSSYAMSWVRQAPGKEPEWVSSI
SGGGSSTYYADSLKGRFTISRDNSKNTLYLQMNSLRAEDTAVYYCSRDPRL
GEPPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 18. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 18. In some embodiments, the antibody or binding protein contains a VHH domain having the amino acid sequence of SEQ ID NO: 18. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH19, shown below (CDR sequences shown in bold):

(VHH19, SEQ ID NO: 19)
EEQLVESGGGLVQPGGSLRLSCAASGFTFSSYAMSWVRQAPGKEPEWVSSI
SGGGGSTYYADSLKGRFTISRDNSKNTLYLQMNSLRAEDTAVYYCARDPRY
GEPPFDYRCQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 19. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 19. In some embodiments, the antibody or binding protein contains a VHH domain having the amino acid sequence of SEQ ID NO: 19. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH20, shown below (CDR sequences shown in bold):

(VHH20, SEQ ID NO: 80)
EVQLVESGGGLVQPGGSLRLSCAASGFTFSSYAMSWVRQAPGKEPEWISSI
SGSGSSTYYADSLKGRFTISRDNSKNTLYLQMNSLRAEDTAVYYCSKDPRF
GEPPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 80. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 80. In some embodiments, the antibody or binding protein contains a VHH domain having the amino acid sequence of SEQ ID NO: 80. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH21, shown below (CDR sequences shown in bold):

(VHH21, SEQ ID NO: 81)
QVQLVESGGGLVQPGGSLRLSCAASGFTFSSYAMSWVRQAPGKEPEWISSI
SGSGSSTYYADSLKGRFTISRDNSKNTLYLQMNSLRAEDTAVYYCSKDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 81. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 81. In some embodiments, the antibody or binding protein contains a VHH domain having the amino acid sequence of SEQ ID NO: 81. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH22, shown below (CDR sequences shown in bold):

(VHH22, SEQ ID NO: 82)
EVQLVESGGGLVQPGGSLRLSCAASGFTFSSYAMSWVRQAPGKEPEWVSSI
SGGGSSTYYADSLKGRFTISRDNSKNTLYLQMDSLRAEDTAVYYCSRDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 82. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 82. In some embodiments, the antibody or binding protein contains a VHH domain having the amino acid sequence of SEQ ID NO: 82. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH23, shown below (CDR sequences shown in bold):

(VHH23, SEQ ID NO: 83)
EVQLVESGGGLVQPGGSLRLSCAASGFTFSSYAMSWVRQAPGKESEWVSSI
SGGGSSTYYADSLKGRFTISRDNSKNTLYLQMNSLRAEDTAVYYCSKDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 83. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 83. In some embodiments, the antibody or binding protein contains a VHH domain having the amino acid sequence of SEQ ID NO: 83. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH24, shown below (CDR sequences shown in bold):

(VHH24, SEQ ID NO: 84)
EVQLVESGGGLVQPGGSLRLSCAASGFTFSSYAMSWVRQAPGKEPEWVSSI
SGGGTSTYYADSLEGRFTISRDNSKNTLYLQMNSLRAEDTAVYYCSKDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 84. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 84. In some embodiments, the antibody or binding protein contains a VHH domain having the amino acid sequence of SEQ ID NO: 84. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH25, shown below (CDR sequences shown in bold):

(VHH25, SEQ ID NO: 85)
EVQLVESGGGLVQPGGSLRLSCAASGFTFSSYAMSWVRQAPGKESEWVSSI
SGSGSSTYYADSLKGRFTISRDNSKNTLYLQMNSLRAEDTAVYYCSKDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 85. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 85. In some embodiments, the antibody or binding protein contains a VHH domain having the amino acid sequence of SEQ ID NO: 85. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH26, shown below (CDR sequences shown in bold):

(VHH26, SEQ ID NO: 86)
EVQLVESGGGLVQPGGSLRLSCAASGFTFSSYAMSWVRQAPGKEPEWISSI
SGSGSSTYYADSLKGRFTISRDNSKNTLYLQMNSLRAEDTAVYYCSKDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 86. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 86. In some embodiments, the antibody or binding protein contains a VHH domain having the amino acid sequence of SEQ ID NO: 86. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH27, shown below (CDR sequences shown in bold):

(VHH27, SEQ ID NO: 87)
EVQLVESGGGLVQPGGSLRLSCAASGFTFSSYAMSWVRQAPGKEPEWVSSI
SGGGSSTYYADSLKGRFTISRDNSKNTLYLQMNSLRAEDTAVYYCSRDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 87. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 87. In some embodiments, the antibody or binding protein contains a VHH domain having the amino acid sequence of SEQ ID NO: 87. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH28, shown below (CDR sequences shown in bold):

(VHH28, SEQ ID NO: 88)
EVQLVESGGGVVQPGRSLRLSCAASGFTFSSYAMSWVRQAPGKEPEWVSSI
SGGGSSTYYADSLKGRFTISRDNSKNTLYLQMNSLRAEDTAVYYCSRDPRF
GEPPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 88. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 88. In some embodiments, the antibody or binding protein contains a VHH domain having the amino acid sequence of SEQ ID NO: 88. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH29, shown below (CDR sequences shown in bold):

(VHH29, SEQ ID NO: 89)
QVQLVESGGGLVQPGGSLRLSCAASGFTFSSYAMSWVRQAPGKEPEWVSSI
SGGGSSTYYADSLKGRFTISRDNSKNTLYLQMNSLRAEDTAVYYCSRDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 89. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 89. In some embodiments, the antibody or binding protein contains a VHH domain having the amino acid sequence of SEQ ID NO: 89. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH30, shown below (CDR sequences shown in bold):

(VHH30, SEQ ID NO: 90)
QVQLVESGGGLVQPGGSLRLSCAASGFIFSSYAMSWVRQAPGKEPEWVSSI
SGGGSSTYYADSLEGRFTISRDNSKNTLYLQMNSLRAEDTAVYYCSKDPRF
FEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 90. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 90. In some embodiments, the antibody or binding protein contains a VHH domain having the amino acid sequence of SEQ ID NO: 90. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH31, shown below (CDR sequences shown in bold):

(VHH31, SEQ ID NO: 91)
QVQLVESGGGVVQPGRSLRLSCAASGFTFSSYGMHWVRQAPGKGLEWVAVI
WYDGNHKYYADSVKGRFTISRDNSKNTLYLQMNSLRAEDTAVYYCARDKGG
ITGTTRNFQHWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 91. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 91. In some embodiments, the antibody or binding protein contains a VHH domain having the amino acid sequence of SEQ ID NO: 91. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH32, shown below (CDR sequences shown in bold):

(VHH32, SEQ ID NO: 92)
QVQLVESGGGLVQPGGSLRLSCAASGFTFSSFAMSWVRQAPGKGLEWVSAI
SGSGGRTHYADSVKGRFTISRDNSKNTLYLQMNSLRAEDTAVYYCAKEALT
GVFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 92. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 92. In some embodiments, the antibody or binding protein contains a VHH domain having the amino acid sequence of SEQ ID NO: 92. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH33, shown below (CDR sequences shown in bold):

(VHH33, SEQ ID NO: 93)
QVQLVESGGGLVQPGGSLRLSCAASGFTFSSSAMSWVRQAPGKGLEWVSAI
SGSGGSTNYVDSVKGRFTVSRDNSKNTLYLQMNRLRAEDTAVYYCAKESMV
RGGPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 93. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 93. In some embodiments, the antibody or binding protein contains a VHH domain having the amino acid sequence of SEQ ID NO: 93. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH34, shown below (CDR sequences shown in bold):

(VHH34, SEQ ID NO: 94)
QVQLVESGGGLVRPGGSLRLSCAASGFTFSSYAMSWVRQAPGKEPEWVSSI
SGGGSSTYYADSLKGRFTISRDNSKNTLYLQMNSLRAEDTAVYYCSRDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 94. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 94. In some embodiments, the antibody or binding protein contains a VHH domain having the amino acid sequence of SEQ ID NO: 94. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH35, shown below (CDR sequences shown in bold):

(VHH35, SEQ ID NO: 95)
QVQLVESGGGVVQPGRSLRLSCAASGFTFSSYAMSWVRQAPGKEPEWVSSI
SGGGSSTYYADSLKGRFTISRDNSKNTLYLQMNSLRAEDTAVYYCSRDPRF
GEPPFDYWGQGTLVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 95. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 95. In some embodiments, the antibody or binding protein contains a VHH domain having the amino acid sequence of SEQ ID NO: 95. In some instances, the anti-GPC3 antibody or binding protein binds both human and mouse GPC3.

In some embodiments, the anti-GPC3 antibody or binding protein contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH36, shown below (CDR sequences shown in bold):

(VHH36, SEQ ID NO: 96)
QVQLVESGGGLVQPGGSLRLSCAASGFTFSSYAMSWVRQAPGKEPEWVSSI
SGGGSSTYYADSLKGRFTISRDNSKNTLYLQMNSLRAEDTAVYYCSRDPRF
GEPPFDYWGQGTTVTVSS.

In some embodiments, the anti-GPC3 antibody or binding protein contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 96. In some embodiments, the antibody or binding protein contains a VHH domain having an amino acid sequence that is at least 95% identical (e.g., at least 95%, 96%, 97%, 98%, 99%, or 100% identical) to the amino acid sequence of SEQ ID NO: 96. In some embodiments, the antibody or binding protein contains a VHH domain having the amino acid sequence of SEQ ID NO: 96. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH1, shown below (CDR sequences shown in bold):

(VHH1, SEQ ID NO: 1)
EVQLVESGGGLVQPGGSLRLSCAASGFTFSSYAMSWVRQAPGKEPEWVSSI
SGGGTSTYYADSLEGRFTISRDNSKNTLYLQMNSLRAEDTAVYYCSKDPRF
GEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 1. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 1. In some embodiments, the antibody or binding protein contains a humanized VHH domain having the amino acid sequence of SEQ ID NO: 1. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH2, shown below (CDR sequences shown in bold):

(VHH2, SEQ ID NO: 2)
QVQLVESGGGLVQPGGSLRLSCAASGFTFSNYLMHWVRQAPGKGLEWLSNI
NSDGSSTYYADSVKGRFTISRDNSKNTLYLQMNSLRAEDTAVYYCTVGAFD
YWGQVTTGTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 2. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 2. In some embodiments, the antibody or binding protein contains a humanized VHH domain having the amino acid sequence of SEQ ID NO: 2. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH3, shown below (CDR sequences shown in bold):

(VHH3, SEQ ID NO: 3)
EVQLVESGGGVVQPGRSLRLSCAASGFTFSNYLMQWVRQAPGKGLVWLSNI
NSDGSSTDYADSVKGRFTISRDNAKNTLYLQMNSLRAEDTAVYYCRVGAFD
YWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 3. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 3. In some embodiments, the antibody or binding protein contains a humanized VHH domain having the amino acid sequence of SEQ ID NO: 3. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH4, shown below (CDR sequences shown in bold):

(VHH4, SEQ ID NO: 4)
QVQLVESGGGLVQPGGSLRLSCAASGFTFSSYAMSWVRQAPGKESEWVSSI
SGSGSSTYYADSLKGRFTISRDNSKNTLYLQMNSLRAEDTAVYYCSKDPRF
GEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 4. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 4. In some embodiments, the antibody or binding protein contains a humanized VHH domain having the amino acid sequence of SEQ ID NO: 4. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH5, shown below (CDR sequences shown in bold):

(VHH5, SEQ ID NO: 5)
EVQLVESGGGLVQPGGSLRLSCAASGFTFSSYAMSWVRQAPGKEPEWVSSI
SGGGSSAYYADSLKGRFTISRDNSKNTLYLQMNSLRAEDTAVYYCSRDPRF
GEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 5. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 5. In some embodiments, the antibody or binding protein contains a humanized VHH domain having the amino acid sequence of SEQ ID NO: 5. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH6, shown below (CDR sequences shown in bold):

(VHH6, SEQ ID NO: 6)
QVQLVESGGGLVQPGGSLRLSCAASGFIFSSYAMSWVRQAPGKEPEWVSSI
SGGGSSTYYADSLEGRFTISRDNSKNTLYLQMNSLRAEDTAVYYCSKDPRF
GEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 6. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 6. In some embodiments, the antibody or binding protein contains a humanized VHH domain having the amino acid sequence of SEQ ID NO: 6. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH7, shown below (CDR sequences shown in bold):

(VHH7, SEQ ID NO: 7)
EVQLVESGGGLVQPGGSLRLSCAASGFTFSSYAMSWVRQAPGKEPEWVSSI
SGGGSSTYYADSLKGRFTISRDNSKNTLYLQMNSLRAEDTAVYYCSRDPRF
GEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 7. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 7. In some embodiments, the antibody or binding protein contains a humanized VHH domain having the amino acid sequence of SEQ ID NO: 7. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH8, shown below (CDR sequences shown in bold):

(VHH8, SEQ ID NO: 8)
EVQLVESGGGVVQPGRSLRLSCAASGFTFSSYAMSWVRQAPGKEPEWVSSI
SGGGSSTYYADSLKGRFTISRDNSKNTLYLQMNSLRAEDTAVYYCSRDPRF
GEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 8. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 8. In some embodiments, the antibody or binding protein contains a humanized VHH domain having the amino acid sequence of SEQ ID NO: 8. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH9, shown below (CDR sequences shown in bold):

(VHH9, SEQ ID NO: 9)
QVQLVESGGGLVQPGGSLRLSCAASGFTFSSYAMSWVRQAPGKEPEWVSSI
SGGGSSTYYADSLKGRFTISRDNSKNTLYLQMNSLRAEDTAVYYCSRDPRF
GEPPLDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 9. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 9. In some embodiments, the antibody or binding protein contains a humanized VHH domain having the amino acid sequence of SEQ ID NO: 9. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH10, shown below (CDR sequences shown in bold):

(VHH10, SEQ ID NO: 10)
QVQLVESGGGLVQPGGSLRLSCAASGFTFSSSAMSWVRQAPGKGLEWVSAI
SGSGGSTNYVDSVKGRFTVSRDNSKNTLYLQMNRLRAEDTAVYYCAKESMV
RGGPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 10. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 10. In some embodiments, the antibody or binding protein contains a humanized VHH domain having the amino acid sequence of SEQ ID NO: 10. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH11, shown below (CDR sequences shown in bold):

(VHH11, SEQ ID NO: 11)
EVQLVESGGGLVQPGGSLRLSCAASGFTFSSYAMSWVRQAPGKEPEWVSSI
SGGGSSTYYADSLKGRFTISRDNSKNTLYLQMNSLRAEDTAVYYCSRDPRF
REPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 11. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 11. In some embodiments, the antibody or binding protein contains a humanized VHH domain having the amino acid sequence of SEQ ID NO: 11. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH12, shown below (CDR sequences shown in bold):

(VHH12, SEQ ID NO: 12)
EVQLVESGGGVVQPGRSLRLSCAASGFTFSSYAMSWVRQAPGKEPEWVSSI
SGGGSSTYYADSLKGRFTISRDNSKNTLYLQMNSLRAEDTAVYYCSRDPRF
GEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 12. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 12. In some embodiments, the antibody or binding protein contains a humanized VHH domain having the amino acid sequence of SEQ ID NO: 12. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH13, shown below (CDR sequences shown in bold):

(VHH13, SEQ ID NO: 13)
EVQLVESGGGLVQPGGSLRLSCAASGFTFSSYAMSWVRQAPGKEPEWVSSI
SGGGGSTYYADSLKGRFTISRDNSKNTLYLQMNRLRAEDTAVYYCARDPMF
GERPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 13. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 13. In some embodiments, the antibody or binding protein contains a humanized VHH domain having the amino acid sequence of SEQ ID NO: 13. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH14, shown below (CDR sequences shown in bold):

(VHH14, SEQ ID NO: 14)
QVQLVESGGGLVQPGGSLRLSCAASGFNFSNYWMHWVRQAPGKELVWVSR
INSDGSSTSYADPVKGRFTISRDNANNMLYLQMNSLRAEDTAMYYCVRVA
LGFDFWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 14. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 14. In some embodiments, the antibody or binding protein contains a humanized VHH domain having the amino acid sequence of SEQ ID NO: 14. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH15, shown below (CDR sequences shown in bold):

(VHH15, SEQ ID NO: 15)
QVQLVESGGGLVQPGGSLRLSCAASGFTFSSYAMSWVRQAPGKGLEWVSAI
SGSGGSTYYADSVKGRFTISRDNSKNTLYLQMNSLRAEDTAVYYCAKEALT
GVFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 15. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 15. In some embodiments, the antibody or binding protein contains a humanized VHH domain having the amino acid sequence of SEQ ID NO: 15. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH16, shown below (CDR sequences shown in bold):

(VHH16, SEQ ID NO: 16)
EVQLVESGGGLVQPGGSLRLSCAASGFTFSSSAMSWVRQAPGKGLEWVSAI
SGSGGSTNYVDSVKGRFTVSRDNSKNTLYLQMNRLRAEDTAVYYCAKESMV
RGGPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 16. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 16. In some embodiments, the antibody or binding protein contains a humanized VHH domain having the amino acid sequence of SEQ ID NO: 16. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH17, shown below (CDR sequences shown in bold):

(VHH17, SEQ ID NO: 17)
EVQLVESGGGLVQPGGSLRLSCAASGFTFSSYAMSWVRQAPGKGLEWVSAI
YSGGSTYYADSVKGRFTISRDNSKNTLYLQMNSLRAEDTAVYYCARGDSSS
SRFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 17. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 17. In some embodiments, the antibody or binding protein contains a humanized VHH domain having the amino acid sequence of SEQ ID NO: 17. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH18, shown below (CDR sequences shown in bold):

(VHH18, SEQ ID NO: 18)
QVQLVESGGGLVQPGGSLRLSCAASGFTFSSYAMSWVRQAPGKEPEWVSSI
SGGGSSTYYADSLKGRFTISRDNSKNTLYLQMNSLRAEDTAVYYCSRDPRL
GEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 18. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 18. In some embodiments, the antibody or binding protein contains a humanized VHH domain having the amino acid sequence of SEQ ID NO: 18. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH19, shown below (CDR sequences shown in bold):

(VHH19, SEQ ID NO: 19)
EEQLVESGGGLVQPGGSLRLSCAASGFTFSSYAMSWVRQAPGKEPEWVSSI
SGGGGSTYYADSLKGRFTISRDNSKNTLYLOMNSLRAEDTAVYYCARDPRY
GEPPFDYRCQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 19. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 19. In some embodiments, the antibody or binding protein contains a humanized VHH domain having the amino acid sequence of SEQ ID NO: 19. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH. VHH20, shown below (CDR sequences shown in bold):

(VHH20, SEQ ID NO: 80)
EVQLVESGGGLVQPGGSLRLSCAASGFTFSSYAMSWVRQAPGKEPEWISSI
SGSGSSTYYADSLKGRFTISRDNSKNTLYLQMNSLRAEDTAVYYCSKDPRF
GEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 80. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 80. In some embodiments, the antibody or binding protein contains a humanized VHH domain having the amino acid sequence of SEQ ID NO: 80. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH21, shown below (CDR sequences shown in bold):

(VHH21, SEQ ID NO: 81)
QVQLVESGGGLVQPGGSLRLSCAASGFTFSSYAMSWVRQAPGKEPEWISSI
SGSGSSTYYADSLKGRFTISRDNSKNTLYLQMNSLRAEDTAVYYCSKDPRF
GEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 81. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 81. In some embodiments, the antibody or binding protein contains a humanized VHH domain having the amino acid sequence of SEQ ID NO: 81. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH22, shown below (CDR sequences shown in bold):

(VHH22, SEQ ID NO: 82)
EVQLVESGGGLVQPGGSLRLSCAASGFTFSSYAMSWVRQAPGKEPEWVSSI
SGGGSSTYYADSLKGRFTISRDNSKNTLYLQMDSLRAEDTAVYYCSRDPRF
GEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 82. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 82. In some embodiments, the antibody or binding protein contains a humanized VHH domain having the amino acid sequence of SEQ ID NO: 82. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH23, shown below (CDR sequences shown in bold):

(VHH23, SEQ ID NO: 83)
EVQLVESGGGLVQPGGSLRLSCAASGFTFSSYAMSWVRQAPGKESEWVSSI
SGGGSSTYYADSLKGRFTISRDNSKNTLYLQMNSLRAEDTAVYYCSKDPRF
GEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 83. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 83. In some embodiments, the antibody or binding protein contains a humanized VHH domain having the amino acid sequence of SEQ ID NO: 83. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH24, shown below (CDR sequences shown in bold):

(VHH24, SEQ ID NO: 84)
EVQLVESGGGLVQPGGSLRLSCAASGFTFSSYAMSWVRQAPGKEPEWVSSI
SGGGTSTYYADSLEGRFTISRDNSKNTLYLQMNSLRAEDTAVYYCSKDPRF
GEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 84. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 84. In some embodiments, the antibody or binding protein contains a humanized VHH domain having the amino acid sequence of SEQ ID NO: 84. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH25, shown below (CDR sequences shown in bold):

(VHH25, SEQ ID NO: 85)
EVQLVESGGGLVQPGGSLRLSCAASGFTFSSYAMSWVRQAPGKESEWVSSI
SGSGSSTYYADSLKGRFTISRDNSKNTLYLQMNSLRAEDTAVYYCSKDPRF
GEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 85. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 85. In some embodiments, the antibody or binding protein contains a humanized VHH domain having the amino acid sequence of SEQ ID NO: 85. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH26, shown below (CDR sequences shown in bold):

(VHH26, SEQ ID NO: 86)
EVQLVESGGGLVQPGGSLRLSCAASGFTFSSYAMSWVRQAPGKEPEWISSI
SGSGSSTYYADSLKGRFTISRDNSKNTLYLQMNSLRAEDTAVYYCSKDPRF
GEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 86. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 86. In some embodiments, the antibody or binding protein contains a humanized VHH domain having the amino acid sequence of SEQ ID NO: 86. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH27, shown below (CDR sequences shown in bold):

(VHH27, SEQ ID NO: 87)
EVOLVESGGGLVQPGGSLRLSCAASGFTFSSYAMSWVRQAPGKEPEWVS
SISGGGSSTYYADSLKGRFTISRDNSKNTLYLQMNSLRAEDTAVYYCSR
DPRFGEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 87. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 87. In some embodiments, the antibody or binding protein contains a humanized VHH domain having the amino acid sequence of SEQ ID NO: 87. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH28, shown below (CDR sequences shown in bold):

(VHH28, SEQ ID NO: 88)
EVQLVESGGGVVQPGRSLRLSCAASGFTFSSYAMSWVRQAPGKEPEWVS
SISGGGSSTYYADSLKGRFTISRDNSKNTLYLQMNSLRAEDTAVYYCSR
DPRFGEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 88. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 88. In some embodiments, the antibody or binding protein contains a humanized VHH domain having the amino acid sequence of SEQ ID NO: 88. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH29, shown below (CDR sequences shown in bold):

(VHH29, SEQ ID NO: 89)
QVQLVESGGGLVQPGGSLRLSCAASGFTFSSYAMSWVRQAPGKEPEWVS
SISGGGSSTYYADSLKGRFTISRDNSKNTLYLQMNSLRAEDTAVYYCSR
DPRFGEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 89. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 89. In some embodiments, the antibody or binding protein contains a humanized VHH domain having the amino acid sequence of SEQ ID NO: 89. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH30, shown below (CDR sequences shown in bold):

(VHH30, SEQ ID NO: 90)
QVQLVESGGGLVQPGGSLRLSCAASGFIFSSYAMSWVRQAPGKEPEWVS
SISGGGSSTYYADSLEGRFTISRDNSKNTLYLQMNSLRAEDTAVYYCSK
DPRFFEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 90. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 90. In some embodiments, the antibody or binding protein contains a humanized VHH domain having the amino acid sequence of SEQ ID NO: 90. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH. VHH31, shown below (CDR sequences shown in bold):

(VHH31, SEQ ID NO: 91)
QVQLVESGGGVVQPGRSLRLSCAASGFTFSSYGMHWVRQAPGKGLEWVA
VIWYDGNHKYYADSVKGRFTISRDNSKNTLYLQMNSLRAEDTAVYYCAR
DKGGITGTTRNFQHWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 91. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 91. In some embodiments, the antibody or binding protein contains a humanized VHH domain having the amino acid sequence of SEQ ID NO: 91. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH32, shown below (CDR sequences shown in bold):

(VHH32, SEQ ID NO: 92)
QVQLVESGGGLVQPGGSLRLSCAASGFTFSSFAMSWVRQAPGKGLEWVS
AISGSGGRTHYADSVKGRFTISRDNSKNTLYLQMNSLRAEDTAVYYCAK
EALTGVFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 92. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 92. In some embodiments, the antibody or binding protein contains a humanized VHH domain having the amino acid sequence of SEQ ID NO: 92. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH33, shown below (CDR sequences shown in bold):

(VHH33, SEQ ID NO: 93)
QVQLVESGGGLVQPGGSLRLSCAASGFTFSSSAMSWVRQAPGKGLEWVS
AISGSGGSTNYVDSVKGRFTVSRDNSKNTLYLQMNRLRAEDTAVYYCAK
ESMVRGGPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 93. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 93. In some embodiments, the antibody or binding protein contains a humanized VHH domain having the amino acid sequence of SEQ ID NO: 93. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH34, shown below (CDR sequences shown in bold):

(VHH34, SEQ ID NO: 94)
QVQLVESGGGLVRPGGSLRLSCAASGFTFSSYAMSWVRQAPGKEPEWVS
SISGGGSSTYYADSLKGRFTISRDNSKNTLYLQMNSLRAEDTAVYYCSR
DPRFGEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 94. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 94. In some embodiments, the antibody or binding protein contains a humanized VHH domain having the amino acid sequence of SEQ ID NO: 94. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH35, shown below (CDR sequences shown in bold):

(VHH35, SEQ ID NO: 95)
QVQLVESGGGVVQPGRSLRLSCAASGFTFSSYAMSWVRQAPGKEPEWVS
SISGGGSSTYYADSLKGRFTISRDNSKNTLYLQMNSLRAEDTAVYYCSR
DPRFGEPPFDYWGQGTLVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 95. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 95. In some embodiments, the antibody or binding protein contains a humanized VHH domain having the amino acid sequence of SEQ ID NO: 95. In some instances, the anti-GPC3 antibody or binding protein binds both human and mouse GPC3.

In some embodiments, the anti-GPC3 antibody or binding protein contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of VHH, VHH36, shown below (CDR sequences shown in bold):

(VHH36, SEQ ID NO: 96)
QVQLVESGGGLVQPGGSLRLSCAASGFTFSSYAMSWVRQAPGKEPEWVS
SISGGGSSTYYADSLKGRFTISRDNSKNTLYLQMNSLRAEDTAVYYCSR
DPRFGEPPFDYWGQGTTVTVSS.

In some embodiments, the anti-GPC3 antibody or binding protein contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 96. In some embodiments, the antibody or binding protein contains a humanized VHH domain having an amino acid sequence that is at least 95% identical (e.g., at least 95%, 96%, 97%, 98%, 99%, or 100% identical) to the amino acid sequence of SEQ ID NO: 96. In some embodiments, the antibody or binding protein contains a humanized VHH domain having the amino acid sequence of SEQ ID NO: 96. In some instances, the anti-GPC3 antibody or binding protein binds both human and mouse GPC3.

In some embodiments, an antibody or antigen-binding fragment is a murine-specific antibody or antigen-binding fragment, e.g., the antibody or binding protein specifically binds the murine antigen. In some embodiments, an antibody or antigen-binding fragment is a rat-specific antibody or antigen-binding fragment, e.g., the antibody or binding protein specifically binds the rat antigen. In some embodiments, an antibody or antigen-binding fragment is a llama-specific antibody or antigen-binding fragment, e.g., the antibody or binding protein specifically binds the llama antigen. In some embodiments, an antibody or antigen-binding fragment is a human-specific antibody or antigen-binding fragment, e.g., the antibody or binding protein specifically binds the human antigen. In some embodiments, an antibody or antigen-binding fragment is human-specific even if the antibody or binding protein is not human or humanized.

In some embodiments, an antibody or antigen-binding fragment is a murine-specific antibody or antigen-binding fragment, e.g., the antibody or binding protein specifically binds the murine antigen. In some embodiments, an antibody or antigen-binding fragment is a rat-specific antibody or antigen-binding fragment, e.g., the antibody, antigen-binding fragment, or binding protein specifically binds the rat antigen. In some embodiments, an antibody or antigen-binding fragment is a llama-specific antibody or antigen-binding fragment, e.g., the antibody or binding protein specifically binds the llama antigen. In some embodiments, an antibody or antigen-binding fragment is a human-specific antibody or antigen-binding fragment, e.g., the antibody or binding protein specifically binds the human antigen. In some embodiments, an antibody or antigen-binding fragment is human-specific even if the antibody or binding protein is not human or humanized.

### Multispecific Antibodies

In another aspect, the present disclosure provides multispecific antibodies, for example, bispecific antibodies (BsAbs; also referred to herein as “engagers”) that may have binding specificities for GPC3 and any other antigen, e.g., for a cell-surface protein, receptor, receptor subunit, or tissue-specific antigen, or other non-GPC3 antigen. Multispecific antibodies typically comprise at least two different variable domains, wherein each variable domain is capable of specifically binding to a separate antigen (i.e., GPC3 and any other antigen). Each antigen-binding domain of a bispecific antibody can comprise a heavy chain variable domain (VH), a light chain variable domain (VL), or a VH and a VL. In the context of a bispecific antigen-binding fragment comprising a first and a second antigen-binding domain, each antigen binding domain comprises at least one CDR that alone, or in combination with one or more additional CDRs and/or framework regions, specifically binds to a particular antigen (i.e., GPC3 and any other antigen).

The first antigen-binding domain and the second antigen-binding domain may be directly or indirectly connected to one another to form a bispecific antigen-binding fragment (i.e., bispecific ScFv), and, optionally, further bound to an Fc domain. Alternatively, the first antigen-binding domain and the second antigen-binding domain may each be connected to a separate Fc domain. Bispecific antigen-binding fragments of the present disclosure may comprise two Fc domains that are each individually part of a separate antibody heavy chain. The first and second Fc domains may be of the same sequence, or the Fc domains may have a mutation in the CH3 domain intended for the facilitation or ease of purification of heterodimeric (i.e., bispecific) molecules.

A multispecific antibody may also be an antibody or antigen-binding fragment thereof that includes at least two separate antigen-binding domains (e.g., two scFvs joined by a linker). The scFvs may bind the same antigen or different antigens.

In some embodiments, multispecific antibodies of the present disclosure are secreted (e.g., released from a cell, for example, into the extracellular milieu).

Multispecific antibodies of the present disclosure can include any anti-GPC3 CDRs or VH domains described herein.

Multispecific antibodies of the present disclosure can comprise binding specificities that are directed towards GPC3 and any other antigen. Any other antigen may be or comprise, for example, an immune cell antigen, such as a T cell activation marker, a pathogenic antigen, or any other non-GPC3 antigen. The antigen binding domains of such bispecific antibodies (e.g., that comprise binding specificity towards an immune cell antigen) may be referred to as “immune cell binding domains”. For example and without limitation, bispecific antibodies of the present disclosure can comprise immune cell binding domains specific for neutrophils, eosinophils, basophils, mast cells, monocytes (e.g., macrophages, dendritic cells, tumor associated macrophages), natural killer cells, and/or lymphocytes (e.g., B cells, T cells). In some embodiments, an immune cell binding domain comprises binding specificity towards an immune cell antigen indicative of the state of the immune cells (e.g., an activated immune cell). In some embodiments, bispecific antibodies of the present disclosure bind both GPC3 and an immune cell antigen. Examples of immune cell antigens include, without limitation, CD1a, CD1b, CD1c, CD1d, CD2, CD3γ, CD3ε, CD4, CD5, CD6, CD7, CD8, CD9, CD10, CD11a, CD11b, CD13, CD14, CD15, CD15s, CD15u, CD16, CDw17, CD19, CD20, CD21, CD22, CD23, CD24, CD25, CD26, CD27, CD28, CD30, CD31, CD32, CD33, CD35, CD36, CD37, CD38, CD39, CD40, CD45RO, CD47, CD49a, CD49b, CD49c, CD49d, CD49e, CD49f, CD50, CD52, CD53, CD56, CD62. CD64, CD68, CD70, CD72. CD73, CD75, CD83. CD85, CD96, CD119, CD124, CD138, CD139, CD152, CD153, CD155, CD180, CD205, CD209, CD244, CD245, and/or CD247. In some embodiments, bispecific antibodies of the present disclosure bind both GPC3 and CD3R. In some embodiments, bispecific antibodies of the present disclosure bind both GPC3 and CD16.

The disclosed multispecific antibodies may be produced by any means known in the art for producing multispecific antibodies, so long as the resulting multispecific antibody retains the functional characteristic of being able to specifically bind GPC3 and at least one other antigen. In some embodiments, the BsAbs may be created using the methods described in Labrijin et al., Proc. Natl. Acad. Sci. USA, 110(13):5145-50 (2013). Briefly, the two parental Abs, each containing single matched point mutations in the CH3 domains, are separately expressed and then mixed under reducing conditions in vitro. This separates the Abs into half-molecules, followed by reassembly, to form bispecific antibodies, and is compatible with large-scale manufacturing of bispecific antibodies. However, this is simply one example of a method for making a multispecific antibody. Those of skill in the art will be aware that other methods of producing multispecific antibodies are available, and the present disclosure is not intended to be limited solely to the methods of making and type of multispecific antibodies disclosed herein.

Other multispecific antibody formats or technologies may be used to make the multispecific antigen-binding molecules of the present disclosure. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific examples of bispecific formats that can be used in the context of the present invention include, without limitation, scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab2 bispecific formats (see, e.g., Klein et al. 2012, mAbs 4:6, 1-1 1, and references cited therein, for a review of the foregoing formats).

Multispecific antibodies can be made from or incorporate the CDRs or variable regions from polyclonal, monoclonal, chimeric, human, partially or fully humanized, and/or recombinant antibodies. Thus, the “parent” antibodies for the disclosed multispecific antibodies are not particularly limited; however, they are preferably fully human. In some embodiments, the parent antibody can be a polyclonal antibody. In some embodiments, the parent antibody can be a monoclonal. In some embodiments, the parent antibody can be a human antibody.

### Chimeric Antigen Receptors

In some aspects, the present disclosure provides chimeric antigen receptors (CARs) and modified T cells or precursors thereof expressing one or more of the disclosed CARs. Thus, in some aspects, the T cell has been genetically modified to express the CAR. In some aspects, CARs of the present disclosure comprise an antigen binding domain, a transmembrane domain, a hinge domain, and an intracellular signaling domain. In some aspects, the CARs comprise an antigen binding domain, a spacer domain, a transmembrane domain, a costimulatory domain, and a signaling domain.

In some aspects, the antigen binding domain may be operably linked to another domain of the CAR, such as the transmembrane domain and/or the intracellular domain, both described elsewhere herein, for expression in the T cell. In some aspects, a first nucleic acid sequence encoding the antigen binding domain is operably linked to a second nucleic acid encoding a transmembrane domain, and further operably linked to a third a nucleic acid sequence encoding an intracellular domain.

In some aspects, the antigen binding domains described herein can be combined with any of the transmembrane domains described herein, any of the intracellular domains or cytoplasmic domains described herein, or any of the other domains described herein that may be included in a CAR of the present invention. In some aspects, the CAR may also include a spacer domain as described herein. In some aspects, each of the antigen binding domain, transmembrane domain, and intracellular domain is separated by a linker.

### Antigen Binding Domain

The antigen binding domain of a CAR is an extracellular region of the CAR for binding to a specific target antigen including proteins, carbohydrates, and glycolipids. In some aspects, the CAR comprises affinity to a target antigen (e.g., GPC3) on a target cell. The target antigen may include any type of protein, or epitope thereof, associated with the target cell. For example, the CAR may comprise affinity to a target antigen on a target cell that indicates a particular disease state of the target cell.

In some aspects, the target cell antigen is or comprises a GPC3 expressed on the cell surface. In some aspects, the CAR has affinity and/or specificity for GPC3, a GPC3 epitope, a GPC3 mutant, and/or a GPC3 fragment.

As described herein, a CAR of the present disclosure having affinity for a specific target antigen (e.g., GPC3) on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin. In some embodiments, the target-specific binding domain is a llama target-specific binding domain, e.g., the target-specific binding domain is of llama origin. In some embodiments, the target-specific binding domain is a rat target-specific binding domain, e.g., the target-specific binding domain is of rat origin. In one embodiment, a CAR of the present disclosure having affinity for GPC3 on a target cell may comprise GPC3-binding domain. In some embodiments, the binding domain is a murine binding domain, e.g., the binding domain is of murine origin. In some embodiments, the binding domain is a human binding domain, e.g., the GPC3-binding domain is of human origin. In some embodiments, the binding domain is a llama binding domain, e.g., the GPC3-binding domain is of llama origin. In some embodiments, the binding domain is a rat binding domain, e.g., the GPC3-binding domain is of rat origin.

In some aspects, a CAR of the present disclosure may have affinity for one or more target antigens on one or more target cells. In some aspects, a CAR may have affinity for one or more target antigens on a target cell. In such aspects, the CAR is a bispecific CAR (e.g., has binding specificities that are directed towards GPC3 and any other antigen, e.g., for a cell-surface protein, receptor, receptor subunit, or tissue-specific antigen), or a multispecific CAR (e.g., has binding specificities that are directed towards GPC3 and any other two or more antigens). In some aspects, the CAR comprises one or more target-specific binding domains that confer affinity for one or more target antigens. In some aspects, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains are present in a CAR, the binding domains may be arranged in tandem and may be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

In some aspects, the antigen binding domain is selected from the group consisting of an antibody and an antigen-binding fragment. In some embodiments, a GPC3 binding domain of the present invention is selected from the group consisting of a GPC3-specific antibody, a GPC3-specific Fab, and a GPC3-specific scFv. In one embodiment, a GPC3-binding domain is a GPC3-specific antibody. In one embodiment, a GPC3-binding domain is a GPC3-specific antigen-binding fragment.

The antigen binding domain can include any domain that binds to the antigen and may include, but is not limited to, a GPC3 protein, poly peptide, variant, mutant, or fragment thereof that is capable of binding to GPC3; a monoclonal antibody; a polyclonal antibody; a synthetic antibody: a human antibody; a humanized antibody: a non-human antibody; and any fragment or scFv thereof. In some embodiments, the antigen binding domain portion comprises a mammalian antibody or a fragment thereof. The choice of antigen binding domain may depend upon the type and number of antigens that are present on the surface of a target cell. As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., GPC3 binding domain) comprises an scFv having the configuration from N-terminus to C-terminus, VH-linker-VL. In some embodiments, the antigen binding domain (e.g., GPC3 binding domain) comprises an scFv having the configuration from N-terminus to C-terminus, VL-linker-VH. Those of skill in the art would be able to select the appropriate configuration for use in the present invention. The linker of an scFv is usually rich in glycine for flexibility, as well as serine or threonine for solubility. The linker can link the heavy chain variable region and the light chain variable region of the extracellular antigen-binding domain. Non-limiting examples of linkers are disclosed in WO 2014/087010.

In some aspects, the antigen binding domain comprises one or more of the following CDRs:
(d) a CDR1 having the amino acid sequence selected from the group consisting of: SYAMS (SEQ ID NO: 20), NYLMH (SEQ ID NO: 23), NYLMQ (SEQ ID NO: 26), SSAMS (SEQ ID NO: 47), NYWMH (SEQ ID NO: 59). SYGMH (SEQ ID NO: 98), and SFAMS (SEQ ID NO: 101) or an amino acid sequence having up to two amino acid substitutions (e.g., conservative amino acid substitutions) relative to SEQ ID NO: 20, SEQ ID NO; 23, SEQ ID NO: 47, SEQ ID NO: 59, SEQ ID NO: 98, or SEQ ID NO: 101; and
(e) a CDR2 having the amino acid sequence selected from the group consisting of: SISGGGTSTYYADSLEG (SEQ ID NO: 21), NINSDGSSTYYADSVKG (SEQ ID NO: 24), NINSDGSSTDYADSVKG (SEQ ID NO: 27), SISGSGSSTYYADSLKG (SEQ ID NO: 30), SISGGGSSAYYADSLKG (SEQ ID NO: 33), SISGGGSSTYYADSLEG (SEQ ID NO: 36), SISGGGSSTYYADSLKG (SEQ ID NO: 39), AISGSGGSTNYVDSVKG (SEQ ID NO: 48), SISGGGGSTYYADSLKG (SEQ ID NO: 57), VSRINSDGSSTSYADPVKG (SEQ ID NO: 60), AISGSGGSTYYADSVKG (SEQ ID NO: 63), AIYSGGSTYYADSVKG (SEQ ID NO: 69), VIWYDGNHKYYADSVKG (SEQ ID NO: 99), and AISGSGGRTHYADSVKG (SEQ ID NO: 102), or an amino acid sequence having up to two amino acid substitutions (e.g., conservative amino acid substitutions) relative to SEQ ID NO: 21, SEQ ID NO: 24, SEQ ID NO: 27, SEQ ID NO: 30, SEQ ID NO: 33, SEQ ID NO; 36, SEQ ID NO: 39, SEQ ID NO: 48, SEQ ID NO: 57, SEQ ID NO: 60, SEQ ID NO: 63, SEQ ID NO: 69, SEQ ID NO: 99, or SEQ ID NO: 102; and
(f) a CDR3 having the amino acid sequence selected from the group consisting of: DPRFGEPPFDY (SEQ ID NO: 22), GAFDY (SEQ ID NO: 25), DPRFGEPPLDY (SEQ ID NO: 46), ESMVRGGPFDY (SEQ ID NO: 49), DPRFREPPFDY (SEQ ID NO: 52), DPMFGERPFDY (SEQ ID NO: 58), VALGFDF (SEQ ID NO: 61), EALTGVFDY (SEQ ID NO: 64), GDSSSSRFDY (SEQ ID NO: 70), DPRLGEPPFDY (SEQ ID NO: 73), DPRYGEPPFDY (SEQ ID NO: 76), DPRFFEPPFDY (SEQ ID NO: 97), and DKGGITGTTRNFQH (SEQ ID NO: 100), or an amino acid sequence having up to two amino acid substitutions (e.g., conservative amino acid substitutions) relative to SEQ ID NO: 22, SEQ ID NO; 25, SEQ ID NO: 46, SEQ ID NO: 49, SEQ ID NO: 52, SEQ ID NO: 58, SEQ ID NO: 61, SEQ ID NO: 64, SEQ ID NO: 70, SEQ ID NO: 73, SEQ ID NO: 76, SEQ ID NO: 97, or SEQ ID NO: 100.

In some embodiments, the antigen binding domain may comprise any of the combinations of CDR-1, CDR-2, and CDR-3 that are disclosed in Table 9.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 1

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 1. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 1. In some embodiments, the antigen binding domain contains a VHH domain having the amino acid sequence of SEQ ID NO: 1.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 2.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 2. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 2. In some embodiments, the antigen binding domain contains a VHH domain having the amino acid sequence of SEQ ID NO: 2.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 3.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 3. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 3. In some embodiments, the antigen binding domain contains a VHH domain having the amino acid sequence of SEQ ID NO: 3.

In some embodiments, the antigen binding domain contains a VHH domain having an amino acid sequence that is at least 85% identical (e.g., at least 85%, 86%, 87%, 88%, 89%, 90, 91%, 92%, 93%, 94%, 95%, 96%, 97%, 98%, 99%, or 100% identical) to the amino acid sequence of SEQ ID NO: 4.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 4. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 4. In some embodiments, the antigen binding domain contains a VHH domain having the amino acid sequence of SEQ ID NO: 4.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 5.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 5. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 5. In some embodiments, the antigen binding domain contains a VHH domain having the amino acid sequence of SEQ ID NO: 5.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 6.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 6. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 6. In some embodiments, the antigen binding domain contains a VHH domain having the amino acid sequence of SEQ ID NO: 6.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 7.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 7. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 7. In some embodiments, the antigen binding domain contains a VHH domain having the amino acid sequence of SEQ ID NO: 7.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 8.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 8. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 8. In some embodiments, the antigen binding domain contains a VHH domain having the amino acid sequence of SEQ ID NO: 8.

In some embodiments, the antigen binding domain contains a VHH domain having an amino acid sequence that is at least 85% identical (e.g., at least 85%, 86%, 87%, 88%, 89%, 90, 91%, 92%, 93%, 94%, 95%, 96%, 97%, 98%, 99%, or 100% identical) to the amino acid sequence of SEQ ID NO: 9.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 9. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 9. In some embodiments, the antigen binding domain contains a VHH domain having the amino acid sequence of SEQ ID NO: 9.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 10.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 10. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 10. In some embodiments, the antigen binding domain contains a VHH domain having the amino acid sequence of SEQ ID NO: 10.

In some embodiments, the antigen binding domain contains a VHH domain having an amino acid sequence that is at least 85% identical (e.g., at least 85%, 86%, 87%, 88%, 89%, 90, 91%, 92%, 93%, 94%, 95%, 96%, 97%, 98%, 99%, or 100% identical) to the amino acid sequence of SEQ ID NO: 11.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 11. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 11. In some embodiments, the antigen binding domain contains a VHH domain having the amino acid sequence of SEQ ID NO: 11.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 12.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 12. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 12. In some embodiments, the antigen binding domain contains a VHH domain having the amino acid sequence of SEQ ID NO: 12.

In some embodiments, the antigen binding domain contains a VHH domain having an amino acid sequence that is at least 85% identical (e.g., at least 85%, 86%, 87%, 88%, 89%, 90, 91%, 92%, 93%, 94%, 95%, 96%, 97%, 98%, 99%, or 100% identical) to the amino acid sequence of SEQ ID NO: 13.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 13. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 13. In some embodiments, the antigen binding domain contains a VHH domain having the amino acid sequence of SEQ ID NO: 13.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 14.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 14. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 14. In some embodiments, the antigen binding domain contains a VHH domain having the amino acid sequence of SEQ ID NO: 14.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 15.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 15. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 15. In some embodiments, the antigen binding domain contains a VHH domain having the amino acid sequence of SEQ ID NO: 15.

In some embodiments, the antigen binding domain contains a VHH domain having an amino acid sequence that is at least 85% identical (e.g., at least 85%, 86%, 87%, 88%, 89%, 90, 91%, 92%, 93%, 94%, 95%, 96%, 97%, 98%, 99%, or 100% identical) to the amino acid sequence of SEQ ID NO: 16.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 16. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 16. In some embodiments, the antigen binding domain contains a VHH domain having the amino acid sequence of SEQ ID NO: 16.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 17.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 17. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 17. In some embodiments, the antigen binding domain contains a VHH domain having the amino acid sequence of SEQ ID NO: 17.

In some embodiments, the antigen binding domain contains a VHH domain having an amino acid sequence that is at least 85% identical (e.g., at least 85%, 86%, 87%, 88%, 89%, 90, 91%, 92%, 93%, 94%, 95%, 96%, 97%, 98%, 99%, or 100% identical) to the amino acid sequence of SEQ ID NO: 18.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 18. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 18. In some embodiments, the antigen binding domain contains a VHH domain having the amino acid sequence of SEQ ID NO: 18.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 19.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 19. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 19. In some embodiments, the antigen binding domain contains a VHH domain having the amino acid sequence of SEQ ID NO: 19.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 80.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 80. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 80. In some embodiments, the antigen binding domain contains a VHH domain having the amino acid sequence of SEQ ID NO: 80.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 81.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 81. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 81. In some embodiments, the antigen binding domain contains a VHH domain having the amino acid sequence of SEQ ID NO: 81.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 82.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 82. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 82. In some embodiments, the antigen binding domain contains a VHH domain having the amino acid sequence of SEQ ID NO: 82.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 83.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 83. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 83. In some embodiments, the antigen binding domain contains a VHH domain having the amino acid sequence of SEQ ID NO: 83.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 84.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 84. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 84. In some embodiments, the antigen binding domain contains a VHH domain having the amino acid sequence of SEQ ID NO: 84.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 85.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 85. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 85. In some embodiments, the antigen binding domain contains a VHH domain having the amino acid sequence of SEQ ID NO: 85.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 86.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 86. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 86. In some embodiments, the antigen binding domain contains a VHH domain having the amino acid sequence of SEQ ID NO: 86.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 87.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 87. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 87. In some embodiments, the antigen binding domain contains a VHH domain having the amino acid sequence of SEQ ID NO: 87.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 88.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 88. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 88. In some embodiments, the antigen binding domain contains a VHH domain having the amino acid sequence of SEQ ID NO: 88.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 89.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 89. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 89. In some embodiments, the antigen binding domain contains a VHH domain having the amino acid sequence of SEQ ID NO: 89.

In some embodiments, the antigen binding domain contains a VHH domain having an amino acid sequence that is at least 85% identical (e.g., at least 85%, 86%, 87%, 88%, 89%, 90, 91%, 92%, 93%, 94%, 95%, 96%, 97%, 98%, 99%, or 100% identical) to the amino acid sequence of SEQ ID NO: 90.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 90. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 90. In some embodiments, the antigen binding domain contains a VHH domain having the amino acid sequence of SEQ ID NO: 90.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 91.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 91. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 91. In some embodiments, the antigen binding domain contains a VHH domain having the amino acid sequence of SEQ ID NO: 91.

In some embodiments, the antigen binding domain contains a VHH domain having an amino acid sequence that is at least 85% identical (e.g., at least 85%, 86%, 87%, 88%, 89%, 90, 91%, 92%, 93%, 94%, 95%, 96%, 97%, 98%, 99%, or 100% identical) to the amino acid sequence of SEQ ID NO: 92.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 92. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 92. In some embodiments, the antigen binding domain contains a VHH domain having the amino acid sequence of SEQ ID NO: 92.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 93.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 93. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 93. In some embodiments, the antigen binding domain contains a VHH domain having the amino acid sequence of SEQ ID NO: 93.

In some embodiments, the antigen binding domain contains a VHH domain having an amino acid sequence that is at least 85% identical (e.g., at least 85%, 86%, 87%, 88%, 89%, 90, 91%, 92%, 93%, 94%, 95%, 96%, 97%, 98%, 99%, or 100% identical) to the amino acid sequence of SEQ ID NO: 94.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 94. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 94. In some embodiments, the antigen binding domain contains a VHH domain having the amino acid sequence of SEQ ID NO: 94.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 95.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 95. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 95. In some embodiments, the antigen binding domain contains a VHH domain having the amino acid sequence of SEQ ID NO: 95.

In some embodiments, the antigen binding domain contains a VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 96.

In some embodiments, the antigen binding domain contains a VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 96. In some embodiments, the antigen binding domain contains a VHH domain having an amino acid sequence that is at least 95% identical (e.g., at least 95%, 96%, 97%, 98%, 99%, or 100% identical) to the amino acid sequence of SEQ ID NO: 96. In some embodiments, the antigen binding domain contains a VHH domain having the amino acid sequence of SEQ ID NO: 96.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 1

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 1. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 1. In some embodiments, the antigen binding domain contains a humanized VHH domain having the amino acid sequence of SEQ ID NO: 1.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 2.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 2. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 2. In some embodiments, the antigen binding domain contains a humanized VHH domain having the amino acid sequence of SEQ ID NO: 2.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 3.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 3. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 3. In some embodiments, the antigen binding domain contains a humanized VHH domain having the amino acid sequence of SEQ ID NO: 3.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 4.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 4. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 4. In some embodiments, the antigen binding domain contains a humanized VHH domain having the amino acid sequence of SEQ ID NO: 4.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 5.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 5. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 5. In some embodiments, the antigen binding domain contains a humanized VHH domain having the amino acid sequence of SEQ ID NO: 5.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 6.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 6. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 6. In some embodiments, the antigen binding domain contains a humanized VHH domain having the amino acid sequence of SEQ ID NO: 6.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 7.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 7. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 7. In some embodiments, the antigen binding domain contains a humanized VHH domain having the amino acid sequence of SEQ ID NO: 7.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 8.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 8. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 8. In some embodiments, the antigen binding domain contains a humanized VHH domain having the amino acid sequence of SEQ ID NO: 8.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 9.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 9. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 9. In some embodiments, the antigen binding domain contains a humanized VHH domain having the amino acid sequence of SEQ ID NO: 9.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 10.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 10. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 10. In some embodiments, the antigen binding domain contains a humanized VHH domain having the amino acid sequence of SEQ ID NO: 10.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 11.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 11. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 11. In some embodiments, the antigen binding domain contains a humanized VHH domain having the amino acid sequence of SEQ ID NO: 11.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 12.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 12. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 12. In some embodiments, the antigen binding domain contains a humanized VHH domain having the amino acid sequence of SEQ ID NO: 12.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 13.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 13. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 13. In some embodiments, the antigen binding domain contains a humanized VHH domain having the amino acid sequence of SEQ ID NO: 13.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 14.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 14. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 14. In some embodiments, the antigen binding domain contains a humanized VHH domain having the amino acid sequence of SEQ ID NO: 14.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 15.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 15. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 15. In some embodiments, the antigen binding domain contains a humanized VHH domain having the amino acid sequence of SEQ ID NO: 15.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 16.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 16. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 16. In some embodiments, the antigen binding domain contains a humanized VHH domain having the amino acid sequence of SEQ ID NO: 16.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 17.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 17. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 17. In some embodiments, the antigen binding domain contains a humanized VHH domain having the amino acid sequence of SEQ ID NO: 17.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 18.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 18. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 18. In some embodiments, the antigen binding domain contains a humanized VHH domain having the amino acid sequence of SEQ ID NO: 18.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 19.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 19. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 19. In some embodiments, the antigen binding domain contains a humanized VHH domain having the amino acid sequence of SEQ ID NO: 19.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 80.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 80. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 80. In some embodiments, the antigen binding domain contains a humanized VHH domain having the amino acid sequence of SEQ ID NO: 80.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 81.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 81. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 81. In some embodiments, the antigen binding domain contains a humanized VHH domain having the amino acid sequence of SEQ ID NO: 81.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 82.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 82. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 82. In some embodiments, the antigen binding domain contains a humanized VHH domain having the amino acid sequence of SEQ ID NO: 82.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 83.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 83. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 83. In some embodiments, the antigen binding domain contains a humanized VHH domain having the amino acid sequence of SEQ ID NO: 83.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 84.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 84. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 84. In some embodiments, the antigen binding domain contains a humanized VHH domain having the amino acid sequence of SEQ ID NO: 84.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 85.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 85. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 85. In some embodiments, the antigen binding domain contains a humanized VHH domain having the amino acid sequence of SEQ ID NO: 85.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 86.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 86. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 86. In some embodiments, the antigen binding domain contains a humanized VHH domain having the amino acid sequence of SEQ ID NO: 86.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 87.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 87. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 87. In some embodiments, the antigen binding domain contains a humanized VHH domain having the amino acid sequence of SEQ ID NO: 87.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 88.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 88. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 88. In some embodiments, the antigen binding domain contains a humanized VHH domain having the amino acid sequence of SEQ ID NO: 88.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 89.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 89. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 89. In some embodiments, the antigen binding domain contains a humanized VHH domain having the amino acid sequence of SEQ ID NO: 89.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 90.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 90. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 90. In some embodiments, the antigen binding domain contains a humanized VHH domain having the amino acid sequence of SEQ ID NO: 90.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 91.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 91. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 91. In some embodiments, the antigen binding domain contains a humanized VHH domain having the amino acid sequence of SEQ ID NO: 91.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 92.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 92. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 92. In some embodiments, the antigen binding domain contains a humanized VHH domain having the amino acid sequence of SEQ ID NO: 92.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 93.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 93. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 93. In some embodiments, the antigen binding domain contains a humanized VHH domain having the amino acid sequence of SEQ ID NO: 93.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 94.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 94. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 94. In some embodiments, the antigen binding domain contains a humanized VHH domain having the amino acid sequence of SEQ ID NO: 94.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 95.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 95. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 95. In some embodiments, the antigen binding domain contains a humanized VHH domain having the amino acid sequence of SEQ ID NO: 95.

In some embodiments, the antigen binding domain contains a humanized VHH domain having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 96.

In some embodiments, the antigen binding domain contains a humanized VHH domain having an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 96. In some embodiments, the antigen binding domain contains a humanized VHH domain having an amino acid sequence that is at least 95% identical (e.g., at least 95%, 96%, 97%, 98%, 99%, or 100% identical) to the amino acid sequence of SEQ ID NO: 96. In some embodiments, the antigen binding domain contains a humanized VHH domain having the amino acid sequence of SEQ ID NO: 96.

In some aspects, tolerable variations in the binding domain will be known to those of skill in the art, while maintaining binding to GPC3.

### Spacer Domain

In some aspects, the CAR comprises a spacer domain. In some aspects, the spacer domain is an oligopeptide or polypeptide that functions to link one or more of the antigen binding domain, transmembrane domain, costimulatory domain, and signaling domain to one or more of the antigen binding domain, transmembrane domain, costimulatory domain, and signaling domain. In some aspects, the spacer domain may be a short amino acid linker comprising 2, 3, 4, 5, 6, 7, 8, 9 or 10 amino acids in length. For example, a glycine-serine doublet. In some aspects, the spacer domain occurs between the intracellular domain and the transmembrane domain of the CAR. In some aspects the spacer domain occurs between the extracellular domain and the transmembrane domain. In some aspects, the spacer domain may comprise up to 300 amino acids, e.g., 10 to 100 amino acids, or 25 to 50 amino acids.

Non-limiting examples of linkers are disclosed in WO 2015/105522.

In some aspects, the spacer domain comprises an immunoglobulin Fc domain. In some aspects, the spacer domain comprises an IgG Fc domain. In some aspects, the spacer domain comprises an IgG4 Fc domain. In some aspects, the IgG4 Fc domain comprises one of the following:

```
  (SEQ ID NO: 171)
ESKYGPPCPPCPAPEFEGGPSVFLFPPKPKDTLMISRTPEVTCVVVDVS
QEDPEVQFNWYVDGVEVHNAKTKPREEQFQSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
  (SEQ ID NO: 172)
ESKYGPPCPPCPGGGSSGGGSGGQPREPQVYTLPPSQEEMTKNQVSLTC
LVKGFYPSDIAVEWESNGQPENNYKTTPPVLDSDGSFFLYSRLTVDKSR
WQEGNVFSCSVMHEALHNHYTQKSLSLSLGK;
  (SEQ ID NO: 173)
ESKYGPPCPSCPAPEFEGGPSVFLFPPKPKDTLMISRTPEVTCVVVDVS
QEDPEVQFNWYVDGVEVHQAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK;
  (SEQ ID NO: 174)
PKDTLMISRTPEVTCVVVDVSQEDPEVQFNWYVDGVEVHQAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSV
MHEALHNHYTQKSLSLSLGK;
or
  (SEQ ID NO: 175)
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLGK.
```

In some aspects, tolerable variations in the IgG4 Fc domain will be known to those of skill in the art. In some aspects, the IgG4 Fc domain comprises an amino acid sequence that has at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 171-175.

### Hinge Domain

In some aspects, the CAR comprises a hinge domain. The hinge domain of the CAR is a hydrophilic region which can be located between the antigen binding domain and the transmembrane domain. In some aspects, this domain may facilitate proper protein folding for the CAR, among other functions. The hinge domain is an optional component for the CAR. In some aspects, the transmembrane domain further comprises a hinge domain. The hinge domain may include a domain selected from Fc fragments of antibodies, hinge regions of antibodies, CH2 regions of antibodies, CH3 regions of antibodies, artificial hinge sequences or combinations thereof. Examples of hinge domains include, without limitation, a CD8a hinge, artificial hinges made of polypeptides which may be as small as, three glycines (Gly), as well as CH1 and CH3 domains of IgGs (such as human IgG4).

In some aspects, the CAR includes a hinge domain that connects the antigen binding domain with the transmembrane domain, which, in turn, connects to the intracellular domain. The hinge domain is preferably capable of supporting the antigen binding domain to recognize and bind to the target antigen on the target cells. In some aspects, the hinge domain is a flexible domain, thus allowing the antigen binding domain to have a structure to optimally recognize the specific structure and density of the target antigens on a cell such as tumor cell. The flexibility of the hinge domain permits the hinge region to adopt many different conformations.

In some embodiments, the hinge domain is an immunoglobulin heavy chain hinge region. In some embodiments, the hinge domain is a polypeptide derived from a receptor (e.g., a CD8-derived hinge region).

In some aspects, the hinge domain can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 an to about 30 aa, from about 30 aa to about 40 aa, or from about 40 aa to about 50 aa.

In some aspects, the hinge domain can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

For example, in some embodiments, a linker or hinge may comprise an IgG4 hinge or derivative thereof, an IgG2 hinge or derivative thereof, a CD28 hinge, or a CD8 hinge. Specific examples of linker and hinge domains are included in Table 2, but are not intended to be limiting.

TABLE 2
Examples of linker and hinge domain sequences

| SEQ | | |
|---|---|---|
| ID NO: | Description | Sequence |
| 193 | G-Linker-1 | GGGGSGGGGSGGGGS |
| 194 | G-Linker-2 | GGGGSGGGGS |
| 195 | G-Linker-3 | GGGGS |
| 196 | G-Linker-4 | GGGSSGGGSG |
| 197 | IgG4 hinge-1 | ESKYGPPCPSCP |
| 198 | IgG4 hinge-2 | ESKYGPPCPPCP |
| 199 | IgG4 hinge + | ESKYGPPCPPCPGGGSSGGGSG |
| | linker | |
| 200 | CD28 hinge | IEVMYPPPYLDNEKSNGTIIHVKGKHLCP |
| | | SPLFPGPSKP |
| 201 | CD8 hinge-1 | AKPTTTPAPRPPTPAPTIASQPLSLRPEA |
| | | CRPAAGGAVHTRGLDFACD |
| 202 | CD8 hinge-2 | TTTPAPRPPTPAPTIASQPLSLRPEACRP |
| | | AAGGAVHTRGLDFACD |
| 203 | IgG4-1 | ESKYGPPCPPCPGGGSSGGGSGGQPREPQ |
| | | VYTLPPSQEEMTKNQVSLTCLVKGFYPSD |
| | | IAVEWESNGQPENNYKTTPPVLDSDGSFF |
| | | LYSRLTVDKSRWQEGNVFSCSVMHEALHN |
| | | HYTQKSLSLSLGK |
| 204 | IgG4-2 | GQPREPQVYTLPPSQEEMTKNQVSLTCLV |
| | | KGFYPSDIAVEWESNGQPENNYKTTPPVL |
| | | DSDGSFFLYSRLTVDKSRWQEGNVFSCSV |
| | | MHEALHNHYTQKSLSLSLGK |

### Transmembrane Domain

In some aspects, CARs of the present disclosure may comprise a transmembrane domain that connects the antigen binding domain of the CAR to the intracellular domain of the CAR. The transmembrane domain of a subject CAR is a region that is capable of spanning the plasma membrane of a T cell. The transmembrane domain is for insertion into a cell membrane, e.g., a eukaryotic cell membrane. In some aspects, the transmembrane domain is interposed between the antigen binding domain and the intracellular domain of a CAR.

In some aspects, the transmembrane domain is naturally associated with one or more of the domains in the CAR. In some aspects, the transmembrane domain can be selected or modified by one or more amino acid substitutions to avoid binding of such domains to the transmembrane domains of the same or different surface membrane proteins, to minimize interactions with other members of the receptor complex. The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. In some aspects, the transmembrane domain is a transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1). TLR2, TLR3, TLR4, TLR5, TLR6, TLR7, TLR8, and TLR9. In some aspects, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some aspects, tolerable variations in the transmembrane domain will be known to those of skill in the art.

Specific examples of transmembrane domains are included in Table 3, but are not intended to be limiting.

TABLE 3
Examples of transmembrane domain sequences

| SEQ | | |
|---|---|---|
| ID NO: | Description | Sequence |
| 181 | CD3z | LCYLLDGILFIYGVILTALFL |
| 182 | CD28-1 | FWVLVVVGGVLACYSLLVTVAFIIFWV |
| 183 | CD28-2 | MFWVLVVVGGVLACYSLLVTVAFIIFWV |
| 184 | CD4 | MALIVLGGVAGLLLFIGLGIFF |
| 185 | CD8-1 | IYIWAPLAGTCGVLLLSLVIT |
| 186 | CD8-2 | IYIWAPLAGTCGVLLLSLVITLY |
| 187 | CD8-3 | IYIWAPLAGTCGVLLLSLVITLYC |
| 188 | CD8-4 | SALSNSIMYFSHFVPVFLPAKPTTTPAPRP |
| | | PTPAPTIASQPLSLRPEACRPAAGGAVHTR |
| | | GLDFACDIYIWAPLAGTCGVLLLSLVITLY |
| | | CNH |
| 189 | CD8-5 | MYFSHFVPVFLPAKPTTTPAPRPPTPAPTI |
| | | ASQPLSLRPEACRPAAGGAVHTRGLDFACD |
| | | IYIWAPLAGTCGVLLLSLVITLYCNH |
| 190 | 4-1BB | IISFFLALTSTALLFLLFFLTLRF |

### Costimulatory Domain

In some aspects, costimulatory signals are necessary to achieve robust CAR-T cell function (e.g., expansion, function, persistence, and anti-tumor activity). These can be provided by incorporating one or more costimulatory domains from one or more costimulatory molecules (e.g., T cell costimulatory molecules). In some aspects, costimulatory domains are selected from the costimulatory molecules of CD3, CD4, CD8, T cell receptor (TCR), CD27, CD28, 4-1BB (CD137), 0X40, CD30, CD40, PD-1, ICOS, lymphocyte function-associated antigen-1 (LFA-1), CD2, CD7, LIGHT, NKG2C, B7-H3, or any fragment thereof.

In some aspects, the costimulatory domain comprises a 4-1BB costimulatory domain. In some aspects, the 4-1BB costimulatory domain comprises

(SEQ ID NO: 176)
KRGRKKLLYIFKQPFMRPVQTTQEEDGCSCRFPEEEEGGCEL.

In some aspects, the costimulatory domain comprises a CD28 costimulatory domain. In some aspects, the CD28 costimulatory domain comprises

(SEQ ID NO: 205)
RSKRSRLLHSDYMNMTPRRPGPTRKHQYPY APPRDFAAYRS.

In some aspects, the costimulatory domain comprises an OX40 costimulatory domain. In some aspects, the OX40 costimulatory domain comprises

(SEQ ID NO: 206)
ALYLLRRDQRLPPDAHKPPGGGSFRTPIQEEQADAHSTLAKI.

In some aspects, tolerable variations in the costimulatory domain will be known to those of skill in the art. In some aspects, the costimulatory domain comprises an amino acid sequence that has at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to SEQ ID NO: 176, SEQ ID NO: 205, or SEQ ID NO: 206.

### Intracellular Signaling Domain

In some aspects, the CAR also includes an intracellular signaling domain. The terms “intracellular signaling domain” and “intracellular domain” are used interchangeably herein. The intracellular signaling domain of the CAR is responsible for activation of at least one of the effector functions of the T cell in which the CAR is expressed. The intracellular signaling domain transduces the effector function signal and directs the T cell to perform its specialized function, e.g., harming and/or destroying a target cell.

In some aspects, the intracellular signaling domain is the cytoplasmic portion of a surface receptor, co-stimulatory molecule, or any molecule that acts in concert to initiate signal transduction in a T cell, as well as any derivative or variant of these elements and any synthetic sequence that has the same functional capability.

In some aspects, the intracellular signaling domain is the z chain of the T cell receptor complex or any of its homologs, e.g., h chain, FcsRfy and b chains, MB 1 (IgA) chain, B29 (Ig) chain, etc., human CD3 ξ chain, CD3 polypeptides (A, d and e), syk family tyrosine kinases (Syk, ZAP 70, etc.), src family tyrosine kinases (Lck, Fyn, Lyn, etc.), and other molecules involved in T cell transduction, such as CD2, CD5 and CD28. In some aspects, the intracellular signaling domain may be human CD3 ξ chain, FcγRIII, FcsRI, cytoplasmic tails of Fc receptors, an immunoreceptor tyrosine-based activation motif (IT AM) bearing cytoplasmic receptors, and combinations thereof.

In some aspects the intracellular signaling domain includes a fragment or domain from one or more molecules or receptors including, but not limited to, TCR, CD3 zeta, CD3 gamma, CD3 delta, CD3 epsilon, CD86, common FcR gamma, FcR beta (Fc Epsilon Rib), CD79a, CD79b, Fcgamma Rlla, DAP 10, DAP 12, T cell receptor (TCR), CD8, CD27, CD28, 4-1BB (CD137), OX9, 0X40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS. ICAM-1, GITR, BAFFR. HVEM (LIGHTR), SLAMF7. NKp80 (KLRF1), CD127, CD 160, CD19, CD4, CD8alpha, CD8beta, IL2R beta, IL2R gamma, IL7R alpha, ITGA4, VLA1, CD49a, ITGA4, IA4, CD49D, ITGA6. VLA-6, CD49f, ITGAD, CD1 Id, ITGAE, CD 103, ITGAL, CD 11 a, LFA-1, ITGAM, CD lib, ITGAX, CD 1 lc, ITGB1, CD29, ITGB2, CD 18, LFA-1, ITGB7, TNFR2, TRANCE/RANKL, DNAM1 (CD226), SLAMF4 (CD244, 2B4), CD84, CD 96 (Tactile), CEACAM1, CRT AM, Ly9 (CD229), CD160 (BY55), PSGL1, CD 100 (SEMA4D), CD69, SLAMF6 (NTB-A, Lyl08), SLAM (SLAMF1, CD150, LPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, TLR10, TLRT1, TLR12, TLR13, other co-stimulatory molecules described herein, any derivative, variant, or fragment thereof, any synthetic sequence of a co stimulatory molecule that has the same functional capability, and any combination thereof.

In some aspects, the intracellular signaling domain is a CD3ξ signaling domain, such as

(SEQ ID NO: 177)
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR

In some embodiments, a CAR of the present disclosure may comprise the intracellular signaling domain of a TLR, including TLR1, TLR2, TLR3, TLR4, TLR5, TLR6, TLR7, TLR8, TLR9, TLR10, TLR11. TLR12, and TLR13. Switching between the various TLRs can alter the cytokine response of monocytic cells. The signaling domains of TLR4 and TLR9 are particularly useful for treating cancer, but for the purposes of the present disclosure, the chimeric receptor could alternatively comprise the signaling domain of TLR1, TLR2, TLR3, TLR5, TLR6, TLR7, TLR8, TLR10, TLR11, TLR12, or TLR13. Examples of TLR signaling domain amino acid sequences are shown in Table 4.

TABLE 4
Examples of TLR signaling domain amino
acid sequences.

| SEQ | | |
|---|---|---|
| ID | | |
| NO: | Description | Sequence |
| 191 | TLR4 | KFYFHLMLLAGCIKYGRGENIYDAFVIYSSQ |
| | | DEDWVRNELVKNLEEGVPPFQLCLHYRDFIP |
| | | GVAIAANIIHEGFHKSRKVIVVVSQHFIQSR |
| | | WCIFEYEIAQTWQFLSSRAGIIFIVLQKVEK |
| | | TLLRQQVELYRLLSRNTYLEWEDSVLGRHIF |
| | | WRRLRKALLDGKSWNPEGTVGTGCNWQEATS |
| | | I |
| 192 | TLR9 | EVQAAVPGLPSRVKCGSPGQLQGLSIFAQDL |
| | | RLCLDEALSWDCFALSLLAVALGLGVPMLHH |
| | | LCGWDLWYCFHLCLAWLPWRGRQSGRDEDAL |
| | | PYDAFVVFDKTQSAVADWVYNELRGQLEECR |
| | | GRWALRLCLEERDWLPGKTLFENLWASVYGS |
| | | RKTLFVLAHTDRVSGLLRASFLLAQQRLLED |
| | | RKDVVVLVILSPDGRRSRYVRLRQRLCRQSV |
| | | LLWPHQPSGQRSFWAQLGMALTRDNHHFYNR |
| | | NFCQGPTAE |

In some aspects, cells expressing the CAR is introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is a T cell.

When a CAR is expressed in vivo, it may temporarily comprise a leader sequence or GMCSFRa signal peptide, such as MLLLVTSLLLCELPHPAFLLIP (SEQ ID NO: 178), which is ultimately cleaved off of the mature CAR. In some instances, the CAR temporarily expresses the signal peptide, MALPVTALLLPLALLLHAARPD (SEQ ID NO: 243).

In some instances, a CAR of this disclosure is VHH3-CD8 hinge-CD28TM-CD28 ICD-4-1BB ICD-CD3z ICD and comprises the amino acid sequence:

(SEQ ID NO: 247)
QVQLVESGGGLVQPGGSLRLSCAASGFIFSSYAMSWVRQAPGKEPEWV
SSISGGGSSTYYADSLEGRFTISRDNSKNTLYLQMNSLRAEDTAVYYC
SKDPRFGEPPFDYWGQGTTVTVSSTTTPAPRPPTPAPTIASQPLSLRP
EACRPAAGGAVHTRGLDFACDFWVLVVVGGVLACYSLLVTVAFIIFWV
RSKRSRLLHSDYMNMTPRRPGPTRKHYQPYAPPRDFAAYRSKRGRKKL
LYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNE
LQKDKMAEAYSEIGMKGERRRGKGHDGLYQGLSTATKDTYDALHMQAL
PPR

In some instances, a CAR of this disclosure is VHH6-CD8 hinge-CD28TM-CD28 ICD-4-1BB ICD-CD3z ICD and comprises the amino acid sequence:

(SEQ ID NO: 248)
QVQLVQSGGGLVQPGGSLRLSCAASYFDFDSYEMSWVRQAPGKGLEWI
GSIYHSGSTYYNPSLKSRVTISRDNSKNTLYLQMNTLRAEDTATYYCA
RVNMDRFDYWGQGTLVTVSSTTTPAPRPPTPAPTIASQPLSLRPEACR
PAAGGAVHTRGLDFACDFWVLVVVGGVLACYSLLVTVAFIIFWVRSKR
SRLLHSDYMNMTPRRPGPTRKHYQPYAPPRDFAAYRSKRGRKKLLYIF
KQPFMRPVQTTQEEDGCSCRFPEEEEGGCELRVKFSRSADAPAYKQGQ
NQLYNELNLGRREEYDVLDKRRGRDPEMGGKPRRKNPQEGLYNELQKD
KMAEAYSEIGMKGERRRGKGHDGLYQGLSTATKDTYDALHMQALPPR

In some instances, a CAR of this disclosure is VHH29-CD8 hinge-CD28TM-CD28 ICD-4-1BB ICD-CD3z ICD and comprises the amino acid sequence:

(SEQ ID NO: 249)
QVQLVESGGGLVQPGGSLRLSCAASGFTFSSYAMSWVRQAPGKEPEWV
SSISGGGSSTYYADSLKGRFTISRDNSKNTLYLQMNSLRAEDTAVYYC
SRDPRFGEPPFDYWGQGTLVTVSSTTTPAPRPPTPAPTIASQPLSLRP
EACRPAAGGAVHTRGLDFACDFWVLVVVGGVLACYSLLVTVAFIIFWV
RSKRSRLLHSDYMNMTPRRPGPTRKHYQPYAPPRDFAAYRSKRGRKKL
LYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNE
LQKDKMAEAYSEIGMKGERRRGKGHDGLYQGLSTATKDTYDALHMQAL
PPR

The disclosure also features a nucleic acid encoding each one of the above-described exemplary CARs.

The present disclosure also provides genetically engineered cells which include and stably express a CAR of the present disclosure. In some aspects, the engineered cells express a dominant negative receptor and/or switch receptor, and/or bispecific antibody, and/or combinations thereof, of the present disclosure. In some aspects, the genetically engineered cells are genetically engineered T-lymphocytes (T cells), naive T cells (TN), or memory T cells (for example, central memory T cells (TCM) capable of giving rise to therapeutically relevant progeny. In some aspects, the genetically engineered cells are autologous cells. In some cases, the genetically engineered cells which include and stably express a CAR of the present disclosure are T cells. In other instances, the genetically engineered cells which include and stably express a CAR of the present disclosure are NK cells. Modified cells (e.g., comprising a subject CAR, dominant negative receptor and/or switch receptor, and/or expresses and secretes a bispecific antibody, and/or combinations thereof) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject CAR, dominant negative receptor and/or switch receptor, and/or bispecific antibody, and/or combinations thereof, of the present disclosure may be expanded ex vivo.

In some aspects, the disclosure is drawn to a population of modified T cells that express a CAR.

In some aspects, the population of cells that express the CAR further express a selection tag. In some aspects, the selection tag is a truncated CD19 sequence (CD19t) (e.g., CGDVEENPGPRMPPPRLLFFLLFLTPMEVRPEEPLVVKVEEGDNAVLQCLKGT SDGPTQQLTWSRESPLKPFLKLSLGLPGLGIHMRPLAIWLFIFNVSQQMGGFY LCQPGPPSEKAWQPGWTVNVEGSGELFRWNVSDLGGLGCGLKNRSSEGPSSP SGKLMSPKLYVWAKDRPEIWEGEPPCVPPRDSLNQSLSQDLTMAPGSTLWLS CGVPPDSVSRGPLSWTHVHPKGPKSLLSLELKDDRPARDMWVMETGLLLPR ATAQDAGKYYCHRGNLTMSFHLEITARPVLWHWLLRTGGWKVSAVTLAYLI FCLCSLVGILHLQRALVLRRKR; SEQ ID NO: 179). In some aspects, the selection tag is a truncated epidermal growth factor receptor sequence (EGFRt). In some aspects, the population of cells that express the CAR comprise a tag that allows for isolation and purification of CAR-expressing cells. Tags such as CD19t and EGFRt may be separated from the mature CAR sequence by a T2A skip sequence (e.g., LEGGGEGRGSLLT; SEQ ID NO: 180).

### Affinity of Antibodies or CARs of the Disclosure

### Thermodynamic Properties of Anti-GPC3 Antibodies, Antigen-Binding Fragments, or CARs

Antibodies or CARs of the disclosure may have an affinity for Glypican 3 of, for example, from 1 nM to 100 nM (e.g., from 10 nM to 90 nM, from 20 nM to 80 nM, from 30 nM to 70 nM, from 40 nM to 60 nM, or about 50 nM). In some embodiments, antibodies, antigen-binding fragments, or CARs of the disclosure have an affinity for Glypican 3 of from about 1 nM to about 100 nM. In some embodiments, antibodies, antigen-binding fragments, or CARs of the disclosure have an affinity for Glypican 3 of from about 1 nM to about 90 nM. In some embodiments, antibodies, antigen-binding fragments, or CARs of the disclosure have an affinity Glypican 3 of from about 1 nM to about 80 nM. In some embodiments, antibodies, antigen-binding fragments, or CARs of the disclosure have an affinity for Glypican 3 of from about 1 nM to 60 nM. In some embodiments, antibodies, antigen-binding fragments, or CARs of the disclosure have an affinity for Glypican 3 of from about 1 nM to 40 nM. In some embodiments, antibodies, antigen-binding fragments, or CARs of the disclosure have an affinity for Glypican 3 of from about 1 nM to 20 nM.

In some embodiments, antibodies, antigen-binding fragments, or CARs of the disclosure have an affinity for Glypican 3 of about 100 nM. In some embodiments, antibodies, antigen-binding fragments, or CARs of the disclosure have an affinity for Glypican 3 of about 95 nM. In some embodiments, antibodies, antigen-binding fragments, or CARs of the disclosure have an affinity for Glypican 3 of about 90 nM. In some embodiments, antibodies, antigen-binding fragments, or CARs of the disclosure have an affinity for Glypican 3 of about 85 nM. In some embodiments, antibodies, antigen-binding fragments, or CARs of the disclosure have an affinity for Glypican 3 of about 80 nM. In some embodiments, antibodies, antigen-binding fragments, or CARs of the disclosure have an affinity for Glypican 3 of about 75 nM. In some embodiments, antibodies, antigen-binding fragments, or CARs of the disclosure have an affinity for Glypican 3 of about 70 nM. In some embodiments, antibodies, antigen-binding fragments, or CARs of the disclosure have an affinity for Glypican 3 of about 65 nM. In some embodiments, antibodies, antigen-binding fragments, or CARs of the disclosure have an affinity for Glypican 3 of about 60 nM. In some embodiments, antibodies, antigen-binding fragments, or CARs of the disclosure have an affinity for Glypican 3 of about 55 nM. In some embodiments, antibodies, antigen-binding fragments, or CARs of the disclosure have an affinity for Glypican 3 of about 50 nM. In some embodiments, antibodies, antigen-binding fragments, or CARs of the disclosure have an affinity for Glypican 3 of about 45 nM. In some embodiments, antibodies, antigen-binding fragments, or CARs of the disclosure have an affinity for Glypican 3 of about 40 nM. In some embodiments, antibodies, antigen-binding fragments, or CARs of the disclosure have an affinity for Glypican 3 of about 35 nM. In some embodiments, antibodies, antigen-binding fragments, or CARs of the disclosure have an affinity for Glypican 3 of about 30 nM. In some embodiments, antibodies, antigen-binding fragments, or CARs of the disclosure have an affinity for Glypican 3 of about 25 nM. In some embodiments, antibodies, antigen-binding fragments, or CARs of the disclosure have an affinity for Glypican 3 of about 20 nM. In some embodiments, antibodies, antigen-binding fragments, or CARs of the disclosure have an affinity for Glypican 3 of about 15 nM. In some embodiments, antibodies, antigen-binding fragments, or CARs of the disclosure have an affinity for Glypican 3 of about 10 nM. In some embodiments, antibodies, antigen-binding fragments, or CARs of the disclosure have an affinity for Glypican 3 of about 5 nM. In some embodiments, antibodies, antigen-binding fragments, or CARs of the disclosure have an affinity for Glypican 3 of about 1 nM.

The specific binding of an antibody, antigen-binding fragments, or CAR described herein to Glypican 3 can be determined by any of a variety of established methods. The affinity can be represented quantitatively by various measurements, including the concentration of antibody, antigen-binding fragment, or CARs needed to achieve half-maximal activation of Glypican 3 in vitro or in vivo (EC₅₀) and the equilibrium constant (K_{D}) of the antibody-, antigen-binding fragment-, or CAR-Glypican 3 complex dissociation. The equilibrium constant, K_{D}, which describes the interaction of Glypican 3 with an antibody, antigen-binding fragment, or CAR described herein is the chemical equilibrium constant for the dissociation reaction of a Glypican 3-antibody, -antigen-binding fragment, or -CAR complex into solvent-separated Glypican 3 and antibody, antigen-binding fragment, or CAR molecules that do not interact with one another.

Antibodies, antigen-binding fragments, or CARs described herein include those that specifically bind to Glypican 3 with a K_{D} value of less than 100 nM (e.g., less than 95 nM, 90 nM, 85 nM, 80 nM, 75 nM, 70 nM. 65 nM. 60 nM. 55 nM. 50 nM, 45 nM, 40 nM, 35 nM, 30 nM 25 nM, 20 nM, 15 nM, 10 nM, 5 nM, 4 nM, 3 nM, 2 nM, or 1 nM). In some embodiments, the antibodies, antigen-binding fragments, or CARs described herein specifically bind to Glypican 3 with a K_{D} value of less than 10 nM (e.g., less than 9 nM, 8 nM, 7 nM, 6 nM. 5 nM, 4 nM, 3 nM, 2 nM, or 1 nM).

Antibodies, antigen-binding fragments, or CARs described herein can also be characterized by a variety of in vitro binding assays. Examples of experiments that can be used to determine the K_{D} or EC₅₀ of an anti-GPC3 antibody, antigen-binding fragment, or CAR include, e.g., surface plasmon resonance, isothermal titration calorimetry, fluorescence anisotropy, ELISA-based assays, gene expression assays, and protein expression assays, among others. ELISA represents a particularly useful method for analyzing antibody, antigen-binding fragment, or CAR activity, as such assays typically require minimal concentrations of binding domains (e.g., antibodies, antigen-binding fragments. CARs). A common signal that is analyzed in a typical ELISA assay is luminescence, which is typically the result of the activity of a peroxidase conjugated to a secondary antibody that specifically binds a primary antibody (e.g., an anti-GPC3 antibody, antigen-binding fragment described herein). Antibodies, antigen-binding fragments, or CARs described herein may bind Glypican 3 and fragments thereof. Antibodies, antigen-binding fragments, or CARs described herein may additionally bind isolated peptides derived from Glypican 3 that structurally pre-organize various residues in a manner that simulates the conformation of the above fragments in the native protein. In a direct ELISA experiment, this binding can be quantified, e.g., by analyzing the luminescence that occurs upon incubation of an HRP substrate (e.g., 2,2′-azino-di-3-ethylbenzthiazoline sulfonate) with an antigen-antibody, antigen-antigen-binding fragment, or antigen-CAR complex bound to a HRP-conjugated secondary antibody.

### Kinetic Properties of Anti-GPC3 Antibodies, Antigen-Binding Fragments, or CARs

In addition to the thermodynamic parameters of a Glypican 3-antibody, antigen-binding fragment, or -CAR interaction, it is also possible to quantitatively characterize the kinetic association and dissociation of an antibody, antigen-binding fragment, or CAR described herein with Glypican 3. This can be done, e.g., by monitoring the rate of antibody-, antigen-binding fragment-, or CAR-antigen complex formation according to established procedures. For example, one can use surface plasmon resonance (SPR) to determine the rate constants for the formation (kₒₙ) and dissociation (k_{off}) of an antibody-, antigen-binding fragment-, or CAR-Glypican 3 complex. These data also enable calculation of the equilibrium constant of (K_{D}) of antibody-, antigen-binding fragment-, or CAR-Glypican 3 complex dissociation, since the equilibrium constant of this unimolecular dissociation can be expressed as the ratio of the k_{off} to kₒₙ values. SPR is a technique that is particularly advantageous for determining kinetic and thermodynamic parameters of antigen-antibody, -antigen-binding fragment, or -CAR interactions since the experiment does not require that one component be modified by attachment of a chemical label. Rather, the antigen is typically immobilized on a solid metallic surface which is treated in pulses with solutions of increasing concentrations of antibody, antigen-binding fragment, or CAR. Antibody-, antigen-binding fragment-, or CAR-antigen binding induces distortion in the angle of reflection of incident light at the metallic surface, and this change in refractive index over time as antibody, antigen-binding fragment, or CAR is introduced to the system can be fit to established regression models in order to calculate the association and dissociation rate constants of an antibody- or antigen-binding-fragment- or CAR-antigen interaction.

Antibodies, antigen-binding fragments, or CARs described herein may exhibit high kₒₙ and low k_{off} values upon interaction with Glypican 3. For example, antibodies, antigen-binding fragments, or CARs described herein may exhibit kₒₙ values in the presence of Glypican 3 of greater than 10⁴ M⁻¹s⁻¹ (e.g., 1.0×10⁴ M⁻¹s⁻¹, 1.5×10⁴ M⁻¹s⁻¹, 2.0×10⁴ M⁻¹s⁻¹, 2.5×10⁴ M⁻¹s⁻¹, 3.0×10⁴ M−s⁻¹, 3.5×10⁴ M⁻¹s⁻¹, 4.0×10⁴ M⁻¹s⁻¹, 4.5×10⁴ M⁻¹s⁻¹, 5.0×10⁴ M−s⁻¹, 5.5×10⁴ M⁻¹s⁻¹, 6.0×10⁴ M⁻¹s⁻¹, 6.5×10⁴ M⁻¹s⁻¹, 7.0×10⁴ M⁻¹s⁻¹. 7.5×10⁴ M⁻¹s⁻¹, 8.0×10⁴ M⁻¹s⁻¹, 8.5×10⁴ M⁻¹s⁻¹. 9.0×10⁴ M⁻¹s⁻¹, 9.5×10⁴ M−s⁻¹, 1.0×10⁵ M⁻¹s⁻¹, 1.5×10⁵ M⁻¹, 2.0×10⁵ M⁻¹s⁻¹, 2.5×10⁵ M⁻¹s⁻¹, 3.0×10⁵ M⁻¹s⁻¹, 3.5×10⁵ M⁻¹s, 4.0×10⁵ M⁻¹s⁻¹, 4.5×10⁵M⁻¹s, 5.0×10⁵ M⁻¹s⁻¹, 5.5×10⁵ M⁻¹s⁻¹, 6.0×10⁵ M-'s⁻¹, 6.5×10⁵ M⁻¹s⁻¹, 7.0×10⁵ M⁻¹s⁻¹, 7.5×10⁵ M⁻¹s⁻¹, 8.0×10⁵ M−s⁻¹, 8.5×10⁵ M^{-s}⁻¹, 9.0×10⁵ M⁻¹s⁻¹, 9.5×10⁵ M⁻¹s⁻¹, or 1.0×10⁶ M⁻¹s⁻¹). Antibodies, antigen-binding fragments, or CARs described herein may exhibit low k_{off} values when bound to Glypican 3. For instance, antibodies, antigen-binding fragments, or CARs described herein may exhibit k_{off} values of less than 10⁻³ s⁻¹ when complexed to Glypican 3 (e.g., 1.0×10⁻³ s⁻¹, 9.5×10⁻⁴ s⁻¹, 9.0×10⁻⁴ s⁻¹. 8.5×10⁻⁴ s⁻¹, 8.0×10⁻⁴ s⁻¹, 7.5×10⁻⁴ s⁻¹, 7.0×10⁻⁴s⁻¹, 6.5×10⁻⁴s⁻¹, 6.0×10⁻⁴s⁻¹, 5.5×10⁻⁴ s⁻¹, 5.0×10⁻⁴ s⁻¹, 4.5×10⁻⁴ s⁻¹, 4.0×10⁻⁴ s⁻¹, 3.5×10⁻⁴ s⁻¹, 3.0×10⁻⁴ s⁻¹, 2.5×10⁻⁴ s⁻¹, 2.0×10⁻⁴ s⁻¹, 1.5×10⁻⁴ s⁻¹, 1.0×10⁻⁴ s⁻¹, 9.5×10⁻⁵ s⁻¹, 9.0×10⁻⁵ s⁻¹, 8.5×10⁻⁵ s⁻¹, 8.0×10⁻⁵ s⁻¹, 7.5×10⁻⁵ s⁻¹, 7.0×10⁻⁵ s⁻¹, 6.5×10⁻⁵ s⁻¹, 6.0×10⁻⁵ s⁻¹, 5.5×10⁻⁵ s⁻¹ 5.0×10⁻⁵ s⁻¹, 4.5×10⁻⁵ s⁻¹, 4.0×10⁻⁵ s⁻¹, 3.5×10⁻⁵ s⁻¹, 3.0×10⁻⁵ s⁻¹, 2.5×10⁻⁵ s⁻¹, 2.0×10⁻⁵ s⁻¹, 1.5×10⁻⁵ s⁻¹, or 1.0×10⁻⁵ s⁻¹).

### Methods for Humanization

Antibodies, antigen-binding fragments, or CARs described herein can include fully human, humanized, primatized, and chimeric antibodies that contain one or more of the CDR sequences shown in Table 9, below. As an example, one strategy that can be used to design humanized antibodies, antigen-binding fragments, or CARs described herein is to align the sequences of the V_{H} and/or V_{L} of an antibody, antigen-binding fragment, or CAR (e.g., of the present disclosure) with the V_{H} and/or V_{L} of a consensus human antibody. Consensus human antibody heavy chain and light chain sequences are known in the art (see, e.g., the “VBASE” human germline sequence database; see also Kabat, et al., Sequences of Proteins of Immunological Interest. Fifth Edition, U.S. Department of Health and Human Services, NIH Publication No. 91-3242 (1991); Tomlinson et al., J. Mol. Biol. 227:776-98 (1992); and Cox et al., Eur. J. Immunol. 24:827-836 (1994); the disclosure of which is incorporated herein by reference). In this way, the variable domain framework residues and CDRs can be identified by sequence alignment (see. Kabat, supra). One can then substitute, for example, one or more of the CDRs of the consensus human antibody with the corresponding CDR(s) of an antibody or antigen-binding fragment or CAR of the disclosure, thereby producing a humanized antibody, antigen-binding fragment, or CAR.

Similarly, this strategy can also be used to produce primatized anti-GPC3 antibodies, antigen-binding fragments, or CARs, as one can substitute, for example, one or more, or all, of the CDRs of a primate antibody consensus sequence with, for example, one or more, or all, of the CDRs of an antibody, antigen-binding fragment, or CAR of the disclosure. Consensus primate antibody sequences known in the art (see. e.g., U.S. Pat. Nos. 5,658,570; 5,681,722; and 5,693,780; the disclosures of each of which are incorporated herein by reference).

In some embodiments, it may be desirable to import particular framework residues in addition to CDR sequences from an anti-GPC3 antibody, antigen-binding fragment, or CAR into the V_{H} and/or V_{L} of a human antibody. For instance, U.S. Pat. No. 6,054,297 identifies several instances when it may be advantageous to retain certain framework residues from a particular antibody heavy chain or light chain variable region in the resulting humanized antibody, antigen-binding fragment, or CAR. In some embodiments, framework residues may engage in non-covalent interactions with the antigen and thus contribute to the affinity of the antibody, antigen-binding fragment, or CAR for the target antigen. In some embodiments, individual framework residues may modulate the conformation of a CDR, and thus indirectly influence the interaction of the antibody, antigen-binding domain, or CAR with the antigen. Certain framework residues may form the interface between V_{H} and V_{L} domains, and may therefore contribute to the global antibody, antigen-binding domain, or CAR structure. In some cases, framework residues may constitute functional glycosylation sites (e.g., Asn-X-Ser/Thr) which may dictate antibody, antigen-binding domain, or CAR structure and antigen affinity upon attachment to carbohydrate moieties. In cases such as those described above, it may be beneficial to retain certain framework residues of an anti-GPC3 antibody, antigen-binding fragment, or CAR in. e.g., a humanized or primatized antibody or antigen-binding fragment or CAR thereof, as various framework residues may promote high epitope affinity and improved biochemical activity of the antibody or antigen-binding fragment or CAR thereof.

Antibodies described herein also include antibody fragments, Fab domains, F(ab′) molecules, F(ab′)₂ molecules, single-chain variable fragments (scFvs), tandem scFv fragments, diabodies, triabodies, dual variable domain immunoglobulins, multi-specific antibodies, bispecific antibodies, and heterospecific antibodies that contain one or more of the CDRs in Table 9, below, or a CDR having at least 85% sequence identity thereto (e.g., at least 86%, 87%, 88%, 89%, 90%, 91%, 92%, 93%, 94%, 95%, 96%, 97%, 98%, 99%, or 100% sequence identity thereto). These molecules can be expressed recombinantly, e.g., by incorporating polynucleotides encoding these proteins into expression vectors for transfection in a eukaryotic or prokaryotic cell using techniques described herein or known in the art, or synthesized chemically, e.g., by solid phase peptide synthesis methods described herein or known in the art.

Antibodies described herein additionally include antibody-like scaffolds that contain, for example, one or more of the CDRs shown in Table 9, below. Examples of antibody-like scaffolds include proteins that contain a tenth fibronectin type III domain (¹⁰Fn3), which contains BC, DE, and FG structural loops analogous to canonical antibodies. The tertiary structure of the ¹⁰Fn3 domain resembles that of the variable region of the IgG heavy chain, and one of skill in the art can graft, e.g., one or more of the CDR sequences shown in Table 9, below, onto the fibronectin scaffold by replacing residues of the BC, DE, and FG loops of 1° fn3 with residues of the corresponding CDR sequence. This can be achieved by recombinant expression of a modified ¹⁰Fn3 domain in a prokaryotic or eukaryotic cell (e.g., using the vectors and techniques described herein). Examples of using the ¹⁰Fn3 domain as an antibody-like scaffold for the grafting of CDRs from antibodies onto the BC, DE, and FG structural loops are reported in WO 2000/034784, WO 2009/142773, WO 2012/088006, and U.S. Pat. No. 8,278,419; the disclosures of each of which are incorporated herein by reference.

### Nucleic Acids and Expression Systems

Anti-GPC3 antibodies, antigen-binding fragments, or CARs described herein can be prepared by any of a variety of established techniques. For instance, an anti-GPC3 antibody or antigen-binding fragment described herein can be prepared by recombinant expression of immunoglobulin light and heavy chain genes in a host cell. To express an antibody or antigen-binding fragment or CAR recombinantly, a host cell can be transfected with one or more recombinant expression vectors carrying DNA fragments encoding the desired antibody chain(s), antigen-binding fragments, and/or additional CAR domains (e.g., transmembrane domains, hinge domains). For example, the light and/or heavy chains of an antibody or an antigen-binding fragment can be expressed in the host cell and, optionally, secreted into the medium in which the host cells are cultured, from which medium the antibodies can be recovered. Standard recombinant DNA methodologies are used to obtain antibody heavy chain genes, light chain genes, and CAR domains and to incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N.Y., 1989), Current Protocols in Molecular Biology (Ausubel et al., eds., Greene Publishing Associates (1989)), and in U.S. Pat. No. 4,816,397; the disclosures of each of which are incorporated herein by reference.

### Vectors for Expression

Viral genomes provide a rich source of vectors that can be used for the efficient delivery of exogenous genes into the genome of a cell (e.g., a eukaryotic or prokaryotic cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., Measles and Sendai), positive strand RNA viruses, such as picomavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments or CARs described herein include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses. D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030); the disclosures of each of which are incorporated herein by reference.

Non-viral vectors, such as plasmids, are also well known in the art and include, but are not limited to prokaryotic and eukaryotic vectors (e.g., yeast- and bacteria-based plasmids), as well as plasmids for expression in mammalian cells. Methods of introducing the vectors into a host cell and isolating and purifying the expressed protein are also well known in the art (e.g., Molecular Cloning; A Laboratory Manual, Second Edition (Sambrook. Fritsch and Maniatis (eds), Cold Spring Harbor, N.Y., 1989)).

### Genome Editing Techniques

In addition to viral vectors, a variety of additional methods have been developed for the incorporation of genes, e.g., those encoding antibody light and heavy chains, single-domain antibodies, single-chain variable fragments (scFvs), tandem scFvs, Fab domains, F(ab′)₂ domains, diabodies, and triabodies, among others, into the genomes of target cells for antibody, antigen-binding fragment, and/or CAR expression. One such method that can be used for incorporating polynucleotides encoding anti-GPC3 antibodies, antigen-binding fragments, or CARs into prokaryotic or eukaryotic cells includes the use of transposons. Transposons are polynucleotides that encode transposase enzymes and contain a polynucleotide sequence or gene of interest flanked by excision sites at the 5′ and 3′ positions. Once a transposon has been delivered into a cell, expression of the transposase gene commences and results in active enzymes that cleave the gene of interest from the transposon. This activity is mediated by the site-specific recognition of transposon excision sites by the transposase. In some embodiments, these excision sites may be terminal repeats or inverted terminal repeats. Once excised from the transposon, the gene of interest can be integrated into the genome of a prokaryotic or eukaryotic cell by transposase-catalyzed cleavage of similar excision sites that exist within nuclear genome of the cell. This allows the gene encoding an anti-GPC3 antibody or antigen-binding fragment or CAR described herein to be inserted into the cleaved nuclear DNA at the excision sites, and subsequent ligation of the phosphodiester bonds that join the gene of interest to the DNA of the prokaryotic or eukaryotic cell genome completes the incorporation process. In some embodiments, the transposon is a retrotransposon, such that the gene encoding the antibody, antigen-binding fragment, or CAR is first transcribed to an RNA product and then reverse-transcribed to DNA before incorporation in the prokaryotic or eukaryotic cell genome. Examples of transposon systems include the piggybac transposon (described in detail in WO 2010/085699) and the sleeping beauty transposon (described in detail in US20050112764); the disclosures of each of which are incorporated herein by reference.

Another useful method for the integration of nucleic acid molecules encoding anti-GPC3 antibodies, antigen-binding fragments, or CARs into the genome of a prokaryotic or eukaryotic cell is the clustered regularly interspaced short palindromic repeats (CRISPR)/Cas system, which is a system that originally evolved as an adaptive defense mechanism in bacteria and archaea against infection by viruses. The CRISPR/Cas system consists of palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can theoretically design a CRISPR/Cas system to cleave any target DNA molecule of interest. This technique has been exploited in order to edit eukaryotic genomes (Hwang et al., Nat. Biotech., 31:227-229 (2013)) and can be used as an efficient means of site-specifically editing eukaryotic or prokaryotic genomes in order to cleave DNA prior to the incorporation of a polynucleotide encoding an anti-GPC3 antibody, antigen-binding fragment, or CAR described herein. The use of CRISPR/Cas to modulate gene expression has been described in U.S. Pat. No. 8,697,359, the disclosure of which is incorporated herein by reference.

Alternative methods for site-specifically cleaving genomic DNA prior to the incorporation of a polynucleotide encoding an anti-GPC3 antibody, antigen-binding fragment, or CAR described herein include the use of zinc finger nucleases and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. Zinc finger nucleases and TALENs for use in genome editing applications are described in Urnov et al. Nat. Rev. Genet., 11:636-646 (2010); and in Joung et al., Nat. Rev. Mol. Cell. Bio. 14:49-55 (2013); incorporated herein by reference. Additional genome editing techniques that can be used to incorporate polynucleotides encoding antibodies, antigen-binding fragments, or CARs described herein into the genome of a prokaryotic or eukaryotic cell include the use of ARCUS™ meganucleases that can be rationally designed so as to site-specifically cleave genomic DNA. The use of these enzymes for the incorporation of polynucleotides encoding anti-GPC3 antibodies, antigen-binding fragments, or CARs described herein into the genome of a prokaryotic or eukaryotic cell is particularly advantageous in view of the structure-activity relationships that have been established for such enzymes. Single-chain meganucleases can thus be modified at certain amino acid positions in order to create nucleases that selectively cleave DNA at desired locations. These single-chain nucleases have been described extensively, e.g., in U.S. Pat. Nos. 8,021,867 and 8,445,251; the disclosures of each of which are incorporated herein by reference.

### Polynucleotide Sequence Elements

To express an anti-GPC3 antibodies, antigen-binding fragments, or CARs described herein, polynucleotides encoding partial or full-length light and heavy chains, e.g., polynucleotides that encode a one or more of the CDR sequences of an antibody, antigen-binding fragment, or CAR described herein, can be inserted into expression vectors such that the genes are operatively linked to transcriptional and translational control sequences. The expression vector and expression control sequences are chosen to be compatible with the expression host cell used. Polynucleotides encoding the light chain gene and the heavy chain of an anti-GPC3 antibody, antigen-binding fragment, or CAR can be inserted into separate vectors, or, optionally, both polynucleotides can be incorporated into the same expression vector using established techniques described herein or known in the art.

In addition to polynucleotides encoding the heavy and light chains of an antibody, or a polynucleotide encoding a single-chain antibody, an antibody fragment, such as a scFv molecule, or a construct described herein, or a CAR, the recombinant expression vectors described herein may carry regulatory sequences that control the expression of the antibody chain genes or CAR domains in a host cell. The design of the expression vector, including the selection of regulatory sequences, may depend on such factors as the choice of the host cell to be transformed or the level of expression of protein desired. For instance, suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Viral regulatory elements, and sequences thereof, are described in detail, for instance, in U.S. Pat. Nos. 5,168,062, 4,510,245, and 4,968,615, the disclosures of each of which are incorporated herein by reference.

In addition to, for example, the antibody chain genes and regulatory sequences, the recombinant expression vectors described herein can carry additional sequences, such as sequences that regulate replication of the vector in host cells (e.g., origins of replication) and selectable marker genes. A selectable marker gene facilitates selection of host cells into which the vector has been introduced (see, e.g., U.S. Pat. Nos. 4,399,216, 4,634,665 and 5,179,017). For example, typically the selectable marker gene confers resistance to cytotoxic drugs, such as G418, puromycin, blasticidin, hygromycin or methotrexate, to a host cell into which the vector has been introduced. Suitable selectable marker genes include the dihydrofolate reductase (DHFR) gene (for use in DHFR″ host cells with methotrexate selection/amplification) and the neo gene (for G418 selection). In order to express the light and heavy chains of an anti-GPC3 antibody, anti-GPC3 antibody fragment, or an anti-GPC3 CAR, the expression vector(s) containing polynucleotides encoding the heavy and light chains can be transfected into a host cell by standard techniques.

### Host Cells for Expression of Anti-GPC3 Antibodies, Antigen-Binding Fragments, or CARs

It is possible to express the antibodies, antigen-binding fragments, or CARs described herein in either prokaryotic or eukaryotic host cells. In some embodiments, expression of antibodies, antigen-binding fragments, or CARs is performed in eukaryotic cells, e.g., mammalian host cells, for high secretion of a properly folded and immunologically active antibody or antigen-binding fragments. Examples of mammalian host cells for expressing the recombinant antibodies, antigen-binding fragments, or CARs described herein include Chinese Hamster Ovary (CHO cells) (including DHFR CHO cells, described in Urlaub and Chasin (1980, Proc. Natl. Acad. Sci. USA 77:4216-4220), used with a DHFR selectable marker, e.g., as described in Kaufman and Sharp (1982, Mol. Biol. 159:601-621), NSO mveloma cells, COS cells, 293 cells, and SP2/0 cells. Additional cell types that may be useful for the expression of antibodies, antigen-binding fragments, or CARs include bacterial cells, such as BL-21(DE3) E. Coli cells, which can be transformed with vectors containing foreign DNA according to established protocols. Additional eukaryotic cells that may be useful for expression of antibodies, antigen-binding fragments, or CARs include yeast cells, such as auxotrophic strains of S. cerevisiae, which can be transformed and selectively grown in incomplete media according to established procedures known in the art. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown.

Antibodies, antigen-binding fragments, or CARs can be recovered from the culture medium using standard protein purification methods. Host cells can also be used to produce portions of intact antibodies, such as Fab fragments or scFv molecules. Also included herein are methods in which the above procedure is varied according to established protocols known in the art. For example, in some embodiments, it may be desirable to transfect a host cell with DNA encoding only the heavy chain of an anti-GPC3 antibody described herein in order to produce an antigen-binding fragment of the antibody.

Once an anti-GPC3 antibody or antigen-binding fragment described herein has been produced by recombinant expression, it can be purified by any method known in the art, such as a method useful for purification of an immunoglobulin molecule, for example, by chromatography (e.g., ion exchange, affinity, particularly by affinity for Glypican 3 after Protein A or Protein G selection, and sizing column chromatography), centrifugation, differential solubility, or by any other standard technique for the purification of proteins. Further, the anti-GPC3 antibodies described herein or fragments thereof can be fused to heterologous polypeptide sequences described herein or otherwise known in the art to facilitate purification or to produce therapeutic conjugates.

Once isolated, an anti-GPC3 single-chain antibody can, if desired, be further purified, e.g., by high performance liquid chromatography (see. e.g., Fisher, Laboratory Techniques in Biochemistry and Molecular Biology (Work and Burdon, eds., Elsevier, 1980); incorporated herein by reference), or by gel filtration chromatography, such as on a Superdex™ 75 column (Pharmacia Biotech AB, Uppsala, Sweden).

### Half-Life Extension of Anti-GPC3 Antibodies, Antigen-Binding Fragments, or CARs

In some embodiments, an anti-GPC3 antibody, antigen-binding fragment, or CAR of the disclosure is conjugated to a second molecule, e g., to extend the half-life of the anti-GPC3 antibody, antigen-binding fragment, or CAR in vivo. Such molecules that can extend half-life of the anti-GPC3 antibody, antigen binding fragment, or CAR are described below, and include polyethylene glycol (PEG), among others. Anti-GPC3 antibodies and fragments thereof, and in some instances CARs, can be conjugated to these half-life extending molecules at, e.g., the N-terminus or C-terminus of a light and/or heavy chain of the antibody using any one of a variety of conjugation strategies known in the art. Examples of pairs of reactive functional groups that can be used to covalently tether an anti-GPC3 antibody or fragment thereof to a half-life extending or other molecule include, without limitation, thiol pairs, carboxylic acids and amino groups, ketones and amino groups, aldehydes and amino groups, thiols and alpha,beta-unsaturated moieties (such as maleimides or dehydroalanine), thiols and alpha-halo amides, carboxylic acids and hydrazides, aldehydes and hydrazides, and ketones and hydrazides.

Anti-GPC3 antibodies can be conjugated to various molecules for the purpose of improving the half-life, solubility, and stability of the protein in aqueous solution. Examples of such molecules include polyethylene glycol (PEG), murine serum albumin (MSA), bovine serum albumin (BSA), and human serum albumin (HSA), among others. For instance, one can conjugate an anti-GPC3 antibody or antigen-binding fragment to carbohydrate moieties in order to evade detection of the antibody antigen-binding fragment by the immune system of the patient receiving treatment. This process of hyperglycosylation reduces the immunogenicity of therapeutic proteins by sterically inhibiting the interaction of the protein with B cell receptors in circulation. Additionally, anti-GPC3 antibodies, antigen-binding fragments, or CARs can be conjugated to molecules that prevent clearance from human serum and improve the pharmacokinetic profile of the antibodies, antigen-binding fragments, or CARs.

Serum albumin is a globular protein that is the most abundant blood protein in mammals. Serum albumin is produced in the liver and constitutes about half of the blood serum proteins. It is monomeric and soluble in the blood. Some of the most crucial functions of serum albumin include transporting hormones, fatty acids, and other proteins in the body, buffering pH, and maintaining osmotic pressure needed for proper distribution of bodily fluids between blood vessels and body tissues. In some embodiments, serum albumin is MSA or HSA. In some embodiments, MSA or HSA is joined to the N- or C-terminus of an antibody or antigen-binding fragment of the disclosure described herein to increase the serum half-life of the antibody or antigen-binding fragment. MSA or HSA can be joined, either directly or through a linker, to the N- or C-terminus of an antibody or antigen-binding fragment of the disclosure. In some embodiments, an antibody or antigen-binding fragment described herein is fused to the N- or C-terminus of a serum albumin through genetic or chemical means, e.g., chemical conjugation. If desired, a linker (e.g., a spacer) can be inserted between the antibody or antigen-binding fragment and the serum albumin.

In some embodiments, the MSA has the amino acid sequence of UniProt ID NO: Q546G4 (SEQ ID NO: 169), or an amino acid sequence that is at least 85% identical (e.g., at least 86%, 87%, 88%, 89%, 90%, 91%, 92%, 93%, 94%, 95%, 96%, 97%, 98%, or 99% identical) to SEQ ID NO: 169, shown below:

MKWVTFLLLLFVSGSAFSRGVFRREAHKSEIAHRYNDLGEQHFKGLV
LIAFSQYLQKCSYDEHAKLVQEVTDFAKTCVADESAANCDKSLHTLF
GDKLCAIPNLRENYGELADCCTKQEPERNECFLQHKDDNPSLPPFERP
EAEAMCTSFKENPTTFMGHYLHEVARRHPYFYAPELLYYAEQYNEILT
QCCAEADKESCLTPKLDGVKEKALVSSVRQRMKCSSMQKFGERAFK
AWAVARLSQTFPNADFAEITKLATDLTKVNKECCHGDLLECADDRAE
LAKYMCENQATISSKLQTCCDKPLLKKAHCLSEVEHDTMPADLPAIA
ADFVEDQEVCKNYAEAKDVFLGTFLYEYSRRHPDYSVSLLLRLAKKY
EATLEKCCAEANPPACYGTVLAEFQPLVEEPKNLVKTNCDLYEKLGE
YGFQNAILVRYTQKAPQVSTPTLVEAARNLGRVGTKCCTLPEDQRLPC
VEDYLSAILNRVCLLHEKTPVSEHVTKCCSGSLVERRPCFSALTVDETY
VPKEFKAETFTFHSDICTLPEKEKQIKKQTALAELVKHKPKATAEQLKT
VMDDFAQFLDTCCKAADKDTCFSTEGPNLVTRCKDALA

In some embodiments, the HSA has the amino acid sequence of UniProt ID NO: P02768 (SEQ ID NO: 170), or an amino acid sequence that is at least 85% identical (e.g., at least 86%, 87%, 88%, 89%, 90%, 91%, 92%, 93%, 94%, 95%, 96%, 97%, 98%, or 99% identical) to SEQ ID NO: 170, shown below:

MKWVTFISLLFLFSSAYSRGVFRRDAHKSEVAHRFKDLGEENFKALVL
IAFAQYLQQCPFEDHVKLVNEVTEFAKTCVADESAENCDKSLHTLFGD
KLCTVATLRETYGEMADCCAKQEPERNECFLQHKDDNPNLPRLVRPE
VDVMCTAFHDNEETFLKKYLYEIARRHPYFYAPELLFFAKRYKAAFTE
CCQAADKAACLLPKLDELRDEGKASSAKQRLKCASLQKFGERAFKA
WAVARLSQRFPKAEFAEVSKLVTDLTKVHTECCHGDLLECADDRADL
AKYICENQDSISSKLKECCEKPLLEKSHCIAEVENDEMPADLPSLAADF
VESKDVCKNYAEAKDVFLGMFLYEYARRHPDYSVVLLLRLAKTYETT
LEKCCAAADPHECYAKVFDEFKPLVEEPQNLIKQNCELFEQLGEYKFQ
NALLVRYTKKVPQVSTPTLVEVSRNLGKVGSKCCKHPEAKRMPCAED
YLSVVLNQLCVLHEKTPVSDRVTKCCTESLVNRRPCFSALEVDETYVP
KEFNAETFTFHADICTLSEKERQIKKQTALVELVKHKPKATKEQLKAV
MDDFAAFVEKCCKADDKETCFAEEGKKLVAASQAALGL

Anti-GPC3 antibodies, antigen-binding fragments, or CARs can be covalently appended directly to a half-life extending or other molecule by chemical conjugation as described. Alternatively, fusion proteins containing anti-GPC3 antibodies, antigen-binding fragments, or CARs can be expressed recombinantly from a cell (e.g., a eukaryotic cell or prokaryotic cell). This can be accomplished, for example, by incorporating a polynucleotide encoding the fusion protein into the genome of a cell (e.g., using techniques described herein or known in the art). Optionally, antibodies and fragments thereof described herein can be joined to a half-life extending molecule by forming a covalent bond between the antibody and a linker. This linker can then be subsequently conjugated to another molecule, or the linker can be conjugated to another molecule prior to ligation to the anti-GPC3 antibody or antigen-binding fragment. Examples of linkers that can be used for the formation of a conjugate include polypeptide linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Fusion proteins containing polypeptide linkers can be made using chemical synthesis techniques, such as those described herein, or through recombinant expression of a polynucleotide encoding the fusion protein in a cell (e.g., a prokaryotic or eukaryotic cell). Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582 (2012)).

### Nucleic Acids Encoding Anti-GPC3 Antibodies, Antigen-Binding Fragments, Binding Proteins, and CARs

This section provides examples of nucleic acids that may be used to encode antibodies, antigen-binding fragments, or binding proteins of the disclosure. The nucleic acid molecules of the disclosure may include one or more alterations. Herein, in a nucleotide, nucleoside, or polynucleotide (such as the nucleic acids of the invention (e.g., an mRNA or an oligonucleotide)), the terms “alteration” or, as appropriate, “alternative” refer to alteration with respect to A, G, U or C ribonucleotides.

The alterations may be various distinct alterations. In some embodiments, where the nucleic acid is an mRNA, the coding region, the flanking regions, and/or the terminal regions may contain one, two, or more (optionally different) nucleoside or nucleotide alterations. In some embodiments, an alternative polynucleotide introduced to a cell may exhibit reduced degradation in the cell, as compared to an unaltered polynucleotide.

The polynucleotides can include any useful alteration, such as to the sugar, the nucleobase, or the internucleoside linkage (e.g., to a linking phosphate, to a phosphodiester linkage, or to the phosphodiester backbone). In certain embodiments, alterations (e.g., one or more alterations) are present in each of the sugar and the internucleoside linkage. Alterations according to the present invention may be alterations of ribonucleic acids (RNAs) to deoxyribonucleic acids (DNAs) (e.g., the substitution of the 2′OH of the ribofuranosyl ring to 2′H), threose nucleic acids (TNAs), glycol nucleic acids (GNAs), peptide nucleic acids (PNAs), locked nucleic acids (LNAs) or hybrids thereof. Additional alterations are described herein.

In certain embodiments, it may be desirable for a nucleic acid molecule introduced into the cell to be degraded intracellularly. For example, degradation of a nucleic acid molecule may be preferable if precise timing of protein production is desired. Thus, in some embodiments, the invention provides an alternative nucleic acid molecule containing a degradation domain, which is capable of being acted on in a directed manner within a cell.

The polynucleotides can optionally include other agents (e.g., RNAi-inducing agents, RNAi agents, siRNAs, shRNAs, miRNAs, antisense RNAs, ribozymes, catalytic DNA, tRNA, RNAs that induce triple helix formation, aptamers, vectors, etc.). In some embodiments, the polynucleotides may include one or more messenger RNAs (mRNAs) having one or more alternative nucleoside or nucleotides (i.e., mRNA molecules). In some embodiments, the polynucleotides may include one or more oligonucleotides having one or more alternative nucleoside or nucleotides. In some embodiments, a composition of the invention includes an mRNA and/or one or more oligonucleotides having one or more alternative nucleoside or nucleotides.

### Modified Nucleic Acids

According to Aduri et al., (Aduri, R. et al., Journal of Chemical Theory and Computation. 3(4):1464-75, (2006)), there are 107 naturally occurring nucleosides, including 1-methyladenosine, 2-methylthio-N6-hydroxynorvalyl carbamoyladenosine, 2-methyladenosine, 2-O-ribosylphosphate adenosine, N6-methyl-N6-threonylcarbamoyladenosine, N6-acetyladenosine, N6-glycinylcarbamoyladenosine, N6-isopentenyladenosine, N6-methyladenosine, N6-threonylcarbamoyladenosine, N6,N6-dimethyladenosine. N6-(cis-hydroxyisopentenyl)adenosine. N6-hydroxynorvalylcarbamoyladenosine, 1,2-O-dimethyladenosine, N6,2-O-dimethyladenosine, 2-O-methyladenosine, N6,N6,O-2-trimethyladenosine, 2-methylthio-N6-(cis-hydroxyisopentenyl) adenosine, 2-methylthio-N6-methyladenosine, 2-methylthio-N6-isopentenyladenosine. 2-methylthio-N6-threonyl carbamoyladenosine, 2-thiocytidine, 3-methylcytidine, N4-acetylcytidine, 5-formylcytidine, N4-methylcytidine, 5-methylcytidine, 5-hydroxymethylcytidine, lysidine, N4-acetyl-2-O-methylcytidine, 5-fonnyl-2-O-methylcytidine, 5,2-O-dimethylcytidine, 2-O-methylcytidine. N4,2-O-dimethylcytidine, N4,N4,2-O-trimethylcytidine, 1-methylguanosine, N2,7-dimethylguanosine, N2-methylguanosine, 2-O-ribosylphosphate guanosine, 7-methylguanosine, under modified hydroxywybutosine, 7-amninomethyl-7-deazaguanosine, 7-cyano-7-deazaguanosine, N2,N2-dimethylguanosine, 4-demethylwyosine, epoxyqueuosine, hydroxywybutosine, isowyosine. N2,7,2-O-trimethylguanosine. N2,2-O-dimethylguanosine, 1,2-O-dimethylguanosine, 2-O-methylguanosine, N2,N2,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine. 3-(3-amino-3-carboxypropyl)uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldihydrouridine, 5-methylaminomethyl-2-thiouridine, 5-(carboxyhydroxymethyl)uridine. 5-(isopentenylaminomethyl)uridine. 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine, 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine. 5-taurinomethyl-2-thiouridine, pseudouridine, 1-methyl-3-(3-amino-3-carboxypropyl)pseudouridine, 1-methylpseudouridine, 3-methylpseudouridine, 2-O-methylpseudouridine, inosine, 1-methylinosine, 1,2-O-dimethylinosine, and 2-O-methylinosine. Each of these may be components of nucleic acids of the present invention.

### Nucleosides Containing Modified Sugars

The alternative nucleosides and nucleotides (e.g., building block molecules), which may be incorporated into a polynucleotide (e.g., RNA or mRNA, as described herein), can be altered on the sugar of the ribonucleic acid. For example, the 2′ hydroxyl group (OH) can be modified or replaced with a number of different substituents. Examples of substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃-s cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where examples of bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Examples of non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone): multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′->2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

### Alterations on the Nucleobase

The present disclosure provides for alternative nucleosides and nucleotides. As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

Examples of non-limiting alterations include an amino group, a thiol group, an alkyl group, a halo group, or any described herein. The alternative nucleotides may by synthesized by any useful method, as described herein (e.g., chemically, enzymatically, or recombinantly to include one or more alternative or alternative nucleosides).

In some embodiments, a nucleic acid of the invention (e.g., an mRNA or an oligonucleotide) includes one or more 2′-OMe nucleotides, 2′-methoxyethyl nucleotides (2′-MOE nucleotides). 2′-F nucleotide, 2′-NH2 nucleotide, 2′fluoroarabino nucleotides (FANA nucleotides), locked nucleic acid nucleotides (LNA nucleotides), or 4′-S nucleotides.

The alternative nucleotide base pairing encompasses not only the standard adenosine-thymine, adenosine-uracil, and guanosine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine, or uracil.

The alternative nucleosides and nucleotides can include an alternative nucleobase. Examples of nucleobases found in RNA include, but are not limited to, adenine, guanine, cytosine, and uracil. Examples of nucleobase found in DNA include, but are not limited to, adenine, guanine, cytosine, and thymine. These nucleobases can be altered or wholly replaced to provide polynucleotide molecules having enhanced properties (e.g., resistance to nucleases and stability), and these properties may manifest through disruption of the binding of a major groove binding partner.

In some embodiments, the alternative nucleobase is an alternative uracil. Examples of nucleobases and nucleosides having an alternative uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine. 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (nnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (xm⁵U), 1-taurinomethyl-pseudouridine, 5-taurinomethyl-2-thio-uridine (cm⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihvdrouridine, 5-methyl-dihydrouridine (m⁵D), 2-thio-dihydrouridine. 2-thio-dihv dropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine.

In preferred embodiments, the nucleic acid is modified to contain 1-methylpseudouridine (m¹ψ) in lieu of uridine at each instance.

In some embodiments, the alternative nucleobase is an alternative cytosine. Examples of nucleobases and nucleosides having an alternative cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C). 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f5Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the alternative nucleobase is an alternative adenine. Examples of nucleobases and nucleosides having an alternative adenine include 2-amino-purine, 2, 6-diaminopurine. 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine. 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms² hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶2Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the alternative nucleobase is an alternative guanine. Examples of nucleobases and nucleosides having an alternative guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o_{2y}W), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁻). 7-deaza-8-aza-guanosine, 6-thio-guanosine, 6-thio-7-deaza-guanosine, 6-thio-7-deaza-8-aza-guanosine, 7-methyl-guanosine (m⁷G). 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2,N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 12′-O-dimethyl-inosine (m¹Im). 2′-O-ribosylguanosine (phosphate) (Gr(p)), 1-thio-guanosine, 06-methyl-guanosine, 2′-F-ara-guanosine, and 2′-F-guanosine.

The nucleobase of the nucleotide can be independently selected from a purine, a pyrimidine, a purine, or pyrimidine analog. For example, the nucleobase can each be independently selected from adenine, cytosine, guanine, uracil, or hypoxanthine. In some embodiments, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl, and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil). 4-thiouracil, 8-halo (e.g., 8-bromo). 8-amino, 8-thiol. 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. When the nucleotides are depicted using the shorthand A, G, C, T or U, each letter refers to the representative base and/or derivatives thereof (e.g., A includes adenine or adenine analogs (e.g., 7-deaza adenine)).

In some embodiments, the polynucleotides of the invention contain 5-methoxy-uracil, uracil. 5-methyl-cytosine, and cytosine as the only uracils and cytosines. In some embodiments, the polynucleotides of the invention contain 5-methoxy-uracil, uracil, 5-trifluoromethyl-cytosine, and cytosine as the only uracils and cytosines. In some embodiments, the polynucleotides of the invention contain 5-methoxy-uracil, uracil, 5-hydroxymethyl-cytosine, and cytosine as the only uracils and cytosines. In some embodiments, the polynucleotides of the invention contain 5-methoxy-uracil, uracil. 5-bromo-cytosine, and cytosine as the only uracils and cytosines. In some embodiments, the polynucleotides of the invention contain 5-methoxy-uracil, uracil, 5-iodo-cytosine, and cytosine as the only uracils and cytosines. In some embodiments, the polynucleotides of the invention contain 5-methoxy-uracil, uracil, 5-methoxy-cytosine, and cytosine as the only uracils and cytosines. In some embodiments, the polynucleotides of the invention contain 5-methoxy-uracil, uracil, 5-ethyl-cytosine, and cytosine as the only uracils and cytosines. In some embodiments, the polynucleotides of the invention contain 5-methoxy-uracil, uracil, 5-phenyl-cytosine, and cytosine as the only uracils and cytosines. In some embodiments, the polynucleotides of the invention contain 5-methoxy-uracil, uracil, 5-ethnyl-cytosine, and cytosine as the only uracils and cytosines. In some embodiments, the polynucleotides of the invention contain 5-methoxy-uracil, uracil, N4-methyl-cytosine, and cytosine as the only uracils and cytosines. In some embodiments, the polynucleotides of the invention contain 5-methoxy-uracil, uracil, 5-fluoro-cytosine, and cytosine as the only uracils and cytosines. In some embodiments, the polynucleotides of the invention contain 5-methoxy-uracil, uracil, N4-acetyl-cytosine, and cytosine as the only uracils and cytosines. In some embodiments, the polynucleotides of the invention contain 5-methoxy-uracil, uracil, pseudoisocytosine, and cytosine as the only uracils and cytosines. In some embodiments, the polynucleotides of the invention contain 5-methoxy-uracil, uracil, 5-formyl-cytosine, and cytosine as the only uracils and cytosines. In some embodiments, the polynucleotides of the invention contain 5-methoxy-uracil, uracil, 5-aminoallyl-cytosine, and cytosine as the only uracils and cytosines. In some embodiments, the polynucleotides of the invention contain 5-methoxy-uracil, uracil, 5-carboxy-cytosine, and cytosine as the only uracils and cytosines.

In some embodiments, the polynucleotides of the invention contain 1-methyl-pseudouracil, uracil, 5-methyl-cytosine, and cytosine as the only uracils and cytosines. In some embodiments, the polynucleotides of the invention contain 1-methyl-pseudouracil, uracil, 5-trifluoromethyl-cytosine, and cytosine as the only uracils and cytosines. In some embodiments, the polynucleotides of the invention contain 1-methyl-pseudouracil, uracil, 5-hydroxymethyl-cytosine, and cytosine as the only uracils and cytosines. In some embodiments, the polynucleotides of the invention contain 1-methyl-pseudouracil, uracil, 5-bromo-cytosine, and cytosine as the only uracils and cytosines. In some embodiments, the polynucleotides of the invention contain 1-methyl-pseudouracil, uracil, 5-iodo-cytosine, and cytosine as the only uracils and cytosines. In some embodiments, the polynucleotides of the invention contain 1-methyl-pseudouracil, uracil, 5-methoxy-cytosine, and cytosine as the only uracils and cytosines. In some embodiments, the polynucleotides of the invention contain 1-methyl-pseudouracil, uracil, 5-ethyl-cytosine, and cytosine as the only uracils and cytosines. In some embodiments, the polynucleotides of the invention contain 1-methyl-pseudouracil, uracil, 5-phenyl-cytosine, and cytosine as the only uracils and cytosines. In some embodiments, the polynucleotides of the invention contain 1-methyl-pseudouracil, uracil, 5-ethnyl-cytosine, and cytosine as the only uracils and cytosines. In some embodiments, the polynucleotides of the invention contain 1-methyl-pseudouracil, uracil, N4-methyl-cytosine, and cytosine as the only uracils and cytosines. In some embodiments, the polynucleotides of the invention contain 1-methyl-pseudouracil, uracil, 5-fluoro-cytosine, and cytosine as the only uracils and cytosines. In some embodiments, the polynucleotides of the invention contain 1-methyl-pseudouracil, uracil, N4-acetyl-cytosine, and cytosine as the only uracils and cytosines. In some embodiments, the polynucleotides of the invention contain 1-methyl-pseudouracil, uracil, pseudoisocytosine, and cytosine as the only uracils and cytosines. In some embodiments, the polynucleotides of the invention contain 1-methyl-pseudouracil, uracil, 5-formyl-cytosine, and cytosine as the only uracils and cytosines. In some embodiments, the polynucleotides of the invention contain 1-methyl-pseudouracil, uracil, 5-aminoallyl-cytosine, and cytosine as the only uracils and cytosines. In some embodiments, the polynucleotides of the invention contain 1-methyl-pseudouracil, uracil, 5-carboxy-cytosine, and cytosine as the only uracils and cytosines.

In some embodiments, the polynucleotides of the invention contain 5-methoxy-uridine, uridine, 5-methyl-cytidine, and cytidine as the only uridines and cytidines. In some embodiments, the polynucleotides of the invention contain 5-methoxy-uridine, uridine, 5-trifluoromethyl-cytidine, and cytidine as the only uridines and cytidines. In some embodiments, the polynucleotides of the invention contain 5-methoxy-uridine, uridine, 5-hydroxymethyl-cytidine, and cytidine as the only uridines and cytidines. In some embodiments, the polynucleotides of the invention contain 5-methoxy-uridine, uridine, 5-bromo-cytidine, and cytidine as the only uridines and cytidines. In some embodiments, the polynucleotides of the invention contain 5-methoxy-uridine, uridine, 5-iodo-cytidine, and cytidine as the only uridines and cytidines. In some embodiments, the polynucleotides of the invention contain 5-methoxy-uridine, uridine, 5-methoxy-cytidine, and cytidine as the only uridines and cytidines. In some embodiments, the polynucleotides of the invention contain 5-methoxy-uridine, uridine, 5-ethyl-cytidine, and cytidine as the only uridines and cytidines. In some embodiments, the polynucleotides of the invention contain 5-methoxy-uridine, uridine, 5-phenyl-cytidine, and cytidine as the only uridines and cytidines. In some embodiments, the polynucleotides of the invention contain 5-methoxy-uridine, uridine, 5-ethnyl-cytidine, and cytidine as the only uridines and cytidines. In some embodiments, the polynucleotides of the invention contain 5-methoxy-uridine, uridine, N4-methyl-cytidine, and cytidine as the only uridines and cytidines. In some embodiments, the polynucleotides of the invention contain 5-methoxy-uridine, uridine, 5-fluoro-cytidine, and cytidine as the only uridines and cytidines. In some embodiments, the polynucleotides of the invention contain 5-methoxy-uridine, uridine, N4-acetyl-cytidine, and cytidine as the only uridines and cytidines. In some embodiments, the polynucleotides of the invention contain 5-methoxy-uridine, uridine, pseudoisocytidine, and cytidine as the only uridines and cytidines. In some embodiments, the polynucleotides of the invention contain 5-methoxy-uridine, uridine, 5-formyl-cytidine, and cytidine as the only uridines and cytidines. In some embodiments, the polynucleotides of the invention contain 5-methoxy-uridine, uridine, 5-aminoallyl-cytidine, and cytidine as the only uridines and cytidines. In some embodiments, the polynucleotides of the invention contain 5-methoxy-uridine, uridine, 5-carboxy-cytidine, and cytidine as the only uridines and cytidines.

In some embodiments, the polynucleotides of the invention contain 1-methyl-pseudouridine, uridine, 5-methyl-cytidine, and cytidine as the only uridines and cytidines. In some embodiments, the polynucleotides of the invention contain 1-methyl-pseudouridine, uridine, 5-trifluoromethyl-cytidine, and cytidine as the only uridines and cytidines. In some embodiments, the polynucleotides of the invention contain 1-methyl-pseudouridine, uridine, 5-hydroxymethyl-cytidine, and cytidine as the only uridines and cytidines. In some embodiments, the polynucleotides of the invention contain 1-methyl-pseudouridine, uridine, 5-bromo-cytidine, and cytidine as the only uridines and cytidines. In some embodiments, the polynucleotides of the invention contain 1-methyl-pseudouridine, uridine, 5-iodo-cytidine, and cytidine as the only uridines and cytidines. In some embodiments, the polynucleotides of the invention contain 1-methyl-pseudouridine, uridine, 5-methoxy-cytidine, and cytidine as the only uridines and cytidines. In some embodiments, the polynucleotides of the invention contain 1-methyl-pseudouridine, uridine, 5-ethyl-cytidine, and cytidine as the only uridines and cytidines. In some embodiments, the polynucleotides of the invention contain 1-methyl-pseudouridine, uridine, 5-phenyl-cytidine, and cytidine as the only uridines and cytidines. In some embodiments, the polynucleotides of the invention contain 1-methyl-pseudouridine, uridine, 5-ethnyl-cytidine, and cytidine as the only uridines and cytidines. In some embodiments, the polynucleotides of the invention contain 1-methyl-pseudouridine, uridine, N4-methyl-cytidine, and cytidine as the only uridines and cytidines. In some embodiments, the polynucleotides of the invention contain 1-methyl-pseudouridine, uridine, 5-fluoro-cytidine, and cytidine as the only uridines and cytidines. In some embodiments, the polynucleotides of the invention contain 1-methyl-pseudouridine, uridine, N4-acetyl-cytidine, and cytidine as the only uridines and cytidines. In some embodiments, the polynucleotides of the invention contain 1-methyl-pseudouridine, uridine, pseudoisocytidine, and cytidine as the only uridines and cytidines. In some embodiments, the polynucleotides of the invention contain 1-methyl-pseudouridine, uridine, 5-formyl-cytidine, and cytidine as the only uridines and cytidines. In some embodiments, the polynucleotides of the invention contain 1-methyl-pseudouridine, uridine, 5-aminoallyl-cytidine, and cytidine as the only uridines and cytidines. In some embodiments, the polynucleotides of the invention contain 1-methyl-pseudouridine, uridine, 5-carboxy-cytidine, and cytidine as the only uridines and cytidines.

In some embodiments, the polynucleotides of the invention contain the uracil of one of the nucleosides of Table 5 and uracil as the only uracils. In other embodiments, the polynucleotides of the invention contain a uridine of Table 5 and uridine as the only uridines.

TABLE 5
Examples of uracil-containing nucleosides
Nucleoside Name
5-methoxy-uridine
1-Methyl-pseudo-uridine
pseudouridine
5-methyl-uridine
5-bromo-uridine
2-thio-uridine
4-thiouridine
2′-O-methyluridine
5-methyl-2-thiouridine
5,2′-O-dimethyluridine
5-aminomethyl-2-thiouridine
5-(1-Propynyl)ara-uridine
2′-O-Methyl-5-(1-propynyl)uridine
5-Vinylarauridine
(Z)-5-(2-Bromo-vinyl)ara-uridine
(E)-5-(2-Bromo-vinyl)ara-uridine
(Z)-5-(2-Bromo-vinyl)uridine
(E)-5-(2-Bromo-vinyl)uridine
5-Cyanouridine
5-Formyluridine
5-Dimethylaminouridine
5-Trideuteromethyl-6-deuterouridine
5-(2-Furanyl)uridine
5-Phenylethynyluridine
4′-Carbocyclic uridine
4′-Ethynyluridine
4′-Azidouridine
2′-Deoxy-2′,2′-difluorouridine
2′-Deoxy-2′-b-fluorouridine
2′-Deoxy-2′-b-chlorouridine
2′-Deoxy-2′-b-bromouridine
2′-Deoxy-2′-b-iodouridine
5′-Homo-uridine
2′-Deoxy-2′-a-mercaptouridine
2′-Deoxy-2′-a-thiomethoxyuridine
2′-Deoxy-2′-a-azidouridine
2′-Deoxy-2′-a-aminouridine
2′-Deoxy-2′-b-mercaptouridine
2′-Deoxy-2′-b-thiomethoxyuridine
2′-Deoxy-2′-b-azidouridine
2′-Deoxy-2′-b-aminouridine
2′-b-Trifluoromethyluridine
2′-a-Trifluoromethyluridine
2′-b-Ethynyluridine
2′-a-Ethynyluridine
1-ethyl-pseudo-uridine
1-propyl-pseudo-uridine
1-iso-propyl-pseudo-uridine
1-(2,2,2-trifluoroethyl)-pseudo-uridine
1-cyclopropyl-pseudo-uridine
1-cyclopropylmethyl-pseudo-uridine
1-phenyl-pseudo-uridine
1-benzyl-pseudo-uridine
1-aminomethyl-pseudo-uridine
pseudo-uridine-1-2-ethanoic acid
1-(3-amino-3-carboxypropyl)pseudo-uridine
1-methyl-3-(3-amino-3-carboxypropyl)pseudo-uridine
6-methyl-pseudo-uridine
6-trifluoromethyl-pseudo-uridine
6-methoxy-pseudo-uridine
6-phenyl-pseudo-uridine
6-iodo-pseudo-uridine
6-bromo-pseudo-uridine
6-chloro-pseudo-uridine
6-fluoro-pseudo-uridine
4-Thio-pseudo-uridine
2-Thio-pseudo-uridine
Alpha-thio-pseudo-uridine
1-Me-alpha-thio-pseudo-uridine
1-butyl-pseudo-uridine
1-tert-butyl-pseudo-uridine
1-pentyl-pseudo-uridine
1-hexyl-pseudo-uridine
1-trifluoromethyl-pseudo-uridine
1-cyclobutyl-pseudo-uridine
1-cyclopentyl-pseudo-uridine
1-cyclohexyl-pseudo-uridine
1-cycloheptyl-pseudo-uridine
1-cyclooctyl-pseudo-uridine
1-cyclobutylmethyl-pseudo-uridine
1-cyclopentylmethyl-pseudo-uridine
1-cyclohexylmethyl-pseudo-uridine
1-cycloheptylmethyl-pseudo-uridine
1-cyclooctylmethyl-pseudo-uridine
1-p-tolyl-pseudo-uridine
1-(2,4,6-trimethyl-phenyl)pseudo-uridine
1-(4-methoxy-phenyl)pseudo-uridine
1-(4-amino-phenyl)pseudo-uridine
1(4-nitro-phenyl)pseudo-uridine
pseudo-uridine-N1-p-benzoic acid
1-(4-methyl-benzyl)pseudo-uridine
1-(2,4,6-trimethyl-benzyl)pseudo-uridine
1-(4-methoxy-benzyl)pseudo-uridine
1-(4-amino-benzyl)pseudo-uridine
1-(4-nitro-benzyl)pseudo-uridine
pseudo-uridine-N1-methyl-p-benzoic acid
1-(2-amino-ethyl)pseudo-uridine
1-(3-amino-propyl)pseudo-uridine
1-(4-amino-butyl)pseudo-uridine
1-(5-amino-pentyl)pseudo-uridine
1-(6-amino-hexyl)pseudo-uridine
pseudo-uridine-N1-3-propionic acid
pseudo-uridine-N1-4-butanoic acid
pseudo-uridine-N1-5-pentanoic acid
pseudo-uridine-N1-6-hexanoic acid
pseudo-uridine-N1-7-heptanoic acid
1-(2-amino-2-carboxyethyl)pseudo-uridine
1-(4-amino-4-carboxybutyl)pseudo-uridine
3-alkyl-pseudo-uridine
6-ethyl-pseudo-uridine
6-propyl-pseudo-uridine
6-iso-propyl-pseudo-uridine
6-butyl-pseudo-uridine
6-tert-butyl-pseudo-uridine
6-(2,2,2-trifluoroethyl)-pseudo-uridine
6-ethoxy-pseudo-uridine
6-trifluoromethoxy-pseudo-uridine
6-phenyl-pseudo-uridine
6-(substituted-phenyl)-pseudo-uridine
6-cyano-pseudo-uridine
6-azido-pseudo-uridine
6-amino-pseudo-uridine
6-ethylcarboxylate-pseudo-uridine
6-hydroxy-pseudo-uridine
6-methylamino-pseudo-uridine
6-dimethylamino-pseudo-uridine
6-hydroxyamino-pseudo-uridine
6-formyl-pseudo-uridine
6-(4-morpholino)-pseudo-uridine
6-(4-thiomorpholino)-pseudo-uridine
1-me-4-thio-pseudo-uridine
1-me-2-thio-pseudo-uridine
1,6-dimethyl-pseudo-uridine
1-methyl-6-trifluoromethyl-pseudo-uridine
1-methyl-6-ethyl-pseudo-uridine
1-methyl-6-propyl-pseudo-uridine
1-methyl-6-iso-propyl-pseudo-uridine
1-methyl-6-butyl-pseudo-uridine
1-methyl-6-tert-butyl-pseudo-uridine
1-methyl-6-(2,2,2-trifluoroethyl)pseudo-uridine
1-methyl-6-iodo-pseudo-uridine
1-methyl-6-bromo-pseudo-uridine
1-methyl-6-chloro-pseudo-uridine
1-methyl-6-fluoro-pseudo-uridine
1-methyl-6-methoxy-pseudo-uridine
1-methyl-6-ethoxy-pseudo-uridine
1-methyl-6-trifluoromethoxy-pseudo-uridine
1-methyl-6-phenyl-pseudo-uridine
1-methyl-6-(substituted phenyl)pseudo-uridine
1-methyl-6-cyano-pseudo-uridine
1-methyl-6-azido-pseudo-uridine
1-methyl-6-amino-pseudo-uridine
1-methyl-6-ethylcarboxylate-pseudo-uridine
1-methyl-6-hydroxy-pseudo-uridine
1-methyl-6-methylamino-pseudo-uridine
1-methyl-6-dimethylamino-pseudo-uridine
1-methyl-6-hydroxyamino-pseudo-uridine
1-methyl-6-formyl-pseudo-uridine
1-methyl-6-(4-morpholino)-pseudo-uridine
1-methyl-6-(4-thiomorpholino)-pseudo-uridine
1-alkyl-6-vinyl-pseudo-uridine
1-alkyl-6-allyl-pseudo-uridine
1-alkyl-6-homoallyl-pseudo-uridine
1-alkyl-6-ethynyl-pseudo-uridine
1-alkyl-6-(2-propynyl)-pseudo-uridine
1-alkyl-6-(1-propynyl)-pseudo-uridine
1-Hydroxymethylpseudouridine
1-(2-Hydroxyethyl)pseudouridine
1-Methoxymethylpseudouridine
1-(2-Methoxyethyl)pseudouridine
1-(2,2-Diethoxyethyl)pseudouridine
(±)1-(2-Hydroxypropyl)pseudouridine
(2R)-1-(2-Hydroxypropyl)pseudouridine
(2S)-1-(2-Hydroxypropyl)pseudouridine
1-Cyanomethylpseudouridine
1-Morpholinomethylpseudouridine
1-Thiomorpholinomethylpseudouridine
1-Benzyloxymethylpseudouridine
1-(2,2,3,3,3-Pentafluoropropyl)pseudouridine
1-Thiomethoxymethylpseudouridine
1-Methanesulfonylmethylpseudouridine
1-Vinylpseudouridine
1-Allylpseudouridine
1-Homoallylpseudouridine
1-Propargylpseudouridine
1-(4-Fluorobenzyl)pseudouridine
1-(4-Chlorobenzyl)pseudouridine
1-(4-Bromobenzyl)pseudouridine
1-(4-Iodobenzyl)pseudouridine
1-(4-Methylbenzyl)pseudouridine
1-(4-Trifluoromethylbenzyl)pseudouridine
1-(4-Methoxybenzyl)pseudouridine
1-(4-Trifluoromethoxybenzyl)pseudouridine
1-(4-Thiomethoxybenzyl)pseudouridine
1-(4-Methanesulfonylbenzyl)pseudouridine
Pseudouridine 1-(4-methylbenzoic acid)
Pseudouridine 1-(4-methylbenzenesulfonic acid)
1-(2,4,6-Trimethylbenzyl)pseudouridine
1-(4-Nitrobenzyl)pseudouridine
1-(4-Azidobenzyl)pseudouridine
1-(3,4-Dimethoxybenzyl)pseudouridine
1-(3,4-Bis-trifluoromethoxybenzyl)pseudouridine
1-Acetylpseudouridine
1-Trifluoroacetylpseudouridine
1-Benzoylpseudouridine
1-Pivaloylpseudouridine
1-(3-Cyclopropyl-prop-2-ynyl)pseudouridine
Pseudouridine 1-methylphosphonic acid diethyl ester
Pseudouridine 1-methylphosphonic acid
Pseudouridine 1-[3-(2-ethoxy)]propionic acid
Pseudouridine 1-[3-{2-(2-ethoxy)-ethoxy}] propionic acid
Pseudouridine TP 1-[3-{2-(2-[2-ethoxy]-ethoxy)-
ethoxy}]propionic acid
Pseudouridine 1-[3-{2-(2-[2-(2-ethoxy)-ethoxy]-ethoxy)-
ethoxy}]propionic acid
Pseudouridine 1-[3-{2-(2-[2-{2(2-ethoxy)-ethoxy}-ethoxy]-
ethoxy)-ethoxy}]propionic acid
1-{3-[2-(2-Aminoethoxy)-ethoxy]-propionyl} pseudouridine
1-[3-(2-{2-[2-(2-Aminoethoxy)-ethoxy]-ethoxy}-ethoxy)-
propionyl]pseudouridine
1-Biotinylpseudouridine
1-Biotinyl-PEG2-pseudouridine
5-Oxyacetic acid-methyl ester-uridine
3-Methyl-pseudo-uridine
5-trifluoromethyl-uridine
5-methyl-amino-methyl-uridine
5-carboxy-methyl-amino-methyl-uridine
5-carboxymethylaminomethyl-2′-OMe-uridine
5-carboxymethylaminomethyl-2-thio-uridine
5-methylaminomethyl-2-thio-uridine
5-methoxy-carbonyl-methyl-uridine
5-methoxy-carbonyl-methyl-2′-OMe-uridine
5-oxyacetic acid-uridine
3-(3-amino-3-carboxypropyl)-uridine
5-(carboxyhydroxymethyl)uridine methyl ester
5-(carboxyhydroxymethyl)uridine
2′-OMe-pseudo-uridine
2′-Azido-2′-deoxy-uridine
2′-Amino-2′-deoxy-uridine
2′-F-5-Methyl-2′-deoxy-uridine
5-iodo-2′-fluoro-deoxyuridine
2′-bromo-deoxyuridine
2,2′-anhydro-uridine
2′-Azido-deoxyuridine
5-Methoxycarbonylmethyl-2-thiouridine
5-Methylaminomethyl-2-thiouridine
5-Carbamoylmethyluridine
5-Carbamoylmethyl-2′-O-methyluridine
1-Methyl-3-(3-amino-3-carboxypropyl) pseudouridine
5-Methylaminomethyl-2-selenouridine
5-Carboxymethyluridine
5-Methyldihydrouridine
5-Taurinomethyluridine
5-Taurinomethyl-2-thiouridine
5-(iso-Pentenylaminomethyl)uridine
5-(iso-Pentenylaminomethyl)-2-thiouridine
5-(iso-Pentenylaminomethyl)-2′-O-methyluridine
2′-O-Methylpseudouridine
2-Thio-2′-O-methyluridine
3,2′-O-Dimethyluridine
5-Methoxy-carbonylmethyl-uridine
5-hydroxy-uridine
5-Isopentenyl-aminomethyl-uridine

In some embodiments, the polynucleotides of the invention contain the cytosine of one of the nucleosides of Table 6 and cytosine as the only cytosines. In other embodiments, the polynucleotides of the invention contain a cytidine of Table 6 and cytidine as the only cytidines.

TABLE 6
Examples of cytosine containing nucleosides
Nucleoside Name
α-thio-cytidine
Pseudoisocytidine
pyrrolo-cytidine
5-methyl-cytidine
N4-acetyl-cytidine
5-Bromo-cytidine
5-Trifluoromethyl-cytidine
5-Hydroxymethyl-cytidine
5-Iodo-cytidine
5-Ethyl-cytidine
5-Methoxy-cytidine
5-Ethynyl-cytidine
5-Fluoro-cytidine
5-Phenyl-cytidine
N4-Bz-cytidine
N4-Methyl-cytidine
5-Pseudo-iso-cytidine
5-Formyl-cytidine
5-Aminoallyl-cytidine
2′-O-methylcytidine
2′-O-Methyl-5-(1-propynyl)cytidine
5-(1-Propynyl)ara-cytidine
5-Ethynylara-cytidine
5-Ethynylcytidine
5-Cyanocytidine
5-(2-Chloro-phenyl)-2-thiocytidine
5-(4-Amino-phenyl)-2-thiocytidine
N4,2′-O-Dimethylcytidine
3′-Ethynylcytidine
4′-Carbocyclic cytidine
4′-Ethynylcytidine
4′-Azidocytidine
2′-Deoxy-2′,2′-difluorocytidine
2′-Deoxy-2′-b-fluorocytidine
2′-Deoxy-2′-b-chlorocytidine
2′-Deoxy-2′-b-bromocytidine
2′-Deoxy-2′-b-iodocytidine
5′-Homo-cytidine
2′-Deoxy-2′-a-mercaptocytidine
2′-Deoxy-2′-a-thiomethoxycytidine
2′-Deoxy-2′-a-azidocytidine
2′-Deoxy-2′-a-aminocytidine
2′-Deoxy-2′-b-mercaptocytidine
2′-Deoxy-2′-b-thiomethoxycytidine
2′-Deoxy-2′-b-azidocytidine
2′-Deoxy-2′-b-aminocytidine
2′-b-Trifluoromethylcytidine
2′-a-Trifluoromethylcytidine
2′-b-Ethynylcytidine
2′-a-Ethynylcytidine
(E)-5-(2-Bromo-vinyl)cytidine
2′-Azido-2′-deoxy-cytidine
2′-Amino-2′-deoxy-cytidine
5-aminoallyl-cytidine
2,2′-anhydro-cytidine
N4-amino-cytidine
2′-O-Methyl-N4-acetyl-cytidine
2′-fluoro-N4-acetyl-cytidine
2′-fluor-N4-Bz-cytidine
2′-O-methyl-N4-Bz-cytidine
N4, 2′-O-Dimethylcytidine
5-Formyl-2′-O-methylcytidine

### Alterations on the Internucleoside Linkage

The alternative nucleotides, which may be incorporated into a polynucleotide molecule, can be altered on the internucleoside linkage (e.g., phosphate backbone). Herein, in the context of the polynucleotide backbone, the phrases “phosphate” and “phosphodiester” are used interchangeably. Backbone phosphate groups can be altered by replacing one or more of the oxygen atoms with a different substituent.

The alternative nucleosides and nucleotides can include the wholesale replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phospharamidite, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl and/or methoxy. As a non-limiting example, two non-bridging oxygens at the same position (e.g., the alpha (α), beta (β) or gamma (γ) position) can be replaced with a sulfur (thio) and a methoxy.

The replacement of one or more of the oxygen atoms at the α position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment. While not wishing to be bound by theory, phosphorothioate linked polynucleotide molecules are expected to also reduce the innate immune response through weaker binding/activation of cellular innate immune molecules.

In specific embodiments, an alternative nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein below.

### Combinations of Alternative Sugars, Nucleobases, and Internucleoside Linkages

The polynucleotides of the invention can include a combination of alterations to the sugar, the nucleobase, and/or the internucleoside linkage. These combinations can include any one or more alterations described herein.

### Synthesis of Polynucleotides

The polynucleotide molecules for use in accordance with the invention may be prepared according to any useful technique, as described herein. The alternative nucleosides and nucleotides used in the synthesis of polynucleotide molecules disclosed herein can be prepared from readily available starting materials using the following general methods and procedures. Where typical or preferred process conditions (e.g., reaction temperatures, times, mole ratios of reactants, solvents, pressures, etc.) are provided, a skilled artisan would be able to optimize and develop additional process conditions. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography (e.g., high performance liquid chromatography (HPLC) or thin layer chromatography).

Preparation of polynucleotide molecules of the present invention can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons (1991), which is incorporated herein by reference in its entirety.

The reactions of the processes described herein can be carried out in suitable solvents, which can be readily selected by one of skill in the art of organic synthesis. Suitable solvents can be substantially nonreactive with the starting materials (reactants), the intermediates, or products at the temperatures at which the reactions are carried out (i.e., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected.

Resolution of racemic mixtures of alternative polynucleotides or nucleic acids (e.g., polynucleotides or mRNA molecules) can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

Alternative nucleosides and nucleotides (e.g., building block molecules) can be prepared according to the synthetic methods described in Ogata et al., J. Org. Chem. 74:2585-2588 (2009); Purmal et al., Nucl. Acids Res. 22(1): 72-78 (1994); Fukuhara et al., Biochemistry, 1(4): 563-568 (1962); and Xu et al., Tetrahedron. 48(9): 1729-1740 (1992), each of which are incorporated by reference in their entirety.

If the polynucleotide includes one or more alternative nucleosides or nucleotides, the polynucleotides of the invention may or may not be uniformly altered along the entire length of the molecule. For example, one or more or all types of nucleotide (e.g., purine or pyrimidine, or any one or more or all of A, G, U. C) may or may not be uniformly altered in a polynucleotide of the invention, or in a given predetermined sequence region thereof. In some embodiments, all nucleotides X in a polynucleotide of the invention (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations, nucleotide alterations, and/or internucleoside linkages (e.g., backbone structures) may exist at various positions in the polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′ or 3′ terminal alteration. The polynucleotide may contain from 1% to 100% alternative nucleosides, nucleotides, or internucleoside linkages (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100. In some embodiments, the remaining percentage is accounted for by the presence of A, G, U, or C.

When referring to percentage incorporation by an alternative nucleoside, nucleotide, or internucleoside linkage, in some embodiments the remaining percentage necessary to total 100% is accounted for by the corresponding natural nucleoside, nucleotide, or internucleoside linkage. In other embodiments, the remaining percentage necessary to total 100% is accounted for by a second alternative nucleoside, nucleotide, or internucleoside linkage.

### Messenger RNA

The present invention features compositions including one or more mRNAs, where each mRNA encodes a polypeptide (e.g., an anti-GPC3 antibody, antigen-binding fragment, binding protein or CAR described herein). mRNAs of the disclosure may each include (i) a 5′-cap structure; (ii) a 5′-UTR; (iii) an open reading frame encoding the polypeptide; (iv) a 3′-untranslated region (3′-UTR); and (v) a poly-A region.

In some embodiments, the mRNA includes from about 30 to about 3,000 (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1.000, from 100 to 1.500, from 100 to 2.000, from 100 to 2.500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2.500, from 1,500 to 3,000, from 2,000 to 3,000, from 2,000 to 2,500, or from 2,500 to 3,000) nucleotides.

mRNA: 5′-Cap

The 5′-cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Alterations to the nucleic acids of the present invention may generate a non-hydrolyzable cap structure preventing decapping and thus increasing mRNA half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with α-thio-guanosine nucleotides according to the manufacturer's instructions to create a phosphorothioate linkage in the 5′-ppp-5′ cap. Additional alternative guanosine nucleotides may be used such as α-methyl-phosphonate and seleno-phosphate nucleotides.

Additional alterations include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

5′-cap structures include those described in International Patent Publication Nos. WO2008/127688, WO2008/016473, and WO2011/015347, each of which is incorporated herein by reference in its entirety.

Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs may be chemically (i.e., non-enzymatically) or enzymatically synthesized and/linked to a nucleic acid molecule.

For example, the Anti-Reverse Cap Analog (ARCA) cap contains two guanosines linked by a 5′-5′-triphosphate group, wherein one guanosine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G)). The 3′-O atom of the other, unaltered, guanosine becomes linked to the 5′-terminal nucleotide of the capped nucleic acid molecule (e.g., an mRNA or mmRNA). The N7- and 3′-O-methlyated guanosine provides the terminal moiety of the capped nucleic acid molecule (e.g., mRNA or mmRNA).

Another example of a cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In some embodiments, the cap is a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the contents of which are herein incorporated by reference in its entirety.

In some embodiments, the cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′-O} G(5′)ppp(5′)G cap analog (see, e.g., the various cap analogs and the methods of synthesizing cap analogs described in Kore et al. Bioorganic & Medicinal Chemistry 21:4570-4574 (2013); the contents of which are herein incorporated by reference in its entirety). In some embodiments, a cap analog of the present invention is a 4-chloro/bromophenoxyethyl analog.

While cap analogs allow for the concomitant capping of a nucleic acid molecule in an in vitro transcription reaction, up to 20% of transcripts remain uncapped. This, as well as the structural differences of a cap analog from endogenous 5′-cap structures of nucleic acids produced by the endogenous, cellular transcription machinery, may lead to reduced translational competency and reduced cellular stability.

Nucleic acids of the invention (e.g., mRNAs of the invention) may also be capped post-transcriptionally, using enzymes. 5′ cap structures produced by enzymatic capping may enhance binding of cap binding proteins, increase half-life, reduce susceptibility to 5′ endonucleases and/or reduce 5′ decapping, as compared to synthetic 5′-cap structures known in the art (or to a wild-type, natural or physiological 5′-cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanosine cap nucleotide wherein the cap guanosine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include 7mG(5′)ppp(5′)N,pN2p (cap 0), 7mG(5′)ppp(5′)NlmpNp (cap 1), 7mG(5′)-ppp(5′)NlmpN2mp (cap 2), and m(7)Gpppm(3)(6,6,2′)Apm(2′)Apm(2′)Cpm(2)(3,2′)Up (cap 4).

According to the present invention, 5′ terminal caps may include endogenous caps or cap analogs. According to the present invention, a 5′ terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the nucleic acids described herein may contain a modified 5′-cap. A modification on the 5′-cap may increase the stability of mRNA, increase the half-life of the mRNA, and could increase the mRNA translational efficiency. The modified 5′-cap may include, but is not limited to, one or more of the following modifications: modification at the 2′ and/or 3′ position of a capped guanosine triphosphate (GTP), a replacement of the sugar ring oxygen (that produced the carbocyclic ring) with a methylene moiety (CH₂), a modification at the triphosphate bridge moiety of the cap structure, or a modification at the nucleobase (G) moiety.

mRNA: Coding Region

Provided are nucleic acids that encode antibodies,antigen-binding fragments, binding proteins, or CARs of the disclosure. As recognized by those skilled in the art, protein fragments, functional protein domains, and homologous proteins are also considered to be within the scope of this present disclosure. For example, provided herein is any protein fragment of a reference protein (meaning a polypeptide sequence at least one amino acid residue shorter than a reference polypeptide sequence but otherwise identical) 10, 20, 30, 40, 50, 60, 70, 80, 90, 100, or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the present disclosure. In certain embodiments, a protein sequence to be utilized in accordance with the present disclosure includes 2, 3, 4, 5, 6, 7, 8, 9, 10, or more mutations as shown in any of the sequences provided or referenced herein.

mRNA: Poly-A Tail

During RNA processing, a long chain of adenosine nucleotides (poly(A) tail) is normally added to mRNA molecules to increase the stability of the mRNA. Immediately after transcription, the 3′ end of the transcript is cleaved to free a 3′ hydroxyl. Then poly(A) polymerase adds a chain of adenosine nucleotides to the RNA. The process, called polyadenylation, adds a poly-A tail that is between 100 and 250 residues long (SEQ ID NO: 251).

Methods for the stabilization of RNA by incorporation of chain-terminating nucleosides at the 3′-terminus include those described in International Patent Publication No. WO2013/103659, incorporated herein in its entirety.

Poly(A) tail deadenylation by 3′ exonucleases is a key step in cellular mRNA degradation in eukaryotes. By blocking 3′ exonucleases, the functional half-life of mRNA can be increased, resulting in increased protein expression. Chemical and enzymatic ligation strategies to modify the 3′ end of mRNA with reverse chirality adenosine (LA10) and/or inverted deoxythymidine (IdT) are known to those of skill in the art and have been demonstrated to extend mRNA half-life in cellular and in vivo studies. In some embodiments, the poly(A)tail of the mRNA includes a 3′ LA10 or IdT modification. For example, as described in International Patent Publication No. WO2017/049275, the tail modifications of which are incorporated by reference in their entirety.

Additional strategies have been explored to further stabilize mRNA, including: chemical modification of the 3′ nucleotide (e.g., conjugation of a morpholino to the 3′ end of the poly(A)tail); incorporation of stabilizing sequences after the poly(A) tail (e.g., a co-polymer, a stem-loop, or a triple helix); and/or annealing of structured oligos to the 3′ end of an mRNA, as described, for example, in International Patent Publication No. WO2017/049286, the stabilized linkages of which are incorporated by reference in their entirety.

Annealing an oligonucleotide (e.g., an oligonucleotide conjugate) with a complex secondary structure (e.g., a triple-helix structure or a stem-loop structure) at the 3′end may provide nuclease resistance and increase half-life of mRNA.

Unique poly(A) tail lengths may provide certain advantages to the RNAs of the present invention. Generally, the length of a poly(A) tail of the present invention is greater than 30 nucleotides in length. In some embodiments, the poly(A) tail is greater than 35 nucleotides in length. In some embodiments, the length is at least 40 nucleotides. In another embodiment, the length is at least 45 nucleotides. In some embodiments, the length is at least 50 nucleotides. In some embodiments, the length is at least 55 nucleotides. In another embodiment, the length is at least 60 nucleotides. In another embodiment, the length is at least 65 nucleotides. In another embodiment, the length is at least 70 nucleotides. In some embodiments, the length is at least 80 nucleotides. In some embodiments, the length is at least 90 nucleotides. In some embodiments, the length is at least 100 nucleotides. In some embodiments, the length is at least 120 nucleotides. In some embodiments, the length is at least 140 nucleotides. In some embodiments, the length is at least 160 nucleotides. In some embodiments, the length is at least 180 nucleotides. In some embodiments, the length is at least 200 nucleotides. In some embodiments, the length is at least 250 nucleotides. In some embodiments, the length is at least 300 nucleotides. In some embodiments, the length is at least 350 nucleotides. In some embodiments, the length is at least 400 nucleotides. In some embodiments, the length is at least 450 nucleotides. In some embodiments, the length is at least 500 nucleotides. In some embodiments, the length is at least 600 nucleotides. In some embodiments, the length is at least 700 nucleotides. In some embodiments, the length is at least 800 nucleotides. In some embodiments, the length is at least 900 nucleotides. In some embodiments, the length is at least 1000 nucleotides. In some embodiments, the length is at least 1100 nucleotides. In some embodiments, the length is at least 1200 nucleotides. In some embodiments, the length is at least 1300 nucleotides. In some embodiments, the length is at least 1400 nucleotides. In some embodiments, the length is at least 1500 nucleotides. In some embodiments, the length is at least 1600 nucleotides. In some embodiments, the length is at least 1700 nucleotides. In some embodiments, the length is at least 1800 nucleotides. In some embodiments, the length is at least 1900 nucleotides. In some embodiments, the length is at least 2000 nucleotides. In some embodiments, the length is at least 2500 nucleotides. In some embodiments, the length is at least 3000 nucleotides.

In some embodiments, the poly(A) tail may be 80 nucleotides, 120 nucleotides, or 160 nucleotides in length. In some embodiments, the poly(A) tail may be 20, 40, 80, 100, 120, 140 or 160 nucleotides in length.

In some embodiments, the poly(A) tail is designed relative to the length of the mRNA. This design may be based on the length of the coding region of the mRNA, the length of a particular feature or region of the mRNA, or based on the length of the ultimate product expressed from the RNA. When relative to any additional feature of the RNA (e.g., other than the mRNA portion which includes the poly(A) tail), poly(A) tail may be 10, 20, 30, 40, 50, 60, 70, 80, 90 or 100% greater in length than the additional feature. The poly(A) tail may also be designed as a fraction of the mRNA to which it belongs. In this context, the poly(A) tail may be 10, 20, 30, 40, 50, 60, 70, 80, or 90% or more of the total length of the construct or the total length of the construct minus the poly(A) tail.

In some embodiments, engineered binding sites and/or the conjugation of nucleic acids or mRNA for poly(A) binding protein may be used to enhance expression. The engineered binding sites may be sensor sequences which can operate as binding sites for ligands of the local microenvironment of the nucleic acids and/or mRNA. As a non-limiting example, the nucleic acids and/or nRNA may include at least one engineered binding site to alter the binding affinity of poly(A) binding protein (PABP) and analogs thereof. The incorporation of at least one engineered binding site may increase the binding affinity of the PABP and analogs thereof.

Additionally, multiple distinct nucleic acids or mRNA may be linked together to the PABP (poly(A) binding protein) through the 3′-end using nucleotides at the 3′-terminus of the poly(A) tail. Transfection experiments can be conducted in relevant cell lines and protein production can be assayed by ELISA at 12 hr, 24 hr, 48 hr, 72 hr, and day 7 post-transfection. As a non-limiting example, the transfection experiments may be used to evaluate the effect on PABP or analogs thereof binding affinity as a result of the addition of at least one engineered binding site.

In some embodiments, a poly(A) tail may be used to modulate translation initiation. While not wishing to be bound by theory, the poly-A tail recruits PABP which in turn can interact with translation initiation complex and thus may be essential for protein synthesis.

In some embodiments, a poly(A) tail may also be used in the present invention to protect against 3′-5′ exonuclease digestion.

In some embodiments, the nucleic acids or mRNA of the present invention are designed to include a poly-A-G Quartet. The G-quartet is a cyclic hydrogen bonded array of four guanosine nucleotides that can be formed by G-rich sequences in both DNA and RNA. In this embodiment, the G-quartet is incorporated at the end of the poly-A tail. The resultant nucleic acid or mRNA may be assayed for stability, protein production and other parameters including half-life at various time points. It has been discovered that the poly-A-G quartet results in protein production equivalent to at least 75% of that seen using a poly-A tail of 120 nucleotides alone (SEQ ID NO: 252).

In some embodiments, the nucleic acids or mRNA of the present invention may include a poly(A) tail and may be stabilized by the addition of a chain terminating nucleoside. The nucleic acids and/or mRNA with a poly(A) tail may further include a 5′cap structure.

In some embodiments, the nucleic acids or mRNA of the present invention may include a poly-A-G Quartet. The nucleic acids and/or mRNA with a poly-A-G Quartet may further include a 5′cap structure.

In some embodiments, the chain terminating nucleoside which may be used to stabilize the nucleic acid or mRNA including a poly(A) tail or poly-A-G Quartet may be, but is not limited to, those described in International Patent Publication No. WO2013103659, incorporated herein by reference in its entirety. In some embodiments, the chain terminating nucleosides which may be used with the present invention includes, but is not limited to, 3′-deoxyadenosine (cordycepin). 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or a —O— methylnucleoside.

In some embodiments, the mRNA which includes a poly(A) tail or a poly-A-G Quartet may be stabilized by an alteration to the 3′region of the nucleic acid that can prevent and/or inhibit the addition of oligo(U) (see, e.g., International Patent Publication No. WO2013/103659, incorporated herein by reference in its entirety).

In yet another embodiment, the mRNA, which includes a poly(A) tail or a poly-A-G Quartet, may be stabilized by the addition of an oligonucleotide that terminates in a 3′-deoxynucleoside, 2′,3′-dideoxynucleoside 3′-O-methylnucleoside, 3′-O-ethylnucleosides, 3′-arabinosides, and other alternative nucleosides known in the art and/or described herein.

mRNA: Stem-Loops

In some embodiments, the nucleic acids of the present invention (e.g., the mRNA of the present invention) may include a stem-loop such as, but not limited to, a histone stem-loop. The stem-loop may be a nucleotide sequence that is about 25 or about 26 nucleotides in length such as, but not limited to, SEQ ID NOs: 7-17 as described in International Patent Publication No. WO2013/103659, incorporated herein by reference in its entirety. The histone stem-loop may be located 3′ relative to the coding region (e.g., at the 3′ terminus of the coding region). As a non-limiting example, the stem-loop may be located at the 3′ end of a nucleic acid described herein.

In some embodiments, the stem-loop may be located in the second terminal region. As a non-limiting example, the stem-loop may be located within an untranslated region (e.g., 3′-UTR) in the second terminal region.

In some embodiments, the nucleic acid such as, but not limited to mRNA, which includes the histone stem-loop may be stabilized by the addition of at least one chain terminating nucleoside. Not wishing to be bound by theory, the addition of at least one chain terminating nucleoside may slow the degradation of a nucleic acid and thus can increase the half-life of the nucleic acid.

In some embodiments, the chain terminating nucleoside may be, but is not limited to, those described in International Patent Publication No. WO2013/103659, incorporated herein by reference in its entirety. In some embodiments, the chain terminating nucleosides which may be used with the present invention includes, but is not limited to, 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or a —O— methylnucleoside.

In some embodiments, the nucleic acid such as, but not limited to mRNA, which includes the histone stem-loop may be stabilized by an alteration to the 3′region of the nucleic acid that can prevent and/or inhibit the addition of oligo(U) (see, e.g., International Patent Publication No. WO2013/103659, incorporated herein by reference in its entirety).

In yet another embodiment, the nucleic acid such as, but not limited to, mRNA, which includes the histone stem-loop may be stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

In some embodiments, the nucleic acids of the present invention may include a histone stem-loop, a poly(A) tail sequence, and/or a 5′-cap structure. The histone stem-loop may be before and/or after the poly-A tail sequence. The nucleic acids including the histone stem-loop and a poly(A) tail sequence may include a chain terminating nucleoside described herein.

In some embodiments, the nucleic acids of the present invention may include a histone stem-loop and a 5′-cap structure. The 5′-cap structure may include, but is not limited to, those described herein and/or known in the art.

In some embodiments, the nucleic acids described herein may include at least one histone stem-loop and a poly(A) sequence or polyadenylation signal. Non-limiting examples of nucleic acid sequences encoding for at least one histone stem-loop and a poly-A sequence or a polyadenylation signal are described in International Patent Publication Nos. WO2013/120497, WO2013/120629, WO2013/120500, WO2013/120627, WO2013/120498, WO2013/120626, WO2013/120499 and WO2013/120628, the contents of each of which are incorporated herein by reference in their entirety. In some embodiments, the nucleic acid encoding for a histone stem-loop and a poly(A) sequence or a polyadenylation signal may code for a pathogen antigen or fragment thereof such as the nucleic acid sequences described in International Patent Publication Nos. WO2013/120499 and WO2013/120628, the contents of both of which are incorporated herein by reference in their entirety. In some embodiments, the nucleic acid encoding for a histone stem-loop and a poly(A) sequence or a polyadenylation signal may code for a therapeutic protein such as the nucleic acid sequences described in International Patent Publication Nos. WO2013/120497 and WO2013/120629, the contents of both of which are incorporated herein by reference in their entirety. In some embodiments, the nucleic acid encoding for a histone stem-loop and a poly(A) sequence or a polyadenylation signal may code for a tumor antigen or fragment thereof such as the nucleic acid sequences described in International Patent Publication Nos. WO2013/120500 and WO2013/120627, the contents of both of which are incorporated herein by reference in their entirety. In some embodiments, the nucleic acid encoding for a histone stem-loop and a poly(A) sequence or a polyadenylation signal may code for an autoimmune self-antigen such as the nucleic acid sequences described in International Patent Publication Nos. WO2013/120498 and WO2013/120626, the contents of both of which are incorporated herein by reference in their entirety.

mRNA: Triple Helices

In some embodiments, nucleic acids of the present invention (e.g., the mRNA of the present invention) may include a triple helix on the 3′ end of the nucleic acid. The 3′ end of the nucleic acids of the present invention may include a triple helix alone or in combination with a poly(A) tail.

In some embodiments, the nucleic acid of the present invention may include at least a first and a second U-rich region, a conserved stem-loop region between the first and second region and an A-rich region. The first and second U-rich region and the A-rich region may associate to form a triple helix on the 3′ end of the nucleic acid. This triple helix may stabilize the nucleic acid, enhance the translational efficiency of the nucleic acid and/or protect the 3′ end from degradation. Triple helices include, but are not limited to, the triple helix sequence of metastasis-associated lung adenocarcinoma transcript 1 (MALAT1), MEN-β and polyadenylated nuclear (PAN) RNA (see, Wilusz et al., Genes & Development 26:2392-2407 (2012); herein incorporated by reference in its entirety).

In some embodiments, the triple helix may be formed from the cleavage of a MALAT1 sequence prior to the cloverleaf structure. While not meaning to be bound by theory, MALAT1 is a long non-coding RNA which, when cleaved, forms a triple helix and a tRNA-like cloverleaf structure. The MALAT1 transcript then localizes to nuclear speckles and the tRNA-like cloverleaf localizes to the cytoplasm (Wilusz et al., Cell. 135(5): 919-932 (2008); incorporated herein by reference in its entirety).

As a non-limiting example, the terminal end of the nucleic acid of the present invention including the MALAT1 sequence can then form a triple helix structure, after RNaseP cleavage from the cloverleaf structure, which stabilizes the nucleic acid (Peart et al., WIREs RNA. 4(5):491-506 (2013); incorporated herein by reference in its entirety).

In some embodiments, the nucleic acids or mRNA described herein include a MALAT1 sequence. In some embodiments, the nucleic acids or mRNA may be polyadenylated. In yet another embodiment, the nucleic acids or mRNA is not polyadenylated but has an increased resistance to degradation compared to unaltered nucleic acids or mRNA.

In some embodiments, the nucleic acids of the present invention may include a MALAT1 sequence in the second flanking region (e.g., the 3′-UTR). As a non-limiting example, the MALAT1 sequence may be human or mouse.

mRNA: Translation Enhancer Elements (TEEs)

The term “translational enhancer element” or “translation enhancer element” (herein collectively referred to as “TEE”) refers to sequences that increase the amount of polypeptide or protein produced from an mRNA. TEEs are conserved elements in the UTR which can promote translational activity of a nucleic acid such as, but not limited to, cap-dependent or cap-independent translation. The conservation of these sequences has been previously shown by Pánek et al. Nucleic Acids Research. 41(16): 7625-7634 (2013); incorporated herein by reference in its entirety) across 14 species including humans.

In some embodiments, the 5′-UTR of the mRNA includes at least one TEE. The TEE may be located between the transcription promoter and the start codon. The mRNA with at least one TEE in the 5′-UTR may include a cap at the 5′-UTR. Further, at least one TEE may be located in the 5′-UTR of mRNA undergoing cap-dependent or cap-independent translation.

The TEEs known may be in the 5′-leader of the Gtx homeodomain protein (Chappell et al., Proc. Natl. Acad. Sci. USA 101:9590-9594 (2004), incorporated herein by reference in their entirety).

In another non-limiting example, TEEs are disclosed as SEQ ID NOs: 1-35 in US Patent Publication No. US20090226470, SEQ ID NOs: 1-35 in US Patent Publication No. US20130177581, SEQ ID NOs: 1-35 in International Patent Publication No. WO2009075886, SEQ ID NOs: 1-5, and 7-645 in International Patent Publication No. WO2012009644, SEQ ID NO: 1 in International Patent Publication No. WO1999024595, SEQ ID NO: 1 in U.S. Pat. No. 6,310,197, and SEQ ID NO: 1 in U.S. Pat. No. 6,849,405, each of which is incorporated herein by reference in its entirety.

The TEE may be an internal ribosome entry site (IRES). HCV-IRES or an IRES element such as, but not limited to, those described in U.S. Pat. No. 7,468,275, US Patent Publication Nos. US20070048776 and US20110124100 and International Patent Publication Nos. WO2007025008 and WO2001055369, each of which is incorporated herein by reference in its entirety. The IRES elements may include, but are not limited to, the Gtx sequences (e.g., Gtx9-nt, Gtx8-nt, Gtx7-nt) described by Chappell et al. (Proc. Natl. Acad. Sci. USA 101:9590-9594 (2004)) and Zhou et al. (PNAS 102:6273-6278 (2005)) and in US Patent Publication Nos. US20070048776 and US20110124100 and International Patent Publication No. WO2007025008, each of which is incorporated herein by reference in its entirety.

Additional examples of TEEs are disclosed in U.S. Pat. Nos. 6,310,197, 6,849,405, 7,456,273, 7,183,395; US Patent Publication Nos. US20090226470. US20070048776, US20110124100, US20090093049, US20130177581; International Patent Publication Nos. WO2009075886, WO2007025008, WO2012009644, WO2001055371 WO1999024595; and European Patent Publications Nos. EP2610341A1 and EP2610340A1; each of which is incorporated herein by reference in its entirety.

In some embodiments, the polynucleotides, primary constructs, alternative nucleic acids and/or mRNA may include at least one TEE that is described in International Patent Publication Nos. WO1999024595, WO2012009644, WO2009075886, WO2007025008, WO1999024595, European Patent Publication Nos. EP2610341A1 and EP2610340A1, U.S. Pat. No. 6,310,197. U.S. Pat. Nos. 6,849,405, 7,456,273, 7,183,395, US Patent Publication No. US20090226470, US20110124100, US20070048776, US20090093049, and US20130177581 each of which is incorporated herein by reference in its entirety. The TEE may be located in the 5′-UTR of the mRNA.

In some embodiments, the polynucleotides, primary constructs, alternative nucleic acids and/or mmRNA may include at least one TEE that has at least 50%, at least 55%, at least 60%, at least 65%, at least 70%, at least 75%, at least 80%, at least 85%, at least 90%, at least 95% or at least 99% identity with the TEEs described in US Patent Publication Nos. US20090226470, US20070048776, US20130177581 and US20110124100, International Patent Publication Nos. WO1999024595, WO2012009644, WO2009075886 and WO2007025008, European Patent Publication No. EP2610341A1 and EP2610340A1, and U.S. Pat. Nos. 6,310,197, 6,849,405, 7,456,273, and 7,183,395, each of which is incorporated herein by reference in its entirety.

Multiple copies of a specific TEE can be present in mRNA. The TEEs in the translational enhancer polynucleotides can be organized in one or more sequence segments. A sequence segment can harbor one or more of the specific TEEs exemplified herein, with each TEE being present in one or more copies. When multiple sequence segments are present in a translational enhancer polynucleotide, they can be homogenous or heterogeneous. Thus, the multiple sequence segments in a translational enhancer polynucleotide can harbor identical or different types of the specific TEEs exemplified herein, identical or different number of copies of each of the specific TEEs, and/or identical or different organization of the TEEs within each sequence segment.

In some embodiments, the 5′-UTR of the mRNA may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 5′-UTR of mRNA of the present invention may be the same or different TEE sequences. The TEE sequences may be in a pattern such as ABABAB or AABBAABBAABB or ABCABCABC or variants thereof repeated once, twice, or more than three times. In these patterns, each letter, A, B, or C represent a different TEE sequence at the nucleotide level.

In some embodiments, the 5′-UTR may include a spacer to separate two TEE sequences. As a non-limiting example, the spacer may be a 15 nucleotide spacer and/or other spacers known in the art. As another non-limiting example, the 5′-UTR may include a TEE sequence-spacer module repeated at least once, at least twice, at least 3 times, at least 4 times, at least 5 times, at least 6 times, at least 7 times, at least 8 times and at least 9 times, or more than 9 times in the 5′-UTR.

In some embodiments, the TEE in the 5′-UTR of the mRNA of the present invention may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in US Patent Publication Nos. US20090226470, US20070048776, US20130177581 and US20110124100, International Patent Publication Nos. WO1999024595. WO2012009644, WO2009075886 and WO2007025008, European Patent Publication Nos. EP2610341A1 and EP2610340A1, and U.S. Pat. Nos. 6,310,197, 6,849,405, 7,456,273, and 7,183,395 each of which is incorporated herein by reference in its entirety. In some embodiments, the TEE in the 5′-UTR of the mRNA of the present invention may include a 5-30 nucleotide fragment, a 5-25 nucleotide fragment, a 5-20 nucleotide fragment, a 5-15 nucleotide fragment, a 5-10 nucleotide fragment of the TEE sequences disclosed in US Patent Publication Nos. US20090226470, US20070048776, US20130177581, and US20110124100, International Patent Publication No. WO1999024595, WO2012009644, WO2009075886, and WO2007025008, European Patent Publication No. EP2610341A1 and EP2610340A1, and U.S. Pat. Nos. 6,310,197, 6,849,405, 7,456,273, and 7,183,395; each of which is incorporated herein by reference in its entirety.

In some embodiments, the TEE in the 5′-UTR of the mRNA of the present invention may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in Chappell et al. (Proc. Natl. Acad. Sci. USA 101:9590-9594 (2004)) and Zhou et al. (PNAS 102:6273-6278 (2005)), in Supplemental Table 1 and in Supplemental Table 2 disclosed by Wellensiek et al. (Nature Methods. 10(8):747-750 (2013)); each of which is herein incorporated by reference in its entirety. In some embodiments, the TEE in the 5′-UTR of the polynucleotides, primary constructs, alternative nucleic acids and/or mmRNA of the present invention may include a 5-30 nucleotide fragment, a 5-25 nucleotide fragment, a 5-20 nucleotide fragment, a 5-15 nucleotide fragment, a 5-10 nucleotide fragment of the TEE sequences disclosed in Chappell et al. (Proc. Natl. Acad. Sci. USA 101:9590-9594, 2004) and Zhou et al. (PNAS 102:6273-6278, 2005), in Supplemental Table 1 and in Supplemental Table 2 disclosed by Wellensiek et al. (Nature Methods. 10(8):747-750 (2013); each of which is incorporated herein by reference in its entirety.

In some embodiments, the TEE used in the 5′-UTR of the mRNA of the present invention is an IRES sequence such as, but not limited to, those described in U.S. Pat. No. 7,468,275 and International Patent Publication No. WO2001055369, each of which is incorporated herein by reference in its entirety.

In some embodiments, the TEEs used in the 5′-UTR of the mRNA of the present invention may be identified by the methods described in US Patent Publication Nos. US20070048776 and US20110124100 and International Patent Publication Nos. WO2007025008 and WO2012009644, each of which is incorporated herein by reference in its entirety.

In some embodiments, the TEEs used in the 5′-UTR of the mRNA of the present invention may be a transcription regulatory element described in U.S. Pat. Nos. 7,456,273 and 7,183,395, US Patent Publication No. US20090093049, and International Publication No. WO2001055371, each of which is incorporated herein by reference in its entirety. The transcription regulatory elements may be identified by methods known in the art, such as, but not limited to, the methods described in U.S. Pat. Nos. 7,456,273 and 7,183,395, US Patent Publication No. US20090093049, and International Publication No. WO2001055371, each of which is incorporated herein by reference in its entirety.

In yet another embodiment, the TEE used in the 5′-UTR of the mRNA of the present invention is an oligonucleotide or portion thereof as described in U.S. Pat. Nos. 7,456,273 and 7,183,395, US Patent Publication No. US20090093049, and International Publication No. WO2001055371, each of which is incorporated herein by reference in its entirety.

The 5′-UTR including at least one TEE described herein may be incorporated in a monocistronic sequence such as, but not limited to, a vector system or a nucleic acid vector. As a non-limiting example, the vector systems and nucleic acid vectors may include those described in U.S. Pat. Nos. 7,456,273 and 7,183,395, US Patent Publication Nos. US20070048776, US20090093049, and US20110124100 and International Patent Publication Nos. WO2007025008 and WO2001055371, each of which is incorporated herein by reference in its entirety.

In some embodiments, the TEEs described herein may be located in the 5′-UTR and/or the 3′-UTR of the mRNA. The TEEs located in the 3′-UTR may be the same and/or different than the TEEs located in and/or described for incorporation in the 5′-UTR.

In some embodiments, the 3′-UTR of the mRNA may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 3′-UTR of the polynucleotides, primary constructs, alternative nucleic acids and/or mmRNA of the present invention may be the same or different TEE sequences. The TEE sequences may be in a pattern such as ABABAB or AABBAABBAABB or ABCABCABC or variants thereof repeated once, twice, or more than three times. In these patterns, each letter, A, B, or C represent a different TEE sequence at the nucleotide level.

In some embodiments, the 3′-UTR may include a spacer to separate two TEE sequences. As a non-limiting example, the spacer may be a 15-nucleotide spacer and/or other spacers known in the art. As another non-limiting example, the 3′-UTR may include a TEE sequence-spacer module repeated at least once, at least twice, at least 3 times, at least 4 times, at least 5 times, at least 6 times, at least 7 times, at least 8 times and at least 9 times or more than 9 times in the 3′-UTR.

mRNA: Heterologous 5′-UTRs

5′-UTRs of an mRNA of the invention may be homologous or heterologous to the coding region found in the mRNA. Multiple 5′ UTRs may be included in mRNA and may be the same or of different sequences. Any portion of the mRNA, including none, may be codon optimized and any may independently contain one or more different structural or chemical alterations, before and/or after codon optimization.

Shown in Lengthy Table 21 in International Patent Publication No. WO 2014/081507, and in Lengthy Table 21 and in Table 22 in International Patent Publication No. WO 2014/081507, the contents of each of which are incorporated herein by reference in their entirety, is a listing of the start and stop site of mRNAs. In Table 21 each 5′-UTR (5′-UTR-005 to 5′-UTR 68511) is identified by its start and stop site relative to its native or wild type (homologous) transcript (ENST; the identifier used in the ENSEMBL database).

To alter one or more properties of the mRNA of the invention, 5′-UTRs which are heterologous to the coding region of the mRNA are engineered into the mRNA. The mRNA (e.g., an mRNA in a composition described herein) is administered to cells, tissue, or organisms, and outcomes such as protein level, localization, and/or half-life are measured to evaluate the beneficial effects the heterologous 5′-UTR may have on mRNA. Variants of the 5′ UTRs may be utilized wherein one or more nucleotides are added or removed to the termini, including A, T, C or G. 5′-UTRs may also be codon-optimized or altered in any manner described herein.

mRNA: RNA Motifs for RNA Binding Proteins

RNA binding proteins (RBPs) can regulate numerous aspects of co- and post-transcription gene expression, such as, but not limited to, RNA splicing, localization, translation, turnover, polyadenylation, capping, alteration, export, and localization. RNA-binding domains (RBDs), such as, but not limited to, RNA recognition motif (RR) and hnRNP K-homology (KH) domains, typically regulate the sequence association between RBPs and their RNA targets (Ray et al. Nature, 499:172-177 (2013); incorporated herein by reference in its entirety). In some embodiments, the canonical RBDs can bind short RNA sequences. In some embodiments, the canonical RBDs can recognize structure RNAs.

In some embodiments, to increase the stability of the mRNA of interest, an mRNA encoding HuR is co-transfected or co-injected along with the mRNA of interest into the cells or into the tissue. These proteins can also be tethered to the mRNA of interest in vitro and then administered to the cells together. Poly A tail binding protein, PABP interacts with eukaryotic translation initiation factor eIF4G to stimulate translational initiation. Co-administration of mRNAs encoding these RBPs along with the mRNA drug and/or tethering these proteins to the mRNA drug in vitro and administering the protein-bound mRNA into the cells can increase the translational efficiency of the mRNA. The same concept can be extended to co-administration of mRNA along with mRNAs encoding various translation factors and facilitators as well as with the proteins themselves to influence RNA stability and/or translational efficiency.

In some embodiments, the nucleic acids and/or mRNA may include at least one RNA-binding motif such as, but not limited to an RNA-binding domain (RBD).

In some embodiments, the RBD may be any of the RBDs, fragments or variants thereof descried by Ray et al. (Nature. 499:172-177 (2013); incorporated herein by reference in its entirety).

In some embodiments, the nucleic acids or mRNA of the present invention may include a sequence for at least one RNA-binding domain (RBDs). When the nucleic acids or mRNA of the present invention include more than one RBD, the RBDs do not need to be from the same species or even the same structural class.

In some embodiments, at least one flanking region (e.g., the 5′-UTR and/or the 3′-UTR) may include at least one RBD. In some embodiments, the first flanking region and the second flanking region may both include at least one RBD. The RBD may be the same or each of the RBDs may have at least 60% (e.g., at least 70%, 80%, or 90%) sequence identity to the other RBD. As a non-limiting example, at least on RBD may be located before, after and/or within the 3′-UTR of the nucleic acid or mRNA of the present invention. As another non-limiting example, at least one RBD may be located before or within the first 300 nucleosides of the 3′-UTR.

In some embodiments, the nucleic acids and/or mRNA of the present invention may include at least one RBD in the first region of linked nucleosides. The RBD may be located before, after, or within a coding region (e.g., the ORF).

In another embodiment, the first region of linked nucleosides and/or at least one flanking region may include at least on RBD. As a non-limiting example, the first region of linked nucleosides may include a RBD related to splicing factors and at least one flanking region may include a RBD for stability and/or translation factors.

In some embodiments, the nucleic acids and/or mRNA of the present invention may include at least one RBD located in a coding and/or non-coding region of the nucleic acids and/or mRNA.

In some embodiments, at least one RBD may be incorporated into at least one flanking region to increase the stability of the nucleic acid and/or mRNA of the present invention.

In some embodiments, an antisense locked nucleic acid (LNA) oligonucleotides and exon-junction complexes (EJCs) may be used in the RNA binding protein motif. The LNA and EJCs may be used around a start codon (−4 to +37 where the A of the AUG codons is +1) in order to decrease the accessibility to the first start codon (AUG).

### Nucleic Acids as Agents for Delivering Anti-GPC3 Antibodies or Binding Proteins

The compositions of the disclosure can be administered not only as antibodies or antigen-binding fragments, but also in the form of nucleic acids. The examples of nucleic acids described herein may be used to deliver antibodies or antigen-binding fragments to a subject. These nucleic acids (e.g., RNAs, such as mRNAs) may be used as therapeutic agents to express antibodies or antigen-binding fragments of the disclosure as a therapy to treat a target disease.

### Pharmaceutical Compositions

Pharmaceutical compositions containing an anti-GPC3 antibody, antigen-binding fragment, binding proteins described herein or nucleic acid encoding the same, or CAR-T cells described herein can be prepared using methods known in the art. Pharmaceutical compositions described herein may contain an anti-GPC3 antibody or antigen-binding fragment, or a nucleic acid encoding the same, or a CAR-T cell described herein in combination with one or more pharmaceutically acceptable excipients. For instance, pharmaceutical compositions described herein can be prepared using physiologically acceptable carriers, excipients, or stabilizers (Remington's Pharmaceutical Sciences (19th ed., 1995), incorporated herein by reference), and in a desired form, e.g., in the form of lyophilized formulations or aqueous solutions. The compositions can also be prepared so as to contain the active agent (e.g., an anti-GPC3 antibody, antigen-binding fragment, or a nucleic acid encoding the same, or CAR-Tcells) at a desired concentration. For example, a pharmaceutical composition described herein may contain at least 10% (e.g., 10%, 20%, 30%, 40%, 50%, 60%, 70%, 80%, 90%, 95%, 97%, 98%, 99%, 99.5%, 99.9%, or 100%) active agent by weight (w/w).

Additionally, an active agent that can be incorporated into a pharmaceutical formulation can itself have a desired level of purity. For example, a polypeptide or nucleic acid described herein may be characterized by a certain degree of purity after isolating the antibody or antigen-binding fragment or CAR-T cell from cell culture media or after chemical synthesis (in the case of antibodies or antigen-binding fragments). An antibody, antigen-biding fragment, nucleic acid, or CAR-T cell described herein may be at least 10% pure prior to incorporating the antibody or antigen-biding fragment or nucleic acid or CAR-T cell into a pharmaceutical composition (e.g., 10%, 20%, 30%, 40%, 50%, 60%, 70%, 80%, 90%, 95%, 97%, 98%, 99%, 99.5%, 99.9%, 99.99%, or 100% pure).

Pharmaceutical compositions can be prepared for storage as lyophilized formulations or aqueous solutions by mixing the active agent having the desired degree of purity with optional pharmaceutically acceptable carriers, excipients or stabilizers typically employed in the art, e.g., buffering agents, stabilizing agents, preservatives, isotonifiers, non-ionic detergents, antioxidants, and other miscellaneous additives. See, e.g., Remington's Pharmaceutical Sciences (19th ed., 1995), incorporated herein by reference). Such additives must be nontoxic to the recipients at the dosages and concentrations employed.

### Buffering Agents

Buffering agents help to maintain the pH in the range which approximates physiological conditions. Suitable buffering agents for use with the pharmaceutical compositions of the disclosure include both organic and inorganic acids and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.), and acetate buffers (e.g., acetic acid-sodium acetate mixture, acetic acid-sodium hydroxide mixture, etc.). Additionally, phosphate buffers, histidine buffers, and trimethylamine salts such as Tris can be used.

### Preservatives

Preservatives can be added to a composition described herein, for example, to inhibit microbial growth. Suitable preservatives for use with the pharmaceutical compositions of the disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonifiers, also known as “stabilizers,” can be added to ensure isotonicity of liquid compositions described herein and include polhydric sugar alcohols, for example trihydric or higher sugar alcohols, such as glycerin, arabitol, xylitol, sorbitol, and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids such as arginine, lysine, glycine, glutamine, asparagine, histidine, alanine, ornithine, L-leucine, 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as HAS, BSA, MSA, gelatin, or immunoglobulins; hydrophilic polymers, such as polyvinylpyrrolidone monosaccharides, such as xylose, mannose, fructose, glucose: disaccharides such as lactose, maltose, sucrose and trisaccharides such as raffinose; and polysaccharides such as dextran.

### Detergents

In some embodiments, non-ionic surfactants or detergents (also known as “wetting agents”) are added to the pharmaceutical composition, for example, to help solubilize the therapeutic agent as well as to protect the therapeutic agent against agitation-induced aggregation, which also permits the formulation to be exposed to shear surface stressed without causing denaturation of the protein. Suitable non-ionic surfactants include, for example and without limitation, polysorbates (20, 80, etc.), polyoxamers (184, 188 etc.), Pluronic polyols, polyoxyethylene sorbitan monoethers (TWEEN®-20, TWEEN®-80, etc.).

### Other Pharmaceutical Carriers

Alternative pharmaceutically acceptable carriers that can be incorporated into a pharmaceutical composition described herein may include dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils, but not limited to. A pharmaceutical composition described herein may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

### Lipid Nanoparticle (LNP) Compositions

The present disclosure provides LNP compositions with advantageous properties. The lipid nanoparticle compositions described herein may be used for the delivery of therapeutic and/or prophylactic agents, e.g., mRNAs, to mammalian cells or organs. For example, the lipid nanoparticles described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In some embodiments, the present application provides pharmaceutical compositions comprising: (a) a delivery agent comprising a lipid nanoparticle; and (b) a polynucleotide encoding an antibody or antigen-binding fragment of the disclosure. In some embodiments, the present application provides pharmaceutical compositions comprising a CAR-T cell.

### Lipid Nanoparticles

In some embodiments, polynucleotides of the present disclosure (e.g., mRNA) are included in a lipid nanoparticle (LNP). Lipid nanoparticles according to the present disclosure may comprise: (i) an ionizable lipid (e.g., an ionizable amino lipid); (ii) a sterol or other structural lipid; (iii) a non-cationic helper lipid or phospholipid; and (iv) a PEG-modified lipid. In some embodiments, lipid nanoparticles according to the present disclosure further comprise one or more polynucleotides of the present disclosure (e.g., mRNA).

The lipid nanoparticles according to the present disclosure can be generated using components, compositions, and methods as are generally known in the art, see, for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

In some embodiments, the lipid nanoparticle comprises an ionizable cationic lipid (e.g., an ionizable amino lipid) at a content of 20-60 mol. %, 25-60 mol. %, 30-60 mol. %, 35-60 mol. %, 40-60 mol. %, 45-60 mol. %, 20-55 mol. %, 25-55 mol. %, 30-55 mol. %, 35-55 mol. %, 40-55 mol. %, 45-55 mol. %. 20-50 mol. %, 25-50 mol. %, 30-50 mol. %, 35-50 mol. %, or 40-50 mol. %. For example, the lipid nanoparticle may comprise an ionizable cationic lipid (e.g., an ionizable amino lipid) at a content of 40-50 mol. %, 45-50 mol. %, 45-46 mol. %, 46-47 mol. %, 47-48 mol. %, 48-49 mol. %, or 49-50 mol. %, for example about 45 mol. %, about 45.5 mol. %, about 46 mol. %, about 46.5 mol. %, about 47 mol. %, about 47.5 mol. %, about 48 mol. %, about 48.5 mol. %, about 49 mol. %, or about 49.5 mol. % ionizable cationic lipid (e.g., an ionizable amino lipid).

In some embodiments, the lipid nanoparticle comprises a non-cationic helper lipid or phospholipid at a content of 5-25 mol. %. For example, the lipid nanoparticle may comprise a non-cationic helper lipid or phospholipid at a content of molar ratio of 5-25 mol. %, 5-20 mol. %, 5-15 mol. %, 10-25 mol. %, 10-20 mol. %. 10-15 mol. %, 5-6 mol. %. 6-7 mol. %. 7-8 mol. %. 8-9 mol. %. 9-10 mol. %. 10-11 mol. %, 11-12 mol. %, 12-13 mol. %, 13-14 mol. %, 14-15 mol. %, 10-14 mol. %, 10-13 mol. %, 10-12 mol. %, 10-11 mol. %, 9-15 mol. %, 9-14 mol. %, 9-13 mol. %, 9-12 mol. %, or 9-11 mol. % non-cationic lipid.

In some embodiments, the lipid nanoparticle comprises a sterol or other structural lipid at a content molar ratio of 25-55 mol. %, 25-50 mol. %, 25-45 mol. %, 25-40 mol. %, 25-35 mol. %, 30-55 mol. %, 30-50 mol. %, 30-45 mol. %, 30-40 mol. %, 30-35 mol. %, 35-55 mol. %, 35-50 mol. %, 35-45 mol. %, 35-40 mol. %, 25-30 mol. %, 30-35 mol. %, 25-28 mol. %, 28-30 mol. %, 30-33 mol. %, 35-38 mol. %, 38-40 mol. %. 36-40 mol. %, 37-40 mol. %, 38-40 mol. %. 38-39 mol. %, 36-40 mol. %, 37-40 mol. %. 36-39 mol. %, or 37-39 mol. %. For example, the lipid nanoparticle may comprise a sterol or other structural lipid at a content of about 30 mol. %, about 30.5 mol. %, about 31.0 mol. %, about 31.5 mol. %, about 32.0 mol. %, about 32.5 mol. %, about 33.0 mol. %, about 33.5 mol. %, about 34.0 mol. %, about 34.5 mol. %, about 35.0 mol. %, about 35.5 mol. %, about 36.0 mol. %, about 36.5 mol. %, about 37.0 mol. %, about 37.5 mol. %, about 38.0 mol. %, about 38.5 mol. %, about 39.0 mol. %, about 39.5 mol. %, about 40.0 mol. %, about 40.5 mol. %, about 41.0 mol. %, about 41.5 mol. %, about 42.0 mol. %, about 42.5 mol. %, about 43.0 mol. %, about 43.5 mol. %, about 44.0 mol. %, about 44.5 mol. %, or about 45.0 mol. %.

In some embodiments, the lipid nanoparticle comprises a PEG-modified lipid at a content of 0.5-15 mol. %. 1.0-15 mol. %, 1.5-15 mol. %, 2.0-15 mol. %, 2.5-15 mol. %, 3.0-15 mol. %, 3.5-15 mol. %, 4.0-15 mol. %, 4.5-15 mol. %, 5.0-15 mol. %, 10-15 mol. %, 0.5-10 mol. %, 0.5-5 mol. %, 0.5-4.5 mol. %, 0.5-4.0 mol. %, 0.5-3.5 mol. %, 0.5-3.0 mol. %, 0.5-2.5 mol. %, 0.5-2.0 mol. %, 0.5-1.5 mol. %, 0.5-1.0 mol. %, 1.0-10 mol. %, 1.0-5 mol. %, 1.0-4.5 mol. %, 1.0-4.0 mol. %, 1.0-3.5 mol. %, 1.0-3.0 mol. %, 1.0-2.5 mol. %. 1.0-2.0 mol. %. 1.0-1.5 mol. %. 1.5-5.0 mol. %. 1.5-4.5 mol. %. 1.5-4.0 mol. %, 1.5-3.5 mol. %, 1.5-3.0 mol. %, 1.5-2.5 mol. %, 1.5-2.0 mol. %, 2.0-5.0 mol. %, 2.0-4.5 mol. %, 2.0-4.0 mol. %, 2.0-3.5 mol. %, 2.0-3.0 mol. %, or 2.0-2.5 mol. %. For example, the lipid nanoparticle may comprise a PEG-modified lipid at a content of a about 0.5 mol. %, about 1.0 mol. %, about 1.5 mol. %, about 2.0 mol. %, about 2.5 mol. %, about 3.0 mol. %, about 3.5 mol. %, about 4.0 mol. %, about 4.5 mol. %, about 5.0 mol. %, about 6.0 mol. %, about 7.0 mol. %, about 8.0 mol. %, about 9.0 mol. %, about 10.0 mol. %, or about 15.0 mol. %.

In some embodiments, the lipid nanoparticle comprises: (i) 20 to 60 mol. % ionizable cationic lipid (e.g., ionizable amino lipid), (ii) 25 to 55 mol. % sterol or other structural lipid, (iii) 5 to 25 mol. % non-cationic lipid (e.g., phospholipid), and (iv) 0.5 to 15 mol. % PEG-modified lipid.

In some embodiments, the lipid nanoparticle comprises: (i) 40 to 50 mol. % ionizable cationic lipid (e.g., ionizable amino lipid), (ii) 30 to 45 mol. % sterol or other structural lipid, (iii) 5 to 15 mol. % non-cationic lipid (e.g., phospholipid), and (iv) 1 to 5 mol. % PEG-modified lipid.

In some embodiments, the lipid nanoparticle comprises: (i) 45 to 50 mol. % ionizable cationic lipid (e.g., ionizable amino lipid), (ii) 35 to 45 mol. % sterol or other structural lipid, (iii) 8 to 12 mol. % non-cationic lipid (e.g., phospholipid), and (iv) 1.5 to 3.5 mol. % PEG-modified lipid.

In the following sections, “Compounds” numbered with an “I-” prefix (e.g., “Compound I-1,” “Compound I-2,” “Compound I-3,” “Compound I-VI,” etc., indicate specific ionizable lipid compounds. Likewise, compounds numbered with a “P-” prefix (e.g., “Compound P-I,” etc.) indicate a specific PEG-modified lipid compound.

### Ionizable Ammo Lipids

In some embodiments, the lipid nanoparticle of the present disclosure comprises an ionizable cationic lipid (e.g., an ionizable amino lipid) that is a compound of Formula (I):

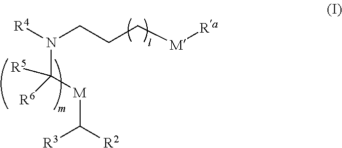

or its N-oxide, or a salt or isomer thereof, wherein R′^{a} is R′^{branched}; wherein
R′^{branched} is:

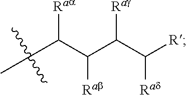

wherein

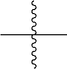

denotes a point of attachment;
wherein R^{αa}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH, wherein n is selected from the group consisting of 1, 2, 3, 4, and 5, and

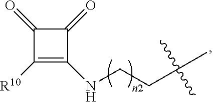

wherein

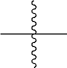

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and

### —OC(O)—;

R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, in Formula (I), R′^{a} is R′^{branched}; R′^{branched} is

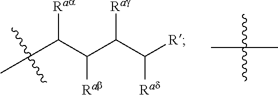

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, in Formula (I), R′^{a} is R′^{branched}; R′^{branched} is

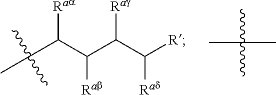

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of the compounds of Formula (I), R′^{a} is R′^{branched};
R′^{branched} is

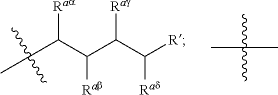

denotes a point of attachment; R^{aα} is C₂₋₁₂ alkyl; R^{aβ}, R^{aγ}, and R^{aδ} are each H; R and R³ are each C₁₋₁₄ alkyl; R⁴ is

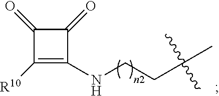

R¹⁰ is NH(C₁₋₆ alkyl); n2 is 2; R⁵ is H; each R⁶ is H; M and M′ are each —C(O)O—; R′ is a C₁₋₁₂ alkyl; l is 5; and m is 7.

In some embodiments of the compounds of Formula (I), R′^{a} is R′^{branched}
R′^{branched} is

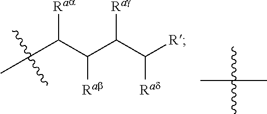

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the compound of Formula (I) is selected from:

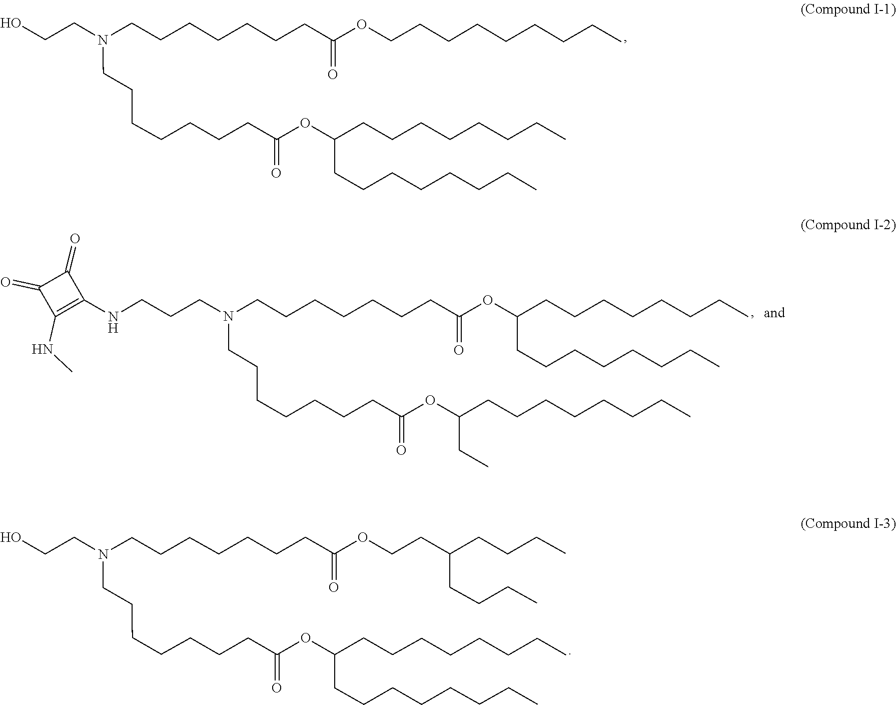 [SMILES: CCCCCCCCCOC(=O)CCCCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the compound of Formula (I) is:

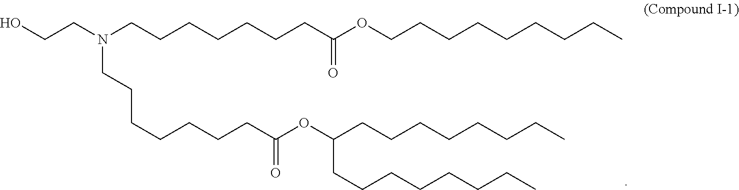 [SMILES: CCCCCCCCCOC(=O)CCCCCCCN(CCO)CCCCCCCC(=O)OC(CCCCCCCC)CCCCCCCC]

In some embodiments, the compound of Formula (I) is:

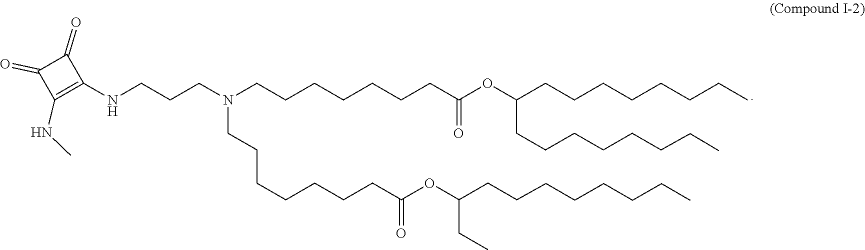 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the compound of Formula (I) is:

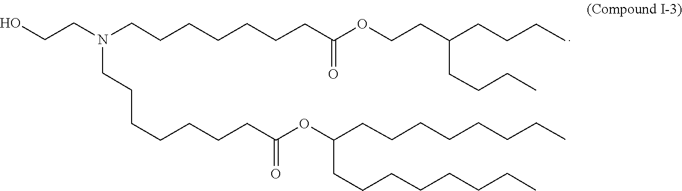 [SMILES: CCCCCCCCC(CCCCCCCC)OC(=O)CCCCCCCN(CCO)CCCCCCCC(=O)OCCC(CCCC)CCCC]

In some aspects, the disclosure relates to a compound of Formula (Ia):

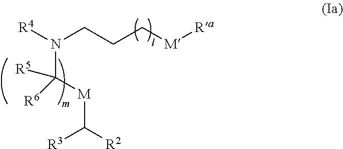

or its N-oxide, or a salt or isomer thereof,
wherein R′^{a} is R′^{branched}; wherein
   R′^{branched} is:

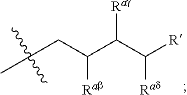

wherein

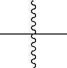

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and
C₂₋₁₄ alkenyl;
   R⁴ is selected from the group consisting of —(CH₂)ₙOH wherein n is selected from the group consisting of 1, 2, 3, 4, and 5, and

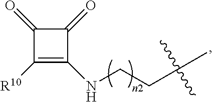

wherein

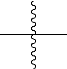

denotes a point of attachment: wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and
—OC(O)—;
   R′ is a C₁₋₁₂ alkyl or C₂₋₁₂ alkenyl;
   l is selected from the group consisting of 1, 2, 3, 4, and 5; and
   m is selected from the group consisting of 5, 6, 7, 8, 9, 10, 11, 12, and 13.

In some aspects, the disclosure relates to a compound of Formula (Ib):

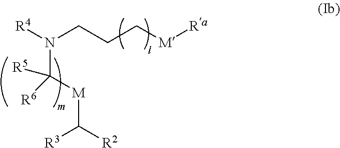

or its N-oxide, or a salt or isomer thereof, wherein R′^{a} is R′^{branched}; wherein
R′^{branched} is:

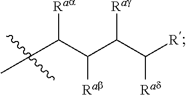

wherein

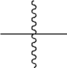

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl. C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and

### —OC(O)—;

R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (I) or (Ib), R′^{a} is R′^{branched}; R′^{branched} is

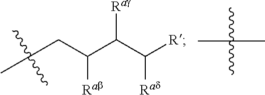

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of Formula (I) or (Ib), R′^{a} is R′^{branched}; R′^{branched} is

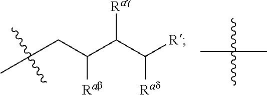

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the disclosure relates to a compound of Formula (Ic):

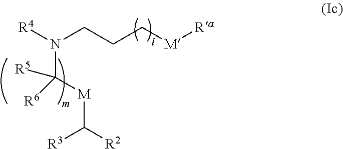

or its N-oxide, or a salt or isomer thereof, wherein R′^{a} is R′^{branched}; wherein
R′^{branched} is:

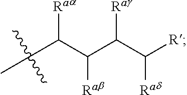

wherein

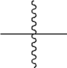

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and
   C₂₋₁₄ alkenyl;
R⁴ is

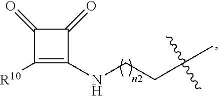

wherein

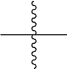

denotes a point of attachment; wherein
R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and

### —OC(O)—;

R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

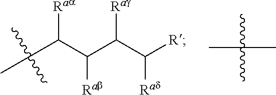

denotes a point of attachment; R^{aβ}, R^{aγ}, and R^{aδ} are each H; R^{a}U is C₂₋₁₂ alkyl; R² and R³ are each C₁₋₁₄ alkyl; R⁴ is

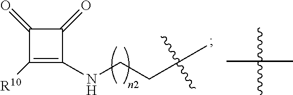

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; l is 5; and m is 7.

In some embodiments, the compound of Formula (Ic) is:

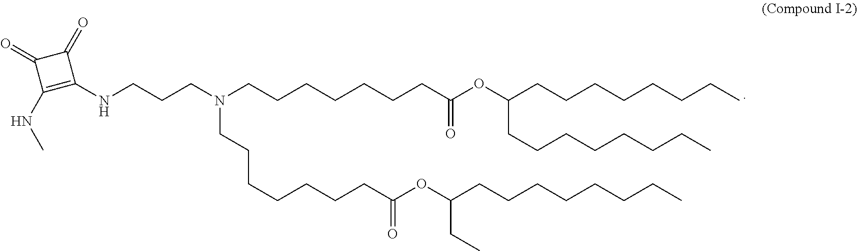 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some aspects, the disclosure relates to a compound of Formula (II):

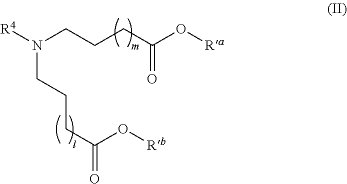

or its N-oxide, or a salt or isomer thereof, wherein R′^{a} is R′^{branched} or R′^{cyclic}; wherein
R′^{branched} is:

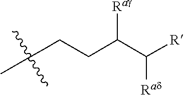

and R′cyclic is:

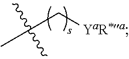

and R′^{b} is:

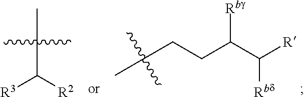

wherein

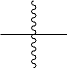

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl:
R⁴ is selected from the group consisting of —(CH₂)ₙOH wherein n is selected from the group consisting of 1, 2, 3, 4, and 5, and

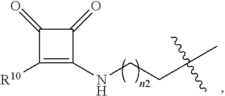

wherein

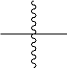

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₂ alkenyl; and
s is 2 or 3;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-a):

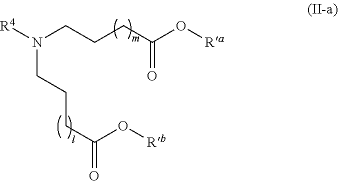

or its N-oxide, or a salt or isomer thereof,
wherein R′^{a} is R′^{branched} or R′^{cyclic}; wherein
R′^{branched} is:

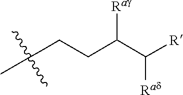

and R′^{b} is:

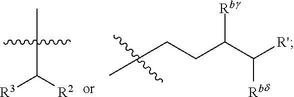

wherein

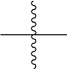

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH wherein n is selected from the group consisting of 1, 2, 3, 4, and 5, and

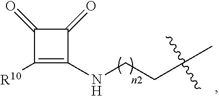

wherein

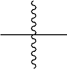

denotes a point of attachment: wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-b):

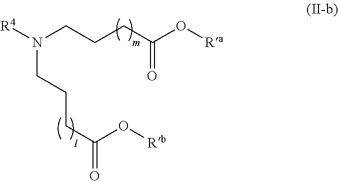

or its N-oxide, or a salt or isomer thereof,
wherein R′^{a} is R′^{branched} or R′^{cyclic}; wherein
R′^{branched} is:

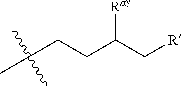

and R′^{b} is:

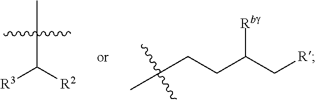

wherein

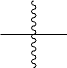

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl:
R⁴ is selected from the group consisting of —(CH₂)ₙOH wherein n is selected from the group consisting of 1, 2, 3, 4, and 5, and

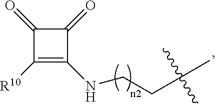

wherein

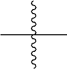

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-c):

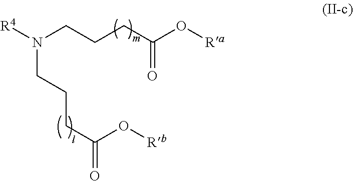

or its N-oxide, or a salt or isomer thereof,
wherein R′^{a} is R′^{branched} or R′^{cyclic}; wherein
R′^{branched} is:

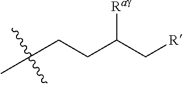

and R′^{b} is:

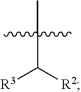

wherein

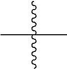

denotes a point of attachment;
wherein R^{aγ} is selected rom the group consisting of C₁₋₁₂ alkyl and C₂₋₁₂ alkenyl;
R² and R³ are each independently selected from the group consisting of C₁₋₁₄ alkyl and
   C₂₋₁₄ alkenyl:
R⁴ is selected from the group consisting of —(CH₂)ₙOH wherein n is selected from the group consisting of 1, 2, 3, 4, and 5, and

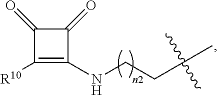

wherein

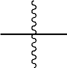

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-d):

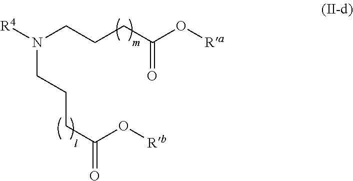

or its N-oxide, or a salt or isomer thereof,
wherein R′^{a} is R′^{branched} or R′^{cyclic}: wherein
R′^{branched} is:

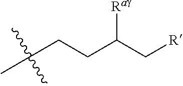

and R′^{b} is:

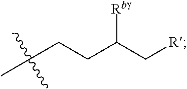

wherein

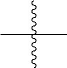

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

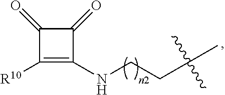

wherein

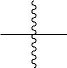

denotes a point of attachment: wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-e):

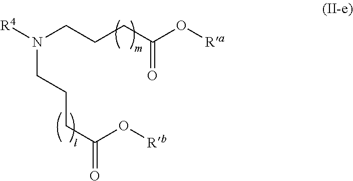

or its N-oxide, or a salt or isomer thereof,
wherein R′^{a} is R′^{branched} or R′^{cyclic}; wherein
R′^{branched} is:

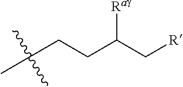

and R′^{b} is:

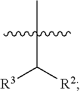

wherein

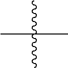

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (II), (II-a), (II-b), (II-c), (II-d), or (II-e), m and l are each independently selected from 4, 5, and 6. In some embodiments of the compound of Formula (II), (II-a), (II-b), (II-c), (II-d), or (II-e), m and l are each 5.

In some embodiments of the compound of Formula (II), (II-a), (II-b), (II-c), (II-d), or (II-e), each R′ independently is a C₁₋₁₂ alkyl. In some embodiments of the compound of Formula (II), (II-a), (II-b), (II-c), (II-d), or (II-e), each R′ independently is a C₂₋₅ alkyl.

In some embodiments of the compound of Formula (II), (II-a), (II-b), (II-c), (II-d), or (II-e), R′^{b} is:

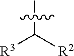

and R² and R³ are each independently a C₁₋₁₄ alkyl. In some embodiments of the compound of Formula (II), (II-a), (II-b), (II-c), (II-d), or (II-e), R′^{b} is:

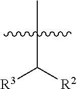

and R² and R³ are each independently a C₆₋₁₀ alkyl. In some embodiments of the compound of Formula (II), (II-a), (II-b), (II-c), (II-d), or (II-e), R′^{b} is:

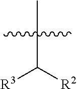

and R² and R³ are each a C₈ alkyl.

In some embodiments of the compound of Formula (II), (II-a), (II-b), (II-c), (II-d), or (II-e), R′^{branched} is:

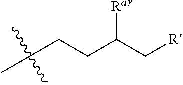

### and R′^{b} is:

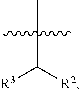

R^{aγ} is a C₁₋₁₂ alkyl and R² and R³ are each independently a C₆₋₁₀ alkyl. In some embodiments of the compound of Formula (II), (II-a), (II-b), (II-c), (II-d), or (II-e) R′^{branched} is:

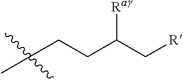

### and R′^{b} is:

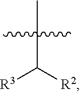

R^{aγ} is a C₂₋₆ alkyl and R² and R³ are each independently a C₆₋₁₀ alkyl. In some embodiments of the compound of Formula (II), (II-a), (II-b), (II-c), (II-d), or (II-e), R′^{branched} is:

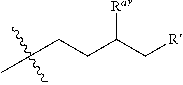

### and R′b is:

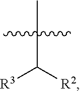

R^{aγ} is a C₂₋₆ alkyl, and R² and R³ are each a C₈ alkyl.

In some embodiments of the compound of Formula (II), (II-a), (II-b), (II-c), (II-d), or (II-e), R′^{branched} is:

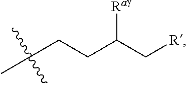

### R′^{b} is:

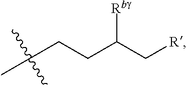

and R^{aγ} and R^{bγ} are each a C₁₋₁₂ alkyl. In some embodiments of the compound of Formula (II), (II-a), (II-b), (II-c), (II-d), or (II-e), R′^{branched} is:

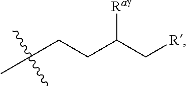

### R′^{b} is:

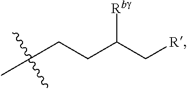

and R^{aγ} and R^{bγ} are each a C₂₋₆ alkyl.

In some embodiments of the compound of Formula (II), (II-a), (II-b), (II-c), (II-d), or (II-e), m and l are each independently selected from 4, 5, and 6 and each R′ independently is a C₁₋₁₂ alkyl. In some embodiments of the compound of Formula (II), (II-a), (II-b), (II-c), (II-d), or (II-e), m and l are each 5 and each R′ independently is a C₂₋₅ alkyl.

In some embodiments of the compound of Formula (II), (II-a), (II-b), (II-c), (II-d), or (II-e), R′^{branched} is:

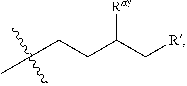

### R′b is:

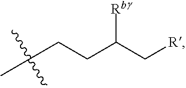

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (II), (II-a), (II-b), (II-c), (II-d), or (II-e), R′^{branched} is:

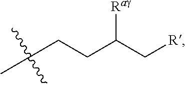

### R′^{b} is:

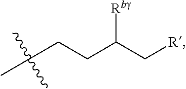

m and l are each 5, each R′ independently is a C₂₋₅ alkyl, and R^{aγ} and R^{bγ} are each a C₂₋₆ alkyl.

In some embodiments of the compound of Formula (II), (II-a), (II-b), (II-c), (II-d), or (II-e), R′^{branched} is:

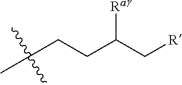

### and R′^{b} is:

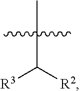

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl. In some embodiments of the compound of Formula (II) (II-a), (II-b), (II-c), (II-d), or (II-e), R′^{branched} is:

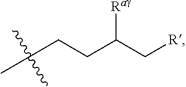

### and R′^{b} is:

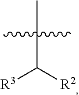

m and l are each 5, R′ is a C₂₋₅ alkyl, R^{aγ} is a C₂₋₆ alkyl, and R² and R³ are each a C₈ alkyl.

In some embodiments of the compound of Formula (II), (II-a), (II-b), (II-c), (II-d), or (II-e), R⁴ is

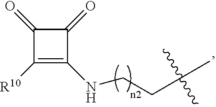

wherein R¹⁰ is NH(C₁₋₆ alkyl) and n2 is 2.

In some embodiments of the compound of Formula (II), (II-a), (II-b), (II-c), (II-d), or (II-e), R⁴ is

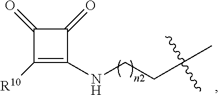

wherein R¹⁰ is NH(CH₃) and n2 is 2.

In some embodiments of the compound of Formula (II), (II-a), (II-b), (II-c), (II-d), or (II-e), R′^{branched} is:

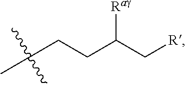

### R′^{b} is:

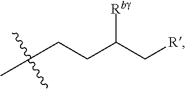

m and l are each independently selected from 4, 5, and 6, each R′ independently is a C₁₋₁₂ alkyl, R^{aγ} and R^{bγ} are each a C₁₋₁₂ alkyl, and R⁴ is

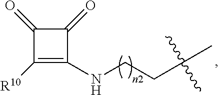

wherein R¹⁰ is NH(C₁₋₆ alkyl), and n2 is 2. In some embodiments of the compound of Formula (II), (II-a), (II-b), (II-c), (II-d), or (II-e), R′^{branched} is:

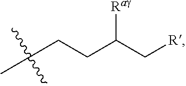

### R′^{b} is:

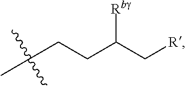

m and l are each 5, each R′ independently is a C₂₋₅ alkyl, R^{aγ} and R^{bγ} are each a C₂₋₆ alkyl, and R⁴ is

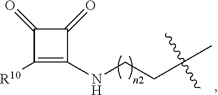

wherein R¹⁰ is NH(CH₃) and n2 is 2.

In some embodiments of the compound of Formula (II), (II-a), (II-b), (II-c), (II-d), or (II-e), R′^{branched} is:

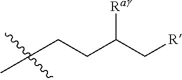

### and R′^{b} is:

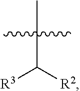

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

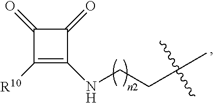

wherein R¹⁰ is NH(C₁₋₆ alkyl) and n2 is 2. In some embodiments of the compound of Formula (II), (II-a), (II-b), (II-c), (II-d), or (II-e), R′^{branched} is:

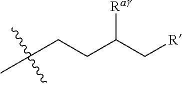

### and R′^{b} is:

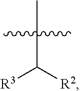

m and l are each 5, R′ is a C₂₋₅ alkyl, R^{aγ} is a C₂₋₆ alkyl, R² and R³ are each a C₈ alkyl, and R⁴ is

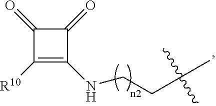

wherein R¹⁰ is NH(CH₃) and n2 is 2.

In some embodiments of the compound of Formula (II), (II-a), (II-b), (II-c), (II-d), or (II-e), R⁴ is —(CH₂)ₙOH and n is 2, 3, or 4. In some embodiments of the compound of Formula (II), (II-a), (II-b), (II-c), (II-d), or (II-e), R⁴ is —(CH₂)ₙOH and n is 2.

In some embodiments of the compound of Formula (II), (II-a), (II-b), (II-c), (II-d), or (II-e), R′^{branched} is:

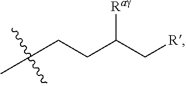

### R′^{b} is:

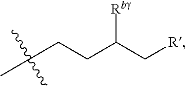

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (II), (II-a), (II-b), (II-c), (II-d), or (II-e), R′^{branched} is:

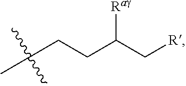

### R′^{b} is:

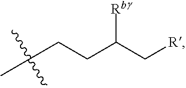

m and l are each 5, each R′ independently is a C₂₋₅ alkyl, R^{aγ} and R^{bγ} are each a C₂₋₆ alkyl, R⁴ is —(CH₂)ₙOH, and n is 2.

In some aspects, the disclosure relates to a compound of Formula (II-f):

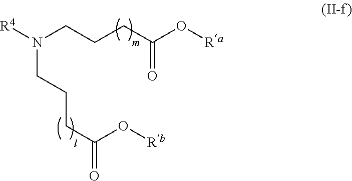

or its N-oxide, or a salt or isomer thereof,
wherein R′^{a} is R′^{branched} or R′^{cyclic}; wherein
R′^{branched} is:

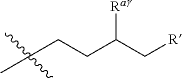

and R′^{b} is:

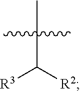

wherein

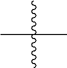

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (II-f), m and l are each 5, and n is 2, 3, or 4.

In some embodiments of the compound of Formula (II-f) R′ is a C₂₋₅ alkyl, R^{aγ} is a C₂₋₆ alkyl, and R² and R³ are each a C₆₋₁₀ alkyl.

In some embodiments of the compound of Formula (II-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some aspects, the disclosure relates to a compound of Formula (II-g):

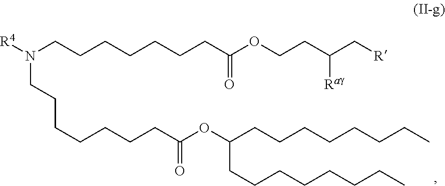

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

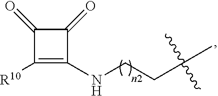

wherein

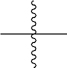

denotes a point of attachment, R¹⁰ is NH(C₁₋₆ alkyl), and n2 is selected from the group consisting of 1, 2, and 3.

In some aspects, the disclosure relates to a compound of Formula (II-h):

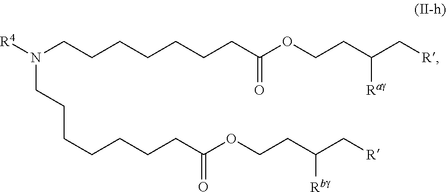

wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

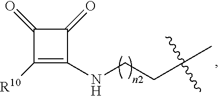

wherein

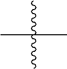

denotes a point of attachment, R¹⁰ is NH(C₁₋₆ alkyl), and n2 is selected from the group consisting of 1, 2, and 3.

In some embodiments of the compound of Formula (II-g) or (II-h), R⁴ is

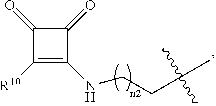

wherein
R¹⁰ is NH(CH₃) and n2 is 2.

In some embodiments of the compound of Formula (II-g) or (II-h), R⁴ is —(CH₂)₂OH.

In some aspects, the disclosure relates to a compound having the Formula (III):

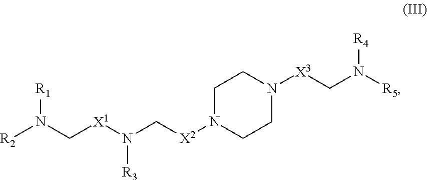

or a salt or isomer thereof, wherein
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl, and wherein;
i) at least one of X¹, X², and X³ is not —CH₂—; and/or
ii) at least one of R₁, R₂, R₃, R₄, and R₅ is —R″MR′.

In some embodiments, R₁, R₂, R₃, R₄, and R₅ are each C₅₋₂₀ alkyl; X¹ is —CH₂—; and X² and X³ are each —C(O)—.

In some embodiments, the compound of Formula (III) is:

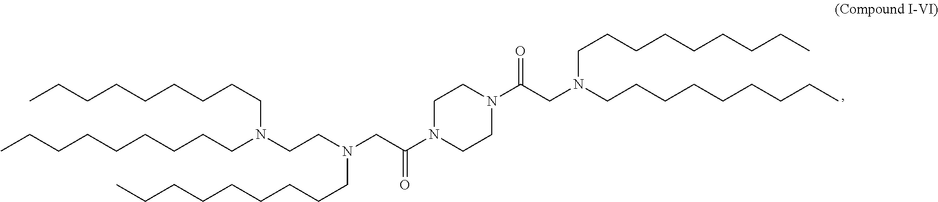 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt or isomer thereof.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the present disclosure comprises 1,2-distearoyl-sn-glycero-3-phosphocholine (DSPC), 1,2-dioleoyl-sn-glycero-3-phosphoethanolamine (DOPE). 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine. 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present disclosure is an analog or variant of DSPC. In certain embodiments, a phospholipid useful or potentially useful in the present disclosure is a compound of Formula (IV):

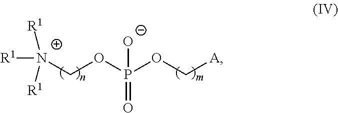

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the Formula:

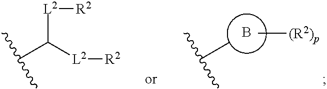

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), —NR^{N}C(O)O, C(O)S, SC(O), C(═NR^{N}), C(═NR^{N})N(R^{N}), NR^{N}C(═NR^{N}), —NR^{N}C(═NR^{N})N(R^{N}), C(S), C(S)N(R^{N}), NR^{N}C(S), NR^{N}C(S)N(R^{N}), S(O), OS(O), —S(O)O, OS(O)O, OS(O)₂, S(O)₂O, OS(O)₂O, N(R^{N})S(O), S(O)N(R^{N}), —N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the Formula:

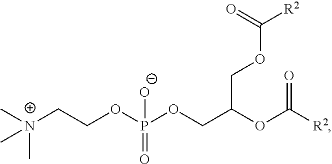

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

### Phospholipid Head Modifications

In certain embodiments, a phospholipid useful or potentially useful in the present disclosure comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following Formulae:

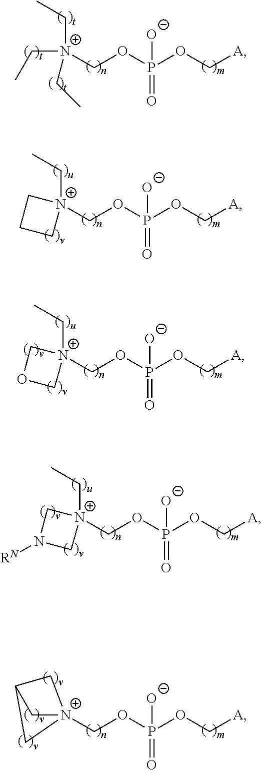

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

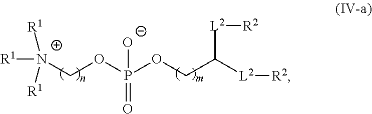

or a salt thereof.

In certain embodiments, a phospholipid useful or potentially useful in the present disclosure comprises a cyclic moiety in place of the glyceride moiety. In certain embodiments, a phospholipid useful in the present disclosure is DSPC, or analog thereof, with a cyclic moiety in place of the glyceride moiety.

In certain embodiments, the compound of Formula (IV) is of Formula (IV-b):

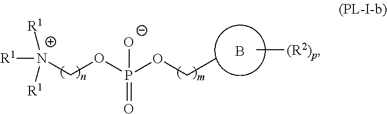

or a salt thereof.

### Phospholipid Tail Modifications

In certain embodiments, a phospholipid useful or potentially useful in the present disclosure comprises a modified tail. In certain embodiments, a phospholipid useful or potentially useful in the present disclosure is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, —C(O), C(O)N(R^{N}), NR^{N}C(O), NR^{N}C(O)N(R^{N}), C(O)O, OC(O), OC(O)O, —OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), —S(O)O, OS(O)O, OS(O)₂, S(O)₂O, OS(O)₂O, N(R^{N})S(O), S(O)N(R^{N}), —N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O.

In certain embodiments, the compound of Formula (IV) is of Formula (IV-c):

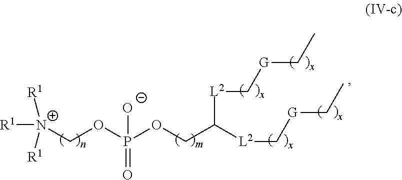

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, N(R^{N}), O, S, —C(O), C(O)N(R^{N}), NR^{N}C(O), NR^{N}C(O)N(R^{N}), C(O)O, OC(O), OC(O)O, —OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}). C(S). C(S)N(R^{N}), NR^{N}C(S). NR^{N}C(S)N(R^{N}), S(O), OS(O), —S(O)O, OS(O)O, OS(O)₂, S(O)₂O, OS(O)₂O, N(R^{N})S(O), S(O)N(R^{N}), —N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O. Each possibility represents a separate embodiment of the present disclosure.

In certain embodiments, a phospholipid useful or potentially useful in the present disclosure comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present disclosure is a compound of Formula (IV), wherein n is 1, 3, 4, 5, 6, 7, 8, 9, or 10. For example, in certain embodiments, a compound of Formula (IV) is of one of the following Formulae:

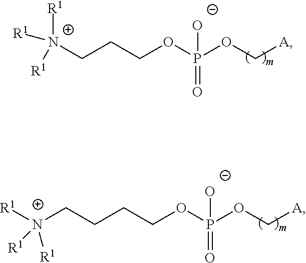

or a salt thereof.

### Alternative Lipids

In certain embodiments, a phospholipid useful or potentially useful in the present disclosure comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the present disclosure is oleic acid.

In certain embodiments, the alternative lipid is one of the following:

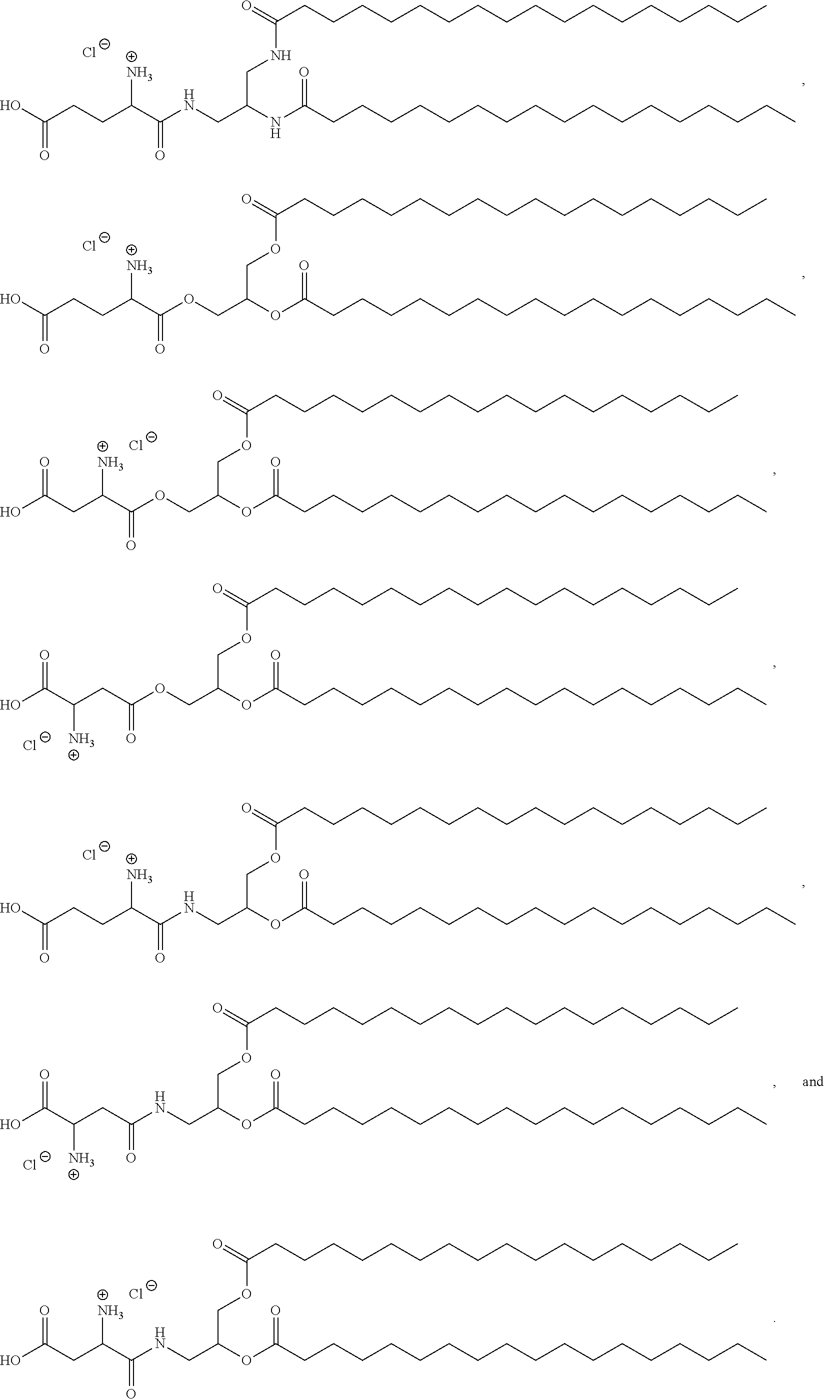

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In one embodiment, a structural lipid comprises a phytosterol, β-sitosterol, β-sitostanol, campesterol, and/or brassicasterol. In some embodiments, a structural lipid comprises β-sitosterol.

In some embodiments, the structural lipids may be one or more of the structural lipids described in U.S. Application No. 62/520,530.

### Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20). PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipahnitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various Formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids. PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

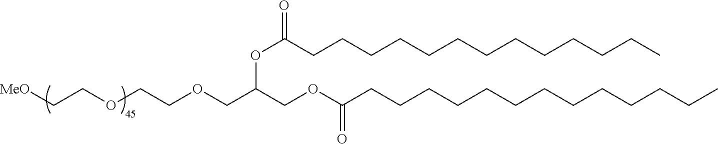

In some embodiments, PEG lipids useful in the present disclosure can be PEGylated lipids described in International Publication No. WO2012099755, the contents of which is herein incorporated by reference in its entirety. Any of these examples of PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present disclosure.

In certain embodiments, a PEG lipid useful in the present disclosure is a compound of Formula (V). Provided herein are compounds of Formula (V):

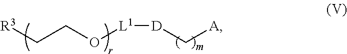

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), —C(O)N(R^{N}), NR^{N}C(O), C(O)O, OC(O), OC(O)O, OC(O)N(R^{N}), NR^{N}C(O)O, or —NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the Formula:

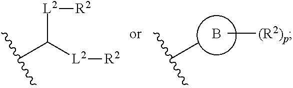

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N})
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, —C(O), C(O)N(R^{N}), NR^{N}C(O), NR^{N}C(O)N(R^{N}), C(O)O, OC(O), OC(O)O, —OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), —S(O)O, OS(O)O, OS(O)₂, S(O)₂O, OS(O)₂O, N(R^{N})S(O), S(O)N(R^{N}), —N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

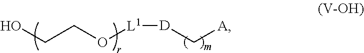

or a salt thereof.

In certain embodiments, a PEG lipid useful in the present disclosure is a PEGylated fatty acid. In certain embodiments, a PEG lipid useful in the present disclosure is a compound of Formula (VI). Provided herein are compounds of Formula (VI):

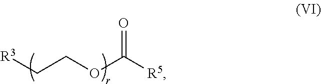

or a salts thereof, wherein:
R³ is-OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), —NR^{N}C(═NR^{N}), NR^{N}C(═NR^{N})N(R^{N}), C(S), C(S)N(R^{N}), NR^{N}C(S), NR^{N}C(S)N(R^{N}), —S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (VI) is of Formula (VI-OH):

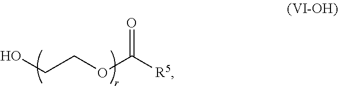

or a salt thereof. In some embodiments, r is 45.

In yet other embodiments the compound of Formula (VI) is:

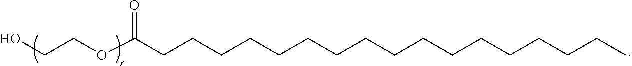

or a salt thereof. In one embodiment, r is 40-50.

In some embodiments, the compound of Formula (VI) is

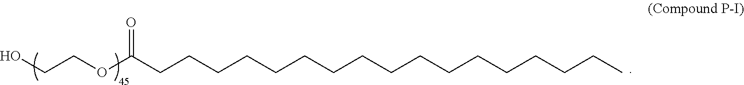

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the present disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a LNP of the present disclosure comprises an ionizable cationic lipid of any of Formula I, II or III, a phospholipid comprising DSPC, a structural lipid, and a PEG lipid comprising PEG-DMG.

In some embodiments, a LNP of the present disclosure comprises an ionizable cationic lipid of any of Formula I, II or III, a phospholipid comprising DSPC, a structural lipid, and a PEG lipid comprising a compound having Formula VI.

In some embodiments, a LNP of the present disclosure comprises an ionizable cationic lipid of Formula I, II or III, a phospholipid comprising a compound having Formula IV, a structural lipid, and the PEG lipid comprising a compound having Formula V or VI.

In some embodiments, a LNP of the present disclosure comprises an ionizable cationic lipid of Formula I, II or III, a phospholipid comprising a compound having Formula IV, a structural lipid, and the PEG lipid comprising a compound having Formula V or VI.

In some embodiments, a LNP of the present disclosure comprises an ionizable cationic lipid of Formula I, II or III, a phospholipid having Formula IV, a structural lipid, and a PEG lipid comprising a compound having Formula VI.

In some embodiments, a LNP of the present disclosure comprises an ionizable cationic lipid of

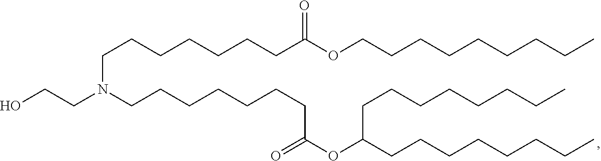 [SMILES: CCCCCCCCCOC(=O)CCCCCCCN(CCO)CCCCCCCC(=O)OC(CCCCCCCC)CCCCCCCC]

and a PEG lipid comprising Formula VI.

In some embodiments, a LNP of the present disclosure comprises an ionizable cationic lipid of

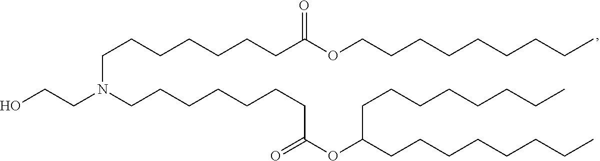 [SMILES: CCCCCCCCCOC(=O)CCCCCCCN(CCO)CCCCCCCC(=O)OC(CCCCCCCC)CCCCCCCC]

and an alternative lipid comprising oleic acid.

In some embodiments, a LNP of the present disclosure comprises an ionizable cationic lipid of

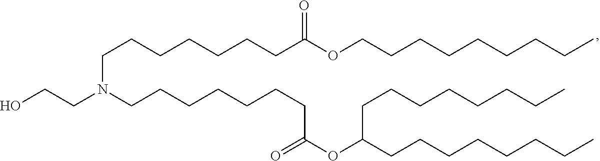 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid comprising a compound having Formula VI.

In some embodiments, a LNP of the present disclosure comprises an ionizable cationic lipid of

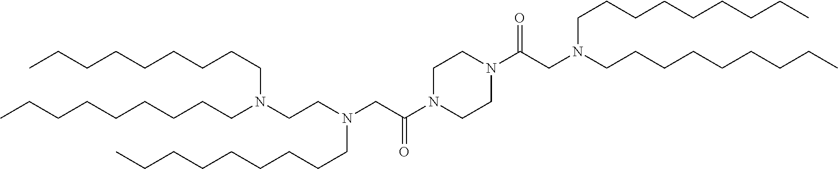 [SMILES: CCCCCCCCCN(CCCCCCCCC)CCN(CCCCCCCCC)CC(=O)N1CCN(C(=O)CN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the present disclosure comprises an ionizable cationic lipid of

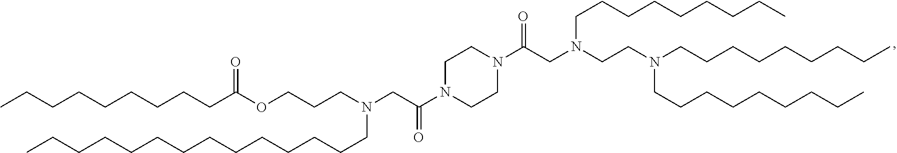 [SMILES: CCCCCCCCCCCCCCN(CCCOC(=O)CCCCCCCCC)CC(=O)N1CCN(C(=O)CN(CCCCCCCCC)CCN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the present disclosure comprises an N:P ratio of from about 2:1 to about 30:1.

In some embodiments, a LNP of the present disclosure comprises an N:P ratio of about 6:1.

In some embodiments, a LNP of the present disclosure comprises an N:P ratio of about 3:1.

In some embodiments, a LNP of the present disclosure comprises a wt/wt ratio of the ionizable cationic lipid component to the RNA of from about 10:1 to about 100:1.

In some embodiments, a LNP of the present disclosure comprises a wt/wt ratio of the ionizable cationic lipid component to the RNA of about 20:1.

In some embodiments, a LNP of the present disclosure comprises a wt/wt ratio of the ionizable cationic lipid component to the RNA of about 10:1.

In some embodiments, a LNP of the present disclosure has a mean diameter from about 50 nm to about 150 nm.

In some embodiments, a LNP of the present disclosure has a mean diameter from about 70 nm to about 120 nm.

### Other Lipid Composition Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/At ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In some embodiments, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In some embodiments, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

### Nanoparticle Compositions

In some embodiments, the pharmaceutical compositions disclosed herein are Formulated as lipid nanoparticles (LNP). Accordingly, the present disclosure also provides nanoparticle compositions comprising (i) a lipid composition comprising a delivery agent such as compound as described herein, and (ii) a polynucleotide encoding a polypeptide. In such nanoparticle composition, the lipid composition disclosed herein can encapsulate the polynucleotide encoding a polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, a lipid nanoparticle comprises an ionizable amino lipid, a structural lipid, a phospholipid, and mRNA. In some embodiments, the LNP comprises an ionizable amino lipid, a PEG-modified lipid, a sterol and a structural lipid. In some embodiments, the LNP has a molar ratio of about 40-50% ionizable amino lipid; about 5-15% structural lipid; about 30-45% sterol; and about 1-5% PEG-modified lipid.

In some embodiments, the lipid nanoparticle comprises 47-49 mol. % ionizable cationic lipid (e.g. ionizable amino lipid. e.g., Compound I-1, Compound I-2, or Compound 1-3), 10-12 mol. % non-cationic lipid (e.g., phospholipid, e.g., DSPC), 38-40 mol. % sterol (e.g., cholesterol) or other structural lipid, and 1-3 mol. % PEG-modified lipid (e.g., PEG-DMG or Compound P-I).

For instance, in some embodiments, the lipid nanoparticle (“LNP-1”) may comprise the following components at the following molar ratios:
(i) 45-50 mol. % Compound I-1
(ii) 35-45 mol. % sterol (e.g., cholesterol);
(iii) 8-12 mol. % phospholipid (e.g., DSPC or DOPE); and
(iv) 1.5-3.5 mol. % PEG-lipid (e.g., Compound P-I or PEG-DMG).

For instance, in some embodiments, the lipid nanoparticle (“LNP-1A”) may comprise the following components at the following molar ratios:
(i) 45-50 mol. % Compound I-1
(ii) 35-45 mol. % Cholesterol;
(iii) 8-12 mol. % DSPC; and
(iv) 1.5-3.5 mol. % PEG-DMG.

For instance, in some embodiments, the lipid nanoparticle (“LNP-1B”) may comprise the following components at the following molar ratios:
(i) 45-50 mol. % Compound I-1
(ii) 35-45 mol. % Cholesterol;
(iii) 8-12 mol. % DSPC; and
(iv) 1.5-3.5 mol. % Compound P-I.

In some embodiments, the lipid nanoparticle (“LNP-2”) may comprise the following:
(i) 45-50 mol. % Compound I-2;
(ii) 35-45 mol. % sterol (e.g., Cholesterol);
(iii) 8-12 mol. % phospholipid (e.g., DSPC or DOPE); and
(iv) 1.5-3.5 mol. % PEG-lipid (e.g., Compound P-I or PEG-DMG).

In some embodiments, the lipid nanoparticle (“LNP-2A”) may comprise the following:
(i) 45-50 mol. % Compound I-2;
(ii) 35-45 mol. % Cholesterol;
(iii) 8-12 mol. % DSPC; and
(iv) 1.5-3.5 mol. % PEG-DMG.

For instance, in some embodiments, the lipid nanoparticle (“LNP-2B”) may comprise the following components at the following molar ratios:
(i) 45-50 mol. % Compound I-2;
(ii) 35-45 mol. % Cholesterol;
(iii) 8-12 mol. % DSPC; and
(iv) 1.5-3.5 mol. % Compound P-I.

In some embodiments, the lipid nanoparticle (“LNP-3”) may comprise the following:
(i) 45-50 mol. % Compound I-3;
(ii) 35-45 mol. % sterol (e.g., Cholesterol);
(iii) 8-12 mol. % phospholipid (e.g., DSPC or DOPE); and
(iv) 1.5-3.5 mol. % PEG-lipid (e.g., Compound P-1 or PEG-DMG).

In some embodiments, the lipid nanoparticle (“LNP-3A”) may comprise the following:
(i) 45-50 mol. % Compound 1-3;
(ii) 35-45 mol. % Cholesterol;
(iii) 8-12 mol. % DSPC; and
(iv) 1.5-3.5 mol. % PEG-DMG.

In some embodiments, the lipid nanoparticle (“LNP-3B”) may comprise the following:
(i) 45-50 mol. % Compound I-3;
(ii) 35-45 mol. % Cholesterol;
(iii) 8-12 mol. % DSPC; and
(iv) 1.5-3.5 mol. % Compound P-I.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle (LNP) may comprise an ionizable amino lipid. As used herein, the term “ionizable amino lipid” has its ordinary meaning in the art and may refer to a lipid comprising one or more charged moieties. In some embodiments, an ionizable amino lipid may be positively charged or negatively charged. An ionizable amino lipid may be positively charged, in which case it can be referred to as “cationic lipid”. In certain embodiments, an ionizable amino lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

The ionizable amino lipid is sometimes referred to in the art as an “ionizable cationic lipid”. In some embodiments, the ionizable amino lipid may have a positively charged hydrophilic head and a hydrophobic tail that are connected via a linker structure.

In addition to these, an ionizable amino lipid may also be a lipid including a cyclic amine group.

In some embodiments, the ionizable amino lipid may be selected from, but not limited to, an ionizable amino lipid described in International Publication Nos. WO2013086354 and WO2013116126; the contents of each of which are herein incorporated by reference in their entirety.

In yet another embodiment, the ionizable amino lipid may be selected from, but not limited to, Formula CLI-CLXXXXII of U.S. Pat. No. 7,404,969; each of which is herein incorporated by reference in their entirety.

In some embodiments, the lipid may be a cleavable lipid such as those described in International Publication No. WO2012170889, herein incorporated by reference in its entirety. In some embodiments, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein. “size” or “mean size” in the context of nanoparticle compositions refers to the mean diameter of a nanoparticle composition.

In some embodiments, the polynucleotide encoding a polypeptide are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the nanoparticles have a diameter from about 10 to 500 nm. In some embodiments, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm. 800 nm, 700 nm. 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition. e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. Adv. Drug Deliv. Rev. 87:68-80 (2015): Silva et al. Curr. Pharm. Technol. 16: 940-954 (2015); Naseri et al. Adv. Pharm. Bull. 5:305-13 (2015); Silva et al. Curr. Pharm. Biotechnol. 16:291-302 (2015), and references cited therein.

In some embodiments, the LNP formulations described herein can additionally comprise a permeability enhancer molecule. Non-limiting permeability enhancer molecules are described in U.S. Pub. No. US20050222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP formulation can also contain a polymer conjugate (e.g., a water-soluble conjugate) as described in, e.g., U.S. Pub. Nos. US20130059360, US20130196948, and US20130072709. Each of the references is herein incorporated by reference in its entirety.

The LNP formulations can comprise a conjugate to enhance the delivery of nanoparticles of the present disclosure in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science. 339: 971-975 (2013), herein incorporated by reference in its entirety). As shown by Rodriguez et al., the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013110028, each of which is herein incorporated by reference in its entirety.

The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, poly acetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028, herein incorporated by reference in its entirety.

In some embodiments, the polynucleotide described herein is Formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids (Aleku et al., Cancer Res. 68:9788-9798 (2008): Strumberg et al., Int. J. Clin. Pharmacol. Ther. 50:76-78 (2012); Santel et al., Gene Ther 13:1222-1234 (2006); Santel et al., Gene Ther. 13:1360-1370 (2006); Gutbier et al., Pulm. Pharmacol. Ther. 23:334-344 (2010); Kaufmann et al. Microvasc. Res. 80:286-293 (2010); Weide et al. J. Immunother. 32:498-507 (2009); Weide et al. J. Immunother. 31:180-188 (2008); Pascolo, Expert Opin. Biol. Ther. 4:1285-1294 (2004); Fotin-Mleczek et al., J. Immunother. 34:1-15 (2011); Song et al., Nature Biotechnol. 23:709-717 (2005); Peer et al., Proc. Natl. Acad. Sci. USA. 6(104):4095-4100 (2007); deFougerolles, Hum. Gene. Ther. 19:125-132 (2008); all of which are incorporated herein by reference in its entirety).

In some embodiments, the polynucleotides described herein are Formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Examples of SLN can be those as described in Intl. Pub. No. WO2013105101, herein incorporated by reference in its entirety.

In some embodiments, the polynucleotides described herein can be Formulated for controlled release and/or targeted delivery. As used herein, “controlled release” refers to a pharmaceutical composition or compound release profile that conforms to a particular pattern of release to effect a therapeutic outcome. In some embodiments, the polynucleotides can be encapsulated into a delivery agent described herein and/or known in the art for controlled release and/or targeted delivery. As used herein, the term “encapsulate” means to enclose, surround or encase. As it relates to the formulation of the compounds of the present disclosure, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the present disclosure can be enclosed, surrounded or encased within the delivery agent. “Partial encapsulation” or “partially encapsulate” means that less than 10, 10, 20, 30, 40 50 or less of the pharmaceutical composition or compound of the present disclosure can be enclosed, surrounded or encased within the delivery agent.

Advantageously, encapsulation can be determined by measuring the escape or the activity of the pharmaceutical composition or compound of the present disclosure using fluorescence and/or electron micrograph. For example, at least 1, 5, 10, 20, 30, 40, 50, 60, 70, 80, 85, 90, 95, 96, 97, 98, 99, 99.9, or greater than 99% of the pharmaceutical composition or compound of the present disclosure are encapsulated in the delivery agent.

In some embodiments, the polynucleotides described herein can be encapsulated in a therapeutic nanoparticle, referred to herein as “therapeutic nanoparticle polynucleotides.” Therapeutic nanoparticles can be Formulated by methods described in, e.g., Intl. Pub. Nos. WO2010005740, WO2010030763, WO2010005721, WO2010005723, and WO2012054923; and U.S. Pub. Nos. US20110262491, US20100104645, US20100087337, US20100068285, US20110274759, US20100068286, US20120288541, US20120140790, US20130123351 and US20130230567; and U.S. Pat. Nos. 8,206,747, 8,293,276, 8,318,208 and 8,318,211, each of which is herein incorporated by reference in its entirety.

In some embodiments, the therapeutic nanoparticle polynucleotide can be Formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the polynucleotides described herein can be Formulated as disclosed in Intl. Pub. No. WO2010075072 and U.S. Pub. Nos. US20100216804, US20110217377, US20120201859 and US20130150295, each of which is herein incorporated by reference in their entirety.

In some embodiments, the therapeutic nanoparticle polynucleotide can be Formulated to be target specific, such as those described in Intl. Pub. Nos. WO2008121949. WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Examples of microfluidic mixers can include, but are not limited to, a slit interdigital micromixer including, but not limited to those manufactured by Microinnova (Allerheiligen bei Wildon, Austria) and/or a staggered herringbone micromixer (SHM) (see, Zhigaltsev et al., Langmuir 28:3633-40 (2012); Belliveau et al., Molecular Therapy-Nucleic Acids. 1:e37 (2012); Chen et al., J. Am. Chem. Soc. 134(16):6948-51 (2012); each of which is herein incorporated by reference in its entirety). Examples of micromixers include Slit Interdigital Microstructured Mixer (SIMM-V2) or a Standard Slit Interdigital Micro Mixer (SSIMM) or Caterpillar (CPMM) or Impinging-jet (IJMM,) from the Institut für Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the polynucleotides described herein can be Formulated in lipid nanoparticles using microfluidic technology (see. Whitesides, George M., Nature 442: 368-373 (2006); and Abraham et al., Science 295: 647-651 (2002); each of which is herein incorporated by reference in its entirety). In some embodiments, the polynucleotides can be Formulated in lipid nanoparticles using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

In some embodiments, the polynucleotides described herein can be Formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 urn, about 20 to about 90 nm, about 20 to about 100 nm, about 30 to about 40 nm, about 30 to about 50 urn, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 urn, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In some embodiments, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the polynucleotides can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 urn, less than 250 um, less than 275 um, less than 300 um, less than 325 um, less than 350 um, less than 375 um, less than 400 um, less than 425 um, less than 450 um, less than 475 um, less than 500 urn, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

The nanoparticles and microparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., Intl. Pub. No. WO2013082111, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), poly cyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

### Methods of Using Anti-GPC3 Antibodies and Binding Proteins

In some aspects, antibodies, antigen-binding fragments, and/or binding proteins (CARs, such as CARs expressed by a T cell) of the present disclosure are administered to a subject in need thereof. In some aspects, the subject in need thereof is a subject with a disease, disorder, and/or condition that may be treated with technologies described herein.

In some aspects, the subject in need thereof is a subject with cancer. Certain cancers that may be treated in accordance with technologies of the present disclosure include, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, and myeloid leukemia), lymphoma (e.g., Burkitt lymphoma (non-Hodgkin lymphoma), cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromocytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

In some aspects, the subject in need thereof is a mammal. In some embodiments, a mammal includes, for example and without limitation, a household pet (e.g., a dog, a cat, a rabbit, a ferret, a hamster), a livestock or farm animal (e.g., a cow, a pig, a sheep, a goat, a pig, a chicken or another poultry), a horse, a monkey, a laboratory animal (e.g., a mouse, a rat, a rabbit) and a human. In a preferred embodiment, the subject in need thereof is a human. Technologies of the present disclosure can be practiced in any subject in need thereof that is likely to benefit from administration of technologies of the present disclosure (e.g., a subject with cancer).

In some embodiments, a subject in need thereof is a human. In some embodiments, the human is male. In some embodiments, the human is female. In some embodiments, the human is an adult (e.g., 18 or more years of age). In some embodiments, the adult is greater than 18 years old, greater than 25 years old, greater than 30 years old, greater than 40 years old, greater than 50 years old, greater than 55 years old, greater than 60 years old, greater than 65 years old, greater than 70 years old, greater than 75 years old, greater than 80 years old, greater than 85 years old, greater than 90 years old, greater than 95 years old, greater than 100 years old, or greater than 105 years old in age. In some embodiments, the human is a child. In some embodiments, the child is greater than 2 years old, greater than 3 years old, greater than 4 years old, greater than 5 years old, greater than 6 years old, greater than 7 years old, greater than 8 years old, greater than 9 years old, greater than 10 years old, greater than 11 years old, greater than 12 years old, greater than 13 years old, greater than 14 years old, greater than 15 years old, or greater than 16 years old in age.

In some aspects, a subject in need thereof is administered a population of T cells (e.g., T cells) comprising a CAR of the present disclosure.

In some aspects, a subject in need thereof is administered an antibody or antigen-binding fragment of the present disclosure.

### Routes of Administration and Dosing

Anti-GPC3 antibodies or antigen-binding fragments of the disclosure, and nucleic acids encoding the same, can be administered to a subject (e.g., a mammalian subject, such as a human) by a variety of routes. In some embodiments, the antibody or nucleic acid is administered to the subject intravenously, subcutaneously, intramuscularly, parenterally, intrathecally, intracerebroventricularly, transdermally, or orally.

T cells expressing CARs of the present disclosure can be administered to a subject in need thereof by any suitable means, which include, for example, parenteral administration. Parental administration can include intravenous, intramuscular, intraarterial, subcutaneous, intratumoral, intrathecal, and/or intraperitoneal administration. In some embodiments, T cells expressing CARs of the present disclosure are administered by systemic infusion.

The most suitable route for administration in any given case will depend on the particular therapeutic agent administered, the patient, pharmaceutical formulation methods, and various patient-specific parameters, such as the patient's age, body weight, sex, severity of the diseases being treated, the patient's diet, and the patient's excretion rate.

An appropriate dosage of anti-GPC3 antibodies or antigen-binding fragments of the disclosure, nucleic acids encoding the same, or T cells expressing CARs of the present disclosure will vary with the particular condition, disease and/or disease being treated, various subject-specific parameters (e.g., age, weight, physical condition of the subject), severity of the particular condition, disease, and/or disorder being treated, the nature of current or combination therapy (if any), the specific route of administration and other factors within the knowledge and expertise of a health practitioner. In some embodiments, a maximally tolerated dose of technologies described herein is to be used, e.g., the highest safe dose according to sound medical judgement. In some embodiments, technologies described herein are administered in an effective amount, e.g., a dose sufficient to provide one or more medically desirable results.

A therapeutic regiment for use in accordance with technologies described herein may include administration of such technologies or compositions comprising such technologies once a day, once every two days, once every three days, twice a week, once a week, once every two weeks, once every three weeks, once every month or four weeks, once every six weeks, once every two months or eight weeks, once every three months or twelve weeks. In some certain embodiments, a subject receives a single dose of a technology described herein. In certain embodiments, a subject receives a plurality of doses of a technology described herein (e.g., at least two, at least three, at least four, at least five, at least six, at least eight, at least ten, or more doses). In some embodiments, technologies described herein are administered over a period of time, such as one week, two weeks, three weeks, four weeks, six weeks, two months, three months, four months, five months, six months, one year or more. Appropriate therapeutic regimens are readily understood by medical practitioners and such regimens may be designed by a medical practitioner for a particular patient (e.g., a patient-specific regimen).

### Kits

Also included herein are kits that contain anti-GPC3 antibodies or antigen-binding fragments and/or nucleic acids encoding the same. In some embodiments, the kits provided herein contain one or more cells engineered to express and secrete an anti-GPC3 antibody, antigen-binding fragment, or CAR of the disclosure, such as a cell containing a nucleic acid molecule of the disclosure or a CAR-T cell.

A kit described herein may include reagents that can be used to produce a pharmaceutical composition of the invention. Optionally, kits described herein may include reagents that can induce the expression of anti-GPC3 antibodies, antigen-binding fragments, or CARs of the present disclosure within cells (e.g., mammalian cells).

Other kits described herein may include tools for engineering a prokaryotic or eukaryotic cell (e.g., a CHO cell or a BL21(DE3) E. Coli cell or an T cell) so as to express and secrete an anti-GPC3 antibody, antigen-binding fragment, or CAR described herein. For example, a kit may contain CHO cells stored in an appropriate media and optionally frozen according to methods known in the art. The kit may also contain a nucleic acid encoding the desired antibody, antigen-binding fragment, or CAR as well as reagents for expressing the antibody, antigen-binding fragment, or CAR in the cell.

A kit described herein may also provide an anti-GPC3 antibody or antigen-binding fragment of the disclosure, or a nucleic acid encoding the same, or a CAR-T cell of the present disclosure in combination with a package insert describing how the antibody, antigen-binding fragment, or nucleic acid may be administered to a subject or how the CAR-T cell may be administered to a subject, for example, for the treatment of a disease, disorder and/or condition (e.g., cancer).

### EXAMPLES

The following examples are put forth so as to provide those of ordinary skill in the art with a description of how the compositions and methods claimed herein can be performed, made, and evaluated, and are intended to be purely examples and are not intended to limit the scope of the present disclosure.

### Example 1: Generation, Screening, and Validation of Anti-GPC3 VHH Domains

The present example demonstrates generation, screening, and validation of anti-GPC3 VHH domains. It is noted that the term “VHH” as used herein refers to the variable heavy chain domain of a heavy chain only antibody produced by the HCAb mice described below.

### Immunization of Heavy Chain Antibody (HCAb) Mice

Six engineered mice that generate heavy-chain antibodies (HCAbs) and two Balb/c mice (control) were immunized with injection of a human GPC3 recombinant protein (Acro Biosystems). Immunization was completed at multiple sites near the draining lymph nodes. Two injections were completed intraperitoneally (one on each side inguinal, 50 μL each), four injections were completed subcutaneously (one each side axillary, 25 μL each; one each side popliteal, 25 μL). Two mice were pre-immunized with Flt3L mRNA at 0.1 mg/kg 7 days before antigen immunization. All mice were primed with antigen in Complete Freund's Adjuvant (CFA) and boosted with human GPC3 or mouse GPC3 with Incomplete Freund's Adjuvant (IFA) alternatively every other week. Amino acid sequences of human and mouse GPC3 recombinant proteins utilized for immunization of HCAbJ mice are shown in Table 7. The spleen and bone marrow were collected three days after a final boost with no adjuvant. B cells from four immunized HCAb mice were purified, total RNA was extracted, and cDNA were made out of the RNA.

TABLE 7
Human GPC3 and Mouse GPC3 recombinant proteins

| | SEQ | |
|---|---|---|
| | ID | |
| GPC3 | NO: | Recombinant GPC3 protein amino acid sequence |
| Human | 154 | QPPPPPPDATCHQVRSFFQRLQPGLKWVPETPVPGSDLQVC |
| GPC3 | | LPKGPTCCSRKMEEKYQLTARLNMEQLLQSASMELKFLIIQ |
| | | NAAVFQEAFEIVVRHAKNYTNAMFKNNYPSLTPQAFEFVG |
| | | EFFTDVSLYILGSDINVDDMVNELFDSLFPVIYTQLMNPGL |
| | | PDSALDINECLRGARRDLKVFGNFPKLIMTQVSKSLQVTRI |
| | | FLQALNLGIEVINTTDHLKFSKDCGRMLTRMWYCSYCQGL |
| | | MMVKPCGGYCNVVMQGCMAGVVEIDKYWREYILSLEEL |
| | | VNGMYRIYDMENVLLGLFSTIHDSIQYVQKNAGKLTTTIG |
| | | KLCAHSQQRQYRSAYYPEDLFIDKKVLKVAHVEHEETLSS |
| | | RRRELIQKLKSFISFYSALPGYICSHSPVAENDTLCWNGQEL |
| | | VERYSQKAARNGMKNQFNLHELKMKGPEPVVSQIIDKLK |
| | | HINQLLRTMSMPKGRVLDKNLDEEGFESGDCGDDEDECIG |
| | | GSGDGMIKVKNQLRFLAELAYDLDVDDAPGNSQQATPKD |
| | | NEISTFHNLGNVH |
| Mouse | 155 | QPPPPPDATCHQVRSFFQRLQPGLKWVPETPVPGSDLQVCL |
| GPC3 | | PKGPTCCSRKMEEKYQLTARLNMEQLLQSASMELKFLIIQ |
| | | NAAVFQEAFEIVVRHAKNYTNAMFKNNYPSLTPQAFEFVG |
| | | EFFTDVSLYILGSDINVDDMVNELFDSLFPVIYTQMMNPGL |
| | | PESVLDINECLRGARRDLKVFGSFPKLIMTQVSKSLQVTRIF |
| | | LQALNLGIEVINTTDHLKFSKDCGRMLTRMWYCSYCQGL |
| | | MMVKPCGGYCNVVMQGCMAGVVEIDKYWREYILSLEEL |
| | | VNGMYRIYDMENVLLGLFSTIHDSIQYVQKNGGKLTTTIG |
| | | KLCAHSQQRQYRSAYYPEDLFIDKKILKVAHVEHEETLSSR |
| | | RRELIQKLKSFINFYSALPGYICSHSPVAENDTLCWNGQEL |
| | | VERYSQKAARNGMKNQFNLHELKMKGPEPVVSQIIDKLK |
| | | HINQLLRTMSVPKGKVLDKSLDEEGLESGDCGDDEDECIG |
| | | SSGDGMVKVKNQLRFLAELAYDLDVDDAPGNKQHGNQK |
| | | DNEITTSHSVGNM |

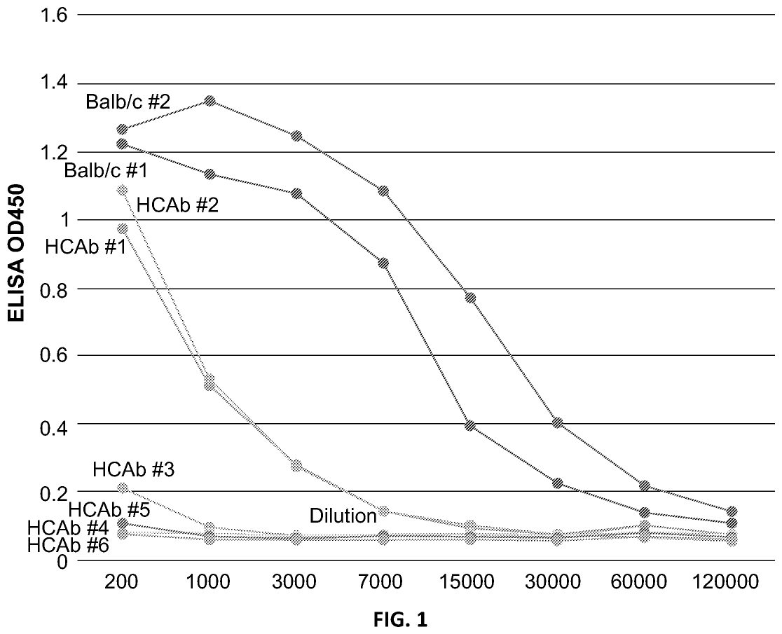

Titers were evaluated using enzyme-linked immunosorbent assay (ELISA) in a 96-well plate. Briefly, wells were coated with 50 μL of 5 μg/mL antigen in PBS for 2 hours at room temperature followed by blocking with 300 μL of PBS with 100 Bovine Serum Albumin (BSA), 1% fat free milk powder, and 0.1% Tween-20 per well for 30 minutes at room temperature. Wells were washed 3 times with PBS with 0.1% Tween-20. 3 μL of serum was diluted to 600 μL in PBS with 1% BSA, 1% fat free milk powder, and 0.1% Tween-20 (200-fold dilution). Additional serum sample dilutions were prepared (1.000-, 3,000-, 7,500-, 15,000-, 30,000-, 60,000-, and 120,000-fold dilutions). 50 μL of each serum dilution was added to wells and incubated for at least 2 hours at room temperature (if incubation was overnight, at 4° C.). Following incubation with the diluted serum samples, the wells were washed four times with 300 μL of PBS with 0.1% Tween-20. 50 μL of rat anti-mouse IgG1 conjugated to HRP (BD Biosciences, catalog #559626, diluted 1/1000 in PBS with 1% BSA, 1% fat free milk powder and 0.1% Tween-20) was added to each well and incubated for 2 hours at room temperature. Following the incubation, each well was washed five times with 300 μL PBS with 0.1% Tween-20 followed by addition of the 50 μL POD substrate (Roche, BM Blue POD substrate soluble, #11484281001). The reaction was incubated for 5 minutes and stopped with 50 μL of 1M H₂SO₄. Results were measured using a plate-reader at 450 nM. It was determined that HCAb mice preconditioned with Flt3 mRNA generated moderate Ab titer (FIG. 1).

The titer was consistently at about 1:3000 with 3× serum neutralization.

### Anti-GPC3 VHH Domain Panning

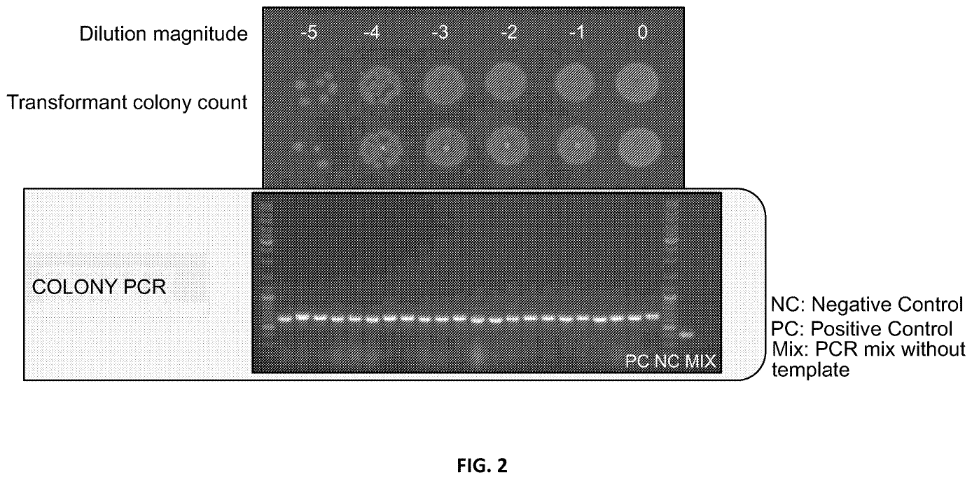

Anti-GPC3 VHH domains were panned using phage particles. The phage display library was generated by a two-step polymerase chain reaction (PCR) protocol that was applied to amplify VHH segments from the cDNA library isolated from immunized mice. In the first step, PCR reactions on cDNA were completed using VHH frame work (FR) 1 primers together with VHH FR4 primers. In the second step, a nested PCR on the VHH-amplicons was completed with similar primers and the amplicons were inserted into phage display vector, pDCL1 phagemid, via SfiI/NotI. The final library contained more than 4×10⁸ clones. 95 clones were picked and digested with SfiI/NotI. 100% of clones contained the correct size VHH insert. The 95 were sent for Sanger sequencing and 85 were confirmed to be VHH (FIG. 2).

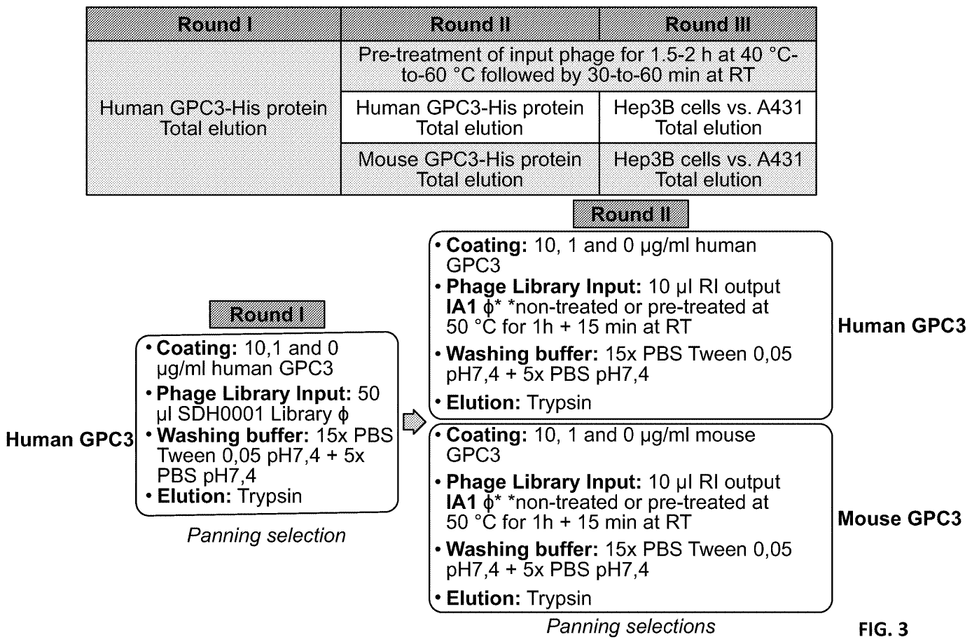

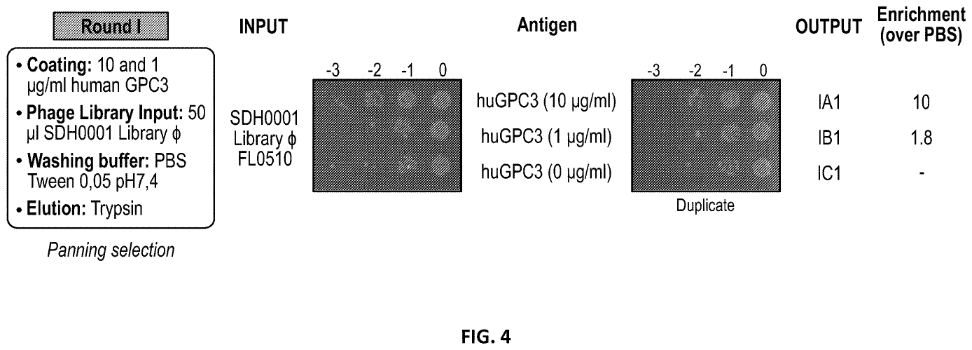

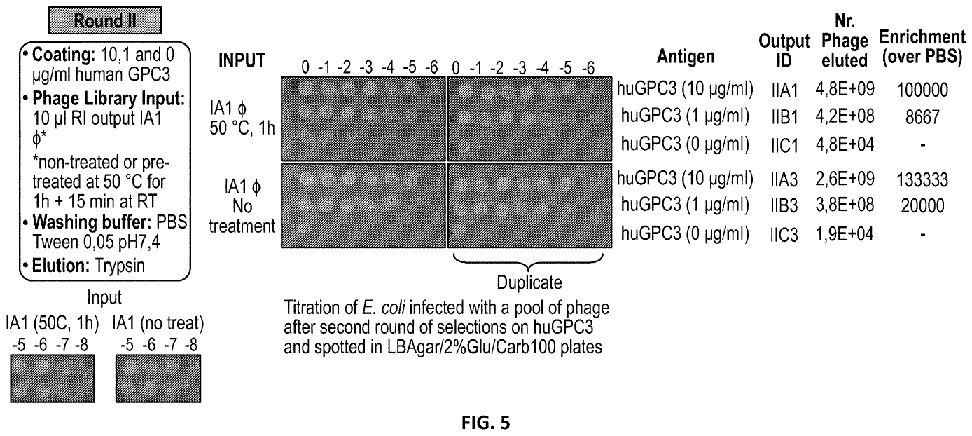

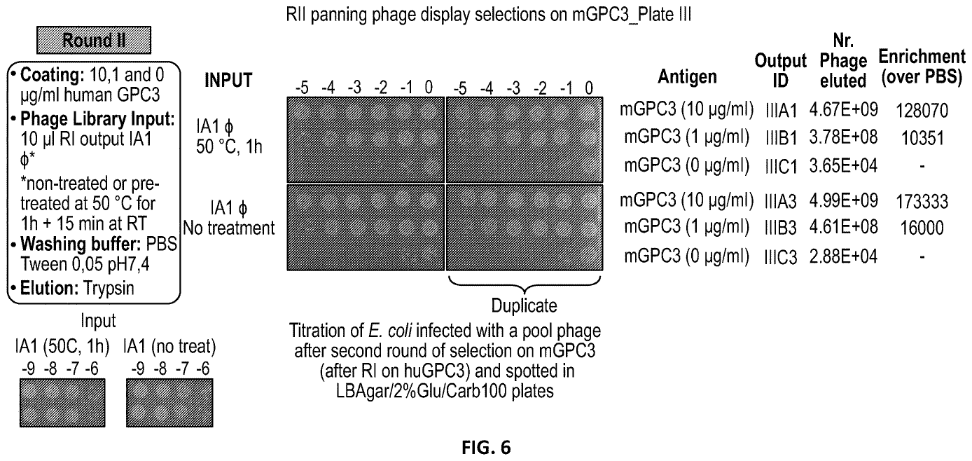

Three rounds of panning were planned as outlined in FIG. 3. The first round of panning was completed using human GPC3-His protein. The total eluent was amplified and heat treated for 1 hour at 50° C. followed by 60 minutes at room temperature. The first round of panning showed about a 10-fold enrichment over PBS (FIG. 4). The second round of panning was completed using two strategies, human GPC3 or mouse GPC3. High enrichments were observed at the second round of panning using human GPC3, but no significant difference was observed in terms of output size and enrichments between phage pretreated at 50° C. and untreated (FIG. 5). FIG. 6 shows enrichment after a second round of phage display panning using mouse GPC3. The planned third round of panning was not completed due to the high enrichment rate achieved with two rounds of panning.

### Anti-GPC3 VHH Domain Selection

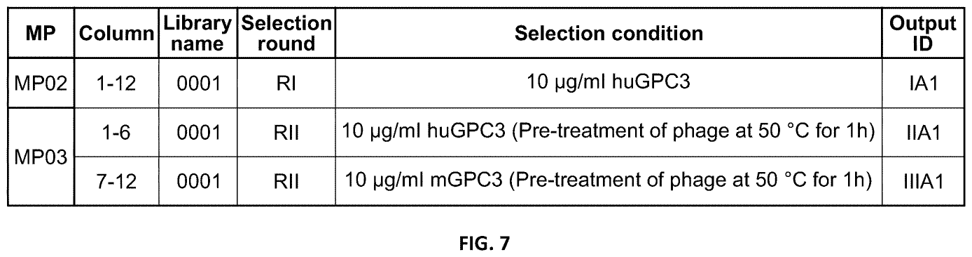

To select anti-GPC3 VHH domains, phage with surface displayed VHH domains and periplasmic extracts (PEs) comprising soluble VHH domains were produced. Two master plates (MP) comprising phage and PEs that underwent either one or two rounds of panning and were selected by phage display under varying conditions were generated (FIG. 7).

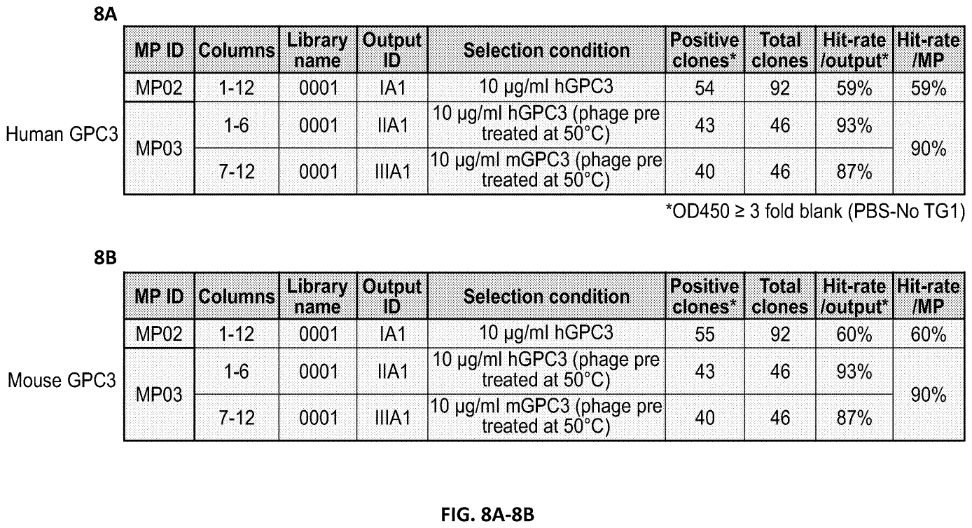

Binding of periplasmic extracts comprising soluble VHH domains was assessed using ELISA. Briefly, wells were coated with either 1 μg/mL human GPC3 (Sino Biological Inc.; cat. nr. 10088-H08H) or mouse GPC3 (Sino Biological Inc.; cat. nr. 50989-M08B-200) and then blocked using 4% Marvel (dry skimmed milk)/PBS. Periplasmic extracts from MP clones were diluted 1:5 and detection was completed using anti-c-myc antibody (Roche, cat. nr. 1monoclonal 1667203001) at 1:1000 dilution and anti-mouse IgG-HRP at 1:5000 dilution (JIR. cat. nr. 715-035-150). Anti-GPC3-VHH-Fc at 2 g/ml followed by anti-human IgG-HRP (JIR) and human GPC3 antibody (R&D, MAB2119) at 2 g/ml followed by anti-mouse IgG-HRP (JIR) were utilized as positive controls, while a blank was used as a negative control. Results demonstrated that periplasmic extracts comprising soluble VHH domains had a high positive rate for binding to both human GPC3 and mouse GPC3 (FIG. 8A-8B).

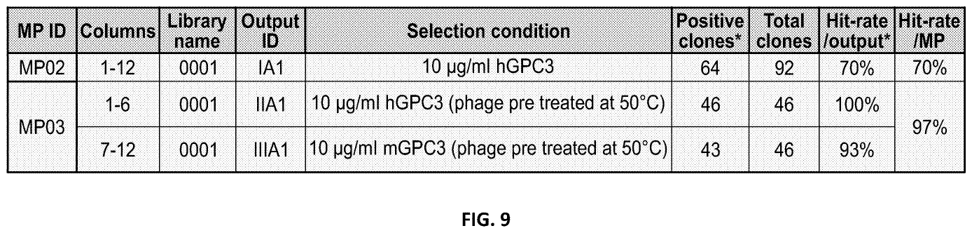

Binding of phage with surface displayed VHH domains was also assessed using ELISA. Briefly, wells were coated with 1 μg/mL human GPC3 (Sino Biological Inc.; cat. nr. 10088-H08H) and then blocked using 4% Marvel (dry skimmed milk)/PBS. Phage from MP clones were diluted 1:10 and detection was completed using an anti-M13-HRP at 1:5000 (GE). Anti-GPC3-VHH-Fc at 2 g/ml followed by anti-human IgG-HRP (JIR) and human GPC3 antibody (R&D, MAB2119) at 2 μg/ml followed by anti-mouse IgG-HRP (JIR) were utilized as positive controls, while a blank (no Phage) was used as a negative control. Results demonstrated that surface attached VHH domains displayed on the phage surface had a high positive rate for binding to both human GPC3 and mouse GPC3 (FIG. 9).

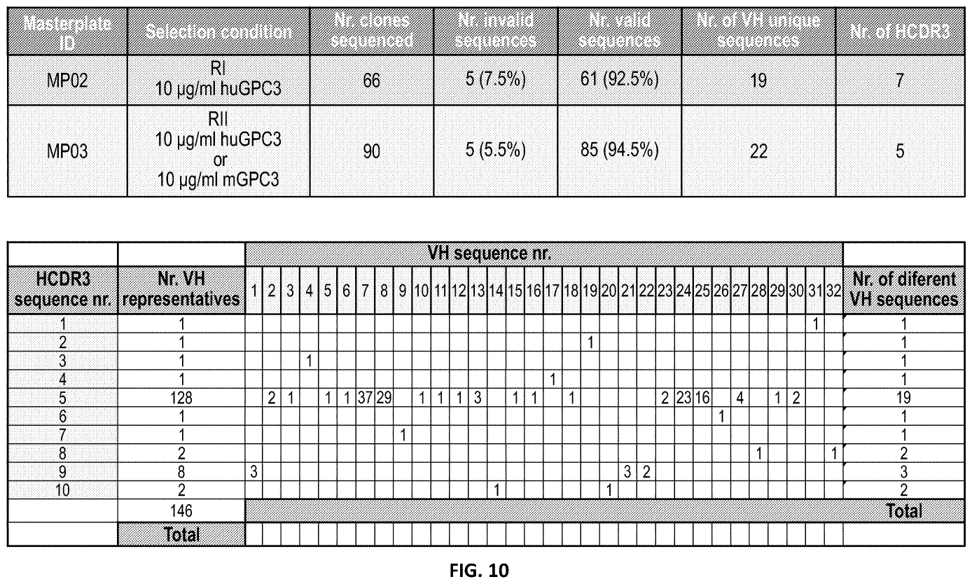

156 ELISA-positive clones for GPC3 binding were then sequenced. 146 valid VHH sequences were recovered and in total, 36 unique VHH domain sequences were identified belonging to 10 different CDR3 families (FIG. 10).

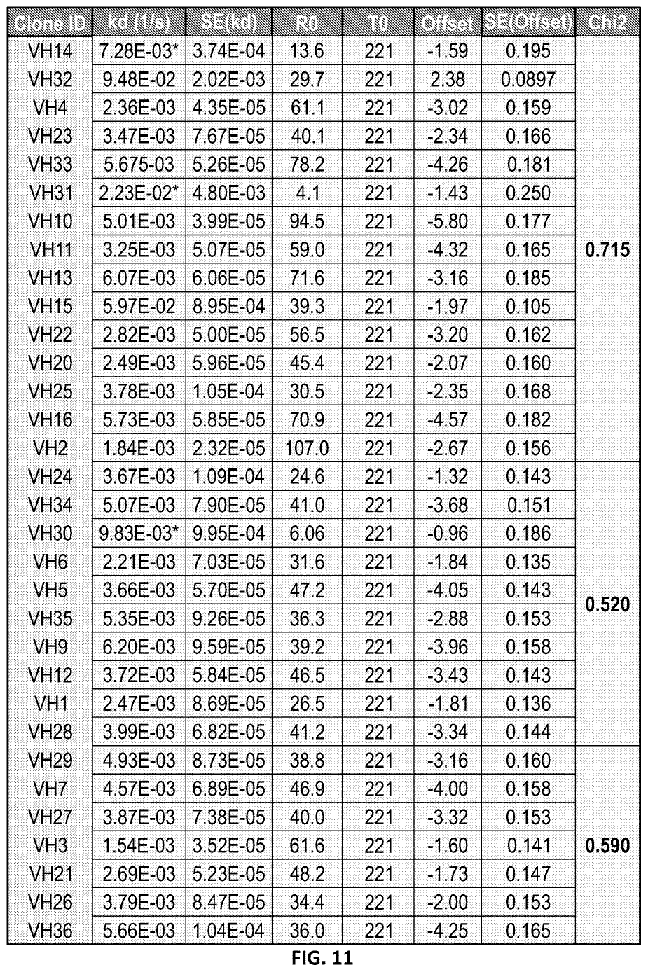

The K_{off} of 32 unique VHH domains identified were then evaluated using periplasmic extracts. Human GPC3 protein in acetate buffer, pH 5.0, was immobilized in a sensor chip CM5 (GE Healthcare; Cat. Nr. BR-1005-30; Lot. Nr.10245232) and successful immobilization was confirmed by using a commercially available human GPC3 antibody (R&D cat. nr. MAB2119) and the anti-GPC3-VHH-Fc (both at 10 μg/mL). Periplasmic extracts from the MP were diluted in Biacore Buffer (0.01 M HEPES pH 7.4, 0.15 M NaCl, 3 mM EDTA, 0.05% P20) and injected over 2 minutes at 30 μL/min. 10 μl of 10 mM NaOH/1M NaCl were injected between samples and off-rates were measured over 5 minutes. Off-rate (K_{D} values) were calculated using BIACORE software 1:1 interaction Langmuir binding model. K_{D} values of each periplasmic extract is shown in FIG. 11.

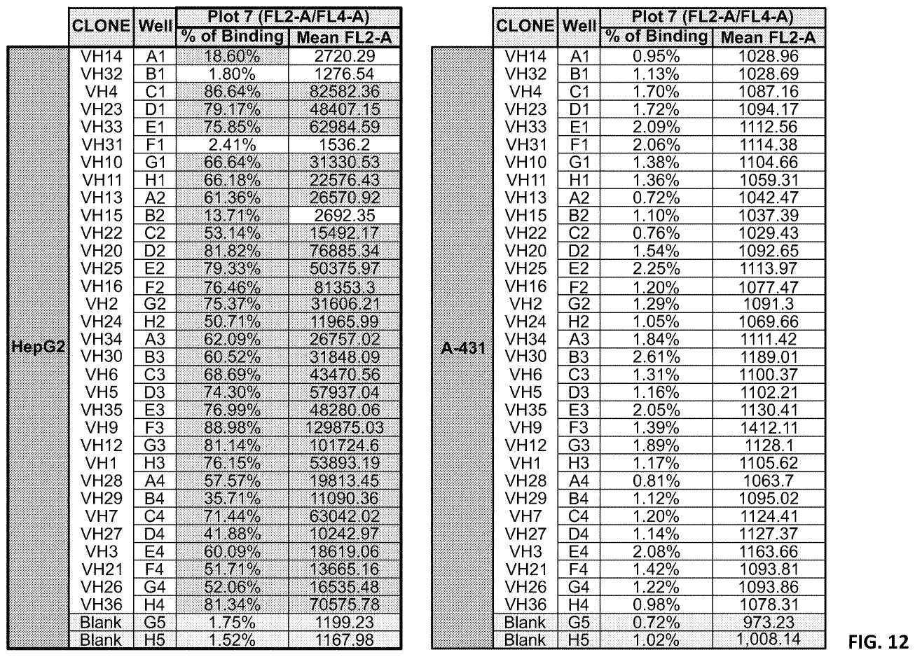

Binding of phage with surface expressed VHH domains to HepG2 cells was assessed using FACS. A-431 cells, which do not express human GPC3, were utilized as a negative control in addition to a blank sample (no phage). In brief, cells were recovered and diluted to a concentration of 2.5×10⁶ cells/mL and then incubated in 4% Marvel/FACS buffer for 20 minutes on ice. The cell suspension was distributed throughout at 96-well, U-bottom plate (85 μL/well) and incubated with phage (15 μL) on ice for 60 minutes with shaking. Detection was completed using anti-M13 biotin (Fitzgerald; cat.nr.61R-M101aBT) at a 1:125 dilution on ice for 30 minutes and Streptavidin-PE (Life technologies; cat.nr.SA10041) at a 1:400 dilution on ice for 20 minutes). From the 32 selected clones, 30 showed binding to HepG2 cells (with percentage of binding between 13.71%-88.98%). None of the clones showed binding to A-431 cells (FIG. 12).

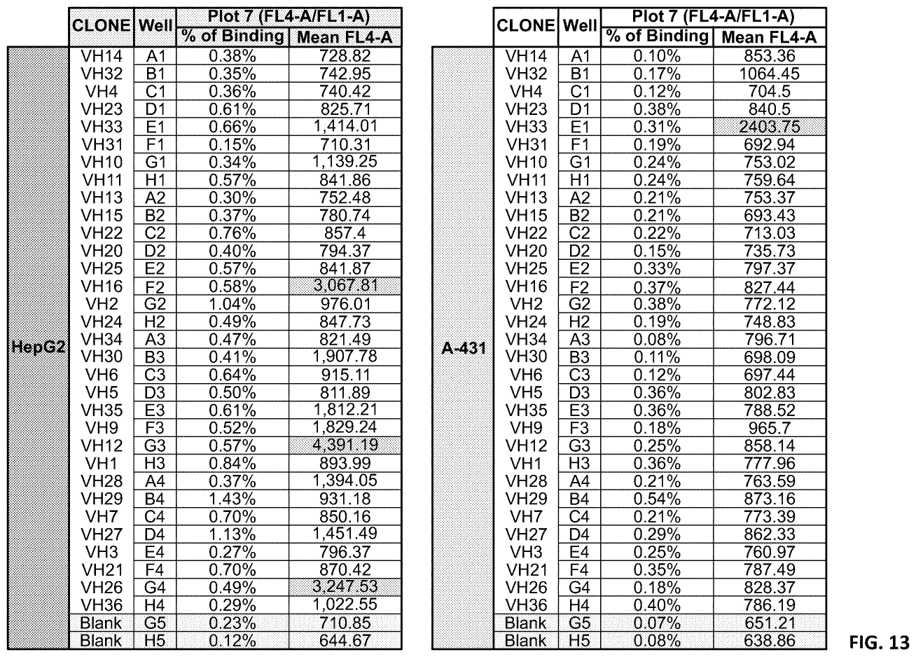

Binding of periplasmic extracts comprising soluble VHH domains to HepG2 cells were also assessed using FACS. A-431 cells were utilized as a negative control in addition to a blank sample (no periplasmic extract). Cells were recovered and diluted in a concentration of 1.0×10⁶ cells/ml and then incubated in FACS buffer for 20 min on ice. Cell suspension was distributed into a 96 well, U-bottom plate (200 μl/well; 2.0×10⁵ cells). Periplasmic extract from the 32 unique clones were incubated at a 1:5 dilution with anti-c-myc antibody (Gentaur; cat.nr.04-CMYC-9E10) at a 1:1000 dilution for 30 minutes at room temperature. Periplasmic extract and anti-c-myc mix was added to the 96 well U-bottom plate with 2.0×10⁵ cells cells/well and incubated for 1 hour on ice. Detection was completed using a goat-anti-mouse IgG-APC (GAM-APC) (BD Biociences, cat.nr.550826) at a 1:500 dilution. Although binding to HepG2 cells was observed for 30 clones as phage format, none of the clones demonstrated binding to HepG2 cells as periplasmic extract (FIG. 13). Without wishing to be bound by any one theory, it is hypothesized this may be due to lone expression of the clones in the periplasmic extract solution.

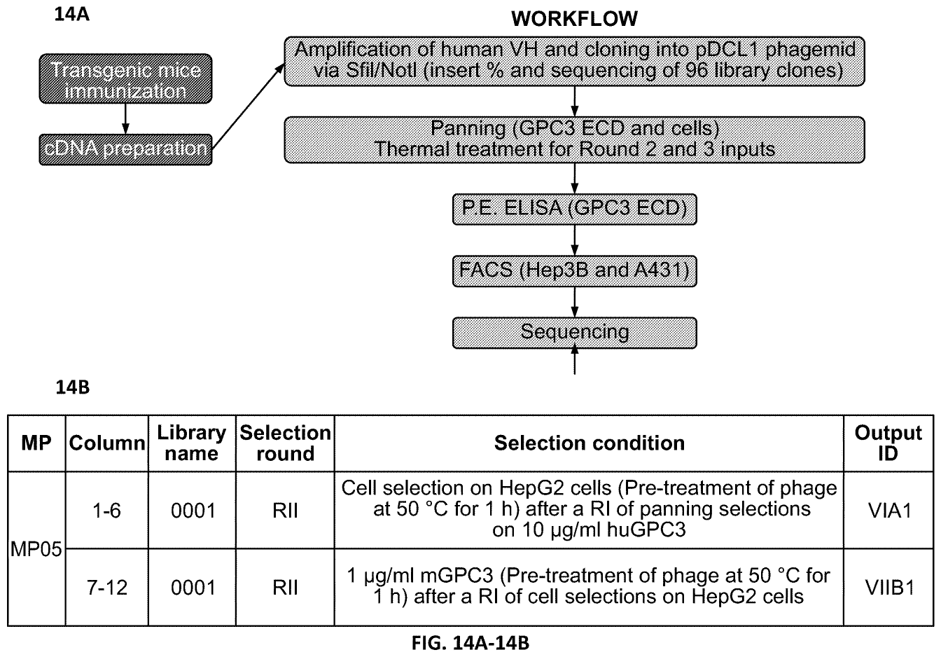

An example of a phage display procedure (FIG. 14A) was repeated to find additional binders in the library. Two panning strategies were utilized. The first strategy comprised a first round utilizing 10 μg/mL human GPC3, followed by a second round utilizing HepG2 cells. The second strategy comprised a first round using HepG2 cells followed by a second round utilizing 1 μg/mL mouse GPC3. Master plate 5 (MP05) was generated as shown in FIG. 14B. Phage FACS and periplasmic extract ELISA were carried out to test the binding of the VHH domains. All binders were sequenced and grouped. In total, 10 additional, unique VHH domain sequences were identified from MP05 and 3 new HCDR3 families were identified. 4 more unique sequences from MP05 were added to the final list of binders for a total of 36 unique VHH domains.

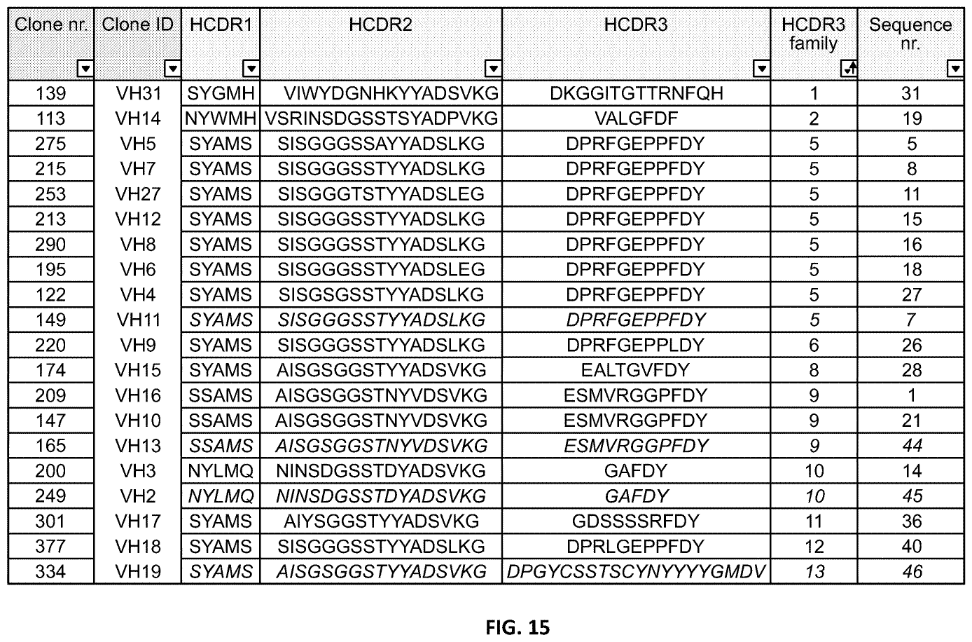

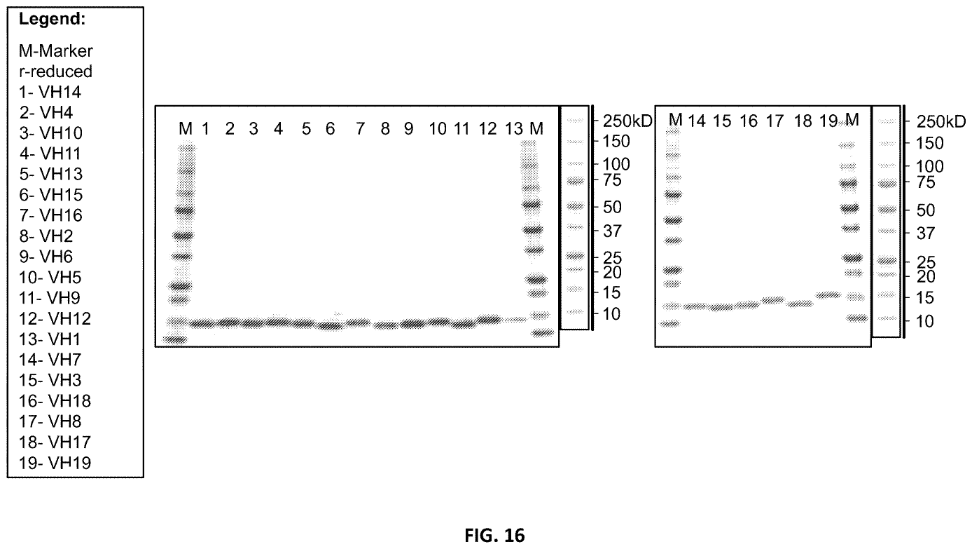

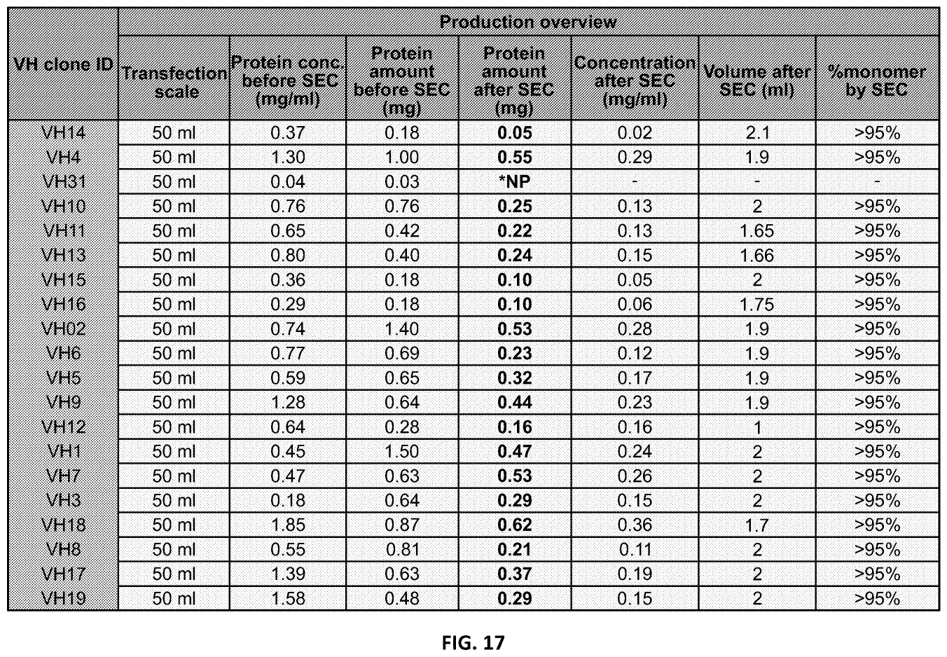

Subsequently, 20 clones (FIG. 15) were selected for recloning into a mammalian expression vector comprising a nucleic acid sequence encoding a His tag for production and purification of the anti-GPC3 VHH domains. Of the 20 clones selected, 19 were successfully produced as a His fusion. These are clones VHH1. VHH2, VHH3, VHH4, VHH5, VHH6, VHH7, VHH8, VHH9, VHH10, VHH11, VHH12, VHH13, VHH14, VHH15, VHH16, VHH17, VHH18, and VHH19. A band of about 15 kDa, corresponding to the VHH-His tagged domains was observed for all 19 VHH-His tagged clones (FIG. 16). No aggregation was observed for any of the 19 clones. A summary of the production of the VHH-His tagged domains is shown in FIG. 17.

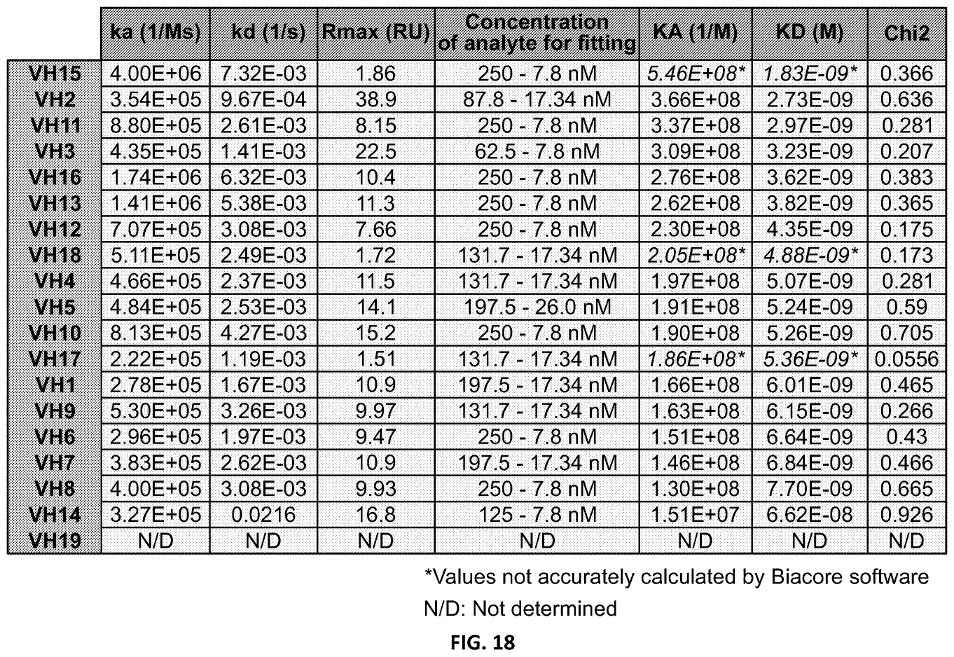

Affinities of the 19 VHH-His tagged domains were then determined by BiaCore assay. Human GPC3 protein (Sino biological cat. nr. 10088-H08H-500) was immobilized on CM5 sensor chip (GE Healthcare) as antigen. Purified VHH-His tagged domains were diluted in BiaCore buffer (0.01 M HEPES pH 7.4, 0.15 M NaCl, 3 mM EDTA, 0.05% P20) to 500 nM and injected over 2 minutes at 30 L/min followed by a half dilution series to 7.8 nM. 10 μl of 10 mM NaOH/1M NaCl were injected between samples and then off-rate wash was 5 minutes. Measured affinities are shown in FIG. 18.

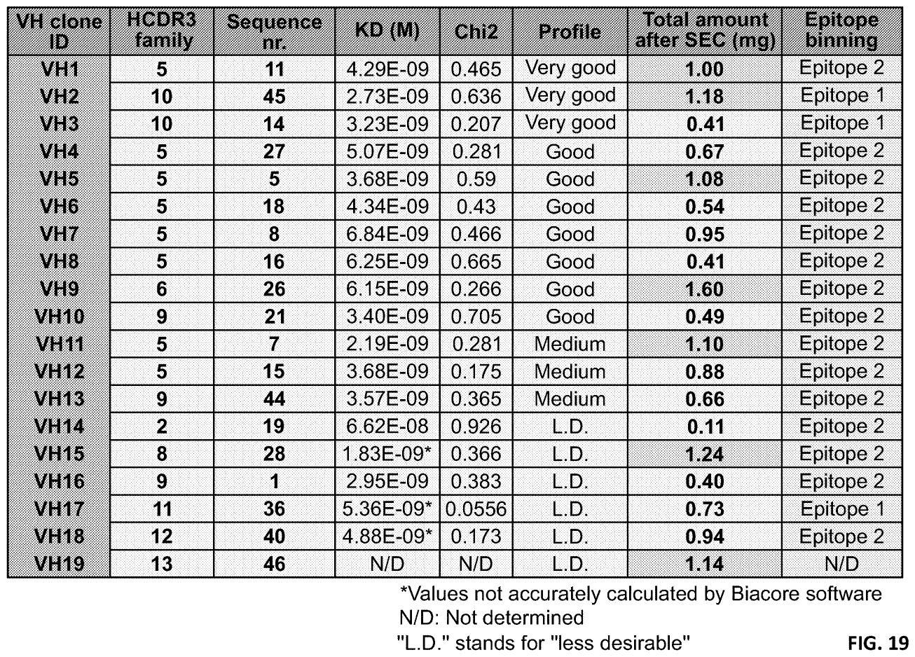

The 19 VHH-His tagged domains were then evaluated for epitope-binding site. All VHHs were injected one at a time to evaluate the epitope binding site for each VHH domain. 20 μg/ml VHH2 or VHH1 (one of the clones with the best K_{D}) was immobilized on CM5 sensor chip at 1000RU. Human GPC3 was injected followed by an off rate wash for 4 minutes. No binding is observed if the VHH binds at the same epitope as VHH2, while a second binding event is observed if the VHH binds at a different epitope. All VHHs were binned into two groups based on the epitopes. VHH2 and VHH3 bind the same epitope and all other VHs bind the second epitope (FIG. 19).

Anti-GPC3 VHH domain selection ultimately led to the identification of a plurality of stable, GPC3-specific VHH domains. The amino acid sequences of the anti-GPC3 VHH domains identified from this study are summarized in Table 8 below and their CDR sequences are summarized in Table 9 below.

TABLE 8
Amino acid Sequences of anti-GPC3 VHH domains.

| | SEQ | |
|---|---|---|
| Antibody | ID | |
| VHH | NO: | Anti-GPC3 VHH domain amino acid sequences |
| VHH1 | 1 | EVQLVESGGGLVQPGGSLRLSCAASGFTFSSYAMSWVRQA |
| | | PGKEPEWVSSISGGGTSTYYADSLEGRFTISRDNSKNTLYL |
| | | QMNSLRAEDTAVYYCSKDPRFGEPPFDYWGQGTTVTVSS |
| VHH2 | 2 | QVQLVESGGGLVQPGGSLRLSCAASGFTFSNYLMHWVRQ |
| | | APGKGLEWLSNINSDGSSTYYADSVKGRFTISRDNSKNTLY |
| | | LQMNSLRAEDTAVYYCTVGAFDYWGQVTTGTVSS |
| VHH3 | 3 | EVQLVESGGGVVQPGRSLRLSCAASGFTFSNYLMQWVRQ |
| | | APGKGLVWLSNINSDGSSTDYADSVKGRFTISRDNAKNTL |
| | | YLQMNSLRAEDTAVYYCRVGAFDYWGQGTLVTVSS |
| VHH4 | 4 | QVQLVESGGGLVQPGGSLRLSCAASGFTFSSYAMSWVRQ |
| | | APGKESEWVSSISGSGSSTYYADSLKGRFTISRDNSKNTLY |
| | | LQMNSLRAEDTAVYYCSKDPRFGEPPFDYWGQGTTVTVSS |
| VHH5 | 5 | EVQLVESGGGLVQPGGSLRLSCAASGFTFSSYAMSWVRQA |
| | | PGKEPEWVSSISGGGSSAYYADSLKGRFTISRDNSKNTLYL |
| | | QMNSLRAEDTAVYYCSRDPRFGEPPFDYWGQGTTVTVSS |
| VHH6 | 6 | QVQLVESGGGLVQPGGSLRLSCAASGFIFSSYAMSWVRQA |
| | | PGKEPEWVSSISGGGSSTYYADSLEGRFTISRDNSKNTLYL |
| | | QMNSLRAEDTAVYYCSKDPRFGEPPFDYWGQGTTVTVSS |
| VHH7 | 7 | EVQLVESGGGLVQPGGSLRLSCAASGFTFSSYAMSWVRQA |
| | | PGKEPEWVSSISGGGSSTYYADSLKGRFTISRDNSKNTLYL |
| | | QMNSLRAEDTAVYYCSRDPRFGEPPFDYWGQGTTVTVSS |
| VHH8 | 8 | EVQLVESGGGVVQPGRSLRLSCAASGFTFSSYAMSWVRQ |
| | | APGKEPEWVSSISGGGSSTYYADSLKGRFTISRDNSKNTLY |
| | | LQMNSLRAEDTAVYYCSRDPRFGEPPFDYWGQGTTVTVSS |
| VHH9 | 9 | QVQLVESGGGLVQPGGSLRLSCAASGFTFSSYAMSWVRQ |
| | | APGKEPEWVSSISGGGSSTYYADSLKGRFTISRDNSKNTLY |
| | | LQMNSLRAEDTAVYYCSRDPRFGEPPLDYWGQGTTVTVSS |
| VHH10 | 10 | QVQLVESGGGLVQPGGSLRLSCAASGFTFSSSAMSWVRQA |
| | | PGKGLEWVSAISGSGGSTNYVDSVKGRFTVSRDNSKNTLY |
| | | LQMNRLRAEDTAVYYCAKESMVRGGPFDYWGQGTLVTV |
| | | SS |
| VHH11 | 11 | EVQLVESGGGLVQPGGSLRLSCAASGFTFSSYAMSWVRQA |
| | | PGKEPEWVSSISGGGSSTYYADSLKGRFTISRDNSKNTLYL |
| | | QMNSLRAEDTAVYYCSRDPRFREPPFDYWGQGTLVTVSS |
| VHH12 | 12 | EVQLVESGGGVVQPGRSLRLSCAASGFTFSSYAMSWVRQ |
| | | APGKEPEWVSSISGGGSSTYYADSLKGRFTISRDNSKNTLY |
| | | LQMNSLRAEDTAVYYCSRDPRFGEPPFDYWGQGTLVTVSS |
| VHH13 | 13 | EVQLVESGGGLVQPGGSLRLSCAASGFTFSSYAMSWVRQA |
| | | PGKEPEWVSSISGGGGSTYYADSLKGRFTISRDNSKNTLYL |
| | | QMNRLRAEDTAVYYCARDPMFGERPFDYWGQGTLVTVSS |
| VHH14 | 14 | QVQLVESGGGLVQPGGSLRLSCAASGFNFSNYWMHWVR |
| | | QAPGKELVWVSRINSDGSSTSYADPVKGRFTISRDNANNM |
| | | LYLQMNSLRAEDTAMYYCVRVALGFDFWGQGTLVTVSS |
| VHH15 | 15 | QVQLVESGGGLVQPGGSLRLSCAASGFTFSSYAMSWVRQ |
| | | APGKGLEWVSAISGSGGSTYYADSVKGRFTISRDNSKNTL |
| | | YLQMNSLRAEDTAVYYCAKEALTGVFDYWGQGTTVTVSS |
| VHH16 | 16 | EVQLVESGGGLVQPGGSLRLSCAASGFTFSSSAMSWVRQA |
| | | PGKGLEWVSAISGSGGSTNYVDSVKGRFTVSRDNSKNTLY |
| | | LQMNRLRAEDTAVYYCAKESMVRGGPFDYWGQGTTVTV |
| | | SS |
| VHH17 | 17 | EVQLVESGGGLVQPGGSLRLSCAASGFTFSSYAMSWVRQA |
| | | PGKGLEWVSAIYSGGSTYYADSVKGRFTISRDNSKNTLYL |
| | | QMNSLRAEDTAVYYCARGDSSSSRFDYWGQGTLVTVSS |
| VHH18 | 18 | QVQLVESGGGLVQPGGSLRLSCAASGFTFSSYAMSWVRQ |
| | | APGKEPEWVSSISGGGSSTYYADSLKGRFTISRDNSKNTLY |
| | | LQMNSLRAEDTAVYYCSRDPRLGEPPFDYWGQGTTVTVSS |
| VHH19 | 19 | EEQLVESGGGLVQPGGSLRLSCAASGFTFSSYAMSWVRQA |
| | | PGKEPEWVSSISGGGGSTYYADSLKGRFTISRDNSKNTLYL |
| | | QMNSLRAEDTAVYYCARDPRYGEPPFDYRCQGTTVTVSS |
| VHH20 | 80 | EVQLVESGGGLVQPGGSLRLSCAASGFTFSSYAMSWVRQA |
| | | PGKEPEWISSISGSGSSTYYADSLKGRFTISRDNSKNTLYLQ |
| | | MNSLRAEDTAVYYCSKDPRFGEPPFDYWGQGTTVTVSS |
| VHH21 | 81 | QVQLVESGGGLVQPGGSLRLSCAASGFTFSSYAMSWVRQ |
| | | APGKEPEWISSISGSGSSTYYADSLKGRFTISRDNSKNTLYL |
| | | QMNSLRAEDTAVYYCSKDPRFGEPPFDYWGQGTLVTVSS |
| VHH22 | 82 | EVQLVESGGGLVQPGGSLRLSCAASGFTFSSYAMSWVRQA |
| | | PGKEPEWVSSISGGGSSTYYADSLKGRFTISRDNSKNTLYL |
| | | QMDSLRAEDTAVYYCSRDPRFGEPPFDYWGQGTLVTVSS |
| VHH23 | 83 | EVQLVESGGGLVQPGGSLRLSCAASGFTFSSYAMSWVRQA |
| | | PGKESEWVSSISGGGSSTYYADSLKGRFTISRDNSKNTLYL |
| | | QMNSLRAEDTAVYYCSKDPRFGEPPFDYWGQGTLVTVSS |
| VHH24 | 84 | EVQLVESGGGLVQPGGSLRLSCAASGFTFSSYAMSWVRQA |
| | | PGKEPEWVSSISGGGTSTYYADSLEGRFTISRDNSKNTLYL |
| | | QMNSLRAEDTAVYYCSKDPRFGEPPFDYWGQGTLVTVSS |
| VHH25 | 85 | EVQLVESGGGLVQPGGSLRLSCAASGFTFSSYAMSWVRQA |
| | | PGKESEWVSSISGSGSSTYYADSLKGRFTISRDNSKNTLYL |
| | | QMNSLRAEDTAVYYCSKDPRFGEPPFDYWGQGTLVTVSS |
| VHH26 | 86 | EVQLVESGGGLVQPGGSLRLSCAASGFTFSSYAMSWVRQA |
| | | PGKEPEWISSISGSGSSTYYADSLKGRFTISRDNSKNTLYLQ |
| | | MNSLRAEDTAVYYCSKDPRFGEPPFDYWGQGTLVTVSS |
| VHH27 | 87 | EVQLVESGGGLVQPGGSLRLSCAASGFTFSSYAMSWVRQA |
| | | PGKEPEWVSSISGGGSSTYYADSLKGRFTISRDNSKNTLYL |
| | | QMNSLRAEDTAVYYCSRDPRFGEPPFDYWGQGTLVTVSS |
| VHH28 | 88 | EVQLVESGGGVVQPGRSLRLSCAASGFTFSSYAMSWVRQ |
| | | APGKEPEWVSSISGGGSSTYYADSLKGRFTISRDNSKNTLY |
| | | LQMNSLRAEDTAVYYCSRDPRFGEPPFDYWGQGTTVTVSS |
| VHH29 | 89 | QVQLVESGGGLVQPGGSLRLSCAASGFTFSSYAMSWVRQ |
| | | APGKEPEWVSSISGGGSSTYYADSLKGRFTISRDNSKNTLY |
| | | LQMNSLRAEDTAVYYCSRDPRFGEPPFDYWGQGTLVTVSS |
| VHH30 | 90 | QVQLVESGGGLVQPGGSLRLSCAASGFIFSSYAMSWVRQA |
| | | PGKEPEWVSSISGGGSSTYYADSLEGRFTISRDNSKNTLYL |
| | | QMNSLRAEDTAVYYCSKDPRFFEPPFDYWGQGTLVTVSS |
| VHH31 | 91 | QVQLVESGGGVVQPGRSLRLSCAASGFTFSSYGMHWVRQ |
| | | APGKGLEWVAVIWYDGNHKYYADSVKGRFTISRDNSKNT |
| | | LYLQMNSLRAEDTAVYYCARDKGGITGTTRNFQHWGQGT |
| | | TVTVSS |
| VHH32 | 92 | QVQLVESGGGLVQPGGSLRLSCAASGFTFSSFAMSWVRQA |
| | | PGKGLEWVSAISGSGGRTHYADSVKGRFTISRDNSKNTLY |
| | | LQMNSLRAEDTAVYYCAKEALTGVFDYWGQGTLVTVSS |
| VHH33 | 93 | QVQLVESGGGLVQPGGSLRLSCAASGFTFSSSAMSWVRQA |
| | | PGKGLEWVSAISGSGGSTNYVDSVKGRFTVSRDNSKNTLY |
| | | LQMNRLRAEDTAVYYCAKESMVRGGPFDYWGQGTTVTV |
| | | SS |
| VHH34 | 94 | QVQLVESGGGLVRPGGSLRLSCAASGFTFSSYAMSWVRQ |
| | | APGKEPEWVSSISGGGSSTYYADSLKGRFTISRDNSKNTLY |
| | | LQMNSLRAEDTAVYYCSRDPRFGEPPFDYWGQGTLVTVSS |
| VHH35 | 95 | QVQLVESGGGVVQPGRSLRLSCAASGFTFSSYAMSWVRQ |
| | | APGKEPEWVSSISGGGSSTYYADSLKGRFTISRDNSKNTLY |
| | | LQMNSLRAEDTAVYYCSRDPRFGEPPFDYWGQGTLVTVSS |
| VHH36 | 96 | QVQLVESGGGLVQPGGSLRLSCAASGFTFSSYAMSWVRQ |
| | | APGKEPEWVSSISGGGSSTYYADSLKGRFTISRDNSKNTLY |
| | | LQMNSLRAEDTAVYYCSRDPRFGEPPFDYWGQGTTVTVSS |

TABLE 9
Exemplary Amino acid Sequences of anti-GPC3 VHH domains CDRs

| Antibody | | | | CDR3 |
|---|---|---|---|---|
| VHH | VHH-CDR1 | VHH-CDR2 | VHH-CDR3 | Family |
| VHH1 | SYAMS | SISGGGTSTYYA | DPRFGEPPFDY | 5 |
| | (SEQ ID NO: 20) | DSLEG | (SEQ ID NO: 22) | |
| | | (SEQ ID NO: 21) | | |
| VHH2 | NYLMH | NINSDGSSTYYA | GAFDY | 10 |
| | (SEQ ID NO: 23) | DSVKG | (SEQ ID NO: 25) | |
| | | (SEQ ID NO: 24) | | |
| VHH3 | NYLMQ | NINSDGSSTDYA | GAFDY | 10 |
| | (SEQ ID NO: 26) | DSVKG | (SEQ ID NO: 25) | |
| | | (SEQ ID NO: 27) | | |
| VH4 | SYAMS | SISGSGSSTYYAD | DPRFGEPPFDY | 5 |
| | (SEQ ID NO: 20) | SLKG | (SEQ ID NO: 22) | |
| | | (SEQ ID NO: 30) | | |
| VHH5 | SYAMS | SISGGGSSAYYA | DPRFGEPPFDY | 5 |
| | (SEQ ID NO: 20) | DSLKG | (SEQ ID NO: 22) | |
| | | (SEQ ID NO: 33) | | |
| VHH6 | SYAMS | SISGGGSSTYYA | DPRFGEPPFDY | 5 |
| | (SEQ ID NO: 20) | DSLEG | (SEQ ID NO: 22) | |
| | | (SEQ ID NO: 36) | | |
| VHH7 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH8 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH9 | SYAMS | SISGGGSSTYYA | DPRFGEPPLDY | 6 |
| | (SEQ ID NO: 20) | DSLKG | (SEQ ID NO: 46) | |
| | | (SEQ ID NO: 39) | | |
| VHH10 | SSAMS | AISGSGGSTNYV | ESMVRGGPFDY | 9 |
| | (SEQ ID NO: 47) | DSVKG | (SEQ ID NO: 49) | |
| | | (SEQ ID NO: 48) | | |
| VHH11 | SYAMS | SISGGGSSTYYA | DPRFREPPFDY | 7 |
| | (SEQ ID NO: 20) | DSLKG | (SEQ ID NO: 52) | |
| | | (SEQ ID NO: 39) | | |
| VHH12 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH13 | SYAMS | SISGGGGSTYYA | DPMFGERPFDY | 3 |
| | (SEQ ID NO: 20) | DSLKG | (SEQ ID NO: 58) | |
| | | (SEQ ID NO: 57) | | |
| VHH14 | NYWMH | VSRINSDGSSTSY | VALGFDF | 2 |
| | (SEQ ID NO: 59) | ADPVKG | (SEQ ID NO: 61) | |
| | | (SEQ ID NO: 60) | | |
| VHH15 | SYAMS | AISGSGGSTYYA | EALTGVFDY | 8 |
| | (SEQ ID NO: 20) | DSVKG | (SEQ ID NO: 64) | |
| | | (SEQ ID NO: 63) | | |
| VHH16 | SSAMS | AISGSGGSTNYV | ESMVRGGPFDY | 9 |
| | (SEQ ID NO: 47) | DSVKG | (SEQ ID NO: 49) | |
| | | (SEQ ID NO: 48) | | |
| VHH17 | SYAMS | AIYSGGSTYYAD | GDSSSSRFDY | 11 |
| | (SEQ ID NO: 20) | SVKG | (SEQ ID NO: 70) | |
| | | (SEQ ID NO: 69) | | |
| VHH18 | SYAMS | SISGGGSSTYYA | DPRLGEPPFDY | 12 |
| | (SEQ ID NO: 20) | DSLKG | (SEQ ID NO: 73) | |
| | | (SEQ ID NO: 39) | | |
| VHH19 | SYAMS | SISGGGGSTYYA | DPRYGEPPFDY | 13 |
| | (SEQ ID NO: 20) | DSLKG | (SEQ ID NO: 76) | |
| | | (SEQ ID NO: 57) | | |
| VHH20 | SYAMS | SISGSGSSTYYAD | DPRFGEPPFDY | 5 |
| | (SEQ ID NO: 20) | SLKG | (SEQ ID NO: 22) | |
| | | (SEQ ID NO: 30) | | |
| VHH21 | SYAMS | SISGSGSSTYYAD | DPRFGEPPFDY | 5 |
| | (SEQ ID NO: 20) | SLKG | (SEQ ID NO: 22) | |
| | | (SEQ ID NO: 30) | | |
| VHH22 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH23 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH24 | SYAMS | SISGGGTSTYYA | DPRFGEPPFDY | 5 |
| | (SEQ ID NO: 20) | DSLEG | (SEQ ID NO: 22) | |
| | | (SEQ ID NO: 21) | | |
| VHH25 | SYAMS | SISGSGSSTYYAD | DPRFGEPPFDY | 5 |
| | (SEQ ID NO: 20) | SLKG | (SEQ ID NO: 22) | |
| | | (SEQ ID NO: 30) | | |
| VHH26 | SYAMS | SISGSGSSTYYAD | DPRFGEPPFDY | 5 |
| | (SEQ ID NO: 20) | SLKG | (SEQ ID NO: 22) | |
| | | (SEQ ID NO: 30) | | |
| VHH27 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH28 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH29 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH30 | SYAMS | SISGGGSSTYYA | DPRFFEPPFDY | 4 |
| | (SEQ ID NO: 20) | DSLEG | (SEQ ID NO: 97) | |
| | | (SEQ ID NO: 36) | | |
| VHH31 | SYGMH | VIWYDGNHKYY | DKGGITGTTRNF | 1 |
| | (SEQ ID NO: 98) | ADSVKG | QH | |
| | | (SEQ ID NO: 99) | (SEQ ID NO: 100) | |
| VHH32 | SFAMS | AISGSGGRTHYA | EALTGVFDY | 8 |
| | (SEQ ID NO: 101) | DSVKG | (SEQ ID NO: 64) | |
| | | (SEQ ID NO: 102) | | |
| VHH33 | SSAMS | AISGSGGSTNYV | ESMVRGGPFDY | 9 |
| | (SEQ ID NO: 47) | DSVKG | (SEQ ID NO: 49) | |
| | | (SEQ ID NO: 48) | | |
| VHH34 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH35 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |
| VHH36 | SYAMS | SISGGGSSTYYA | DPRFGEPPFDY | 5 |
| | (SEQ ID NO: 20) | DSLKG | (SEQ ID NO: 22) | |
| | | (SEQ ID NO: 39) | | |

### Anti-GPC3 VHH Domain Characterization

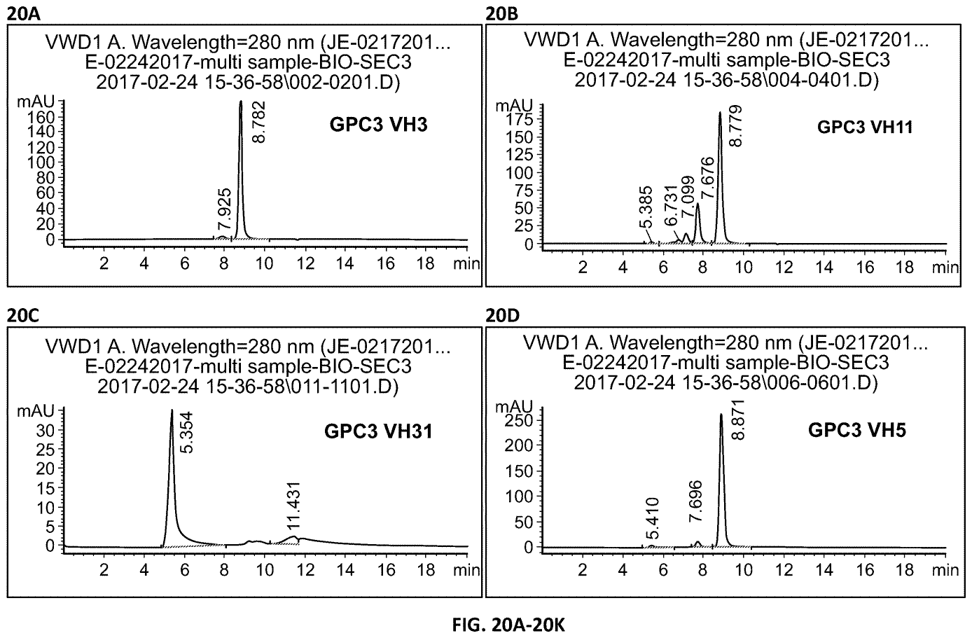

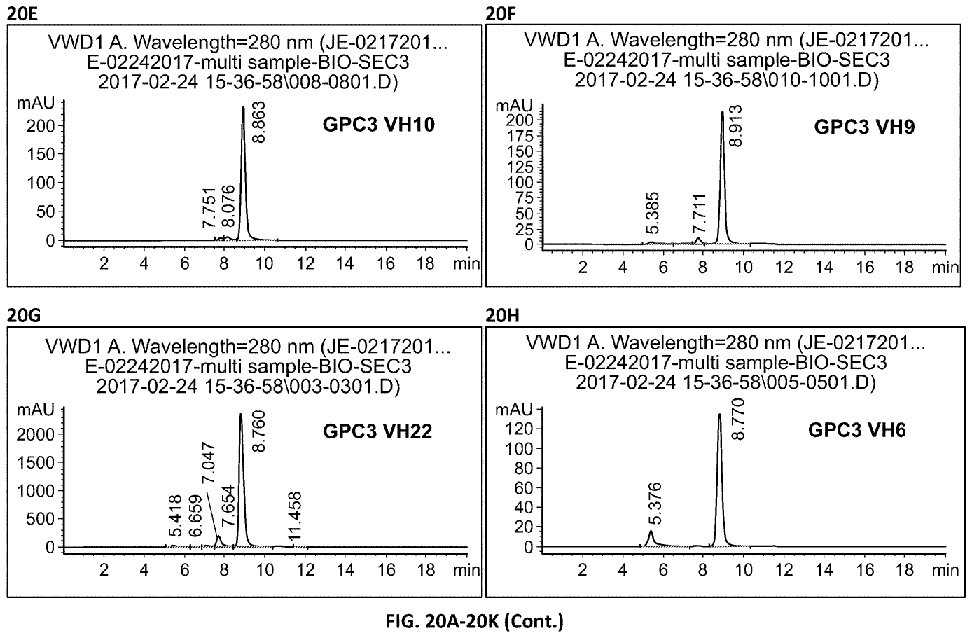

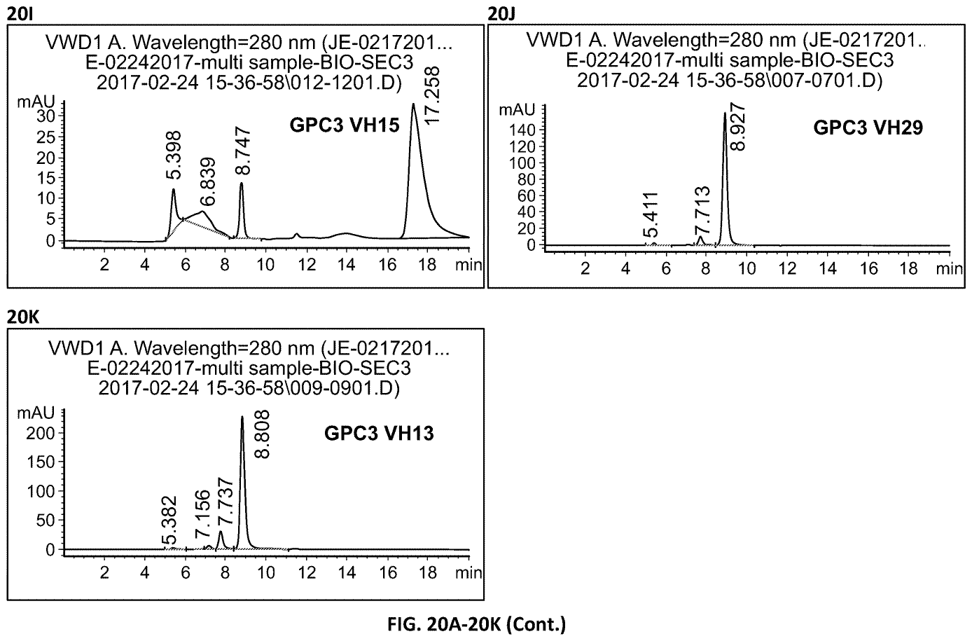

Characterization of anti-GPC3 VHH domains for native molecule homogeneity and determination of molecular weight was conducted using Size Exclusion Chromatography with Light Scattering (SEC-LS) for a subset of anti-GPC3 VHH domains, VHH3, VHH11, VHH31, VHH5, VHH10, VHH9, VHH22, VHH6, VHH29, VHH13, and VHH15. Size exclusion chromatography (SEC) was carried out on a Zenix SEC 300 4.6×300 mm (Sepax Technologies) in 20 mM sodium phosphate pH 7.2, 150 mM NaCl (PBS). 0.05% Sodium Azide at a flow rate of 0.35 ml/min using an Agilent 1260 UPLC. SEC profiles of anti-GPC3 VHH domains tested indicated all, except VHH31 and VHH15, were relatively homogenous. Each anti-GPC3 VH domain (except VHH31 and VHH15) eluted as a monomeric peak at an elution time of around 8.8 minutes. The monomeric peaks were both sharp and symmetric, indicating that these molecules were mono-dispersed. VHH31 eluted at 5.35 minutes, indicating a higher molecular weight suggestive that the molecule was aggregated. VHH15 eluted at multiple peaks in addition to the monomeric peak at 8.8 minutes, indicating that VH15 was not mono-dispersed (FIG. 20A-20K).

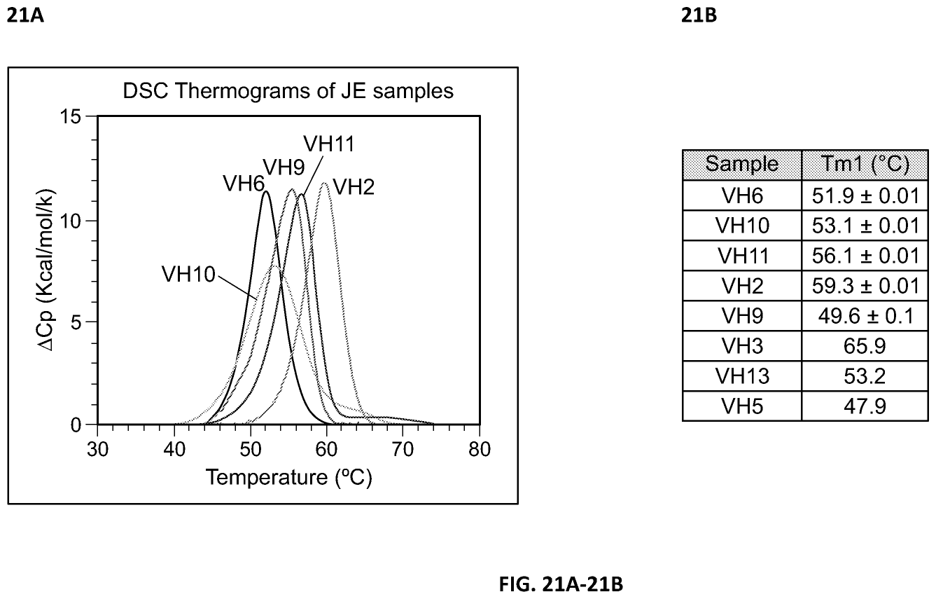

A subset of anti-GPC3 VHH domains were further evaluated for stability using differential scanning calorimetry (DSC). Melting temperatures of the tested VHH domains were determined to be between about 47° C. and 66° C. (FIG. 21A-21B).

Surface Plasmon Resonance (SPR) binding studies were performed using a Biacore T200 instrument (Cytiva) at 25° C. The target molecule (either human GPC3 (huGPC3) or mouse GPC3 (mGPC3)) was immobilized on CM5 sensorchips using the Biacore amine coupling kit according to manufacturer's instructions. Briefly, the chip was activated with a 50 μL injection of 1:1 N-hydroxsuccinimide (NHS): 1-Ethyl-3(3-dimethylaminopropyl)-carbodiimide hydrochloride (EDC). Human GPC3, diluted to 10 μg/ml in 10 mM sodium acetate. pH 4.0 was injected over one quadrant of the activated chip. A second quadrant was injected with mGPC3 diluted to 10 μg/ml in 10 mM sodium acetate, pH 4.0. A third quadrant was exposed to 10 mM sodium acetate, pH 4.0 as a control surface. Excess free amine groups were capped with a 50 μL injection of 1 M Ethanolamine. Immobilization levels were set to 1000 RU. All samples were prepared in Biacore buffer (HBS-EP+). This same buffer was used as the running buffer during sample analysis. Upon completion of each association and dissociation cycle, surfaces were regenerated with 10 mM glycine pH 2.5 solution. The association rates (ka), dissociation rate constants (kd), and affinity constants (K_{D}) were calculated using Biacore T200 evaluation software. Each fit was evaluated by the agreement between experimental data and the calculated fits, where the Chi² values were below 10% of Rmax. Surface densities of the molecule were optimized to minimize mass transfer while maintaining enough response. Results demonstrated that all anti-GPC3 VHH domains were cross human and mouse GPC3 binders with K_{D} at single digit nM range (FIG. 22).

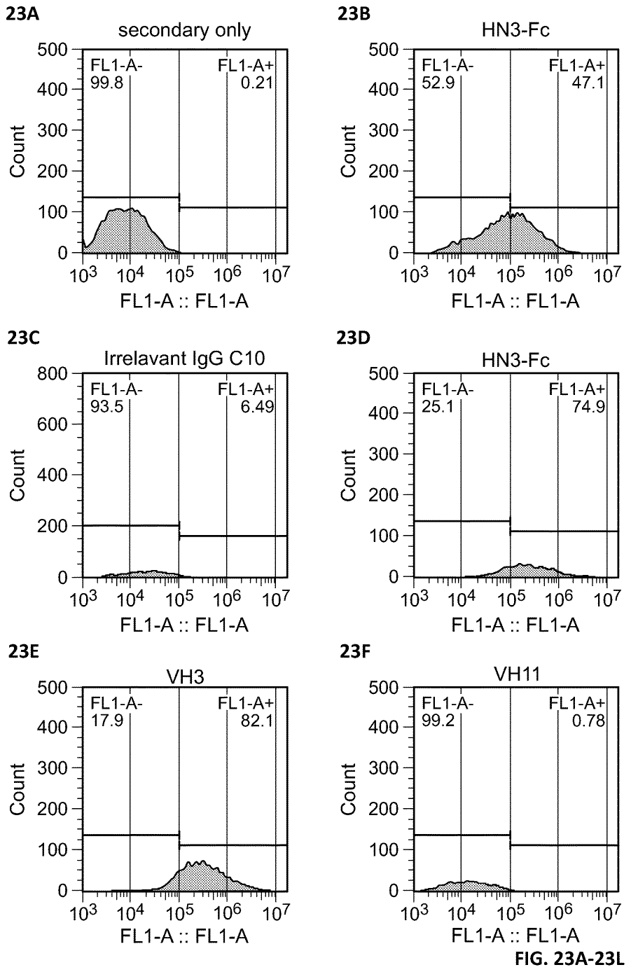

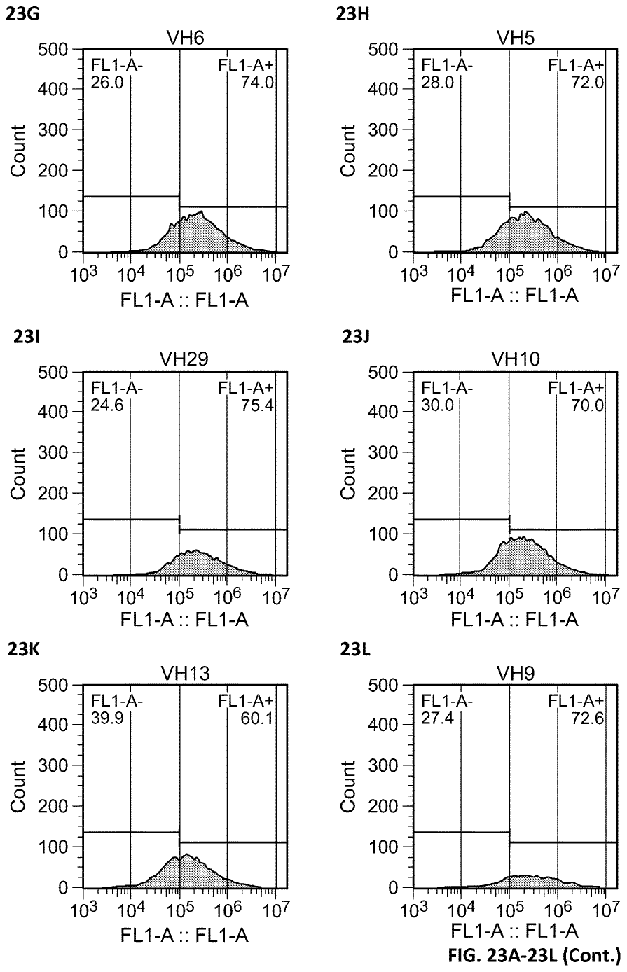

Fluorescence Activated Cell Sorting (FACS) was also conducted to confirm that the VHH molecules bind GPC3 on the cell surface. 1×10⁵ cells in 100 μl FACS buffer (PBS, 2% FBS, 1 mM EDTA) were incubated with 1 μg/ml V_{H} molecules on ice for 1 hour. Next cells were washed three times with FACS buffer, then resuspended in FACS buffer containing anti-His-FITC (GenScript, NJ) at 0.5 μg/mL, or anti-human Fc FitC (Abcam) for any Fc containing constructs. Cells were incubated with the secondary antibody for an additional 15 min at 4° C. and washed 3 times with FACS buffer and resuspended in 200 μL of FACS buffer. The fluorescent staining of the cellular bound antibody on the cells was quantified by determining the mean channel fluorescence by FACS. Cellular fluorescence was determined on Accuri C₆ (Becton-Dickinson, Franklin Lakes, NJ), (FIG. 23A-23L).

### Example 2: Anti-GPC3 VHH Domain Inclusion in Chimeric Antigen Receptors

The present example demonstrates use of anti-GPC3 VHH domains in third generation Chimeric Antigen Receptors (CARs). mRNAs encoding third generation CARs comprising either of the VHH29, VHH6, or VHH3 anti-GPC3 VHH domains and a CD8 hinge, CD28 transmembrane domain (TM), CD28 intracellular domain, 4-1BB intracellular domain, and CD3z intracellular domain (anti-GPC3 VHH CARs) were generated. Examples of CAR amino acid and nucleotide sequences are shown in Tables 10 and 11, respectively. Note that the signal sequence (MALPVTALLLPLALLLHAARPD (SEQ ID NO: 243)) and the Flag tag (DYKDDDDK (SEQ ID NO: 244)) are not part of the final CAR and is so is not part of the mature CAR that is expressed on the surface of the cell (e.g., T cell, NK cell). Thus, this disclosure encompasses the amino acid sequences in Table 10 below without both the signal sequence and the Flag tag (i.e., the N-terminal 30 amino acids). Similarly, the disclosure features nucleic acids encoding the CARs of Table 11, but that lack the nucleic acid encoding the both the signal sequence and the Flag tag (i.e., the 5′ most 90 nucleic acids, i.e., ATGGCCCTGCCTGTGACCGCCCTGCTGCTGCCTCTGGCCCTGCTCCTGCAC GCCGCCAGACCTGACGACTACAAGGACGACGACGACAAGCA (SEQ ID NO: 245)).

TABLE 10
Examples of CAR amino acid sequences

| | SEQ ID | |
|---|---|---|
| CAR | NO: | CAR amino acid sequences |
| VHH29- | 153 | MALPVTALLLPLALLLHAARPDDYKDDDDKQVQLVESG |
| CD8 hinge | | GGLVQPGGSLRLSCAASGFTFSSYAMSWVRQAPGKEPEW |
| (Control) | | VSSISGGGSSTYYADSLKGRFTISRDNSKNTLYLQMNSLRA |
| | | EDTAVYYCSRDPRFGEPPFDYWGQGTLVTVSSTTTPAPRPP |
| | | TPAPTIASQPLSLRPEACRPAAGGAVHTRGLDFACD |
| VHH3-CD8 | 158 | MALPVTALLLPLALLLHAARPDDYKDDDDKQVQLVESG |
| hinge- | | GGLVQPGGSLRLSCAASGFIFSSYAMSWVRQAPGKEPEWV |
| CD28TM- | | SSISGGGSSTYYADSLEGRFTISRDNSKNTLYLQMNSLRAE |
| CD28 ICD- | | DTAVYYCSKDPRFGEPPFDYWGQGTTVTVSSTTTPAPRPPT |
| 4-1BB ICD- | | PAPTIASQPLSLRPEACRPAAGGAVHTRGLDFACDFWVLV |
| CD3z ICD | | VVGGVLACYSLLVTVAFIIFWVRSKRSRLLHSDYMNMTPR |
| | | RPGPTRKHYQPYAPPRDFAAYRSKRGRKKLLYIFKQPFMR |
| | | PVQTTQEEDGCSCRFPEEEEGGCELRVKFSRSADAPAYKQ |
| | | GQNQLYNELNLGRREEYDVLDKRRGRDPEMGGKPRRKNP |
| | | QEGLYNELQKDKMAEAYSEIGMKGERRRGKGHDGLYQG |
| | | LSTATKDTYDALHMQALPPR |
| VHH6-CD8 | 159 | MALPVTALLLPLALLLHAARPDDYKDDDDKQVQLVQSG |
| hinge- | | GGLVQPGGSLRLSCAASYFDFDSYEMSWVRQAPGKGLEW |
| CD28TM- | | IGSIYHSGSTYYNPSLKSRVTISRDNSKNTLYLQMNTLRAE |
| CD28 ICD- | | DTATYYCARVNMDRFDYWGQGTLVTVSSTTTPAPRPPTP |
| 4-1BB ICD- | | APTIASQPLSLRPEACRPAAGGAVHTRGLDFACDFWVLVV |
| CD3z ICD | | VGGVLACYSLLVTVAFIIFWVRSKRSRLLHSDYMNMTPRR |
| | | PGPTRKHYQPYAPPRDFAAYRSKRGRKKLLYIFKQPFMRP |
| | | VQTTQEEDGCSCRFPEEEEGGCELRVKFSRSADAPAYKQG |
| | | QNQLYNELNLGRREEYDVLDKRRGRDPEMGGKPRRKNPQ |
| | | EGLYNELQKDKMAEAYSEIGMKGERRRGKGHDGLYQGLS |
| | | TATKDTYDALHMQALPPR |
| VHH29- | 160 | MALPVTALLLPLALLLHAARPDDYKDDDDKQVQLVESG |
| CD8 hinge- | | GGLVQPGGSLRLSCAASGFTFSSYAMSWVRQAPGKEPEW |
| CD28TM- | | VSSISGGGSSTYYADSLKGRFTISRDNSKNTLYLQMNSLRA |
| CD28 ICD- | | EDTAVYYCSRDPRFGEPPFDYWGQGTLVTVSSTTTPAPRPP |
| 4-1BB ICD- | | TPAPTIASQPLSLRPEACRPAAGGAVHTRGLDFACDFWVL |
| CD3z ICD | | VVVGGVLACYSLLVTVAFIIFWVRSKRSRLLHSDYMNMTP |
| | | RRPGPTRKHYQPYAPPRDFAAYRSKRGRKKLLYIFKQPFM |
| | | RPVQTTQEEDGCSCRFPEEEEGGCELRVKFSRSADAPAYK |
| | | QGQNQLYNELNLGRREEYDVLDKRRGRDPEMGGKPRRK |
| | | NPQEGLYNELQKDKMAEAYSEIGMKGERRRGKGHDGLY |
| | | QGLSTATKDTYDALHMQALPPR |

TABLE 11
Examples of CAR nucleotide sequences.

| | SEQ ID | |
|---|---|---|
| CAR | NO: | CAR nucleotide sequences |
| VHH29- | 161 | ATGGCCCTGCCTGTGACCGCCCTGCTGCTGCCTCTG |
| CD8 hinge | | GCCCTGCTCCTGCACGCCGCCAGACCTGACGACTAC |
| (Control) | | AAGGACGACGACGACAAGCAGGTGCAGCTGGTGGAGA |
| | | GCGGCGGCGGCCTGGTGCAGCCTGGCGGCAGCCTGAGA |
| | | CTGAGCTGCGCCGCCAGCGGCTTCACCTTCAGCAGCTAC |
| | | GCCATGAGCTGGGTGAGACAGGCCCCTGGCAAGGAGCC |
| | | TGAGTGGGTGAGCAGCATCTCTGGTGGAGGCAGCAGCA |
| | | CCTACTACGCCGACAGCCTGAAGGGCAGATTCACCATC |
| | | AGCAGAGACAACAGCAAGAACACCCTGTACCTGCAGAT |
| | | GAACAGCCTGAGAGCCGAGGACACCGCCGTGTACTACT |
| | | GCAGCAGAGATCCTAGATTCGGCGAGCCTCCTTTCGACT |
| | | ACTGGGGCCAGGGCACCCTGGTGACCGTGAGCAGCACC |
| | | ACCACCCCTGCCCCTAGACCTCCTACCCCAGCCCCTACC |
| | | ATCGCCAGCCAGCCTCTGAGCCTGCGCCCTGAAGCCTGT |
| | | AGGCCAGCCGCCGGAGGAGCCGTGCACACCAGAGGCCT |
| | | GGACTTCGCCTGCGAC |
| VHH3-CD8 | 166 | ATGGCCCTGCCTGTGACCGCCCTGCTGCTGCCTCTG |
| hinge- | | GCATTGCTATTGCACGCCGCCAGACCTGACGACTAC |
| CD28TM- | | AAGGACGACGACGACAAGCAGGTGCAGCTGGTGGAGA |
| CD28 ICD- | | GCGGCGGCGGCCTGGTGCAGCCTGGCGGCAGCCTGAGA |
| 4-1BB ICD- | | CTGAGCTGCGCCGCCAGCGGCTTCATCTTCAGCAGCTAC |
| CD3z ICD | | GCCATGAGCTGGGTGAGACAGGCCCCTGGCAAGGAGCC |
| | | TGAGTGGGTGAGCAGCATCTCAGGTGGCGGCAGCAGCA |
| | | CCTACTACGCCGACAGCCTGGAGGGCAGATTCACCATC |
| | | AGCAGAGACAACAGCAAGAACACCCTGTACCTGCAGAT |
| | | GAACAGCCTGAGAGCCGAGGACACCGCCGTGTACTACT |
| | | GCAGCAAGGACCCTAGATTCGGCGAGCCTCCTTTCGACT |
| | | ACTGGGGCCAGGGCACCACCGTGACCGTGAGCAGCACC |
| | | ACCACCCCTGCCCCTAGACCTCCTACGCCTGCCCCTACC |
| | | ATCGCCAGCCAGCCTCTGAGCTTGAGGCCTGAAGCCTGT |
| | | CGGCCAGCTGCCGGTGGCGCTGTACACACCAGAGGCCT |
| | | GGACTTCGCCTGCGACTTCTGGGTGCTGGTGGTGGTGGG |
| | | CGGCGTGCTGGCCTGCTACAGCCTGCTGGTGACCGTGGC |
| | | CTTCATCATCTTCTGGGTGAGAAGCAAGAGAAGCAGAC |
| | | TGCTGCACAGCGACTACATGAACATGACCCCTAGAAGA |
| | | CCTGGCCCTACCAGAAAGCACTACCAGCCTTACGCCCCT |
| | | CCTAGAGACTTCGCCGCCTACAGATCCAAGAGGGGCAG |
| | | AAAGAAGCTGCTGTACATCTTCAAGCAGCCTTTCATGCG |
| | | GCCTGTGCAGACCACCCAGGAGGAGGACGGCTGCAGCT |
| | | GCAGATTCCCTGAGGAGGAGGAGGGCGGCTGCGAGCTG |
| | | AGAGTGAAGTTCAGCAGAAGCGCCGACGCCCCTGCCTA |
| | | CAAGCAGGGCCAGAACCAGCTGTACAACGAGCTGAACC |
| | | TGGGCAGAAGAGAGGAGTACGACGTGCTGGACAAGAG |
| | | AAGAGGCCGTGACCCTGAGATGGGCGGCAAGCCTAGAA |
| | | GAAAGAACCCTCAGGAGGGCTTGTACAACGAACTTCAG |
| | | AAGGACAAGATGGCCGAGGCCTACAGCGAGATCGGCAT |
| | | GAAGGGCGAGAGAAGAAGAGGCAAGGGCCACGACGGC |
| | | CTGTACCAGGGCCTGAGCACCGCCACCAAGGACACCTA |
| | | CGACGCCCTGCACATGCAGGCCCTGCCTCCTAGA |
| VHH6-CD8 | 167 | ATGGCCCTGCCTGTGACCGCCCTGCTGCTGCCTCTG |
| hinge- | | GCTTTGCTCCTCCACGCCGCCAGACCTGACGACTAC |
| CD28TM- | | AAGGACGACGACGACAAGCAGGTGCAGCTGGTGCAGA |
| CD28 ICD- | | GCGGCGGCGGCCTGGTGCAGCCTGGCGGCAGCCTGAGA |
| 4-1BB ICD- | | CTGAGCTGCGCCGCCAGCTACTTCGACTTCGACAGCTAC |
| CD3z ICD | | GAGATGAGCTGGGTGAGACAGGCCCCTGGCAAGGGCCT |
| | | GGAGTGGATCGGCAGCATCTACCACAGCGGCAGCACCT |
| | | ACTACAACCCTAGCCTGAAGTCCAGAGTGACCATCAGC |
| | | AGAGACAACAGCAAGAACACCCTGTACCTGCAGATGAA |
| | | TACCCTGAGAGCCGAGGACACCGCAACGTATTACTGCG |
| | | CCAGAGTGAACATGGACAGATTCGACTACTGGGGCCAG |
| | | GGCACCCTGGTGACCGTGAGCAGCACCACCACCCCTGC |
| | | CCCTAGACCTCCTACCCCAGCCCCGACCATCGCCAGCCA |
| | | GCCTCTGTCTCTGCGGCCTGAAGCCTGCAGACCGGCTGC |
| | | AGGCGGAGCAGTGCACACCAGAGGCCTGGACTTCGCCT |
| | | GCGACTTCTGGGTGCTGGTGGTGGTGGGCGGCGTGCTGG |
| | | CCTGCTACAGCCTGTTAGTAACTGTGGCCTTCATCATTTT |
| | | CTGGGTCAGAAGCAAGAGAAGCAGACTGCTGCACAGCG |
| | | ACTACATGAACATGACCCCTAGAAGACCTGGCCCTACC |
| | | AGAAAGCACTACCAGCCTTACGCCCCTCCTAGAGACTTC |
| | | GCCGCCTACAGGAGTAAGAGAGGCAGAAAGAAGCTGCT |
| | | GTACATCTTCAAGCAGCCTTTCATGAGGCCTGTGCAGAC |
| | | CACCCAGGAGGAGGACGGCTGCAGCTGCAGATTCCCTG |
| | | AGGAAGAGGAAGGCGGCTGCGAGCTGAGAGTGAAGTTC |
| | | AGCAGAAGCGCCGACGCCCCTGCCTACAAGCAGGGCCA |
| | | GAACCAGCTGTACAACGAGCTGAACCTGGGCAGAAGAG |
| | | AGGAGTACGACGTGCTGGACAAGAGAAGAGGCAGGGA |
| | | TCCTGAGATGGGCGGCAAGCCTAGAAGAAAGAACCCTC |
| | | AGGAGGGCCTATACAACGAACTGCAGAAGGACAAGATG |
| | | GCCGAGGCCTACAGCGAGATCGGCATGAAGGGCGAGAG |
| | | ACGGAGGGGAAAGGGACACGACGGCCTGTACCAGGGCC |
| | | TGTCCACCGCCACCAAGGACACCTACGACGCCCTGCAC |
| | | ATGCAGGCCCTTCCTCCTAGA |
| VHH29- | 168 | ATGGCCCTGCCTGTGACCGCCCTGCTGCTGCCTCTGGCC |
| CD8 hinge- | | CTGTTACTACACGCCGCCAGACCTGACGACTACAAGGA |
| CD28TM- | | CGACGACGACAAGCAGGTGCAGCTGGTGGAGAGCGGCG |
| CD28 ICD- | | GCGGCCTGGTGCAGCCTGGCGGCAGCCTGAGACTGAGC |
| 4-1BB ICD- | | TGCGCCGCCAGCGGCTTCACCTTCAGCAGCTACGCCATG |
| CD3z ICD | | AGCTGGGTGAGACAGGCCCCTGGCAAGGAGCCTGAGTG |
| | | GGTGAGCAGCATCTCTGGAGGAGGCAGCAGCACCTACT |
| | | ACGCCGACAGCCTGAAGGGCAGATTCACCATCAGCAGA |
| | | GACAACAGCAAGAACACCCTGTACCTGCAGATGAACAG |
| | | CCTGAGAGCCGAGGACACCGCCGTGTACTACTGCAGCA |
| | | GGGATCCTAGATTCGGCGAGCCTCCTTTCGACTACTGGG |
| | | GCCAGGGCACCCTGGTGACCGTGAGCAGCACCACCACC |
| | | CCTGCCCCTAGACCTCCTACACCAGCACCTACCATCGCC |
| | | AGCCAGCCTCTGAGCCTCAGGCCTGAGGCCTGCCGCCCA |
| | | GCTGCTGGCGGAGCCGTGCACACCAGAGGCCTGGACTT |
| | | CGCCTGCGACTTCTGGGTGCTGGTGGTGGTGGGCGGCGT |
| | | GCTGGCCTGCTACAGCCTGCTGGTTACTGTTGCCTTCAT |
| | | CATCTTCTGGGTGAGAAGCAAGAGAAGCAGACTGCTGC |
| | | ACAGCGACTACATGAACATGACCCCTAGAAGACCTGGC |
| | | CCTACCAGAAAGCACTACCAGCCTTACGCCCCTCCTAGA |
| | | GACTTCGCCGCCTACAGAAGCAAGCGAGGCAGAAAGAA |
| | | GCTGCTGTACATCTTCAAGCAGCCTTTCATGCGGCCTGT |
| | | GCAGACCACCCAGGAGGAGGACGGCTGCAGCTGCAGAT |
| | | TCCCTGAGGAGGAGGAGGGCGGCTGCGAGCTGAGAGTG |
| | | AAGTTCAGCAGAAGCGCCGACGCCCCTGCCTACAAGCA |
| | | GGGCCAGAACCAGCTGTACAACGAGCTGAACCTGGGCA |
| | | GAAGAGAGGAGTACGACGTGCTGGACAAGAGAAGAGG |
| | | CAGAGATCCTGAGATGGGCGGCAAGCCTAGAAGAAAGA |
| | | ACCCTCAGGAGGGCTTGTATAACGAGTTGCAGAAGGAC |
| | | AAGATGGCCGAGGCCTACAGCGAGATCGGCATGAAGGG |
| | | CGAGAGAAGAAGAGGCAAGGGCCACGACGGCCTGTACC |
| | | AGGGCCTGAGCACCGCCACCAAGGACACCTACGACGCC |
| | | CTGCACATGCAGGCCCTGCCTCCTAGA |

The bolded sequence encodes the signal sequence and Flag tag.

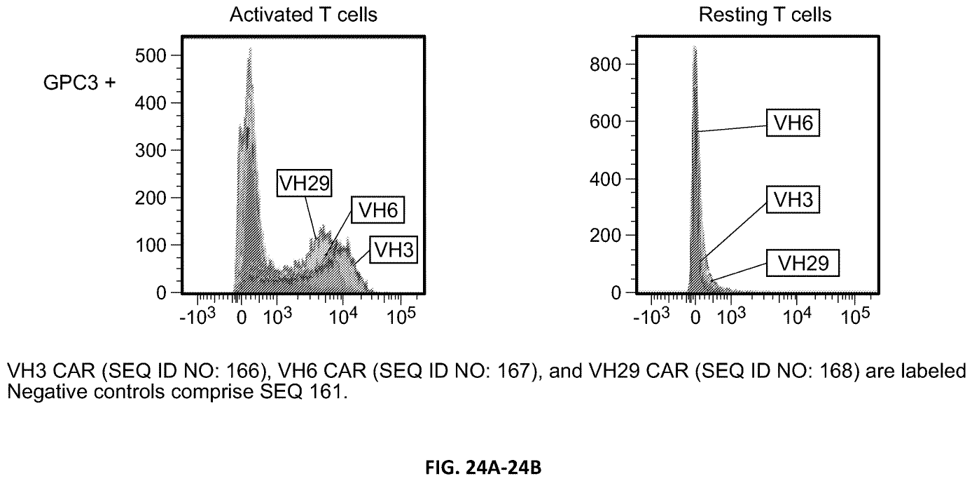

To determine whether each of these mRNA constructs encoding anti-GPC3 VHH CARs transfects activated T cells, human T cells were purified from Peripheral Blood Mononuclear Cells (PBMCs) using negative selection and subsequently activated by CD3/CD28 beads for 3 days. Activated T cells were then transfected with 1 μg β-sitosterol formulated mRNA and incubated for 24 hours. Cells were washed with FACS buffer, stained with 1 μg/mL biotin-GPC3 followed by 1 g/mL SA-FitC. Cells were then washed and expression of anti-GPC3 VHH CARs assessed using FACS. FIG. 24A-24B demonstrates mRNAs encoding the anti-GPC3 VHH CARs transfects activated T cells.

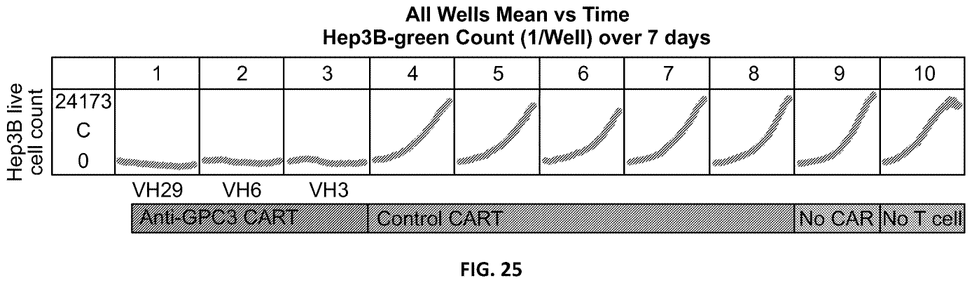

GPC3 has been shown to play an important role in the control of cell growth through the induction of apoptosis. To assess the biological activity of T cells expressing anti-GPC3 VHH CARs, 2,000 Hep3B cells transduced with a Green Fluorescent Protein (GFP)-encoding lentivirus were allowed to adhere to the surface of a 96-well plate. Human T cells were purified from PBMCs and activated by CD3/CD28 beads for 3 days. Activated T cells were transfected with 1 μg formulated mRNAs encoding anti-GPC3 VHH CARs comprising either of VHH29, VHH6, or VHH3 anti-GPC3 VHH domains, and incubated for 24 hours to generate anti-GPC3 VHH CAR T cells. About 20,000 transfected T cells were added to each well with Hep3B cells and monitored for cell proliferation using an IncuCyte®. Anti-GPC3 VHH CAR T cells inhibited Hep3B cell growth compared to controls (control CAR T, no CAR, no T cells) (FIG. 25).

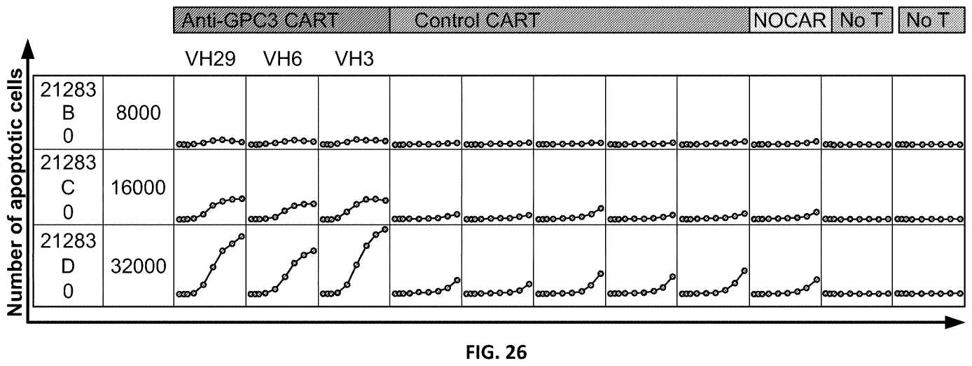

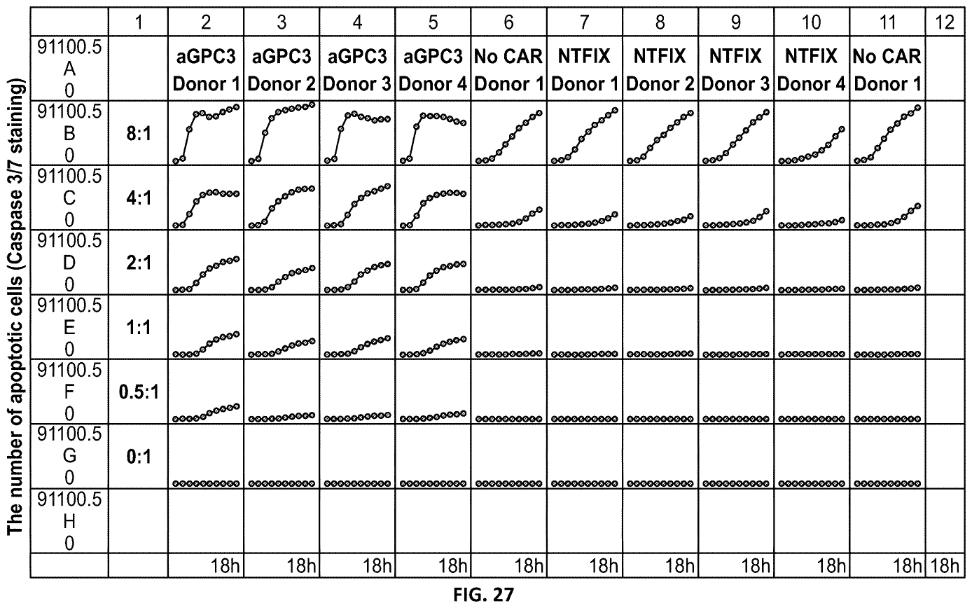

Biological activity of anti-GPC3 VHH CAR T cells was further evaluated for their effect on HepG2 apoptosis. 8,000, 16,000, or 32,000 HepG2 cells were seeded in each well of a plate. 80,000 T cells either transfected with anti-GPC3 VHH CARs comprising either of VHH29, VHH6, or VHH3 anti-GPC3 VH domains or a control CAR. No T cell or untransfected T cells (no CAR expressed) were also utilized as negative controls. T cells were added to each well with HepG2 cells. Cells were monitored over 19 hours (four images of each well taken every 3 hours) for apoptotic cells. Anti-GPC3 VHH CAR T cells killed HepG2 cells faster than the control CAR T cells (T cells expressing an anti-CD33 CAR or untransfected T cells). Over a longer incubation time, T cells expressing an anti-CD33 CAR (control CAR T cells) killed HepG2 cells (FIG. 26). The activity of anti-GPC3 VHH CAR T cells on HepG2 apoptosis was dose-dependent for all four PBMC donors tested (FIG. 27).

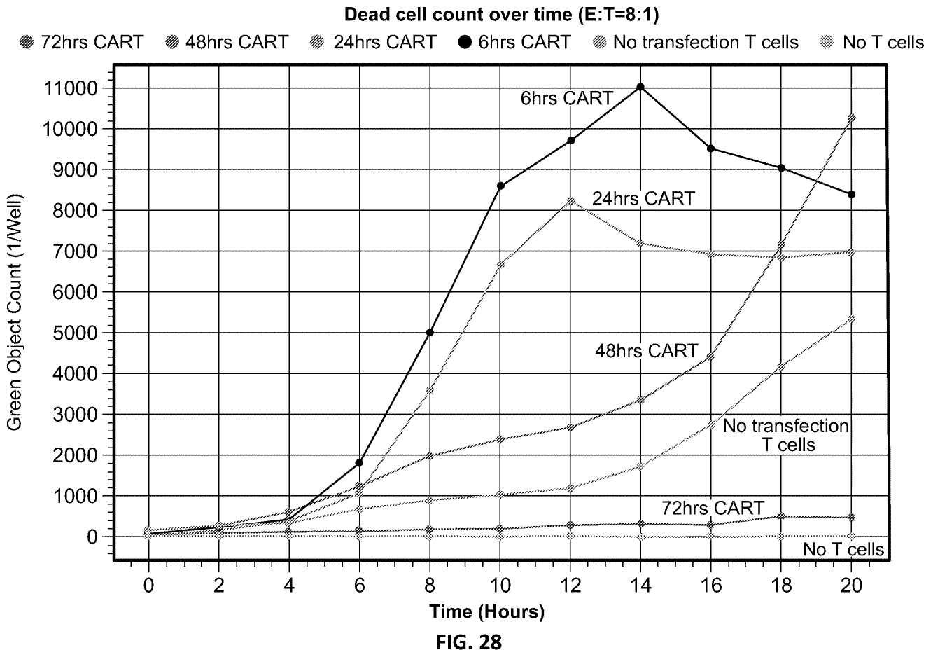

The efficiency of inducing apoptosis by anti-GPC3 VHH CAR T cells (comprising the anti-GPC3 VHH domain, VHH29) was also evaluated over time. Briefly, human T cells were purified from PBMC with negative selection and activated by CD3/CD28 for 3 days. The activated T cells were transfected with β-sitosterol formulated mRNA encoding the anti-GPC3 VHH CAR (1 μg LNP/1×10⁶ cells) and incubated for 0, 24, 48, or 72 hours to generate anti-GPC3 VHH CAR T cells. HepG2-red cells were made by transducing HepG2 cells with a non-perturbing, nuclear restricted red fluorescent label driven off an EF1α promoter (Incucyte) Nuclight Red Lentivirus EF1α, Puro). Stable cell lines were created under puromycin selection following product manual from Essenbioscience. 5,000 HepG2-red cells were seeded in each well of a plate and various numbers of the anti-GPC3 VHH CAR T cells were added along with 1× Capase 3/7 green. Cells were monitored over 22 hours (four images of each well taken every 2 hours) for apoptotic cells as indicated by green signal produced by Caspase 3/7 green. The killing efficiency of the anti-GPC3 VHH CAR T cells peaked about 6 hours post-transfection and decreased over time (FIG. 28).

### Example 3: In Vivo Pharmacokinetics of Anti-GPC3 VHH Containing CAR T Cells

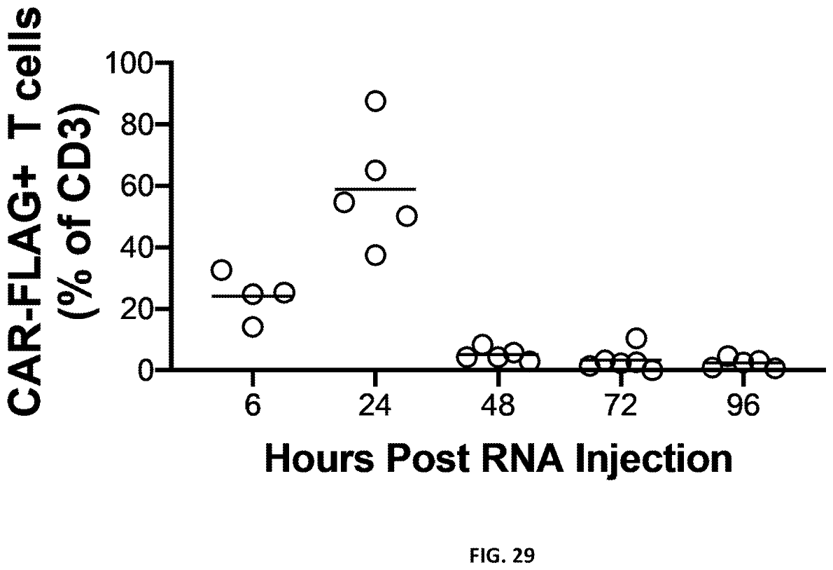

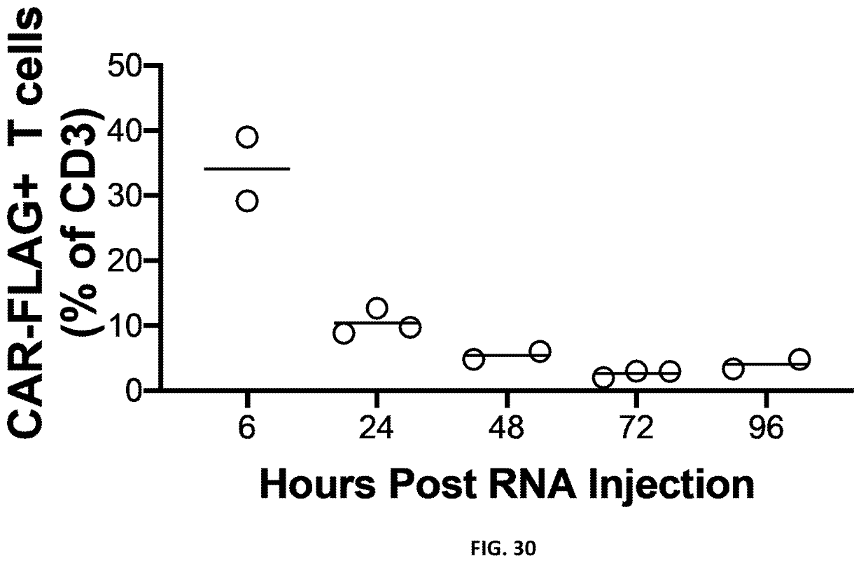

The present example demonstrates in vivo pharmacokinetics of anti-GPC3 VHH CAR T cells. Anti-GPC3 VHH CAR in this experiment included the anti-GPC3 VHH29 VHH domain. 30 male NOD scid gamma mice (NSG mice) between 6-10 weeks of age were utilized. 10×10⁶ PBMC were intraperitoneal injected into each NSG mouse. Seven days were subsequently allowed for engraftment. Following the engraftment period, formulated mRNA encoding FLAG-tagged anti-GPC3 VHH CAR was intravenously injected as a single dose at 0.5 mg/kg into the NSG mice. The mice were observed over the course of the experiment for symptoms of Graft-Versus-Host Disease (GVHD). Body weights were assessed twice per week over the course of the experiment. Blood and spleen samples were obtained at 6, 24, 48, 72, and 96 hours after mRNA injection encoding the anti-GPC3 VHH CAR (6 blood samples, 3 spleen samples per time point). CAR expression over time was evaluated using flow cytometry on blood and spleen samples for anti-FLAG (CAR expression) and markers, hCD45, CD3, CD4, and CD8. FIG. 29 shows that FLAG-tagged anti-GPC3 VHH containing CAR reached peak expression in blood 24 hours post injection. FIG. 30 demonstrates that shows that FLAG-tagged anti-GPC3 VHH CAR reached peak expression spleen 6 hours post injection.

What is claimed is:

1. A single-domain antibody that specifically binds Glypican 3 (GPC3) and comprises the following complementarity-determining regions (CDRs):
```
  (SEQ ID NO: 20)
(a) a CDR-1 comprising the amino acid sequence SYAMS,
  (SEQ ID NO: 21)
a CDR-2 comprising the amino acid sequence SISGGGTSTYYADSLEG,
and
(SEQ ID NO: 22)
a CDR-3 comprising the amino acid sequence DPRFGEPPFDY;
  (SEQ ID NO: 23)
(b) a CDR-1 comprising the amino acid sequence NYLMH,
  (SEQ IDN O: 24)
a CDR-2 comprising the amino acid sequence NINSDGSSTYYADSVKG,
and
  (SEQ ID NO: 25)
a CDR-3 comprising the amino acid sequence GAFDY;
(SEQ ID NO: 26)
(c) a CDR-1 comprising the amino acid sequence NYLMQ,
  (SEQ ID NO: 27)
a CDR-2 comprising the amino acid sequence NINSDGSSTDYADSVKG,
and
  (SEQ ID NO: 25)
a CDR-3 comprising the amino acid sequence GAFDY;
(SEQ ID NO: 20)
(d) a CDR-1 comprising the amino acid sequence SYAMS,
  (SEQ ID NO: 30)
a CDR-2 comprising the amino acid sequence SISGSGSSTYYADSLKG,
and
  (SEQ ID NO: 22)
a CDR-3 comprising the amino acid sequence DPRFGEPPFDY;
(SEQ ID NO: 20)
(e) a CDR-1 comprising the amino acid sequence SYAMS,
  (SEQ ID NO: 33)
a CDR-2 comprising the amino acid sequence SISGGGSSAYYADSLKG,
and
  (SEQ ID NO: 22)
a CDR-3 comprising the amino acid sequence DPRFGEPPFDY;
  (SEQ ID NO: 20)
(f) a CDR-1 comprising the amino acid sequence SYAMS,
  (SEQ ID NO: 36)
a CDR-2 comprising the amino acid sequence SISGGGSSTYYADSLEG,
and
  (SEQ ID NO: 22)
a CDR-3 comprising the amino acid sequence DPRFGEPPFDY;
  (SEQ ID NO: 20)
(g) a CDR-1 comprising the amino acid sequence SYAMS,
  (SEQ ID NO: 39)
a CDR-2 comprising the amino acid sequence SISGGGSSTYYADSLKG,
and
  (SEQ ID NO: 22)
a CDR-3 comprising the amino acid sequence DPRFGEPPFDY;
  (SEQ ID NO: 20)
(h) a CDR-1 comprising the amino acid sequence SYAMS,
  (SEQ ID NO: 39)
a CDR-2 comprising the amino acid sequence SISGGGSSTYYADSLKG,
and
  (SEQ ID NO: 46)
a CDR-3 comprising the amino acid sequence DPRFGEPPLDY;
  (SEQ ID NO: 47)
(i) a CDR-1 comprising the amino acid sequence SSAMS,
  (SEQ ID NO: 48)
a CDR-2comprising the amino acid sequence AISGSGGSTNYVDSVKG,
and
  (SEQ ID NO: 49)
a CDR-3 comprising the amino acid sequence ESMVRGGPFDY;
  (SEQ ID NO: 20)
(j) a CDR-1 comprising the amino acid sequence SYAMS,
  (SEQ ID NO: 39)
a CDR-2 comprising the amino acid sequence SISGGGSSTYYADSLKG,
and
  (SEQ ID NO: 52)
a CDR-3 comprising the amino acid sequence DPRFREPPFDY;
  (SEQ ID NO: 20)
(k) a CDR-1 comprising the amino acid sequence SYAMS,
  (SEQ ID NO: 57)
a CDR-2 comprising the amino acid sequence SISGGGGSTYYADSLKG,
and
  (SEQ ID NO: 58)
a CDR-3 comprising the amino acid sequence DPMFGERPFDY;
  (SEQ ID NO: 59)
(l) a CDR-1 comprising the amino acid sequence NYWMH,
  (SEQ ID NO: 60)
a CDR-2 comprising the amino acid sequence VSRINSDGSSTSYADPVKG,
and
  (SEQ ID NO: 61)
a CDR-3 comprising the amino acid sequence VALGFDF;
  (SEQ ID NO: 20)
(m) a CDR-1 comprising the amino acid sequence SYAMS,
  (SEQ ID NO: 63)
a CDR-2 comprising the amino acid sequence AISGSGGSTYYADSVKG,
and
  (SEQ ID NO: 64)
a CDR-3 comprising the amino acid sequence EALTGVFDY;
  (SEQ ID NO: 20)
(n) a CDR-1 comprising the amino acid sequence SYAMS,
  (SEQ ID NO: 69)
a CDR-2 comprising the amino acid sequence AIYSGGSTYYADSVKG,
and
  (SEQ ID NO: 70)
a CDR-3 comprising the amino acid sequence GDSSSSRFDY;
  (SEQ ID NO: 20)
(o) a CDR-1 comprising the amino acid sequence SYAMS,
  (SEQ ID NO: 39)
a CDR-2 comprising the amino acid sequence SISGGGSSTYYADSLKG,
and
  (SEQ ID NO: 73)
a CDR-3 comprising the amino acid sequence DPRLGEPPFDY;
  (SEQ ID NO: 20)
(p) a CDR-1 comprising the amino acid sequence SYAMS,
  (SEQ ID NO: 57)
a CDR-2 comprising the amino acid sequence SISGGGGSTYYADSLKG,
and
  (SEQ ID NO: 76)
a CDR-3 comprising the amino acid sequence DPRYGEPPFDY;
  (SEQ ID NO: 20)
(q) a CDR-1 comprising the amino acid sequence SYAMS,
  (SEQ ID NO: 36)
a CDR-2 comprising the amino acid sequence SISGGGSSTYYADSLEG,
and
  (SEQ ID NO: 97)
a CDR-3 comprising the amino acid sequence DPRFFEPPFDY;
  (SEQ ID NO: 98)
(r) a CDR-1 comprising the amino acid sequence SYGMH,
  (SEQ ID NO: 99)
a CDR-2 comprising the amino acid sequence VIWYDGNHKYYADSVKG,
and
  (SEQ ID NO: 100)
a CDR-3 comprising the amino acid sequence DKGGITGTTRNFQH;
or
  (SEQ ID NO: 101)
(s) a CDR-1 comprising the amino acid sequence SFAMS,
  (SEQ ID NO: 102)
a CDR-2 comprising the amino acid sequence AISGSGGRTHYADSVKG,
and
  (SEQ ID NO: 64)
a CDR-3 comprising the amino acid sequence EALTGVFDY.
```

2. The single-domain antibody of claim 1, wherein the single-domain antibody comprises a heavy chain comprising an amino acid sequence that is at least 85% identical to the amino acid sequence of any one of SEQ ID NOs: 1-19 or 80-96.

3. A multispecific antibody, comprising a GPC3-binding domain comprising the single-domain antibody of claim 1 or claim 2, and a second binding domain that specifically binds a second antigen.

4. The multispecific antibody of claim 3, wherein the multispecific antibody is a bispecific antibody.

5. The multispecific antibody of claim 3 or 4, wherein the second antigen is an antigen on a T cell or NK cell.

6. A chimeric antigen receptor (CAR), comprising a GPC3-binding domain comprising the single-domain antibody of claim 1 or 2, or the multispecific antibody of any one of claims 3 to 5, wherein the CAR further comprises a transmembrane domain, at least one costimulatory domain, and an intracellular signaling domain.

7. The CAR of claim 6, wherein the CAR comprises an amino acid sequence selected from any one of SEQ ID NOs: 158-160, but lacking both the signal sequence and Flag tag.

8. A T cell or NK cell expressing the CAR of claim 6 or 7.

9. A binding protein that specifically binds to Glypican 3 (GPC3) and comprises the following complementarity-determining regions (CDRs):
```
  (SEQ ID NO: 20)
(a) a CDR-1 comprising the amino acid sequence SYAMS,
  (SEQ ID NO: 21)
a CDR-2 comprising the amino acid sequence SISGGGTSTYYADSLEG,
and
  (SEQ ID NO: 22)
a CDR-3 comprising the amino acid sequence DPRFGEPPFDY;
  (SEQ ID NO: 23)
(b) a CDR-1 comprising the amino acid sequence NYLMH,
  (SEQ ID NO: 24)
a CDR-2 comprising the amino acid sequence NINSDGSSTYYADSVKG,
and
  (SEQ ID NO: 25)
a CDR-3 comprising the amino acid sequence GAFDY;
  (SEQ ID NO: 26)
(c) a CDR-1 comprising the amino acid sequence NYLMQ,
  (SEQ ID NO: 27)
a CDR-2 comprising the amino acid sequence NINSDGSSTDYADSVKG,
and
  (SEQ ID NO: 25)
a CDR-3 comprising the amino acid sequence GAFDY;
  (SEQ ID NO: 20)
(d) a CDR-1 comprising the amino acid sequence SYAMS,
  (SEQ ID NO: 30)
a CDR-2 comprising the amino acid sequence SISGSGSSTYYADSLKG,
and
  (SEQ ID NO: 22)
a CDR-3 comprising the amino acid sequence DPRFGEPPFDY;
  (SEQ ID NO: 20)
(e) a CDR-1 comprising the amino acid sequence SYAMS,
  (SEQ ID NO: 33)
a CDR-2 comprising the amino acid sequence SISGGGSSAYYADSLKG,
and
  (SEQ ID NO: 22)
a CDR-3 comprising the amino acid sequence DPRFGEPPFDY;
  (SEQ ID NO: 20)
(f) a CDR-1 comprising the amino acid sequence SYAMS,
  (SEQ ID NO: 36)
a CDR-2 comprising the amino acid sequence SISGGGSSTYYADSLEG,
  (SEQ ID NO: 22)
and a CDR-3 comprising the amino acid sequence DPRFGEPPFDY;
  (SEQ ID NO: 20)
(g) a CDR-1 comprising the amino acid sequence SYAMS,
  (SEQ ID NO: 39)
a CDR-2 comprising the amino acid sequence SISGGGSSTYYADSLKG,
and
  (SEQ ID NO: 22)
a CDR-3 comprising the amino acid sequence DPRFGEPPFDY;
  (SEQ ID NO: 20)
(h) a CDR-1 comprising the amino acid sequence SYAMS,
  (SEQ ID NO: 39)
a CDR-2 comprising the amino acid sequence SISGGGSSTYYADSLKG,
and
  (SEQ ID NO: 46)
a CDR-3 comprising the amino acid sequence DPRFGEPPLDY;
  (SEQ ID NO: 47)
(i) a CDR-1 comprising the amino acid sequence SSAMS,
  (SEQ ID NO: 48)
a CDR-2comprising the amino acid sequence AISGSGGSTNYVDSVKG,
and
  (SEQ ID NO: 49)
a CDR-3 comprising the amino acid sequence ESMVRGGPFDY;
  (SEQ ID NO: 20)
(j) a CDR-1 comprising the amino acid sequence SYAMS,
  (SEQ ID NO: 39)
a CDR-2 comprising the amino acid sequence SISGGGSSTYYADSLKG,
and
  (SEQ ID NO: 52)
a CDR-3 comprising the amino acid sequence DPRFREPPFDY;
  (SEQ ID NO: 20)
(k) a CDR-1 comprising the amino acid sequence SYAMS,
  (SEQ ID NO: 57)
a CDR-2 comprising the amino acid sequence SISGGGGSTYYADSLKG,
  (SEQ ID NO: 58)
and a CDR-3 comprising the amino acid sequence DPMFGERPFDY;
  (SEQ ID NO: 59)
(l) a CDR-1 comprising the amino acid sequence NYWMH,
(SEQ ID NO: 60)
a CDR-2 comprising the amino acid sequence VSRINSDGSSTSYADPVKG,
and
  (SEQ ID NO: 61)
a CDR-3 comprising the amino acid sequence VALGFDF;
  (SEQ ID NO: 20)
(m) a CDR-1 comprising the amino acid sequence SYAMS,
  (SEQ ID NO: 63)
a CDR-2 comprising the amino acid sequence AISGSGGSTYYADSVKG,
  (SEQ ID NO: 64)
a CDR-3 comprising the amino acid sequence EALTGVFDY;
and
  (SEQ ID NO: 20)
(n) a CDR-1 comprising the amino acid sequence SYAMS,
  (SEQ ID NO: 69)
a CDR-2 comprising the amino acid sequence AIYSGGSTYYADSVKG,
and
  (SEQ ID NO: 70)
a CDR-3 comprising the amino acid sequence GDSSSSRFDY;
  (SEQ ID NO: 20)
(o) a CDR-1 comprising the amino acid sequence SYAMS,
  (SEQ ID NO: 39)
a CDR-2 comprising the amino acid sequence SISGGGSSTYYADSLKG,
and
  (SEQ ID NO: 73)
a CDR-3 comprising the amino acid sequence DPRLGEPPFDY;
  (SEQ ID NO: 20)
(p) a CDR-1 comprising the amino acid sequence SYAMS,
  (SEQ ID NO: 57)
a CDR-2 comprising the amino acid sequence SISGGGGSTYYADSLKG,
and
  (SEQ ID NO: 76)
a CDR-3 comprising the amino acid sequence DPRYGEPPFDY;
  (SEQ ID NO: 20)
(q) a CDR-1 comprising the amino acid sequence SYAMS,
  (SEQ ID NO: 36)
a CDR-2 comprising the amino acid sequence SISGGGSSTYYADSLEG,
  (SEQ ID NO: 97)
a CDR-3 comprising the amino acid sequence DPRFFEPPFDY;
  (SEQ ID NO: 98)
(r) a CDR-1 comprising the amino acid sequence SYGMH,
  (SEQ ID NO: 99)
a CDR-2 comprising the amino acid sequence VIWYDGNHKYYADSVKG,
and
and (SEQ ID NO: 100)
a CDR-3 comprising the amino acid sequence DKGGITGTTRNFQH;
or
(SEQ ID NO: 101)
s) a CDR-1 comprising the amino acid sequence SFAMS,
  (SEQ ID NO: 102)
a CDR-2 comprising the amino acid sequence AISGSGGRTHYADSVKG,
and
(SEQ ID NO: 64)
a CDR-3 comprising the amino acid sequence EALTGVFDY.
```

10. The binding protein of claim 9, wherein the binding protein comprises a heavy chain comprising an amino acid sequence that is at least 85% identical to the amino acid sequence of any one of SEQ ID NOs: 1-19 or 80-96.

11. A nucleic acid encoding the single-domain antibody of claim 1 or 2; the multispecific antibody of any one of claims 3 to 5; the CAR of claim 6 or 7, or the binding protein of claim 9 or 10, optionally wherein the nucleic acid is an mRNA.

12. The nucleic acid of claim 11, wherein the nucleic acid comprises, in the 5′-to-3′ direction:
(a) a 5′ cap structure;
(b) a 5′ untranslated region (UTR);
(c) an open reading frame encoding a protein sequence comprising the single-domain antibody, the multispecific antibody, the CAR, or the binding protein, wherein the open reading frame consists of nucleosides selected from the group s consisting of (i) uridine or a modified uridine, (ii) cytidine or a modified cytidine, (iii) adenosine or a modified adenosine, and (iv) guanosine or a modified guanosine;
(d) a 3′ UTR; and
(e) a 3′ tailing sequence of linked nucleosides.

13. The nucleic acid of claim 12, wherein the open reading frame of nucleosides is selected from the group consisting of (i) a modified uridine, (ii) cytidine, (iii) adenosine, and (iv) guanosine.

14. The nucleic acid of claim 13, wherein the modified uridine is 1-methylpseudouridine.

15. The nucleic acid of any one of claims 12 to 14, wherein the 3′ tailing sequence of linked nucleosides is a poly-adenylate (polyA) tail or a polyA-G quartet.

16. The nucleic acid of any one of claims 12 to 15, wherein the 5′ cap structure is Cap0, Cap1, ARCA, inosine, 1-methyl-guanosine, 2′fluoroguanosine, 7-deaza-guanosine, 8-oxo-guanosine, 2-amino-guanosine, LNA-guanosine, or 2-azidoguanosine.

17. A pharmaceutical composition comprising the single-domain antibody of claim 1 or 2: the multispecific antibody of any one of claims 3 to 5; the CAR of claim 6 or 7, the binding protein of claim 9 or 10; or the nucleic acid of any one of claims 11 to 16.

18. The pharmaceutical composition of claim 17, wherein the pharmaceutical composition comprises a plurality of lipid nanoparticles encapsulating the nucleic acid, optionally wherein the plurality of lipid nanoparticles has a mean particle size of from 80 nm to 160 nm; and further optionally wherein the plurality of lipid nanoparticles has a polydispersity index (PDI) of from 0.02 to 0.2 and/or a lipid:nucleic acid ratio of from 10 to 20; and further optionally wherein the lipid nanoparticles comprise a neutral lipid, an ionizable lipid, a polyethyleneglycol (PEG) lipid, and/or a sterol.

19. A host cell comprising the single-domain antibody of claim 1 or 2; the multispecific antibody of any one of claims 3 to 5; or the CAR of claim 6 or 7, the binding protein of any one of claims 10 to 12; or the nucleic acid of any one of claims 11 to 16, optionally wherein the host cell is a CHO cell, a COS cell, a 293 cell, a NIH3T3 cell, or a HEK cell.

20. A method of treating cancer, comprising administering to a human subject in need thereof a therapeutically effective amount of the single-domain antibody of claim 1 or 2; the multispecific antibody of any one of claims 3 to 6; the T cell or NK cell of claim 8; the binding protein of any one of claims 10 to 12; the nucleic acid of any one of claims 11 to 16; or the pharmaceutical composition of claim 17 or 18, optionally wherein the cancer is Hepatocellular Cell Carcinoma (HCC), a squamous cell lung cancer, a head and neck squamous cell cancer, or a breast cancer.
